(19) United States
(12) Patent Application Publication
(10) Pub. No.: US 2022/0281841 A1
(43) Pub. Date: Sep. 8, 2022

(54) ETHYNYLHETEROCYCLES AS RHO-ASSOCIATED COILED-COIL KINASE (ROCK) INHIBITORS

(71) Applicant: Angion Biomedica Corp., Uniondale, NY (US)

(72) Inventors: An-Hu Li, Commack, NY (US); Satish Kumar Sakilam, Harrison, NJ (US); Satishkumar Gadhiya, Jamaica, NY (US); Dong Sung Lim, Fair Lawn, NJ (US); Yao Zong, Port Washington, NY (US); Shashikanth Ponnala, Secaucus, NJ (US); Ying Zhang, Glen Cove, NY (US); Dawoon Jung, Tenafly, NJ (US); Lambertus J.W.M. Oehlen, Westbury, NY (US)

(21) Appl. No.: 17/629,061

(22) PCT Filed: Jul. 21, 2020
(86) PCT No.: PCT/US2020/042907

§ 371 Date: Jan. 21, 2022

Related U.S. Application Data
(60) Provisional application No. 62/877,007, filed on Jul. 22, 2019.

(51) Int. Cl. C07D 401/14; C07D 403/14; C07D 471/04; C07D 487/04; C07D 417/14; C07F 9/6558

(52) U.S. Cl. CPC C07D 401/14; C07D 403/14; C07D 471/04; C07D 487/04; C07D 417/14; C07F 9/65583

(57) ABSTRACT

The present invention provides compounds having formula (I): and pharmaceutically acceptable salts thereof, wherein Cy1, Cy2, Cy3, R, R¹, R², and R³ are as described generally and in classes and subclasses herein, and additionally provides pharmaceutical compositions thereof, and methods for the use thereof for the treatment of any of a number of conditions or diseases in which inhibiting ROCK1, ROCK2, or ROCK1/2 has a therapeutically useful role.

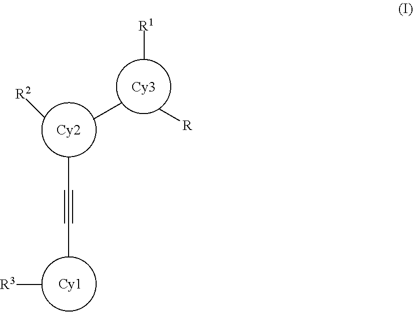

### CROSS REFERENCE TO RELATED APPLICATIONS

This application claims the benefit of U.S. Provisional Application No. 62/877,007, filed Jul. 22, 2019, which is herein incorporated by reference in its entirety.

### BACKGROUND

The Rho-associated coiled-coil kinase (ROCK) family members, consisting of Rho-associated kinase 1 (ROCK1) and Rho-associated kinase 2 (ROCK2), are serine-threonine kinases that are activated by Rho GTPases. Both ROCK1 and ROCK2 are involved in a wide range of cellular processes including actin cytoskeleton organization, smooth muscle cell contraction, adhesion, migrations, proliferation, apoptosis and fibrosis (Loirand, G. Rho Kinases in Health and Disease: From Basic Science to Translational Research. Pharmacol. Rev. 2015, 67(4), 1074-95). The ROCK signaling cascade, modulated by fibrogenic growth factors including TGFβ1, angiotensin I, PDGF and endothelin-I, participates in epithelial to mesenchymal transition (Hu, Y. B., Li, X., Liang, G. N., Deng, Z. H., Jiang, H. Y., Zhou, J. H. Roles of Rho/Rock signaling pathway in silica-induced epithelial-mesenchymal transition in human bronchial epithelial cells. Biomed. Environ. Sci. 2013, 26(7), 571-6) Evidence for the potential role of this pathway in renal fibrosis comes from early studies that used pharmacologic inhibition of ROCK with Y-27632 or fasudil, which are selective but ROCK1/2 dual inhibitors, i.e., they inhibit both ROCK 1 and ROCK2 but not other kinases. Use of ROCK1/2 dual inhibitors prevented tubulointerstitial fibrosis in obstructive renal disease, mitigated nephropathy in subtotally nephrectomized, spontaneously hypertensive rats and attenuated glomerulosclerosis in Dahl salt-sensitive rats (Komers, R., Oyama, T. T., Beard, D. R., Tikellis, C., Xu, B., Lotspeich, D. F., Anderson, S. Rho kinase inhibition protects kidneys from diabetic nephropathy without reducing blood pressure. Kidney Int. 2011, 79(4), 432-42. Nagatoya, K., Moriyama, T., Kawada, N., Takeji, M., Oseto, S., Murozono, T., Ando, A., Imai, E., Hori, M. Y-27632 prevents tubulointerstitial fibrosis in mouse kidneys with unilateral ureteral obstruction. Kidney Int. 2002, 61(5), 1684-95. Baba, I., Egi, Y., Utsumi, H., Kakimoto, T., Suzuki, K. Inhibitory effects of fasudil on renal interstitial fibrosis induced by unilateral ureteral obstruction. Mol. Med. Rep. 2015, 12(6), 8010-20. Kolavennu, V., Zeng, L., Peng, H., Wang, Y., Danesh, F. R. Targeting of RhoA/ROCK signaling ameliorates progression of diabetic nephropathy independent of glucose control. Diabetes 2008, 57(3), 714-23).

Regardless of the fact that the two ROCK isoforms are similar, a growing body of evidence from more recent studies with ROCK isoform transgenic animals and ROCK isoform-selective pharmacological inhibitors support the notion that ROCK1 and ROCK2 each have unique functions. Shi et al. (Shi, J., Wu, X., Surma, M., Vemula, S., Zhang, L., Yang, Y., Kapur, R., Wei, L. Distinct roles for ROCK1 and ROCK2 in the regulation of cell detachment. Cell Death Dis. 2013, 4(2), e483. doi: 10.1038/cddis.2013.10), using both genetic and pharmacological approaches, demonstrated that ROCK1, via regulation of MLC2 phosphorylation, is involved in destabilizing the actin cytoskeleton in fibroblasts (i.e., ROCK1 signaling is antifibrotic), whereas ROCK2, via regulation of cofilin phosphorylation, is required for stabilizing fibroblast actin cytoskeleton (i.e., ROCK2 signaling is profibrotic). Consistent with this finding, genome-wide expression profiling of fibroblasts treated with the ROCK2 selective inhibitor, KD025 (SLx-2119), revealed decreased expression of several profibrotic mRNA including that of CTGF (Boerma, M., Fu, Q., Wang, J., Loose, D. S., Bartolozzi, A., Ellis, J. L., McGonigle, S., Paradise, E., Sweetnam, P., Fink, L. M., Vozenin-Brotons, M. C., Hauer-Jensen, M. Comparative gene expression profiling in three primary human cell lines after treatment with a novel inhibitor of Rho kinase or atorvastatin. Blood Coagul. Fibrinolysis 2008, 19(7), 709-718). In a separate study (Zanin-Zhorov, A., Weiss, J. M., Nyuydzefe, M. S., Chen, W., Scher, J. U., Mo, R., Depoil, D., Rao, N., Liu, B., Wei, J., Lucas, S., Koslow, M., Roche, M., Schueller, O., Weiss, S., Poyurovsky, M. V., Tonra, J., Hippen, K. L., Dustin, M. L., Blazar, B. R., Liu, C. J., Waksal, S. D., Selective oral ROCK2 inhibitor down-regulates IL-21 and IL-17 secretion in human T cells via STATS-dependent mechanism. Proc. Natl. Acad. Sci. USA. 2014, 111(47), 16814-9), KD025 administration decreased expression of pro-inflammatory, fibrosis-linked cytokines and mitigated murine autoimmune disease. Further evidence appearing to support a driving role for ROCK2 in fibrosis, and pertinent to renal disease, is the finding that ROCK1 knockout mice were not protected against ureteral obstruction-related renal fibrosis at either the early (day 5) or late (day 10) disease stage as determined by histology and expression of both mRNA and protein levels of aSMA, collagen types I and III and fibronectin (Fu, P., Liu, F., Su, S., Wang, W., Huang, X. R., Entman, M. L., Schwartz, R. J., Wei, L., Lan, H. Y. Signaling mechanism of renal fibrosis in unilateral ureteral obstructive kidney disease in ROCK1 knockout mice. J. Am. Soc. Nephrol. 2006, 17(11), 3105-14). Although Baba et al. (Baba, I., Egi, Y., Suzuki, K. Partial deletion of the ROCK2 protein fails to reduce renal fibrosis in a unilateral ureteral obstruction model in mice. Mol. Med. Rep. 2016, 13(1), 231-6), demonstrated that half-deletion of ROCK2 also did not prevent UUO-induced renal fibrosis, the discrepancy regarding these data and the one published by Shi et al. (Shi, J., Wu, X., Surma, M., Vemula, S., Zhang, L., Yang, Y., Kapur, R., Wei, L. Distinct roles for ROCK1 and ROCK2 in the regulation of cell detachment. Cell Death Dis. 2013; 4 (2), e483. doi: 10.1038/cddis.2013.10), could be attributed to different strain and incomplete genetic ablation (homozygous vs heterozygous) of the ROCK2 isozyme.

Efficacy aside, need for use of an isoform-selective approach derives from the perspective of drug safety. Since ROCK plays a central role in the organization of the actin cytoskeleton, it might be anticipated that (unnecessary) inhibition of both its isoforms in a chronic setting such as chronic kidney disease (CKD) could cause severe adverse events. Indeed, systemic inhibition of ROCK does bear the risk of significant hypotension and such a strategy needs to be evaluated in terms of risk to benefit ratio (www.hsric.nihr.ac.uk/topicsinetarsudil-for-open-angle-glaucoma-or-ocular-hypertension/; //en.wikipedia.org/wiki/Fasudil). For diseases such as glaucoma, which is amenable to local treatment, ROCK isoform selectivity is not mandated and ROCK1/2 dual inhibitors such as netarsudil are dosed into the eye via the intravitreous or intracameral routes (www.hsric.nihr.ac.uk/topicsinetarsudil-for-open-angle-glaucoma-or-ocular-hypertension/). Furthermore, drug load in glaucoma is small. With hyperacute indications such as cerebral vasospasm, dosing with fasudil (en.wikipedia.orgiwiki/Fasudil) might not pose a significant risk, albeit its use remains to be approved in the United States. Finally, in contrast to use of ROCK1/2 dual inhibitors, the ROCK2-selective inhibitor KD025 has been found to have no hemodynamic or other side effects over 12-16 weeks of dosing in healthy volunteers and patients (clinicaltrials.gov/ct2/results?term=KD025&Search=Search).

All citations in the present application are incorporated herein by reference in their entireties. The citation of any reference herein should not be construed as an admission that such reference is available as “Prior Art” to the instant application.

### SUMMARY OF CERTAIN ASPECTS OF THE INVENTION

As discussed above; there remains a need for the development of novel therapeutics that are capable of inhibiting ROCK1, ROCK2, or ROCK1/2 activities. In certain embodiments, the present disclosure is directed toward the identification of small organic molecules that exhibit ROCK1, ROCK2, or ROCK1/2 (dual ROCK1 and ROCK2) inhibitory activities and are thus useful in the treatment or prevention of conditions or diseases in which inhibition of ROCK1, ROCK2, or ROCK1/2 is desirable.

In general, provided compounds have the structure shown in Formula I:

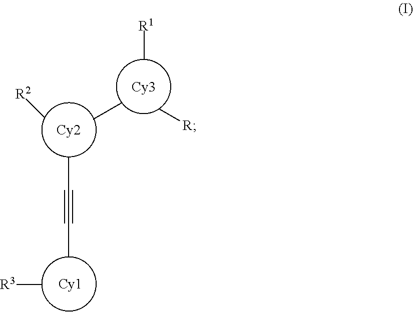

or a pharmaceutically acceptable salt thereof, wherein,

Cy1; Cy2, and Cy3 each independently represents an aryl, heteroaryl, or heterocyclic, each of which is optionally fused with a 3-8 membered cycloalkyl, a 3-8 membered heterocycloalkyl, a 6-membered aryl, or a 5-6 membered heteroaryl;

R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONR^{l}R^{m}, —NR^{k}CONR^{l}R^{m}, —OCONRⁿR^{o}, or —NR^{p}CO₂R^{q};

R is an heterocyclic, aromatic, or heteroaromatic; optionally substituted with one or more independent hydrogen, deuterium, halo, —CN, —NO₂, aliphatic, alicyclic, heteroaliphatic, heterocyclic, aromatic, heteroaromatic, —OR′, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{k}CO₂R^{p};

R^{a}, R^{b}; R^{c}, R^{d}, R^{e}, R^{f}, R^{g}, R^{h}, Rⁱ, R^{j}, R^{k}, R^{l}, R^{m}, Rⁿ, R^{o}, R^{p} and R^{q}, for each occurrence, is independently selected from hydrogen, deuterium, halo, —CN, —NO₂, an optionally substituted aliphatic, alicyclic, heteroaliphatic, heterocyclic, aromatic, or heteroaromatic; wherein each optional substituent is independently selected from one or more hydrogen, deuterium, halo, —CN, —NO₂, aliphatic, alicyclic, heteroaliphatic, heterocyclic, aromatic, heteroaromatic, —OR^{aa}, —NR^{bb}R^{cc}, —S(═O)_{w}R^{dd}, —S(═O)_{w}NR^{ee}R^{ff}, —C(═O)R^{gg}, —CO₂R^{hh}, —CONRⁱⁱR^{jj}, —NR^{kk}CONR^{ll}R^{mm}, —OCONRⁿⁿR^{oo}, or —NR^{ll}CO₂R^{pp}; or R^{b} and R^{c}, R^{e} and R^{f}, Rⁱ and R^{j}, R^{l} and R^{m}, or Rⁿ and R^{o}; when attached to the same nitrogen, may optionally form a heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, aliphatic, alicyclic, heteroaliphatic, heterocyclic, aromatic, or heteroaromatic;

R^{aa}, R^{bb}, R^{cc}, R^{dd}, R^{ee}, R^{ff}, R^{gg}, R^{hh}, Rⁱⁱ, R^{jj}, R^{kk}, R^{ll}, R^{mm}, Rⁿⁿ, R^{oo}, and R^{pp}, for each occurrence, is independently selected from hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic;

w is 0, 1, or 2.

In one embodiment, the compound has the structure shown in Formula Ia:

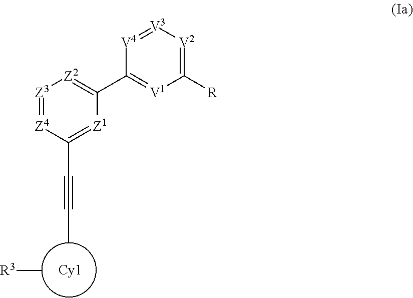

wherein V¹, V², V³ and V⁴ are each independently N or C—R¹, wherein two R¹ groups on adjacent carbon atoms together with the carbons to which they are attached may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂;

Z¹, Z², Z³ and Z⁴ are each independently N or C—R², wherein two R² groups on adjacent carbon atoms together with the carbons to which they are attached may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O), CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂, and wherein all other substituents are as defined in Formula I.

In one embodiment, the compound has the structure shown in Formula Ib:

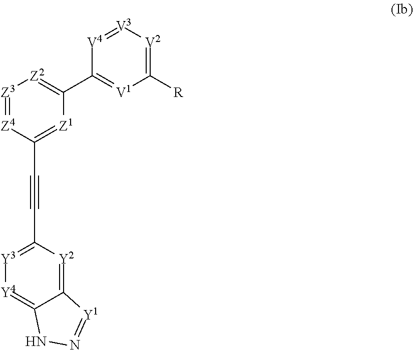

wherein Y¹, Y², Y³ and Y⁴ is each independently N or C—R³, wherein two R³ groups on adjacent carbon atoms together with the carbons they are attached to may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂;

and definitions of V¹, V², V³, V⁴, Z¹, Z², Z³, and Z⁴ are the same with those in Formula Ia and R and R³ have the same meaning with those in Formula I.

In one embodiment, the compound has the structure shown in Formula Ic or Id:

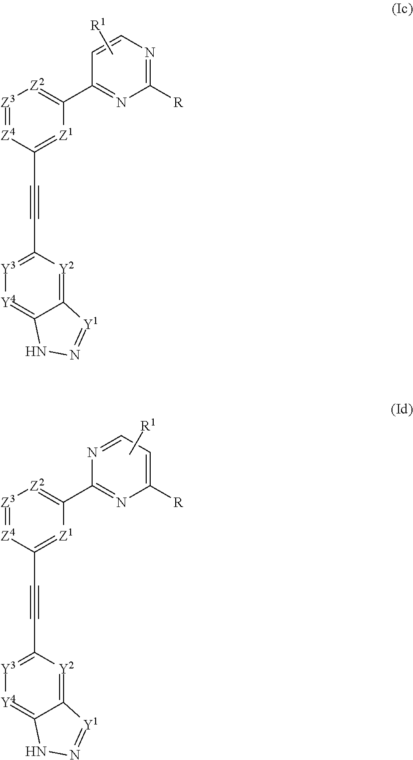

wherein the definitions of Z¹, Z², Z³, and Z⁴ are the same with those in Formula Ia, Y³, and Y⁴ are the same with those in Formula Ib, and R and IV have the same meaning with those in Formula I; and wherein all other substituents are as defined in Formula I.

In some embodiments, the present disclosure provides a compound of Formula II:

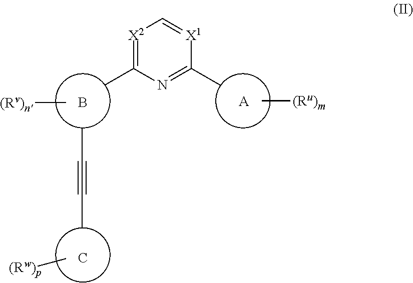

or a pharmaceutically acceptable salt thereof, wherein each of X¹, X², Ring A, Ring B, Ring C, R^{u}, R^{v}, R^{w}, m, n, and p is defined infra.

In another aspect, the present disclosure provides compositions including pharmaceutical compositions of any of the compounds disclosed herein. Pharmaceutical compositions in one embodiment may comprise one or more compounds of the invention, and a carrier, diluent or excipient.

In some embodiments, the present disclosure provides pharmaceutically acceptable compositions comprising a compound of Formula II, or a pharmaceutically acceptable salt thereof, and a pharmaceutically acceptable carrier, diluent, or excipient. Such pharmaceutically acceptable compositions are described infra.

In another aspect, the present disclosure provides methods for the use of any of the compounds disclosed herein for inhibiting ROCK1, ROCK2, or ROCK1/2 activities in a patient or in a biological sample. In one embodiment the compounds of the invention have antifibrotic activities. The compounds and pharmaceutical compositions of the invention have properties of inhibiting ROCK1, ROCK2, or ROCK1/2 activities and are useful in the treatment of any disease, disorder or condition in which prophylactic or therapeutic administration of ROCK1, ROCK2, or ROCK1/2 inhibitors would be useful.

In some embodiments, the present disclosure provides a method of inhibiting ROCK1 and/or ROCK2, the method comprising contacting a biological sample with a compound of Formula II, or a pharmaceutically acceptable salt thereof.

In another aspect, the present disclosure provides methods for the use of any of the compounds disclosed herein for treating or lessening the severity of a disease or condition associated with ROCK1, ROCK2, or ROCK1/2 activity. In certain embodiments, the method is for treating or lessening the severity of a disease or condition selected from fibrotic liver disease, hepatic ischemia-reperfusion injury, cerebral infarction, ischemic heart disease, renal disease or lung (pulmonary) fibrosis. In certain embodiments, the method is for treating or lessening the severity of a disease or condition selected from liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemia/reperfusion injury; stroke; cerebrovascular disease; myocardial ischemia; atherosclerosis; renal failure; renal fibrosis or idiopathic pulmonary fibrosis. In certain exemplary embodiments, the method is for the treatment of wounds for acceleration of healing; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; acute or chronic heart failure, renal failure secondary to chronic diabetes and/or hypertension; amyotrophic lateral sclerosis, muscular dystrophy, glaucoma, corneal scarring, macular degeneration, diabetic retinopathy and/or diabetes mellitus.

In some embodiments, the present disclosure provides a method of treating a disease or disorder associated with or mediated by ROCK1 and/or ROCK2, the method comprising administering to a patient in need thereof a compound of Formula II, or a pharmaceutically acceptable salt thereof. Diseases and/or disorders associated with or mediated by ROCK1 and/or ROCK2 are described in greater detail, infra.

These and other aspects of the present disclosure will be apparent from the brief description of the drawing and detailed description of certain aspects of the invention, below.

### Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic”, as used herein with reference to Formula I and subgenera thereof, includes both saturated and unsaturated, straight chain (i.e., unbranched) or branched aliphatic hydrocarbons, which are optionally substituted with one or more functional groups. As will be appreciated by one of ordinary skill in the art, “aliphatic” as used with reference to Formula I and subgenera thereof, is intended herein to include, but is not limited to, alkyl, alkenyl, or alkynyl moieties. Thus, as used herein with reference to Formula I and subgenera thereof, the term “alkyl” includes straight and branched alkyl groups. An analogous convention applies to other generic terms such as “alkenyl”, “alkynyl” and the like. Furthermore, as used herein with reference to Formula I and subgenera thereof, the terms “alkyl”, “alkenyl”, “alkynyl” and the like encompass both substituted and unsubstituted groups. In certain embodiments, as used herein with reference to Formula I and subgenera thereof, “lower alkyl” is used to indicate those alkyl groups (substituted, unsubstituted, branched or unbranched) having 1-6 carbon atoms. “Lower alkenyl” and “lower alkynyl” respectively include corresponding 1-6 carbon moieties.

The term “aliphatic” or “aliphatic group”, as used herein with reference to Formula II and subgenera thereof, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “unsaturated”, as used herein with reference to Formula II and subgenera thereof, means that a moiety has one or more units of unsaturation.

As used herein, the term “partially unsaturated”, as used herein with reference to Formula II and subgenera thereof, refers to a ring moiety that includes at least one double or triple bond. The term “partially unsaturated”, as used herein with reference to Formula II and subgenera thereof, is intended to encompass rings having multiple sites of unsaturation, but is not intended to include aryl or heteroaryl moieties, as herein defined.

The term “lower alkyl”, as used herein with reference to Formula II and subgenera thereof, refers to a C₁₋₄ straight or branched alkyl group. Exemplary lower alkyl groups are methyl, ethyl, propyl, isopropyl, butyl, isobutyl, and tert-butyl.

In certain embodiments of Formula I and subgenera thereof, the alkyl, alkenyl and alkynyl groups contain 1-20; 2-20; 3-20; 4-20; 5-20; 6-20; 7-20 or 8-20 aliphatic carbon atoms. In certain other embodiments of Formula I and subgenera thereof, the alkyl, alkenyl, and alkynyl groups contain 1-10; 2-10; 3-10; 4-10; 5-10; 6-10; 7-10 or 8-10 aliphatic carbon atoms. In yet other embodiments of Formula I and subgenera thereof, the alkyl, alkenyl, and alkynyl groups contain 1-8; 2-8; 3-8; 4-8; 5-8; 6-20 or 7-8 aliphatic carbon atoms. In still other embodiments of Formula I and subgenera thereof, the alkyl, alkenyl, and alkynyl groups contain 1-6; 2-6; 3-6; 4-6 or 5-6 aliphatic carbon atoms. In yet other embodiments of Formula I and subgenera thereof, the alkyl, alkenyl, and alkynyl groups contain 1-4; 2-4 or 3-4 carbon atoms. Illustrative aliphatic groups used with reference to Formula I and subgenera thereof thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups used with reference to Formula I and subgenera thereof include, but are not limited to, for example, ethenyl, propenyl, butenyl, 1-methyl-2-buten-1-yl, and the like. Representative alkynyl groups used with reference to Formula I and subgenera thereof include, but are not limited to, ethynyl, 2-propynyl (propargyl), 1-propynyl and the like.

The term “alicyclic”, as used herein with reference to Formula I and subgenera thereof, refers to compounds which combine the properties of aliphatic and cyclic compounds and include but are not limited to monocyclic, or polycyclic aliphatic hydrocarbons and bridged cycloalkyl compounds, which are optionally substituted with one or more functional groups. As will be appreciated by one of ordinary skill in the art, the term “alicyclic” used with reference to Formula I and subgenera thereof is intended herein to include, but is not limited to, cycloalkyl, cycloalkenyl, and cycloalkynyl moieties, which are optionally substituted with one or more functional groups. Illustrative alicyclic groups used with reference to Formula I and subgenera thereof thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy”, “alkoxyl”, “alkyloxy”, or “alkyloxyl”, as used herein with reference to Formula I and subgenera thereof, refers to a saturated (i.e., O-alkyl) or unsaturated (i.e., O-alkenyl and O-alkynyl) group attached to the parent molecular moiety through an oxygen atom. In certain embodiments of Formula I and subgenera thereof, the alkoxy group contains 1-20; 2-20; 3-20; 4-20; 5-20; 6-20; 7-20 or 8-20 aliphatic carbon atoms. In certain other embodiments of Formula I and subgenera thereof, the alkoxy group contains 1-10; 2-10; 3-10; 4-10; 5-10; 6-10; 7-10 or 8-10 aliphatic carbon atoms. In yet other embodiments of Formula I and subgenera thereof, the O-alkyl, O-alkenyl, and O-alkynyl groups contain 1-8; 2-8; 3-8; 4-8; 5-8; 6-20 or 7-8 aliphatic carbon atoms. In still other embodiments of Formula I and subgenera thereof, the alkoxy group contains 1-6; 2-6; 3-6; 4-6 or 5-6 aliphatic carbon atoms. In yet other embodiments of Formula I and subgenera thereof, the alkoxy group contains 1-4; 2-4 or 3-4 aliphatic carbon atoms. Examples of alkoxy, include but are not limited to, methoxy, ethoxy, propoxy, isopropoxy, n-butoxy, butoxy, sec-butoxy, tert-butoxy, neopentoxy, n-hexoxy and the like.

The term “thioalkyl” as used herein with reference to Formula I and subgenera thereof refers to a saturated (i.e., S-alkyl) or unsaturated (i.e., S-alkenyl and S-alkynyl) group attached to the parent molecular moiety through a sulfur atom. In certain embodiments of Formula I and subgenera thereof, the thioalkyl group contains 1-20 aliphatic carbon atoms. In certain other embodiments of Formula I and subgenera thereof, the thioalkyl group contains 1-10 aliphatic carbon atoms. In yet other embodiments of Formula I and subgenera thereof, the S-alkyl, S-alkenyl, and S-alkynyl groups contain 1-8 aliphatic carbon atoms. In still other embodiments of Formula I and subgenera thereof, the thioalkyl group contains 1-6 aliphatic carbon atoms. In yet other embodiments of Formula I and subgenera thereof, the thioalkyl group contains 1˜4 aliphatic carbon atoms. Examples of thioalkyl include, but are not limited to, methylthio, ethylthio, propylthio, isopropylthio, n-butylthio, and the like.

The term “alkylamino”, as used herein with reference to Formula I and subgenera thereof, refers to a group having the structure —NHR′ wherein R′ is aliphatic or alicyclic, as defined herein with reference to Formula I and subgenera thereof. The term “aminoalkyl”, as used herein with reference to Formula I and subgenera thereof, refers to a group having the structure H₂NR′—, wherein R′ is aliphatic or alicyclic, as defined herein with reference to Formula I and subgenera thereof. In certain embodiments, the aliphatic or alicyclic group of Formula I and subgenera thereof contains 1-20 aliphatic carbon atoms. In certain other embodiments of Formula I and subgenera thereof, the aliphatic or alicyclic group contains 1-10 aliphatic carbon atoms. In still other embodiments of Formula I and subgenera thereof, the aliphatic or alicyclic group contains 1-6 aliphatic carbon atoms. In yet other embodiments of Formula I and subgenera thereof, the aliphatic or alicyclic group contains 1-4 aliphatic carbon atoms. In yet other embodiments of Formula I and subgenera thereof, R′ is an alkyl, alkenyl, or alkynyl group containing 1-8 aliphatic carbon atoms. Examples of alkylamino include, but are not limited to, methylamino (e.g., —NHCH₃), ethylamino (e.g., —NHCH₂CH₃), iso-propylamino (e.g., —NHCH(CH₃)₂) and the like.

Some examples of substituents of the above-described aliphatic (and other) moieties of compounds of Formula I and subgenera thereof include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein for Formula I and subgenera thereof may be substituted or unsubstituted, branched or unbranched, saturated or unsaturated, and wherein any of the aryl or heteroaryl substituents described above and herein for Formula I and subgenera thereof may be substituted or unsubstituted. Additional examples of generally applicable substituents are illustrated by the specific embodiments shown in the Examples that are described herein for Formula I and subgenera thereof.

In general, the term “aromatic” or “aromatic moiety”, as used herein with reference to Formula I and subgenera thereof, refers to a stable mono- or polycyclic, unsaturated moiety having preferably 3-14 carbon atoms, each of which may be substituted or unsubstituted. In certain embodiments of Formula I and subgenera thereof, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic” for Formula I and subgenera thereof, and is encompassed by the term “alicyclic” for Formula I and subgenera thereof.

In general, the term “heteroaromatic” or “heteroaromatic moiety”, as used herein with reference to Formula I and subgenera thereof, refers to a stable mono- or polycyclic, unsaturated moiety having preferably 3-14 carbon atoms, each of which may be substituted or unsubstituted; and comprising at least one heteroatom selected from O, S and N within the ring (i.e., in place of a ring carbon atom). In certain embodiments of Formula I and subgenera thereof, the term “heteroaromatic moiety” refers to a planar ring comprising at least one heteroatom, having p-orbitals perpendicular to the plane of the ring at each ring atom, and satisfying the Hückel's rule where the number of pi electrons in the ring is (4n+2) wherein n is an integer.

It will also be appreciated that aromatic and heteroaromatic moieties, as defined herein with reference to Formula I and subgenera thereof, may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic”, as used herein with reference to Formula I and subgenera thereof, are interchangeable. Substituents for such groups include, but are not limited to, any of the previously mentioned substituents, i.e., the substituents recited for aliphatic moieties, or for other moieties as disclosed herein for Formula I and subgenera thereof, resulting in the formation of a stable compound.

The term “aryl”, as used herein with reference to Formula I and subgenera thereof, does not differ significantly from the common meaning of the term in the art, and refers to an unsaturated cyclic moiety comprising at least one aromatic ring. In certain embodiments of Formula I and subgenera thereof, “aryl” refers to a mono- or bicyclic carbocyclic ring system having one or two aromatic rings including, but not limited to phenyl, naphthyl, tetrahydronaphthyl, indanyl, indenyl and the like.

The term “aryl”, as used herein with reference to Formula II and subgenera thereof, refers to monocyclic and bicyclic ring systems having a total of five to fourteen ring members, wherein at least one ring in the system is aromatic and wherein each ring in the system contains three to seven ring members. The term “aryl” with reference to Formula II and subgenera thereof may be used interchangeably with the term “aryl ring”. In certain embodiments of Formula II and subgenera thereof, “aryl” refers to an aromatic ring system which includes, but not limited to, phenyl, biphenyl, naphthyl, anthracyl and the like, which may bear one or more substituents. Also included within the scope of the term “aryl” used in reference to Formula II and subgenera thereof is a group in which an aromatic ring is fused to one or more non-aromatic rings, such as indanyl, phthalimidyl, naphthimidyl, phenanthridinyl, or tetrahydronaphthyl, and the like.

The term “heteroaryl”, as used herein with reference to Formula I and subgenera thereof, does not differ significantly from the common meaning of the term in the art, and refers to a cyclic aromatic radical having from five to twelve ring atoms of which one ring atom is selected from S, O and N; zero, one, two, three, four, or five ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, isoquinolinyl, and the like.

The term “heteroaryl” as used herein with reference to Formula II and subgenera thereof refers to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” as used herein with reference to Formula II and subgenera thereof refers to nitrogen, oxygen, or sulfur, and includes any oxidized form of nitrogen or sulfur, and any quaternized form of a basic nitrogen. Heteroaryl groups on compounds of Formula II or subgenera thereof include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein with reference to Formula II and subgenera thereof, also include groups in which a heteroaromatic ring is fused to one or more aryl, cycloaliphatic, or heterocyclyl rings, where the radical or point of attachment is on the heteroaromatic ring. Nonlimiting examples of heteroaryl rings on compounds of Formula II and subgenera thereof include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group for use in compounds of Formula II and subgenera thereof may be mono- or bicyclic. The term “heteroaryl” used in reference to compounds of Formula II and subgenera thereof may be used interchangeably with the terms “heteroaryl ring”, “heteroaryl group”, or “heteroaromatic”, any of which terms include rings that are optionally substituted.

It will be appreciated that aryl and heteroaryl groups (including bicyclic aryl groups) as defined herein for Formula I and subgenera thereof can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein for Formula I and subgenera thereof may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein for Formula I and subgenera thereof may be substituted or unsubstituted. Additionally, it will be appreciated, that any two adjacent groups as described herein for Formula I and subgenera thereof taken together may represent a 4, 5, 6, or 7-membered substituted or unsubstituted alicyclic or heterocyclic moiety. Additional examples of generally applicable substituents are illustrated by the specific embodiments shown in the Examples that are described herein for Formula I and subgenera thereof

The term “cycloalkyl”, as used herein with reference to Formula I and subgenera thereof, refers specifically to groups having three to twelve, preferably three to ten carbon atoms. Suitable cycloalkyls for Formula I and subgenera thereof include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein for Formula I and subgenera thereof may be substituted or unsubstituted, branched or unbranched, saturated or unsaturated, and wherein any of the aromatic, heteroaromatic, aryl or heteroaryl substituents described above and herein for Formula I and subgenera thereof may be substituted or unsubstituted. Additional examples of generally applicable substituents are illustrated by the specific embodiments shown in the Examples that are described herein for Formula I and subgenera thereof.

The term “heteroaliphatic”, as used herein with reference to Formula I and subgenera thereof, refers to aliphatic moieties in which one or more carbon atoms in the main chain have been substituted with a heteroatom. Thus, a heteroaliphatic group of a compound of Formula I and subgenera thereof refers to an aliphatic chain which contains one or more oxygen, sulfur, nitrogen, phosphorus or silicon atoms, e.g., in place of carbon atoms. Heteroaliphatic moieties of a compound of Formula I and subgenera thereof may be linear or branched, and saturated or unsaturated. In certain embodiments, heteroaliphatic moieties of a compound of Formula I and subgenera thereof are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ); —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein for Formula I and subgenera thereof may be substituted or unsubstituted, branched or unbranched, saturated or unsaturated, and wherein any of the aromatic, heteroaromatic, aryl or heteroaryl substituents described above and herein for Formula I and subgenera thereof may be substituted or unsubstituted. Additional examples of generally applicable substituents are illustrated by the specific embodiments shown in the Examples that are described herein for Formula I and subgenera thereof.

The term “heterocycloalkyl”, “heterocycle” or “heterocyclic”, as used herein with reference to Formula I and subgenera thereof, refers to compounds which combine the properties of heteroaliphatic and cyclic compounds and include, but are not limited to, saturated, unsaturated and partially saturated mono- or polycyclic cyclic ring systems having 5-16 atoms wherein at least one ring atom is a heteroatom selected from O, S and N (wherein the nitrogen and sulfur heteroatoms may be optionally oxidized), wherein the ring systems are optionally substituted with one or more functional groups, as defined herein for Formula I and subgenera thereof. In certain embodiments of Formula I and subgenera thereof, the term “heterocycloalkyl”, “heterocycle” or “heterocyclic” refers to a non-aromatic or partially aromatic 5-12 membered ring or a polycyclic group wherein at least one ring atom is a heteroatom selected from O, S and N (wherein the nitrogen and sulfur heteroatoms may be optionally oxidized), including, but not limited to a bi- or tri-cyclic group, comprising fused rings having between one and four heteroatoms independently selected from O, S and N, wherein (i) each 5-membered ring has 0 to 2 double bonds, each 6-membered ring has 0 to 3 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings for Formula I and subgenera thereof may be fused to an aryl or heteroaryl ring. Representative heterocycles for Formula I and subgenera thereof include, but are not limited to, heterocycles such as furanyl, thiofuranyl, pyranyl, pyrrolyl, pyrazolyl, imidazolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiadiazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, indolinyl, oxoindolinyl, and benzofused derivatives thereof. In certain embodiments of Formula I and subgenera thereof, a “substituted heterocycle, or heterocycloalkyl or heterocyclic” group is utilized and as used herein, refers to a heterocycle, or heterocycloalkyl or heterocyclic group, as defined above for Formula and subgenera thereof, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein for Formula I and subgenera thereof may be substituted or unsubstituted, branched or unbranched, saturated or unsaturated, and wherein any of the aromatic, heteroaromatic, aryl or heteroaryl substituents described above and herein for Formula I and subgenera thereof may be substituted or unsubstituted. Additional examples or generally applicable substituents are illustrated by the specific embodiments shown in the Examples, which are described herein for Formula I and subgenera thereof

Additionally, it will be appreciated that any of the alicyclic or heterocyclic moieties described above and herein for Formula I and subgenera thereof may comprise an aryl or heteroaryl moiety fused thereto. Additional examples of generally applicable substituents are illustrated by the specific embodiments shown in the Examples that are described herein for Formula I and subgenera thereof.

As used herein with reference to Formula II and subgenera thereof, the terms “heterocycle”, “heterocyclyl”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle of a compound of Formula II and subgenera thereof, the term “nitrogen” includes a substituted nitrogen. As an example, with reference to Formula II and subgenera thereof, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring of compounds of Formula II and subgenera thereof can be attached to its pendant group at any heteroatom or carbon atom that results in a stable structure and any of the ring atoms can be optionally substituted. Examples of such saturated or partially unsaturated heterocyclic radicals for use in compounds of Formula II and subgenera thereof include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein with reference to Formula II and subgenera thereof, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group of Formula II and subgenera thereof may be mono- or bicyclic.

The terms “halo” and “halogen” as used herein refer to an atom selected from fluorine, chlorine, bromine and iodine.

The term “haloalkyl” denotes an alkyl group, as defined above for Formula I and subgenera thereof, having one, two, or three halogen atoms attached thereto and is exemplified by such groups as chloromethyl, bromoethyl, trifluoromethyl, and the like.

The term “amino”, as used herein with reference to Formula I and subgenera thereof, refers to a primary (—NH₂), secondary (—NHRₓ), tertiary (—NRₓR_{y}) or quaternary (—N⁺RₓR_{y}R_{z}) amine, where Rₓ, R_{y} and R_{z} are independently an aliphatic, alicyclic, heteroaliphatic, heterocyclic, aromatic or heteroaromatic moiety, as defined herein for Formula I and subgenera thereof. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein with reference to Formula I and subgenera thereof, refers to a group having the general formula —C(═O)R, where R is an aliphatic, alicyclic, heteroaliphatic, heterocyclic, aromatic or heteroaromatic moiety, as defined herein for Formula I and subgenera thereof.

The term “C₂₋₆alkenylidene”, as used herein with reference to Formula I and subgenera thereof, refers to a substituted or unsubstituted, linear or branched unsaturated divalent radical consisting solely of carbon and hydrogen atoms, having from two to six carbon atoms, having a free valence “-” at both ends of the radical, and wherein the unsaturation is present only as double bonds and wherein a double bond can exist between the first carbon of the chain and the rest of the molecule.

As used herein with reference to Formula I and subgenera thereof, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like as used with reference to Formula I and subgenera thereof encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aromatic”, “heteroaromatic”, “aryl”, “heteroaryl” as used with reference to Formula I and subgenera thereof and the like encompass both substituted and unsubstituted groups.

As described herein, compounds of Formula II and subgenera thereof may contain “optionally substituted” moieties. In general, the term “substituted”, whether preceded by the term “optionally” or not, means that one or more hydrogens of the designated moiety of compounds of Formula II, and subgenera thereof, are replaced with a suitable substituent. Unless otherwise indicated, an “optionally substituted” group of Formula II and subgenera thereof may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group of a compound of Formula II, and subgenera thereof, are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄ S(O)₂OR^{o}; —(CH₂)₀₋₄OS(O)₂R^{o}; —S(O)₂NR^{o}₂; —(CH₂)₀₋₄ S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)_{0- 2}SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens; and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group of a compound of Formula II, and subgenera thereof, include the following: ═O, ═S, ═NNR*2, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below; or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group of a compound of Formula II, and subgenera thereof, include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group of a compound of Formula II, and subgenera thereof, include —R^{†}, —NR^{†}₂, —C(O)NR^{†}₂, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of IV, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The phrase, “pharmaceutically acceptable derivative(s)”, as used herein with reference to Formula I and subgenera thereof, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives of compounds of Formula I and subgenera thereof thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present compounds of Formula I and subgenera thereof. Certain exemplary pharmaceutical compositions and pharmaceutically acceptable derivatives of compounds of Formula I and subgenera thereof will be discussed in more detail herein below.

As used herein with reference to compounds of Formula II and subgenera thereof, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of compounds of Formula II and subgenera thereof include those derived from suitable inorganic and organic acids and bases. Examples of pharmaceutically acceptable, nontoxic acid addition salts for use in salts of compounds of Formula II and subgenera thereof are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts of compounds of Formula II and subgenera thereof include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts of compounds of Formula II and subgenera thereof derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures of compounds of Formula II and subgenera thereof depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of Formula II and subgenera thereof. Unless otherwise stated, all tautomeric forms of the compounds of Formula II and subgenera thereof are within the scope of the disclosure. Additionally, unless otherwise stated, compounds of Formula II and subgenera thereof are also meant to include compounds that differ only in the presence of one or more isotopically enriched atoms. For example, compounds of Formula II and subgenera thereof having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of Formula II and subgenera thereof comprise one or more deuterium atoms.

The term “tautomerization” refers to the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The term “tautomer” as used herein, refers to the compounds produced by the proton shift. For example, compounds of formula A and B can exist as a tautomer as shown below:

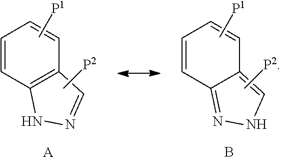

Thus, the present disclosure encompasses the substituted indazolyl compounds, in which the proton on the nitrogen can be attached to either of the two nitrogen atoms.

By the term “protecting group”, as used herein with reference to Formula I and subgenera thereof, it is meant that a particular functional moiety, e.g., O, S, or N, is temporarily blocked so that a reaction can be carried out selectively at another reactive site in a multifunctional compound. In preferred embodiments of Formula I and subgenera thereof, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein for compounds of Formula I and subgenera thereof, oxygen, sulfur, nitrogen and carbon protecting groups may be utilized. For example, in certain embodiments, as detailed herein for compounds of Formula I and subgenera thereof, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilyl ether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments of Formula I and subgenera thereof, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein for compounds of Formula I and subgenera thereof, however, it will be appreciated that the present disclosure is not intended to be limited to these protecting groups; rather, a variety of additional equivalent protecting groups can be readily identified using the above criteria and utilized in the present disclosure. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein, the term “isolated” when applied to the compounds of Formula I and subgenera thereof, refers to such compounds that are (i) separated from at least some components with which they are associated in nature or when they are made and/or (ii) produced, prepared or manufactured by the hand of man.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### DETAILED DESCRIPTION OF CERTAIN PREFERRED EMBODIMENTS OF THE INVENTION

The present disclosure provides compounds that inhibit ROCK1, ROCK2, or ROCK1/2 activities. ROCK1/2 refers to both ROCK1 and ROCK2 kinases.

Compounds of this disclosure include those generally set forth above and described specifically herein, and are illustrated in part by the various classes, subgenera and species disclosed herein.

Additionally, the present disclosure provides pharmaceutically acceptable derivatives of the provided compounds, and methods of treating a subject using these compounds, pharmaceutical compositions thereof, or either of these in combination with one or more additional therapeutic agents.

### 1) General Description of Compounds of Formula I

In certain embodiments, provided compounds include compounds of the general Formula I as further defined below:

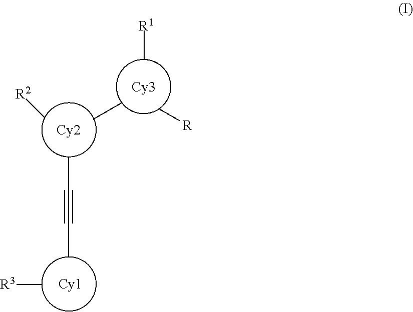

or a pharmaceutically acceptable salt thereof, wherein,

Cy1, Cy2, and Cy3 each independently represents an aryl, heteroaryl, or heterocyclic, which is optionally fused with a 3-8 membered cycloalkyl, 3-8 membered heterocycloalkyl, 6-membered aryl, or 5-6 membered heteroaryl;

R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};

R is an optionally substituted heterocyclic, aromatic, or heteroaromatic; wherein, the optional substituents are selected from one or more independent hydrogen, deuterium, halo; —CN, —NO₂, aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{k}CO₂R^{p};

R^{a}, R^{b}, R^{c}, R^{d}, R^{e}, R^{f}, R^{g}, R^{h}, Rⁱ, R^{j}, R^{k}, R^{l}, R^{m}, Rⁿ, R^{o}, R^{p} and R^{q} for each occurrence, is independently selected from hydrogen, deuterium, halo, —CN, —NO₂, an optionally substituted aliphatic, alicyclic, heteroaliphatic, heterocyclic, aromatic, or heteroaromatic; wherein each optional substituent is independently selected from one or more hydrogen, deuterium, halo, —CN, —NO₂, aliphatic, alicyclic, heteroaliphatic, heterocyclic, aromatic, heteroaromatic, —OR^{aa}, —NR^{bb}R^{cc}, —S(═O)_{w}R^{dd}, —S(═O)_{w}NR^{ee}R^{ff}, —C(═O)R^{gg}, —CO₂R^{hh}, —CONRⁱⁱR^{jj}, —NR^{kk}CONR^{ll}R^{mm}, —OCONRⁿⁿR^{oo}, or —NR^{kk}CO₂R^{pp}; or R^{b} and R^{c}, R^{e} and R^{f}, Rⁱ and R^{j}, R^{l} and R^{m}, or Rⁿ and R^{o}; when attached to the same nitrogen, may optionally form a heterocyclic ring, optionally containing 1-5 additional heteroatoms selected from O, S(O)_{w}, or N as the ring atoms, and may be optionally substituted with one or more hydrogen, deuterium, halo, —CN, —NO₂, aliphatic, alicyclic, heteroaliphatic, heterocyclic, aromatic, or heteroaromatic;

R^{aa}, R^{bb}, R^{cc}, R^{dd}, R^{ee}, R^{ff}, R^{gg}, R^{hh}, Rⁱⁱ, R^{jj}, R^{kk}, R^{ll}, R^{mm}, Rⁿⁿ, R^{oo}, and R^{pp}, for each occurrence, is independently selected from hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic; and

w is 0, 1, or 2.

In certain embodiments, Cy1 is a monocyclic or bicyclic or tricyclic aryl, heteroaryl, or heterocyclic. In certain embodiments; Cy1 is selected from phenyl, pyridinyl, pyridonyl; pyrimidinyl, pyrazinyl, pyridazinyl, triazinyl, tetrazinyl, quinolinyl, quinazolinyl, quinoxalinyl, cinnolinyl, isoquinolinyl, indolyl; aza-indolyl, indolinonyl, indolinyl, oxoindolinyl, 4,5,6,7-tetrahydro-1H-indazolyl, pyrazolyl, imidazolyl, triazolyl, tetrazolyl, oxazolyl, thiazolyl, benzimidazolyl, indazolyl, aza-indazolyl, benzoxazolyl, or benzothiazolyl.

In certain embodiments, Cy2 and Cy3 each independently represents a monocyclic aromatic, a bicyclic aromatic, a monocyclic heteroaromatic, a bicyclic heteroaromatic, a monocyclic heterocyclic or a bicyclic heterocyclic. In certain embodiments, Cy2 and Cy3 is each independently selected from phenyl, naphthyl, pyridinyl, pyridonyl, pyrimidinyl, pyrazinyl, pyridazinyl, triazinyl, tetrazinyl, quinolinyl, quinazolinyl, quinoxalinyl, cinnolinyl, indolyl, aza-indolyl, indolinonyl, indolinyl, oxoindolinyl, 4,5,6,7-tetrahydro-1H-indazolyl, pyrazolyl, imidazolyl, triazolyl, tetrazolyl, oxazolyl, thiazolyl, benzimidazolyl, indazolyl, benzoxazolyl, or benzothiazolyl.

In certain embodiments, R is a heterocyclic group, such as but not limited to azetidinyl, pyrrolidinyl, piperidinyl, piperazinyl, 5,6,7,8-tetrahydro-[1,2,4]triazolo[4,3-a]pyrazinyl, 4,5,6,7-tetrahydro-1H-pyrazolo[4,3-c]pyridinyl, indolinyl, isoindolinyl, aza-indolinyl, aza-isoindolinyl, dihydroindazolyl, tetrahydroquinolinyl, tetrahydroisoquinolinyl, aza-tetrahydroquinolinyl or aza-tetrahydroisoquinolinyl.

In certain embodiments, a compound of Formula I has the structure of Formula Ia:

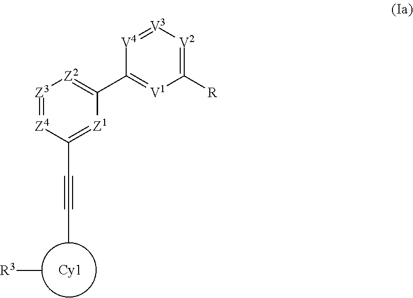

wherein V¹, V², V³ and V⁴ are each independently N or C—R¹, wherein two R′ groups on adjacent carbon atoms together with the carbons they are attached to may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂;

Z¹, Z², Z³ and Z⁴ is each independently N or C—R², wherein two R² groups on adjacent carbon atoms together with the carbons they are attached to may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂;

and definitions of R, R¹, R², Cy1, and R³ are the same with those in Formula I.

In certain embodiments, a compound of Formula I has the structure of Formula Ib:

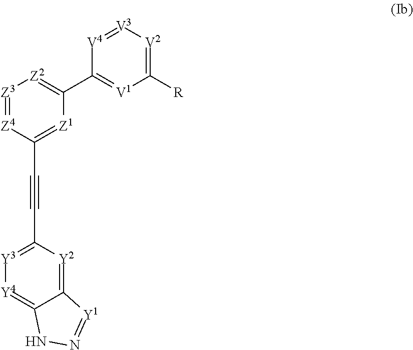

wherein Y¹, Y², Y³ and Y⁴ is each independently N or C—R³, wherein two R³ groups on adjacent carbon atoms together with the carbons they are attached to may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂;

and definitions of V¹, V², V², V⁴, Z¹, Z², Z³, and Z⁴ are the same with those in Formula Ia and R and R³ have the same meaning with those in Formula I.

In certain embodiments, a compound of Formula I has the structure of Formula Ic or Id:

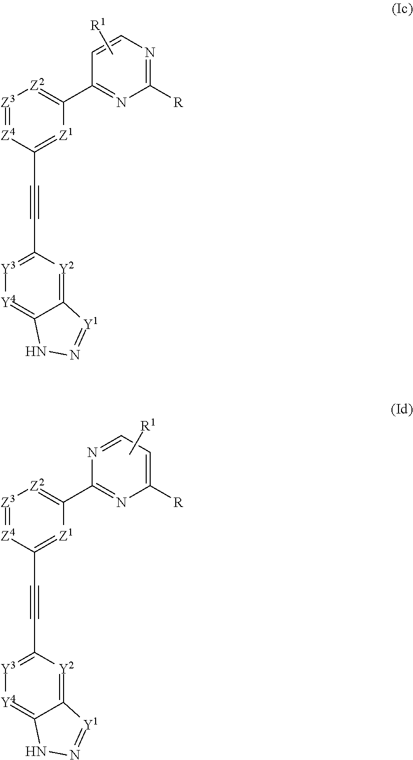

wherein the definitions of Z¹, Z², Z³, and Z⁴ are the same with those in Formula Ia, the definitions of Y¹, Y²; Y³, and Y⁴ are the same with those in Formula Ib, and R and R¹ have the same meaning with those in Formula I.

In certain embodiments, a compound of Formula I has the structure of Formula Ie, If, Ig, Ih, Ii, or Ij:

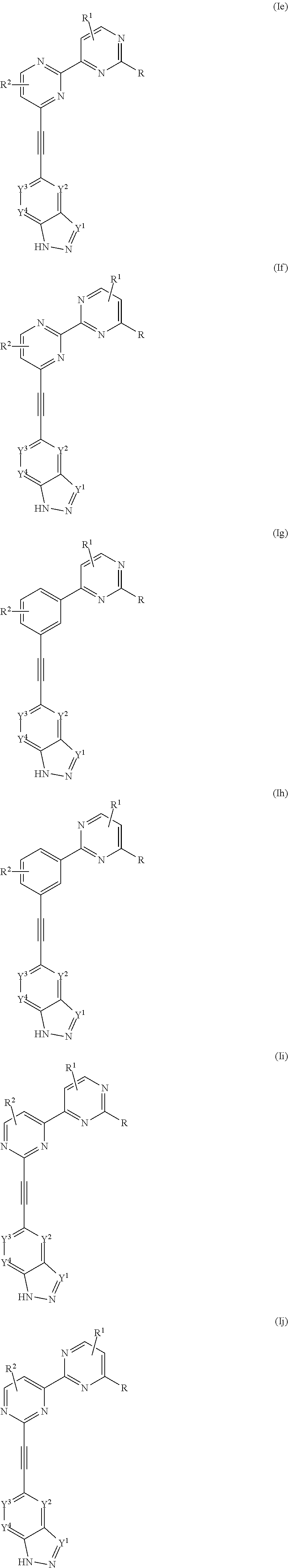

wherein the definitions of Y¹, Y², Y³, and Y⁴ are the same with those in Formula Ib and R, R¹, and R² have the same meaning with those in Formula I.

In certain embodiments, a compound of Formula I has the structure of Formula Ik, Il, Im, In, Io, or Ip:

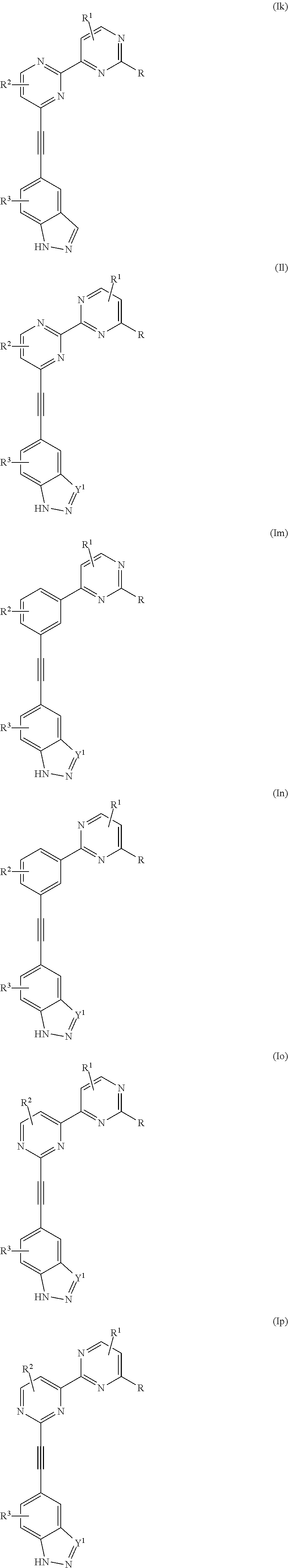

wherein R, R¹, R², and R³ have the same meaning with those in Formula I, and the R³ group can be connected to any carbon atom in the indazolyl ring.

In one embodiment, the compound of Formula I is selected from the following:
5-Methoxy-2-(4-(pyridin-4-ylethynyl)-[2,4′-bipyrimidin]-2′-yl)isoindoline;
2-(4-((TH-pyrazol-4-yl)ethynyl)-[2,4′-bipyrimidin]-2′-yl)-5-methoxyisoindoline;
5-((2′-(5-methoxyisoindolin-2-yl)-[2,4′-bipyrimidin]-4-yl)ethynyl)-H-indazole;
6-((2′-(5-methoxyisoindolin-2-yl)-[2,4′-bipyrimidin]-4-yl)ethynyl)isoquinolin-1-amine;
3-fluoro-5-((2′-(5-methoxyisoindolin-2-yl)-[2,4′-bipyrimidin]-4-yl)ethynyl)-H-indazole;
7-fluoro-5-((2′-(5-methoxyisoindolin-2-yl)-[2,4′-bipyrimidin]-4-yl)ethynyl)-1H-indazole;
5-((2′-(5-methoxyisoindolin-2-yl)-[2,4′-bipyrimidin]-4-yl)ethynyl)isoindolin-1-one;
methyl 4-((2′-(5-methoxyisoindolin-2-yl)-[2,4′-bipyrimidin]-4-yl)ethynyl)benzoate;
4-((2′-(5-methoxyisoindolin-2-yl)-[2,4′-bipyrimidin]-4-yl)ethynyl)benzonitrile;
4-((2′-(5-methoxyisoindolin-2-yl)-[2,4′-bipyrimidin]-4-yl)ethynyl)benzoic acid;
4-((2′-(5-methoxyisoindolin-2-yl)-[2,4′-bipyrimidin]-4-yl)ethynyl)-N-methylbenzamide;
5-((2′-(isoindolin-2-yl)-[2,4′-bipyrimidin]-4-yl)ethynyl)-1H-indazole;
5-((2′-(5-fluoroisoindolin-2-yl)-[2,4′-bipyrimidin]-4-yl)ethynyl)-1H-indazole;
7-fluoro-5-((2′-(5-fluoroisoindolin-2-yl)-[2,4′-bipyrimidin]-4-yl)ethynyl)-1H-indazole;
5-((2′-(6-methoxy-1,3-dihydro-2H-pyrrolo[3,4-c]pyridin-2-yl)-[2,4′-bipyrimidin]-4-yl)ethynyl)-1H-indazole;
2-(4-((TH-indazol-5-yl)ethynyl)-[2,4′-bipyrimidin]-2′-yl)-2,3-dihydro-H-pyrrolo[3,4-c]pyridin-6-ol;
5-((2′-(6-chloro-1,3-dihydro-2H-pyrrolo[3,4-c]pyridin-2-yl)-[2,4′-bipyrimidin]-4-yl)ethynyl)-1H-indazole;
5-((6-(2-(5-methoxyisoindolin-2-yl)pyrimidin-4-yl)pyridin-2-yl)ethynyl)-1H-indazole;
7-fluoro-5-((6-(2-(5-methoxyisoindolin-2-yl)pyrimidin-4-yl)pyridin-2-yl)ethynyl)-1H-indazole;
5-((6-(2-(5-fluoroisoindolin-2-yl)pyrimidin-4-yl)pyridin-2-yl)ethynyl)-1H-indazole;
7-fluoro-5-((6-(2-(5-fluoroisoindolin-2-yl)pyrimidin-4-yl)pyridin-2-yl)ethynyl)-1H-indazole;
2-((2-(4-(6-((1H-indazol-5-yl)ethynyl)pyridin-2-yl)pyrimidin-2-yl)isoindolin-5-yl)oxy)-N,N-dimethylethanamine;
5-((6-(2-(5-(4-methylpiperazin-1-yl)isoindolin-2-yl)pyrimidin-4-yl)pyridin-2-yl)ethynyl)-1H-indazole;
5-((3-fluoro-5-(2-(5-methoxyisoindolin-2-yl)pyrimidin-4-yl)phenyl)ethynyl)-1H-indazole;
5-((3-fluoro-5-(2-(5-fluoroisoindolin-2-yl)pyrimidin-4-yl)phenyl)ethynyl)-1H-indazole;
5-((3-(2-(5-chloroisoindolin-2-yl)pyrimidin-4-yl)-5-fluorophenyl)ethynyl)-1H-indazole;
5-((3-(2-(5-bromoisoindolin-2-yl)pyrimidin-4-yl)-5-fluorophenyl)ethynyl)-1H-indazole;
2-((2-(4-(3-((1H-indazol-5-yl)ethynyl)-5-fluorophenyl)pyrimidin-2-yl)isoindolin-5-yl)oxy)-N,N-dimethylethanamine;
5-((3-fluoro-5-(2-(5-(4-methylpiperazin-1-yl)isoindolin-2-yl)pyrimidin-4-yl)phenyl)ethynyl)-1H-indazole;
5-((2′-(5-bromoisoindolin-2-yl)-[2,4′-bipyrimidin]-4-yl)ethynyl)-1H-indazole;
5-((2′-(5-chloroisoindolin-2-yl)-[2,4′-bipyrimidin]-4-yl)ethynyl)-1H-indazole;
5-((2′-(5-cyanoisoindolin-2-yl)-[2,4′-bipyrimidin]-4-yl)ethynyl)-1H-indazole;
5-((2′-(5-(fluoromethyl)isoindolin-2-yl)-[2,4′-bipyrimidin]-4-yl)ethynyl)-1H-indazole;
5-((2′-(5-(difluoromethyl)isoindolin-2-yl)-[2,4′-bipyrimidin]-4-yl)ethynyl)-1H-indazole;
5-((2′-(5-(trifluoromethyl)isoindolin-2-yl)-[2,4′-bipyrimidin]-4-yl)ethynyl)-1H-indazole;
5-((2′-(5-(difluoromethoxyl)isoindolin-2-yl)-[2,4′-bipyrimidin]-4-yl)ethynyl)-1H-indazole;
5-((2′-(5-(triifluoromethoxyl)isoindolin-2-yl)-[2,4′-bipyrimidin]-4-yl)ethynyl)-1H-indazole;
7-fluoro-5-((2′-(isoindolin-2-yl)-[2,4′-bipyrimidin]-4-yl)ethynyl)-1H-indazole;
7-fluoro-5-((2′-(5-chloroisoindolin-2-yl)-[2,4′-bipyrimidin]-4-yl)ethynyl)-1H-indazole;
7-fluoro-5-((2′-(5-bromoisoindolin-2-yl)-[2,4′-bipyrimidin]-4-yl)ethynyl)-1H-indazole;
7-fluoro-5-((2′-(5-cyanoisoindolin-2-yl)-[2,4′-bipyrimidin]-4-yl)ethynyl)-1H-indazole;
7-fluoro-5-((2′-(5-difluoromethoxyisoindolin-2-yl)-[2,4′-bipyrimidin]-4-yl)ethynyl)-1H-indazole;
7-fluoro-5-((2′-(5-trifluoromethoxyisoindolin-2-yl)-[2,4′-bipyrimidin]-4-yl)ethynyl)-1H-indazole;
7-fluoro-5-((2′-(5-(fluoromethyl)isoindolin-2-yl)-[2,4′-bipyrimidin]-4-yl)ethynyl)-1H-indazole;
7-fluoro-5-((2′-(5-(difluoromethyl)isoindolin-2-yl)-[2,4′-bipyrimidin]-4-yl)ethynyl)-1H-indazole;
7-fluoro-5-((2′-(5-(trifluoromethyl)isoindolin-2-yl)-[2,4′-bipyrimidin]-4-yl)ethynyl)-1H-indazole;
3-fluoro-5-((2′-(isoindolin-2-yl)-[2,4′-bipyrimidin]-4-yl)ethynyl)-1H-indazole;
3-fluoro-5-((2′-(5-chloroisoindolin-2-yl)-[2,4′-bipyrimidin]-4-yl)ethynyl)-1H-indazole;
3-fluoro-5-((2′-(5-bromoisoindolin-2-yl)-[2,4′-bipyrimidin]-4-yl)ethynyl)-1H-indazole;
3-fluoro-5-((2′-(5-cyanoisoindolin-2-yl)-[2,4′-bipyrimidin]-4-yl)ethynyl)-1H-indazole;
3-fluoro-5-((2′-(5-difluoromethoxyisoindolin-2-yl)-[2,4′-bipyrimidin]-4-yl)ethynyl)-1H-indazole;
3-fluoro-5-((2′-(5-trifluoromethoxyisoindolin-2-yl)-[2,4′-bipyrimidin]-4-yl)ethynyl)-1H-indazole;
3-fluoro-5-((2′-(5-(fluoromethyl)isoindolin-2-yl)-[2,4′-bipyrimidin]-4-yl)ethynyl)-1H-indazole;
3-fluoro-5-((2′-(5-(difluoromethyl)isoindolin-2-yl)-[2,4′-bipyrimidin]-4-yl)ethynyl)-1H-indazole; and
3-fluoro-5-((2′-(5-(trifluoromethyl)isoindolin-2-yl)-[2,4′-bipyrimidin]-4-yl)ethynyl)-1H-indazole.

In one embodiment, a compound of Formula Ic is selected from among:
5-Methoxy-2-(4-(pyridin-4-ylethynyl)-[2,4′-bipyrimidin]-2′-yl)isoindoline;
2-(4-((1H-pyrazol-4-yl)ethynyl)-[2,4′-bipyrimidin]-2′-yl)-5-methoxyisoindoline;
5-((2′-(5-methoxyisoindolin-2-yl)-[2,4′-bipyrimidin]-4-yl)ethynyl)-1H-indazole;
6-((2′-(5-methoxyisoindolin-2-yl)-[2,4′-bipyrimidin]-4-yl)ethynyl)isoquinolin-1-amine;
3-fluoro-5-((2′-(5-methoxyisoindolin-2-yl)-[2,4′-bipyrimidin]-4-yl)ethynyl)-1H-indazole;
7-fluoro-5-((2′-(5-methoxyisoindolin-2-yl)-[2,4′-bipyrimidin]-4-yl)ethynyl)-1H-indazole;
5-((2′-(5-methoxyisoindolin-2-yl)-[2,4′-bipyrimidin]-4-yl)ethynyl)isoindolin-1-one;
methyl 4-((2′-(5-methoxyisoindolin-2-yl)-[2,4′-bipyrimidin]-4-yl)ethynyl)benzoate;
4-((2′-(5-methoxyisoindolin-2-yl)-[2,4′-bipyrimidin]-4-yl)ethynyl)benzonitrile;
4-((2′-(5-methoxyisoindolin-2-yl)-[2,4′-bipyrimidin]-4-yl)ethynyl)benzoic acid;
4-((2′-(5-methoxyisoindolin-2-yl)-[2,4′-bipyrimidin]-4-yl)ethynyl)-N-methylbenzamide;
5-((2′-(isoindolin-2-yl)-[2,4′-bipyrimidin]-4-yl)ethynyl)-1H-indazole;
5-((2′-(5-fluoroisoindolin-2-yl)-[2,4′-bipyrimidin]-4-yl)ethynyl)-1H-indazole;
7-fluoro-5-((2′-(5-fluoroisoindolin-2-yl)-[2,4′-bipyrimidin]-4-yl)ethynyl)-1H-indazole;
5-((2′-(6-methoxy-1,3-dihydro-2H-pyrrolo[3,4-c]pyridin-2-yl)-[2,4′-bipyrimidin]-4-yl)ethynyl)-1H-indazole;
2-(4-((TH-indazol-5-yl)ethynyl)-[2,4′-bipyrimidin]-2′-yl)-2,3-dihydro-H-pyrrolo[3,4-c]pyridin-6-ol;
5-((2′-(6-chloro-1,3-dihydro-2H-pyrrolo[3,4-c]pyridin-2-yl)-[2,4′-bipyrimidin]-4-yl)ethynyl)-1H-indazole;
5-((6-(2-(5-methoxyisoindolin-2-yl)pyrimidin-4-yl)pyridin-2-yl)ethynyl)-1H-indazole;
7-fluoro-5-((6-(2-(5-methoxyisoindolin-2-yl)pyrimidin-4-yl)pyridin-2-yl)ethynyl)-1H-indazole;
5-((6-(2-(5-fluoroisoindolin-2-yl)pyrimidin-4-yl)pyridin-2-yl)ethynyl)-1H-indazole;
7-fluoro-5-((6-(2-(5-fluoroisoindolin-2-yl)pyrimidin-4-yl)pyridin-2-yl)ethynyl)-1H-indazole;
2-((2-(4-(6-((TH-indazol-5-yl)ethynyl)pyridin-2-yl)pyrimidin-2-yl)isoindolin-5-yl)oxy)-N,N-dimethylethanamine;
5-((6-(2-(5-(4-methylpiperazin-1-yl)isoindolin-2-yl)pyrimidin-4-yl)pyridin-2-yl)ethynyl)-1H-indazole;
5-((3-fluoro-5-(2-(5-methoxyisoindolin-2-yl)pyrimidin-4-yl)phenyl)ethynyl)-1H-indazole;
5-((3-fluoro-5-(2-(5-fluoroisoindolin-2-yl)pyrimidin-4-yl)phenyl)ethynyl)-1H-indazole;
5-((3-(2-(5-chloroisoindolin-2-yl)pyrimidin-4-yl)-5-fluorophenyl)ethynyl)-1H-indazole;
5-((3-(2-(5-bromoisoindolin-2-yl)pyrimidin-4-yl)-5-fluorophenyl)ethynyl)-1H-indazole;
2-((2-(4-(3-((1H-indazol-5-yl)ethynyl)-5-fluorophenyl)pyrimidin-2-yl)isoindolin-5-yl)oxy)-N,N-dimethylethanamine;
5-((3-fluoro-5-(2-(5-(4-methylpiperazin-1-yl)isoindolin-2-yl)pyrimidin-4-yl)phenyl)ethynyl)-1H-indazole; and
5-((2′-(5-bromoisoindolin-2-yl)-[2,4′-bipyrimidin]-4-yl)ethynyl)-H-indazole.

In one embodiment, a pharmaceutical composition is provided comprising one or more compounds of any one of the foregoing formulas, and a pharmaceutically acceptable carrier, excipient, vehicle or diluent.

In one embodiment, the compound of Formula I has ROCK1, ROCK2, or ROCK1/2 inhibitory activities. In one embodiment, the compound has antifibrotic activity.

In one embodiment, a method of modulating ROCK1, ROCK2, or ROCK1/2 activities in a patient or in a biological sample is provided, which method comprises administering to said patient, or contacting said biological sample with a composition as described above or any compounds as described herein.

In one embodiment, a method is provided for treating a condition, disease or disorder in which ROCK1, ROCK2, or ROCK1/2 plays a role. In one embodiment, the method is for treating or lessening the severity of a disease or condition selected from renal fibrosis, fibrotic liver disease, hepatic ischemia-reperfusion injury, cerebral infarction, ischemic heart disease, renal disease or lung (pulmonary) fibrosis. In one embodiment, the method is for treating or lessening the severity of a disease or condition selected from liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemia/reperfusion injury; stroke; cerebrovascular disease; myocardial ischemia; atherosclerosis; renal failure; renal fibrosis and idiopathic pulmonary fibrosis. In one embodiment, the method is for the treatment of wounds for acceleration of healing; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; acute or chronic heart failure, renal failure secondary to chronic diabetes and/or hypertension; amyotrophic lateral sclerosis, muscular dystrophy, glaucoma, corneal scarring, macular degeneration, diabetic retinopathy and/or diabetes mellitus.

With regard to the foregoing compounds of Formula I, a number of important subclasses of each of the foregoing formulas deserve separate mention; these subclasses include subclasses of the foregoing classes in which:

i) Cy1 is a monocyclic or bicyclic or tricyclic aryl, heteroaryl, or heterocyclic;

ii) Cy1 is phenyl, pyridinyl, pyridonyl, pyrimidinyl, pyrazinyl, pyridazinyl, triazinyl, tetrazinyl, quinolinyl, quinazolinyl, quinoxalinyl, cinnolinyl, isoquinolinyl, indolyl, aza-indolyl, indolinonyl, indolinyl, oxoindolinyl, 4,5,6,7-tetrahydro-1H-indazolyl, pyrazolyl, imidazolyl, triazolyl, tetrazolyl, oxazolyl, thiazolyl, benzimidazolyl, indazolyl, aza-indazolyl, benzoxazolyl, or benzothiazolyl;

iii) Cy2 is a monocyclic or bicyclic or tricyclic aryl, heteroaryl, or heterocyclic;

iv) Cy2 is phenyl, pyridinyl, pyridonyl, pyrimidinyl, pyrazinyl, pyridazinyl, triazinyl, tetrazinyl, quinolinyl, quinazolinyl, quinoxalinyl, cinnolinyl, isoquinolinyl, indolyl, aza-indolyl, indolinonyl, indolinyl, oxoindolinyl, 4,5,6,7-tetrahydro-1H-indazolyl, pyrazolyl, imidazolyl, triazolyl, tetrazolyl, oxazolyl, thiazolyl, benzimidazolyl, indazolyl, aza-indazolyl, benzoxazolyl, or benzothiazolyl;

v) Cy3 is a monocyclic or bicyclic or tricyclic aryl, heteroaryl, or heterocyclic;

vi) Cy3 is phenyl, pyridinyl, pyridonyl, pyrimidinyl, pyrazinyl, pyridazinyl, triazinyl, tetrazinyl, quinolinyl, quinazolinyl, quinoxalinyl, cinnolinyl, isoquinolinyl, indolyl, aza-indolyl, indolinonyl, indolinyl, oxoindolinyl, 4,5,6,7-tetrahydro-1H-indazolyl, pyrazolyl, imidazolyl, triazolyl, tetrazolyl, oxazolyl, thiazolyl, benzimidazolyl, indazolyl, aza-indazolyl, benzoxazolyl, or benzothiazolyl;

vii) Cy1 is phenyl, indazolyl, tetrahydroindazolyl, pyrazolyl, quinolinyl, or isoquinolinyl;

viii) Cy2 is phenyl, pyrimidinyl, or pyridinyl;

ix) Cy3 is phenyl, pyrimidinyl, or pyridinyl;

x) Cy2 and Cy3 together is a bipyrimidinyl;

xi) Cy2 and Cy3 together is 2,4′-bipyrimidinyl, 4,4′-bipyrimidinyl, or 2,4′,1,6′-bipyrimidinyl;

xii) R is a heterocyclic group;

xiii) R is a heterocyclic group, such as but not limited to azetidinyl, pyrrolidinyl, piperidinyl, piperazinyl, 5,6,7,8-tetrahydro-[1,2,4]triazolo[4,3-a]pyrazinyl, 4,5,6,7-tetrahydro-1H-pyrazolo[4,3-c]pyridinyl, indolinyl, isoindolinyl, aza-indolinyl, aza-isoindolinyl, dihydroindazolyl, tetrahydroquinolinyl, tetrahydroisoquinolinyl, aza-tetrahydroquinolinyl or aza-tetrahydroisoquinolinyl.

xiv) R is isoindolinyl, aza-isoindolinyl, azetidinyl, piperidinyl, piperazinyl, 5,6,7,8-tetrahydro-[1,2,4]triazolo[4,3-a]pyrazinyl, or 4,5,6,7-tetrahydro-1H-pyrazolo[4,3-c]pyridinyl;

xv) Cy1 is a monocyclic or bicyclic or tricyclic aryl, heteroaryl, or heterocyclic group; Cy2 and Cy3 independently represent a monocyclic or bicyclic aromatic, or a monocyclic or bicyclic heteroaromatic; R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};

xvi) Cy1 is indazolyl; Cy2 and Cy3 independently represent a monocyclic or bicyclic aromatic, or a monocyclic or bicyclic heteroaromatic; R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};

xvii) Cy1 is a monocyclic or bicyclic or tricyclic aryl, heteroaryl, or heterocyclic group; Cy2 and Cy3 independently is pyrimidinyl; R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};

xviii) Cy1 is a monocyclic or bicyclic or tricyclic aryl, heteroaryl, or heterocyclic group; Cy2 and Cy3 independently is pyridinyl; R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};

xix) Cy1 is indazolyl; Cy2 and Cy3 independently is pyrimidinyl; R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};

xx) Cy1 is indazolyl; Cy2 and Cy3 independently is pyridinyl; R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q}.

It will be appreciated that for each of the classes and subclasses described above and herein, any one or more occurrences of aliphatic and/or heteroaliphatic may independently be substituted or unsubstituted, linear or branched, saturated or unsaturated; any one or more occurrences of alicyclic and/or heteroalicyclic may independently be substituted or unsubstituted, saturated or unsaturated; and any one or more occurrences of aryl and/or heteroaryl may independently be substituted or unsubstituted.

The reader will also appreciate that all possible combinations of the variables described in i)-through xx) above (e.g., R, R¹, R², R³, Cy1, Cy2, and Cy3, among others) are considered part of the disclosure. Thus, the disclosure encompasses any and all compounds of Formula I generated by taking any possible permutation of variables R, R¹, R², R³, Cy1, Cy2, and Cy3, and other variables/substituents as further defined for R, R¹, R², R³, Cy1, Cy2, and Cy3, described in i) through xx) above.

For example, an exemplary combination of variables described in i) through xx) above includes those compounds of Formula (I) wherein:

Cy1 is an indazoyl, tetrahydro-indazolyl, aza-indazolyl, isoquinolinyl, indolinyl, or oxoindolinyl;

Cy2 and Cy3 are independently selected from phenyl, naphthyl, pyridinyl, pyridonyl, pyrimidinyl, pyrazinyl, pyridazinyl, triazinyl, tetrazinyl, quinolinyl, quinazolinyl, quinoxalinyl, cinnolinyl, indolyl, aza-indolyl, pyrazolyl, imidazolyl, triazolyl, tetrazolyl, oxazolyl, thiazolyl, benzimidazolyl, indazolyl, benzoxazolyl, or benzothiazolyl;

R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};

R is an optionally substituted heterocyclic, aromatic, or heteroaromatic; wherein, the optional substituents are selected from hydrogen, deuterium, halo, —CN, —NO₂, aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{k}CO₂R^{p};

Selected R includes, but not limited to azetidinyl, pyrrolidinyl, piperidinyl, piperazinyl, 5,6,7,8-tetrahydro-[1,2,4]triazolo[4,3-a]pyrazinyl, 4,5,6,7-tetrahydro-1H-pyrazolo[4,3-c]pyridinyl, indolinyl, isoindolinyl, aza-indolinyl, aza-isoindolinyl, dihydroindazolyl, tetrahydroindazolyl, tetrahydroquinolinyl, tetrahydroisoquinolinyl, aza-tetrahydroquinolinyl, or aza-tetrahydroisoquinolinyl;

R^{a}, R^{b}, R^{c}, R^{d}, R^{e}, R^{f}, R^{g}, R^{h}, Rⁱ, R^{j}, R^{k}, R^{l}, R^{m}, Rⁿ, R^{o}, R^{p} and R^{q}, for each occurrence, is independently selected from hydrogen, deuterium, halo, —CN, —NO₂, an optionally substituted aliphatic, alicyclic, heteroaliphatic, heterocyclic, aromatic, or heteroaromatic; wherein, the optional substituents are selected from hydrogen, deuterium, halo, —CN, —NO₂, aliphatic, alicyclic, heteroaliphatic, heterocyclic, aromatic, heteroaromatic, —OR^{aa}, —NR^{bb}R^{cc}, —S(═O)_{w}R^{dd}, —S(═O)_{w}NR^{ee}R^{ff}, —C(═O)R^{gg}, —CO₂R^{hh}, —CONRⁱⁱR^{jj}, —NR^{kk}CONR^{ll}R^{mm}, —OCONRⁿⁿR^{oo}, or —NR^{kk}CO₂R^{pp}; or R^{b} and R^{c}, R^{e} and R^{f}, Rⁱ and R^{j}, R^{l} and R^{m}, or Rⁿ and R^{o}, when attached to the same nitrogen, may optionally form a heterocyclic ring, optionally containing 1-5 additional heteroatoms selected from O, S(O)_{w}, or N as the ring atoms, and may be optionally substituted with one or more hydrogen, deuterium, halo, —CN, —NO₂, aliphatic, alicyclic, heteroaliphatic, heterocyclic, aromatic, or heteroaromatic; R^{aa}, R^{bb}, R^{cc}, R^{dd}, R^{ee}, R^{ff}, R^{gg}, R^{hh}, RRⁱⁱ, R^{jj}, R^{kk}, R^{ll}, R^{mm}, Rⁿⁿ, R^{oo}, and R^{pp}, for each occurrence, is independently selected from hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic; and

w is 0, 1, or 2.

In some embodiments, the present disclosure provides a compound of Formula II:

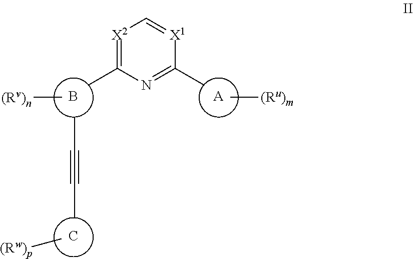

or a pharmaceutically acceptable salt thereof, wherein:
each of X¹ and X² is selected from CH and N, wherein only one of X¹ and X² is N;
Ring A is selected from a 4- to 7-membered saturated or partially unsaturated heterocyclic ring comprising 1-2 heteroatoms independently selected from nitrogen, oxygen, and sulfur, or a 5- to 6-membered saturated heterocyclic ring comprising 1-2 heteroatoms independently selected from nitrogen, oxygen and sulfur fused to a group independently selected from phenyl and a 5- or 6-membered heteroaryl ring comprising 1-3 heteroatoms independently selected from nitrogen, oxygen, and sulfur;
Ring B is selected from phenyl and a 6-membered heteroaryl ring comprising 1-2 nitrogen atoms:
Ring C is selected from phenyl, a 5- to 6-membered heteroaryl ring comprising 1-3 heteroatoms independently selected from nitrogen, oxygen, and sulfur, and a 9- to 10-membered heteroaryl ring comprising 1-3 heteroatoms independently selected from nitrogen, oxygen, and sulfur;
each R^{u} is independently selected from halogen, OR^{u}, and an optionally substituted group selected from C₁₋₆ aliphatic, phenyl, a 3- to 7-membered saturated or partially unsaturated heterocyclic ring comprising 1-3 heteroatoms independently selected from nitrogen, oxygen, and sulfur, and a 5- to 6-membered heteroaryl ring comprising 1-3 heteroatoms independently selected from nitrogen, oxygen, and sulfur;
each R^{v} is independently selected from halogen, CN, CO₂R″, C(O)NR″₂, NR″₂, OR″, SR″, and optionally substituted C₁₋₆ aliphatic;
each R^{w} is independently selected from halogen, CN, CO₂R″, C(O)NR″₂, NR″₂, OR″, SR″, and optionally substituted C₁₋₆ aliphatic, or
two independent occurrences of R^{w}, taken together with their intervening atom(s), form an optionally substituted 5-membered saturated or partially unsaturated heterocyclic ring comprising 1-2 heteroatoms independently selected from nitrogen, oxygen, and sulfur;
each R″ is independently selected from hydrogen or an optionally substituted group selected from C₁₋₆ aliphatic, phenyl, and a 3- to 7-membered saturated or partially unsaturated heterocyclic ring comprising 1-3 heteroatoms independently selected from nitrogen, oxygen, and sulfur; and
each of m, n, and p is independently 0-4.

As defined above for Formula II, each of X¹ and X² is selected from CH and N, wherein only one of X¹ and X² is N. In some embodiments of Formula II, X¹ is N and X² is CH. In some embodiments, X¹ is CH and X² is N.

As defined above for Formula II, Ring A is selected from a 4- to 7-membered saturated or partially unsaturated heterocyclic ring comprising 1-2 heteroatoms independently selected from nitrogen, oxygen, and sulfur, or a 5- to 6-membered saturated heterocyclic ring comprising 1-2 heteroatoms independently selected from nitrogen, oxygen and sulfur fused to a group independently selected from phenyl and a 5- or 6-membered heteroaryl ring comprising 1-3 heteroatoms independently selected from nitrogen, oxygen, and sulfur.

In some embodiments of Formula II, Ring A is

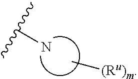

In some embodiments of Formula II, Ring A is a 4- to 7-membered saturated or partially unsaturated heterocyclic ring comprising 1-2 heteroatoms independently selected from nitrogen, oxygen, and sulfur. In some embodiments of Formula II, Ring A is a 4-membered saturated heterocyclic ring comprising 1 heteroatom selected from nitrogen, oxygen, and sulfur. In some embodiments of Formula II, Ring A is a 5-membered saturated or partially unsaturated heterocyclic ring comprising 1 heteroatom selected from nitrogen, oxygen, and sulfur. In some embodiments of Formula II, Ring A is a 5-membered saturated heterocyclic ring comprising 1 heteroatom selected from nitrogen, oxygen, and sulfur. In some embodiments of Formula II, Ring A is a 6-membered saturated or partially unsaturated heterocyclic ring comprising 1-2 heteroatoms independently selected from nitrogen, oxygen, and sulfur. In some embodiments of Formula II, Ring A is a 6-membered saturated heterocyclic ring comprising 1-2 heteroatoms independently selected from nitrogen, oxygen, and sulfur. In some embodiments of Formula II, Ring A is selected from azetidinyl, pyrrolidinyl, piperidinyl, and piperazinyl.

In some embodiments of Formula II, Ring A is a 5- to 6-membered saturated heterocyclic ring comprising 1-2 heteroatoms independently selected from nitrogen, oxygen and sulfur fused to a group independently selected from phenyl and a 5- or 6-membered heteroaryl ring comprising 1-3 heteroatoms independently selected from nitrogen, oxygen, and sulfur. In some embodiments of Formula II, Ring A is a 5-membered saturated heterocyclic ring comprising 1-2 heteroatoms independently selected from nitrogen, oxygen and sulfur fused to a group independently selected from phenyl and a 5- or 6-membered heteroaryl ring comprising 1-3 heteroatoms independently selected from nitrogen, oxygen, and sulfur.

In some embodiments of Formula II, Ring A is a 5-membered saturated heterocyclic ring comprising 1-2 heteroatoms independently selected from nitrogen, oxygen and sulfur fused to a group independently selected from phenyl and a 5- or 6-membered heteroaryl ring comprising 1-3 heteroatoms independently selected from nitrogen, oxygen, and sulfur. In some embodiments of Formula II, Ring A is a 5-membered saturated heterocyclic ring comprising 1-2 heteroatoms independently selected from nitrogen, oxygen and sulfur fused to a phenyl group. In some embodiments of Formula II, Ring A is a 5-membered saturated heterocyclic ring comprising 1 heteroatom selected from nitrogen, oxygen and sulfur fused to a phenyl group. In some embodiments of Formula II, Ring A is a 5-membered saturated heterocyclic ring comprising 1 nitrogen atom fused to a phenyl group. In some such embodiments of Formula II, Ring A is isoindolinyl.

In some embodiments of Formula II, Ring A is a 5-membered saturated heterocyclic ring comprising 1-2 heteroatoms independently selected from nitrogen, oxygen and sulfur fused to a 5- or 6-membered heteroaryl ring comprising 1-3 heteroatoms independently selected from nitrogen, oxygen, and sulfur. In some embodiments of Formula II, Ring A is a 5-membered saturated heterocyclic ring comprising 1 heteroatom selected from nitrogen, oxygen and sulfur fused to a 5- or 6-membered heteroaryl ring comprising 1-3 heteroatoms independently selected from nitrogen, oxygen, and sulfur. In some embodiments of Formula II, Ring A is a 5-membered saturated heterocyclic ring comprising 1 nitrogen atom fused to a 5- or 6-membered heteroaryl ring comprising 1-3 heteroatoms independently selected from nitrogen, oxygen, and sulfur. In some embodiments of Formula II, Ring A is a 5-membered saturated heterocyclic ring comprising 1 nitrogen atom fused to a 6-membered heteroaryl ring comprising 1-3 nitrogen atoms. In some embodiments of Formula II, Ring A is a 5-membered saturated heterocyclic ring comprising 1 nitrogen atom fused to a 6-membered heteroaryl ring comprising 1-2 nitrogen atoms. In some embodiments of Formula II, Ring A is a 5-membered saturated heterocyclic ring comprising 1 nitrogen atom fused to a 6-membered heteroaryl ring comprising 1 nitrogen atom. In some embodiments of Formula II, Ring A is 2,3-dihydro-1H-pyrrolo[3,4-c]pyridinyl.

In some embodiments of Formula II, Ring A is a 5-membered saturated heterocyclic ring comprising 1 nitrogen atom fused to a 5-membered heteroaryl ring comprising 1-3 heteroatoms independently selected from nitrogen, oxygen, and sulfur. In some embodiments of Formula II, Ring A is a 5-membered saturated heterocyclic ring comprising 1 nitrogen atom fused to a 5-membered heteroaryl ring comprising 1-2 heteroatoms independently selected from nitrogen, oxygen, and sulfur. In some embodiments of Formula II, Ring A is selected from 2,4,5,6-tetrahydropyrrolo[3,4-c]pyrazolyl and 5,6-dihydro-4H-pyrrolo[3,4-d]thiazolyl.

In some embodiments of Formula II, Ring A is a 6-membered saturated heterocyclic ring comprising 1-2 heteroatoms independently selected from nitrogen, oxygen and sulfur fused to a group independently selected from phenyl and a 5- or 6-membered heteroaryl ring comprising 1-3 heteroatoms independently selected from nitrogen, oxygen, and sulfur. In some embodiments of Formula II, Ring A is 5,6,7,8-tetrahydro-[1,2,4]triazolo[4,3-a]pyrazinyl.

In some embodiments of Formula II, Ring A is selected from

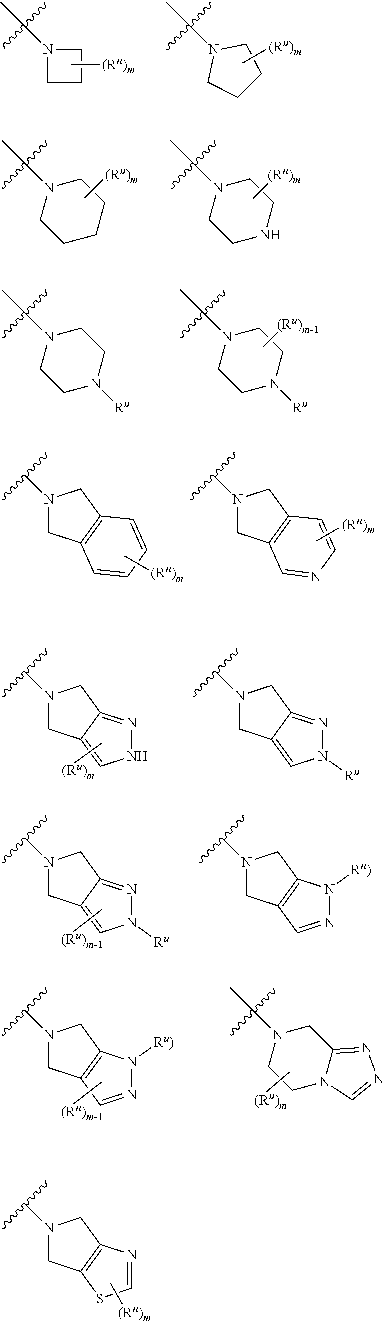

As defined above for Formula II, Ring B is selected from phenyl and a 6-membered heteroaryl ring comprising 1-2 nitrogen atoms. In some embodiments of Formula II, Ring B is phenyl. In some embodiments of Formula II, Ring B is a 6-membered heteroaryl ring comprising 1-2 nitrogen atoms. In some embodiments of Formula II, Ring B is a 6-membered heteroaryl ring comprising 1 nitrogen atom. In some embodiments of Formula II, Ring B is a 6-membered heteroaryl ring comprising 2 nitrogen atoms. In some embodiments of Formula II, Ring B is selected from phenyl, pyridinyl and pyrimidinyl. In some embodiments of Formula II, Ring B is selected from

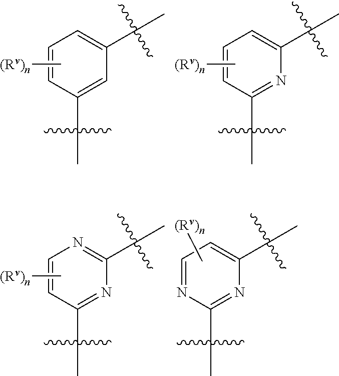

As defined above for Formula II, Ring C is selected from phenyl, a 5- to 6-membered heteroaryl ring comprising 1-3 heteroatoms independently selected from nitrogen, oxygen, and sulfur, and a 9- to 10-membered heteroaryl ring comprising 1-3 heteroatoms independently selected from nitrogen, oxygen, and sulfur. In some embodiments of Formula II, Ring C is phenyl.

In some embodiments of Formula II, Ring C is a 5- to 6-membered heteroaryl ring comprising 1-3 heteroatoms independently selected from nitrogen, oxygen, and sulfur. In some embodiments of Formula II, Ring C is a 5-membered heteroaryl ring comprising 1-3 heteroatoms independently selected from nitrogen, oxygen, and sulfur. In some embodiments of Formula II, Ring C is a 5-membered heteroaryl ring comprising 1-2 heteroatoms independently selected from nitrogen, oxygen, and sulfur. In some embodiments of Formula II, Ring C is pyrazolyl.

In some embodiments of Formula II, Ring C is a 6-membered heteroaryl ring comprising 1-3 nitrogen atoms. In some embodiments of Formula II, Ring C is a 6-membered heteroaryl ring comprising 1-2 nitrogen atoms. In some embodiments of Formula II, Ring C is pyridinyl.

In some embodiments of Formula II, Ring C is a 9- to 10-membered heteroaryl ring comprising 1-3 heteroatoms independently selected from nitrogen, oxygen, and sulfur. In some embodiments of Formula II, Ring C is a 9-membered heteroaryl ring comprising 1-3 heteroatoms independently selected from nitrogen, oxygen, and sulfur. In some embodiments of Formula II, Ring C is a 9-membered heteroaryl ring comprising 1-2 heteroatoms independently selected from nitrogen, oxygen, and sulfur. In some embodiments of Formula II, Ring C is a 9-membered heteroaryl ring comprising 2-3 heteroatoms independently selected from nitrogen, oxygen, and sulfur. In some embodiments of Formula II, Ring C is a 9-membered heteroaryl ring comprising 1-2 nitrogen atoms. In some embodiments of Formula II, Ring C is a 9-membered heteroaryl ring comprising 2-3 nitrogen atoms. In some embodiments of Formula II, Ring C is selected from indazolyl and pyrazolo[3,4-b]pyridinyl.

In some embodiments of Formula II, Ring C is a 10-membered heteroaryl ring comprising 1-3 nitrogen atoms. In some embodiments of Formula II, Ring C is a 10-membered heteroaryl ring comprising 1-2 nitrogen atoms. In some embodiments of Formula II, Ring C is a 10-membered heteroaryl ring comprising 1 nitrogen atom. In some embodiments of Formula II, Ring C is quinolinyl or isoquinolinyl.

In some embodiments of Formula II, Ring C is selected from

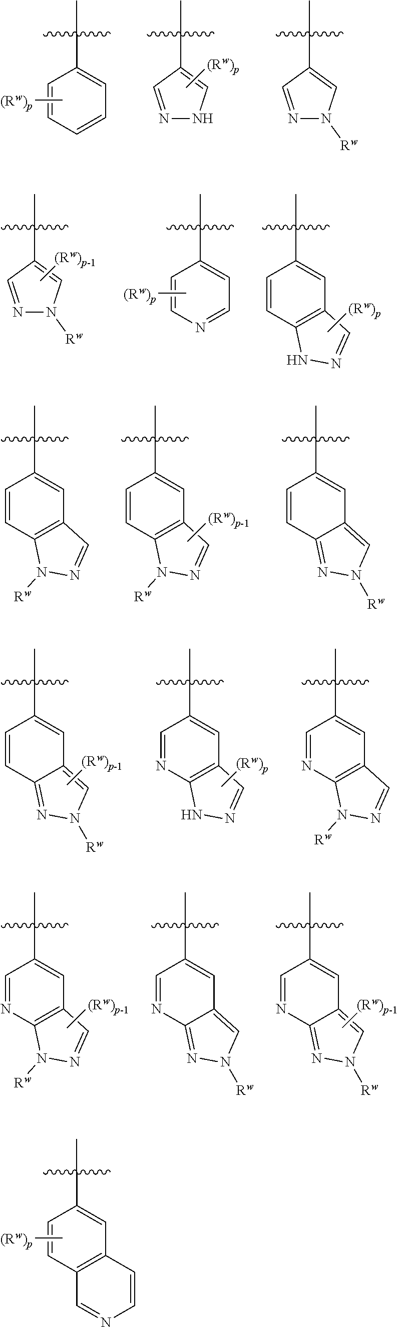

As defined above for Formula II, each R^{u} is independently selected from halogen, OR″, and an optionally substituted group selected from C₁₋₆ aliphatic, phenyl, a 3- to 7-membered saturated or partially unsaturated heterocyclic ring comprising 1-3 heteroatoms independently selected from nitrogen, oxygen, and sulfur, and a 5- to 6-membered heteroaryl ring comprising 1-3 heteroatoms independently selected from nitrogen, oxygen, and sulfur.

In some embodiments of Formula II, R^{u} is independently selected from halogen, OR″, and optionally substituted C₁₋₆ aliphatic.

In some embodiments of Formula II, R^{u} is an optionally substituted group selected from C₁₋₆ aliphatic, phenyl, a 3- to 7-membered saturated or partially unsaturated heterocyclic ring comprising 1-3 heteroatoms independently selected from nitrogen, oxygen, and sulfur, and a 5- to 6-membered heteroaryl ring comprising 1-3 heteroatoms independently selected from nitrogen, oxygen, and sulfur.

In some embodiments of Formula II, R^{u} is an optionally substituted group selected from phenyl, a 3- to 7-membered saturated or partially unsaturated heterocyclic ring comprising 1-3 heteroatoms independently selected from nitrogen, oxygen, and sulfur, and a 5- to 6-membered heteroaryl ring comprising 1-3 heteroatoms independently selected from nitrogen, oxygen, and sulfur.

In some embodiments of Formula II, R^{u} is halogen. In some such embodiments of Formula II, R^{u} is fluoro, chloro, or bromo.

In some embodiments of Formula II, R^{u} is OR″. In some embodiments of Formula II, R^{u} is OH. In some embodiments of Formula II, R^{u} is OR″, wherein R″ is C₁₋₆ aliphatic. In some such embodiments of Formula II, R^{u} is OCH₃.

In some embodiments of Formula II, R^{u} is OR″, wherein R″ is optionally substituted C₁₋₆ aliphatic. In some embodiments of Formula II, R^{u} is OR″, wherein R″ is C₁₋₆ aliphatic optionally substituted with —(CH₂)₀₋₄N(R^{o})₂. In some embodiments of Formula II, R^{u} is OR″, wherein R″ is C₁₋₆ aliphatic optionally substituted with —(CH₂)₀₋₄N(R^{o})₂, and each R^{o} is independently selected from hydrogen and —CH₃. In some embodiments of Formula II, R^{u} is OR″, wherein R″ is C₂₋₄ aliphatic optionally substituted with —(CH₂)₀₋₄N(R^{o})₂. In some embodiments of Formula II, R^{u} is OR″, wherein R″ is C₂₋₄ aliphatic optionally substituted with —(CH₂)₀₋₄N(R^{o})₂, and each R^{o} is independently selected from hydrogen and —CH₃. In some embodiments of Formula II, R^{u} is OR″, wherein R″ is C₂₋₄ aliphatic optionally substituted with —N(R^{o})₂. In some embodiments of Formula II, R^{u} is OR″, wherein R″ is C₂₋₄ aliphatic optionally substituted with —N(R^{o})₂, and each R^{o} is independently selected from hydrogen and —CH₃.

In some embodiments of Formula II, R^{u} is OR″, wherein R″ is C₁₋₆ aliphatic optionally substituted with —(CH₂)₀₋₄OR^{o}. In some embodiments of Formula II, R^{u} is OR″, wherein R″ is C₁₋₆ aliphatic optionally substituted with —(CH₂)₀₋₄OR^{o}, and R^{o} is selected from hydrogen and —CH₃. In some embodiments of Formula II, R^{u} is OR″, wherein R″ is C₂₋₄ aliphatic optionally substituted with —(CH₂)₀₋₄OR^{o}. In some embodiments of Formula II, R^{u} is OR″, wherein R″ is C₂₋₄ aliphatic optionally substituted with —(CH₂)₀₋₄OR^{o}₂, and R^{o} is selected from hydrogen and —CH₃. In some embodiments of Formula II, R^{u} is OR″, wherein R″ is C₂₋₄ aliphatic optionally substituted with —OR^{o}. In some embodiments of Formula II, R^{u} is OR″, wherein R″ is C₂₋₄ aliphatic optionally substituted with —OR^{o} and R^{o} is selected from hydrogen and —CH₃.

In some embodiments of Formula II, R^{u} is OR″, wherein R″ is C₁₋₆ aliphatic optionally substituted with —(CH₂)₀₋₄C(O)N(R^{o})₂. In some embodiments of Formula II, R^{u} is OR″, wherein R″ is C₁₋₆ aliphatic optionally substituted with —(CH₂)₀₋₄C(O)N(R^{o})₂, and each R^{o} is independently selected from hydrogen and C₁₋₃ aliphatic. In some embodiments of Formula II, R^{u} is OR″, wherein R″ is C₁₋₃ aliphatic optionally substituted with —(CH₂)₀₋₄C(O)N(R^{o})₂. In some embodiments of Formula II, R^{u} is OR″, wherein R″ is C₁₋₃ aliphatic optionally substituted with —(CH₂)₀₋₄C(O)N(R^{o})₂, and each R^{o} is independently selected from hydrogen and C₁₋₃ aliphatic. In some embodiments of Formula II, R^{u} is OR″, wherein R″ is C₁₋₃ aliphatic optionally substituted with —C(O)N(R^{o})₂. In some embodiments of Formula II, R^{u} is OR″, wherein R″ is C₁₋₃ aliphatic optionally substituted with —C(O)N(R^{o})₂, and each R^{o} is independently selected from hydrogen and C₁₋₃ aliphatic.

In some embodiments of Formula II, R^{u} is OR″, wherein R″ is C₁₋₆ aliphatic optionally substituted with —(CH₂)₀₋₄R^{o}. In some embodiments of Formula II, R^{u} is OR″, wherein R″ is C₁₋₆ aliphatic optionally substituted with —(CH₂)₀₋₄R^{o}, and R^{o} is a 5- to 6-membered saturated heterocyclic ring having 1-2 heteroatoms independently selected from nitrogen, oxygen, and sulfur. In some embodiments of Formula II, R^{u} is OR″, wherein R″ is C₁₋₃ aliphatic optionally substituted with —(CH₂)₀₋₄R^{o}, and R^{o} is a 5- to 6-membered saturated heterocyclic ring having 1-2 heteroatoms independently selected from nitrogen, oxygen, and sulfur. In some embodiments of Formula II, R^{u} is OR″, wherein R″ is C₁₋₃ aliphatic optionally substituted with —(CH₂)₀₋₄R^{o}, and R^{o} is a 6-membered saturated heterocyclic ring having 1-2 heteroatoms independently selected from nitrogen, oxygen, and sulfur. In some embodiments of Formula II, R^{u} is OR″, wherein R″ is C₁₋₃ aliphatic optionally substituted with —R^{o}, and R^{o} is selected from morpholinyl and piperazinyl.

In some embodiments of Formula II, R^{u} is optionally substituted C₁₋₆ aliphatic. In some embodiments of Formula II, R^{u} is C₁₋₆ aliphatic. In some embodiments of Formula II, R^{u} is C₁₋₃ aliphatic. In some embodiments of Formula II, R^{U} is selected from —CH₃, CH₂CH₃, and —CH₂CH₂CH₃.

In some embodiments of Formula II, R^{u} is C₁₋₆ aliphatic optionally substituted with halogen. In some embodiments of Formula II, R^{u} is C₁₋₃ aliphatic optionally substituted with halogen. In some embodiments of Formula II, R^{u} is —CF₃.

In some embodiments of Formula II, R^{u} is optionally substituted phenyl. In some embodiments of Formula II, R^{u} is phenyl optionally substituted with halogen, —C≡N, —(CH₂)₀₋₄OR^{o}, or —(CH₂)₀₋₄C(O)OR^{o}. In some embodiments of Formula II, R^{u} is phenyl substituted with a group selected from halogen, —C≡N, —OR^{o}, or —C(O)OR^{o}, wherein R^{o} is selected from hydrogen and —CH₃.

In some embodiments of Formula II, R^{u} is an optionally substituted 3- to 7-membered saturated or partially unsaturated heterocyclic ring comprising 1-3 heteroatoms independently selected from nitrogen, oxygen, and sulfur. In some embodiments of Formula II, R^{u} is an optionally substituted 4- to 6-membered saturated or partially unsaturated heterocyclic ring comprising 1-2 heteroatoms independently selected from nitrogen, oxygen, and sulfur. In some embodiments of Formula II, R^{u} is an optionally substituted 4- to 6-membered saturated or partially unsaturated heterocyclic ring comprising 1-2 heteroatoms independently selected from nitrogen, oxygen, and sulfur. In some embodiments of Formula II, R^{u} is an optionally substituted 4- to 6-membered saturated heterocyclic ring comprising 1-2 heteroatoms independently selected from nitrogen, oxygen, and sulfur. In some embodiments of Formula II, R^{u} is an optionally substituted 6-membered saturated heterocyclic ring comprising 1-2 heteroatoms independently selected from nitrogen, oxygen, and sulfur. In some embodiments of Formula II, R^{u} is a 6-membered saturated heterocyclic ring comprising 1-2 heteroatoms independently selected from nitrogen, oxygen, and sulfur, optionally substituted with —(CH₂)₀₋₄R^{o}. In some embodiments of Formula II, R^{u} is a 6-membered saturated heterocyclic ring comprising 1-2 heteroatoms independently selected from nitrogen, oxygen, and sulfur, optionally substituted with —(CH₂)₀₋₄R^{o}, wherein R^{o} is C₁₋₆ aliphatic substituted with —(CH₂)₀₋₂OR^{●}. In some embodiments of Formula II, R″ is a 6-membered saturated heterocyclic ring comprising 1-2 heteroatoms independently selected from nitrogen, oxygen, and sulfur, optionally substituted with —R^{o}, wherein R^{o} is C₁₋₃ aliphatic optionally substituted with —(CH₂)₀₋₂OR^{●}. In some embodiments of Formula II, R^{u} is selected from piperidinyl, morphonlinyl, and piperazinyl, each of which may be optionally substituted with —(CH₂)₀₋₄R^{o}, wherein R^{o} is C₁₋₆ aliphatic optionally substituted with —(CH₂)₀₋₂OR^{●}.

In some embodiments of Formula II, R^{u} is an optionally substituted 5- to 6-membered heteroaryl ring comprising 1-3 heteroatoms independently selected from nitrogen, oxygen, and sulfur. In some embodiments of Formula II, R^{u} is an optionally substituted 5-membered heteroaryl ring comprising 1-3 heteroatoms independently selected from nitrogen, oxygen, and sulfur. In some embodiments of Formula II, R^{u} is an optionally substituted 5-membered heteroaryl ring comprising 1-2 heteroatoms independently selected from nitrogen, oxygen, and sulfur. In some embodiments of Formula II, R^{u} is selected from imidazolyl and thiazolyl.

In some embodiments of Formula II, R^{u} is selected from halogen, —OH, —OCH₃, —CH₃, —CH₂CH₂CH₃, —CF₃, phenyl,

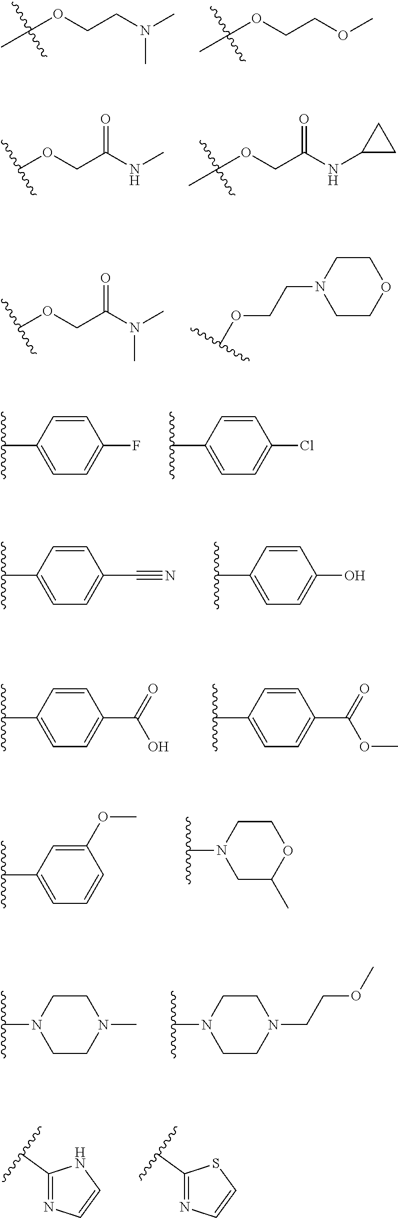

As defined above for Formula II, each R^{v} is independently selected from halogen, CN, CO₂R″, C(O)NR″₂, NR″₂, OR″, SR″, and optionally substituted C₁₋₆ aliphatic. In some embodiments of Formula II, R^{v} is halogen.

As defined above for Formula II, each R^{w} is independently selected from halogen, CN, CO₂R″, C(O)NR″₂, NR″₂, OR″, SR″, and optionally substituted C₁₋₆ aliphatic, or two independent occurrences of R^{w}, taken together with their intervening atom(s), form an optionally substituted 5-membered heterocyclic ring comprising 1-2 heteroatoms independently selected from nitrogen, oxygen, and sulfur. In some embodiments of Formula II, R^{w} is selected from halogen, CN, CO₂R″, C(O)NR″₂, NR″₂, OR″, SR″, oxo, and optionally substituted C₁₋₆ aliphatic.

In some embodiments of Formula II, R^{w} is —C≡N. In some embodiments of Formula II, R^{w} is halogen.

In some embodiments of Formula II, R^{w} is CO₂R″. In some embodiments of Formula II, R^{w} is CO₂R″, wherein R″ is selected from hydrogen and C₁₋₆ aliphatic. In some embodiments of Formula II, R^{w} is CO₂R″, wherein R″ is selected from hydrogen and C₁₋₃ aliphatic. In some embodiments of Formula II, R^{w} is CO₂R″, wherein R″ is selected from hydrogen and CH₃.

In some embodiments of Formula II, R^{w} is C(O)NR″₂. In some embodiments of Formula II, R^{w} is C(O)NR″₂, wherein R″ is selected from hydrogen and C₁₋₆ aliphatic. In some embodiments of Formula II, R^{w} is C(O)NR″₂, wherein R^{w} is selected from hydrogen and C₁₋₃ aliphatic. In some embodiments of Formula II, R^{w} is C(O)NR″₂, wherein R″ is selected from hydrogen and CH₃.

In some embodiments of Formula II, R^{w} is optionally substituted C₁₋₆ aliphatic. In some embodiments of Formula II, R^{w} is C₁₋₆ aliphatic optionally substituted with —OP(O)(OR^{o})₂. In some embodiments of Formula II, R^{w} is C₁₋₆ aliphatic optionally substituted with —OP(O)(OR^{o})₂, wherein R^{o} is selected from hydrogen and C₁₋₃ aliphatic. In some embodiments of Formula II, R^{w} is C₁₋₆ aliphatic optionally substituted with —OP(O)(OR^{o})₂, wherein R^{o} is selected from hydrogen and CH₃.

In some embodiments of Formula II, R^{w} is NR″₂. In some embodiments of Formula II, R^{w} is NH₂.

In some embodiments of Formula II, each R^{w} is independently selected from halogen, CN, CO₂R″, C(O)NR″₂, NR″₂, OR″, SR″, and optionally substituted C₁₋₆ aliphatic, wherein two independent occurrences of R^{w}, taken together with their intervening atom(s), form an optionally substituted 5-membered heterocyclic ring comprising 1-2 heteroatoms independently selected from nitrogen, oxygen, and sulfur. In some embodiments of Formula II, two independent occurrences of R^{w}, taken together with their intervening atom(s), form an optionally substituted 5-membered heterocyclic ring comprising 1-2 heteroatoms independently selected from nitrogen, oxygen, and sulfur. In some such embodiments of Formula II, two independent occurrences of R^{w}, taken together with their intervening atom(s), form a pyrrolidin-2-onyl ring.

In some embodiments of Formula II, R^{w} is selected from halogen, —CH₃, —C≡N, —NH₂, —CO₂H, —CO_{2H}, —CO₂CH₃, —C(O)NHCH₃, and —CH₂OP(O)(OR^{o})₂.

As defined above for Formula II, each R″ is independently selected from hydrogen or an optionally substituted group selected from C₁₋₆ aliphatic, phenyl, and a 3- to 7-membered saturated or partially unsaturated heterocyclic ring comprising 1-3 heteroatoms independently selected from nitrogen, oxygen, and sulfur. In some embodiments of Formula II, R″ is hydrogen. In some embodiments of Formula II, each R″ is independently selected from hydrogen or an optionally substituted group selected from C₁₋₆ aliphatic, phenyl, and a 3- to 7-membered saturated or partially unsaturated heterocyclic ring comprising 1-3 heteroatoms independently selected from nitrogen, oxygen, and sulfur.

In some embodiments of Formula II, each R″ is hydrogen.

In some embodiments of Formula II, R″ is optionally substituted C₁₋₆ aliphatic. In some embodiments of Formula II, R″ is C₁₋₆ aliphatic. In some embodiments of Formula II, R″ is C₁₋₃ aliphatic. In some embodiments of Formula II, R″ is selected from hydrogen, CH₃, CH₂CH₃, and

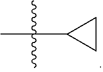 [SMILES: *C1CC1]

In some embodiments of Formula II, R″ is C₁₋₆ aliphatic optionally substituted with a group selected from —(CH₂)₀₋₄R^{o}, —(CH₂)₀₋₄OR^{o}, —(CH₂)₀₋₄N(R^{o})₂, and —(CH₂)₀₋₄C(O)N(R^{o})₂. In some embodiments of Formula II, R″ is C₁₋₆ aliphatic optionally substituted with a group selected from —R^{o}, —OR^{o}, —N(R^{o})₂, and —C(O)N(R^{o})₂.

In some embodiments of Formula II, the R″ group of R^{u} is selected from hydrogen, CH₃, —CH₂CH₂R^{o}, —CH₂CH₂OR^{o}, —CH₂CH₂N(R^{o})₂, and —CH₂C(O)N(R^{o})₂.

In some embodiments of Formula II, the R″ group of R^{w} is selected from hydrogen and CH₃.

As defined above for Formula II, each of m, n, and p is independently 0-4. In some embodiments of Formula II, m is 0. In some embodiments of Formula II, m is 1. In some embodiments of Formula II, n is 0. In some embodiments of Formula II, n is 1. In some embodiments of Formula II, p is 0. In some embodiments of Formula II, p is 1. In some embodiments of Formula II, p is 2.

In some embodiments, the present disclosure provides a compound of Formula II-a:

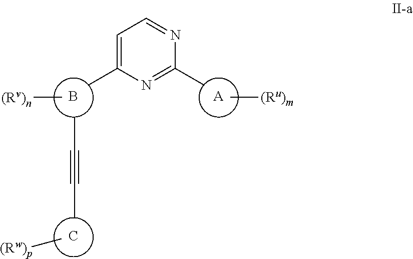

or a pharmaceutically acceptable salt thereof, wherein each of Ring A, Ring B, Ring C, R^{u}, R^{v}, R^{w}, m, n, and p is as described above and defined herein for Formula II.

In some embodiments, the present disclosure provides a compound of Formula II-b.

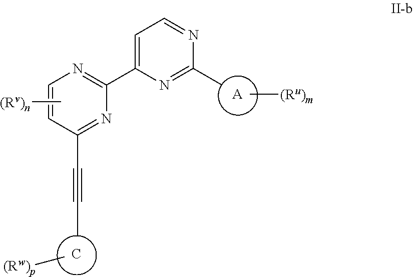

or a pharmaceutically acceptable salt thereof, wherein each of Ring A, Ring C, R^{u}, R^{v}, R^{w}, m, n, and p is as described above and defined herein for Formula II.

In some embodiments, the present disclosure provides a compound of Formula II-c:

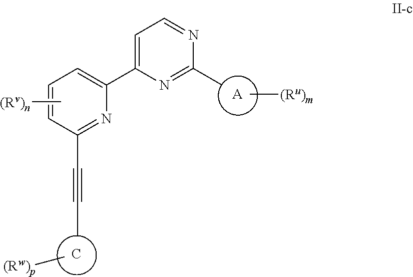

or a pharmaceutically acceptable salt thereof, wherein each of Ring A, Ring C, R^{u}, R^{v}, R^{w}, m, n, and p is as described above and defined herein for Formula II.

In some embodiments, the present disclosure provides a compound of Formula II-d:

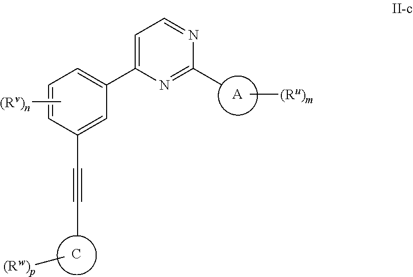

or a pharmaceutically acceptable salt thereof, wherein each of Ring A, Ring C, R^{u}, R^{v}, R^{w}, m, n, and p is as described above and defined herein for Formula II.

In some embodiments, the present disclosure provides a compound of Formula II-e:

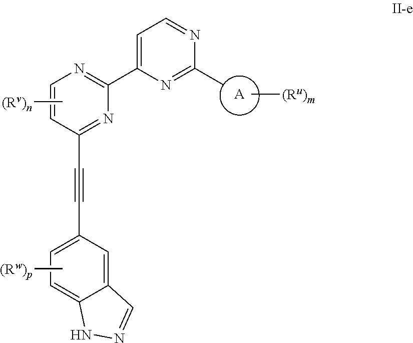

or a pharmaceutically acceptable salt thereof, wherein each of Ring A, R^{u}, R^{v}, R^{w}, m, n, and p is as described above and defined herein for Formula II.

In some embodiments, the present disclosure provides a compound of Formula II-f:

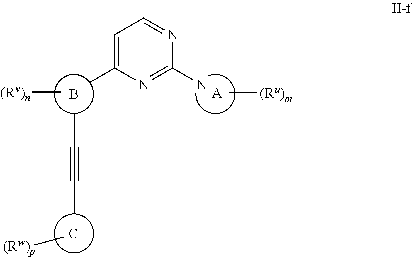

or a pharmaceutically acceptable salt thereof, wherein each of Ring A, Ring B, Ring C, R^{u}, R^{v}, R^{w}, m, n, and p is as described above and defined herein for Formula II.

In some embodiments, the present disclosure provides a compound of Formula II-g:

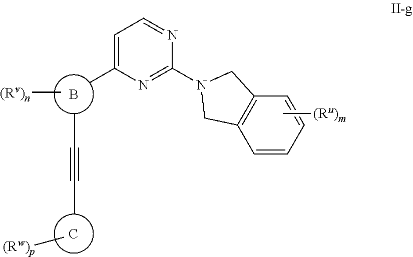

or a pharmaceutically acceptable salt thereof, wherein each of Ring B, Ring C, R^{u}, R^{v}, R^{w}, m, n, and p is as described above and defined herein for Formula II.

In some embodiments, the present disclosure provides a compound of Formula II-h:

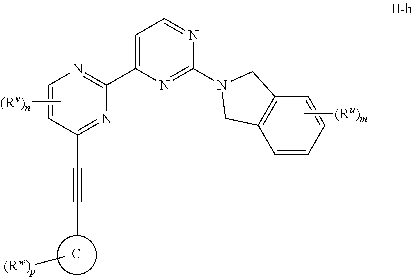

or a pharmaceutically acceptable salt thereof, wherein each of Ring C, R^{u}, R^{v}, R^{w}, m, n, and p is as described above and defined herein for Formula II.

In some embodiments, the present disclosure provides a compound selected from the group consisting of:

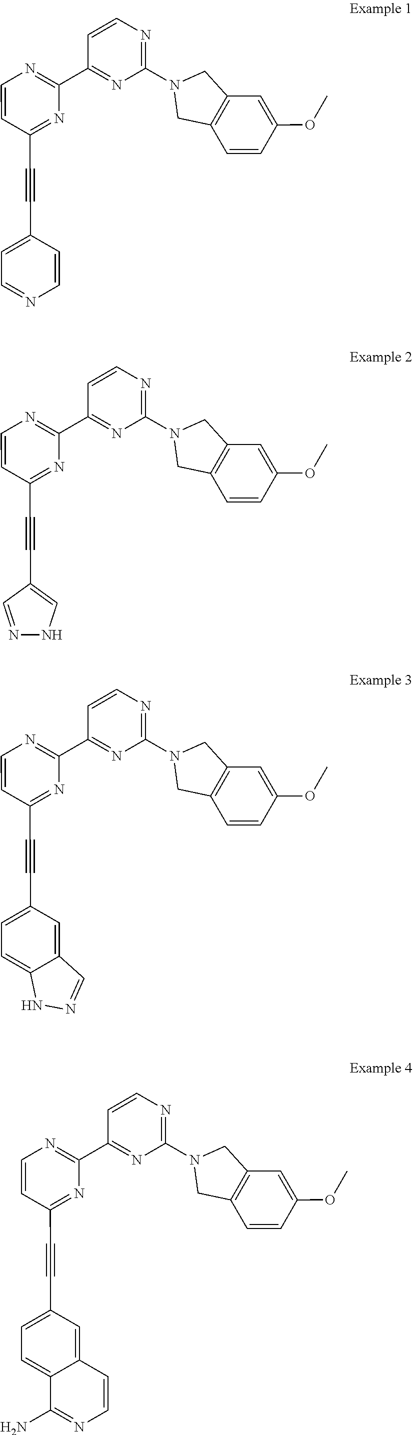 [SMILES: COc1ccc2c(c1)CN(c1nccc(-c3nccc(C#Cc4ccncc4)n3)n1)C2 ; COc1ccc2c(c1)CN(c1nccc(-c3nccc(C#Cc4cn[nH]c4)n3)n1)C2 ; COc1ccc2c(c1)CN(c1nccc(-c3nccc(C#Cc4ccc5[nH]ncc5c4)n3)n1)C2 ; COc1ccc2c(c1)CN(c1nccc(-c3nccc(C#Cc4ccc5c(N)nccc5c4)n3)n1)C2]

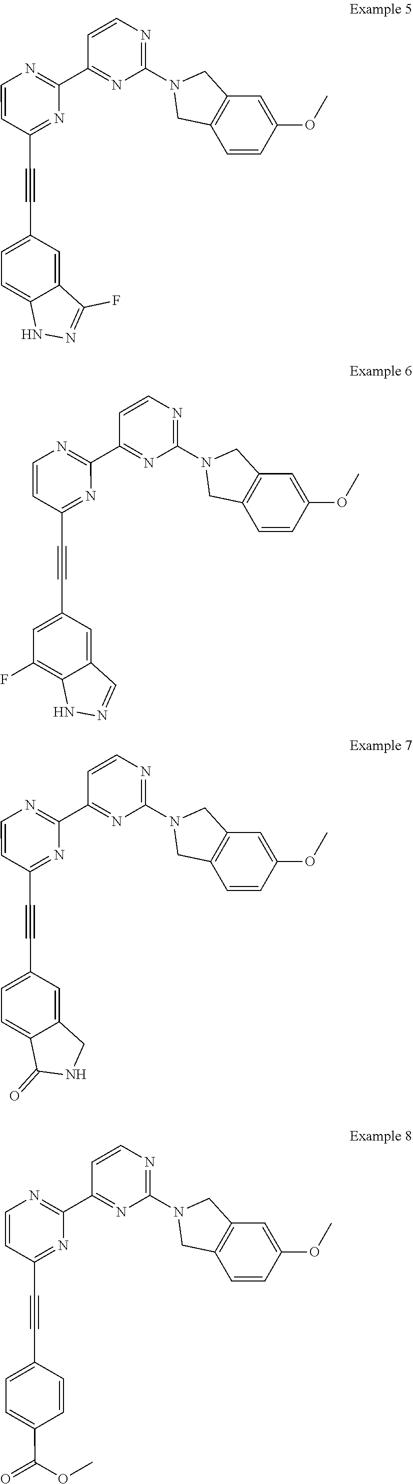

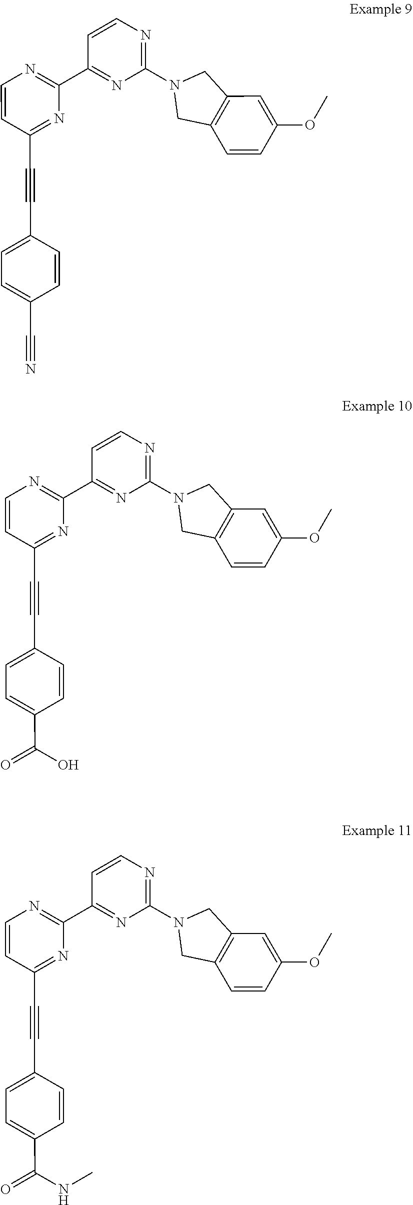 [SMILES: COc1ccc2c(c1)CN(c1nccc(-c3nccc(C#Cc4ccc(C#N)cc4)n3)n1)C2 ; COc1ccc2c(c1)CN(c1nccc(-c3nccc(C#Cc4ccc(C(=O)O)cc4)n3)n1)C2 ; CNC(=O)c1ccc(C#Cc2ccnc(-c3ccnc(N4Cc5ccc(OC)cc5C4)n3)n2)cc1]

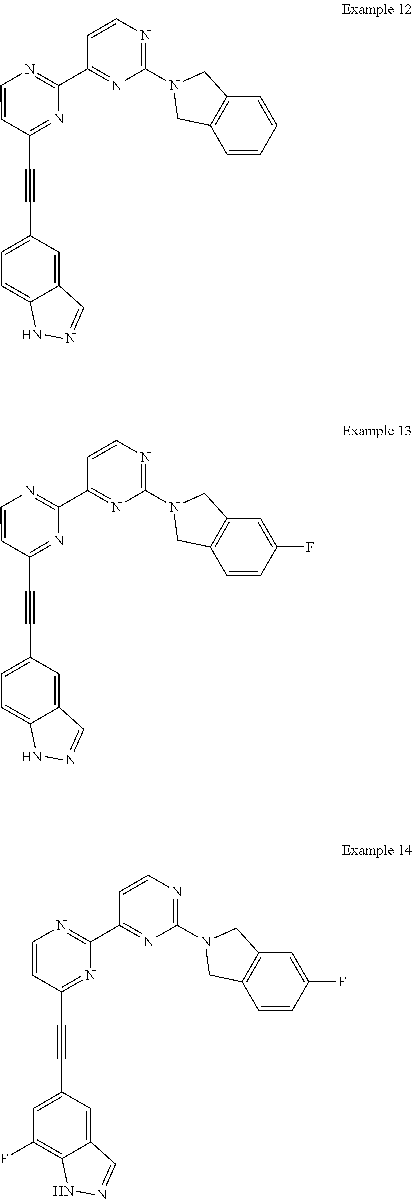 [SMILES: C(#Cc1ccnc(-c2ccnc(N3Cc4ccccc4C3)n2)n1)c1ccc2[nH]ncc2c1 ; Fc1ccc2c(c1)CN(c1nccc(-c3nccc(C#Cc4ccc5[nH]ncc5c4)n3)n1)C2 ; Fc1ccc2c(c1)CN(c1nccc(-c3nccc(C#Cc4cc(F)c5[nH]ncc5c4)n3)n1)C2]

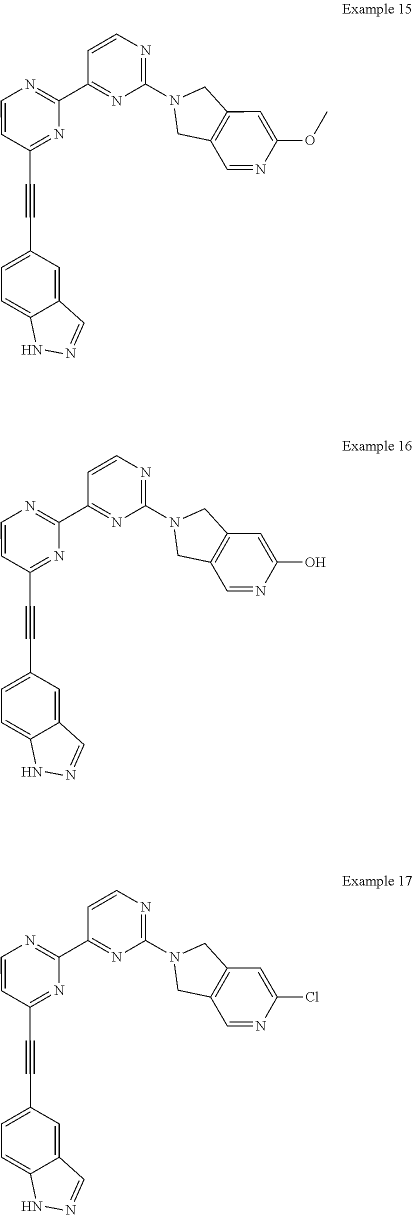 [SMILES: COc1cc2c(cn1)CN(c1nccc(-c3nccc(C#Cc4ccc5[nH]ncc5c4)n3)n1)C2 ; Oc1cc2c(cn1)CN(c1nccc(-c3nccc(C#Cc4ccc5[nH]ncc5c4)n3)n1)C2 ; Clc1cc2c(cn1)CN(c1nccc(-c3nccc(C#Cc4ccc5[nH]ncc5c4)n3)n1)C2]

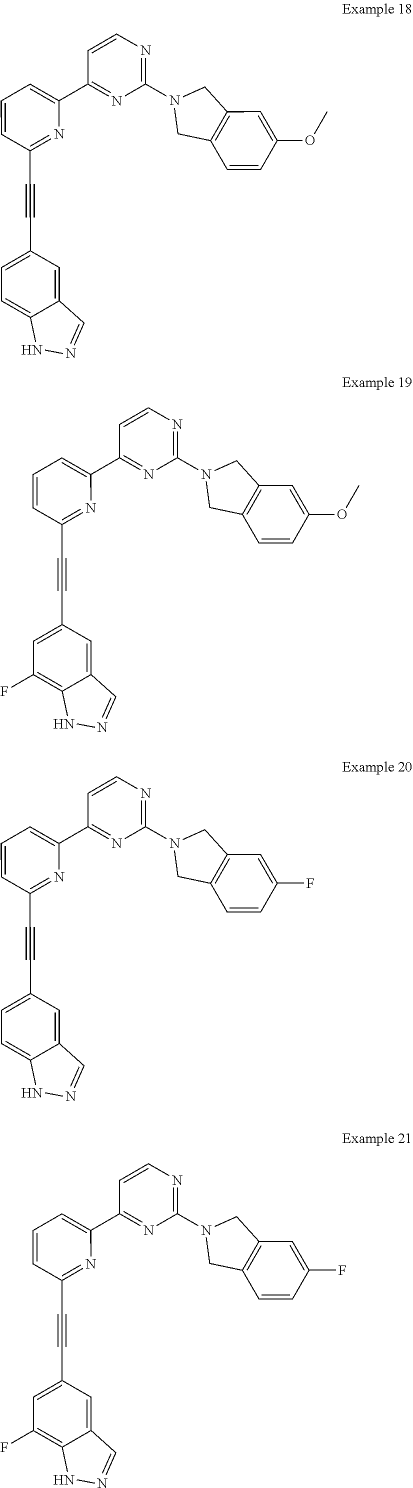 [SMILES: COc1ccc2c(c1)CN(c1nccc(-c3cccc(C#Cc4ccc5[nH]ncc5c4)n3)n1)C2 ; COc1ccc2c(c1)CN(c1nccc(-c3cccc(C#Cc4cc(F)c5[nH]ncc5c4)n3)n1)C2 ; Fc1ccc2c(c1)CN(c1nccc(-c3cccc(C#Cc4ccc5[nH]ncc5c4)n3)n1)C2 ; Fc1ccc2c(c1)CN(c1nccc(-c3cccc(C#Cc4cc(F)c5[nH]ncc5c4)n3)n1)C2]

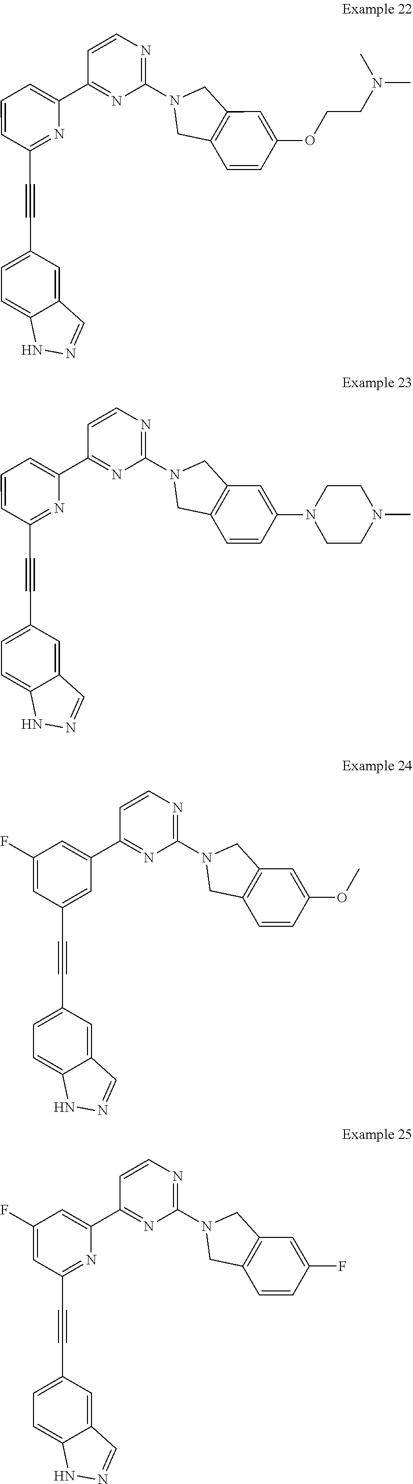 [SMILES: CN(C)CCOc1ccc2c(c1)CN(c1nccc(-c3cccc(C#Cc4ccc5[nH]ncc5c4)n3)n1)C2 ; CN1CCN(c2ccc3c(c2)CN(c2nccc(-c4cccc(C#Cc5ccc6[nH]ncc6c5)n4)n2)C3)CC1 ; COc1ccc2c(c1)CN(c1nccc(-c3cc(F)cc(C#Cc4ccc5[nH]ncc5c4)c3)n1)C2 ; Fc1cc(C#Cc2ccc3[nH]ncc3c2)nc(-c2ccnc(N3Cc4ccc(F)cc4C3)n2)c1]

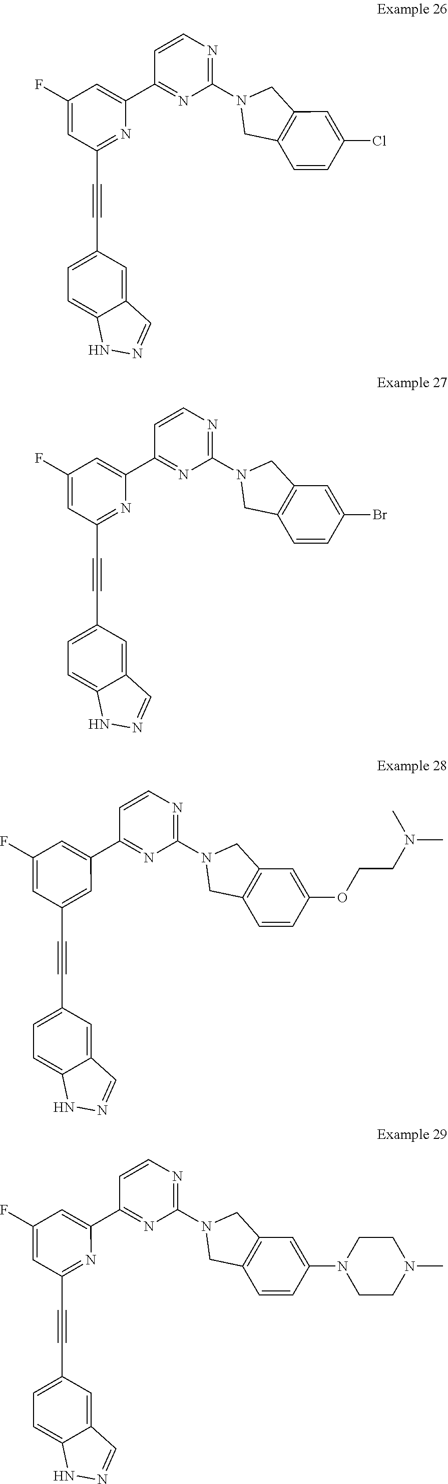 [SMILES: Fc1cc(C#Cc2ccc3[nH]ncc3c2)nc(-c2ccnc(N3Cc4ccc(Cl)cc4C3)n2)c1 ; Fc1cc(C#Cc2ccc3[nH]ncc3c2)nc(-c2ccnc(N3Cc4ccc(Br)cc4C3)n2)c1 ; CN(C)CCOc1ccc2c(c1)CN(c1nccc(-c3cc(F)cc(C#Cc4ccc5[nH]ncc5c4)c3)n1)C2 ; CN1CCN(c2ccc3c(c2)CN(c2nccc(-c4cc(F)cc(C#Cc5ccc6[nH]ncc6c5)n4)n2)C3)CC1]

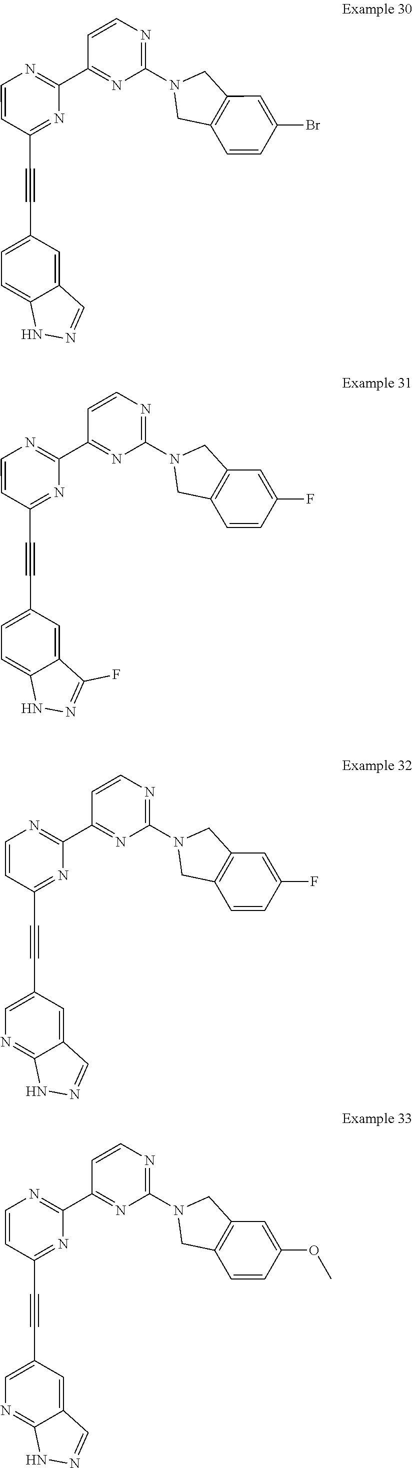 [SMILES: Brc1ccc2c(c1)CN(c1nccc(-c3nccc(C#Cc4ccc5[nH]ncc5c4)n3)n1)C2 ; Fc1ccc2c(c1)CN(c1nccc(-c3nccc(C#Cc4ccc5[nH]nc(F)c5c4)n3)n1)C2 ; Fc1ccc2c(c1)CN(c1nccc(-c3nccc(C#Cc4cnc5[nH]ncc5c4)n3)n1)C2 ; COc1ccc2c(c1)CN(c1nccc(-c3nccc(C#Cc4cnc5[nH]ncc5c4)n3)n1)C2]

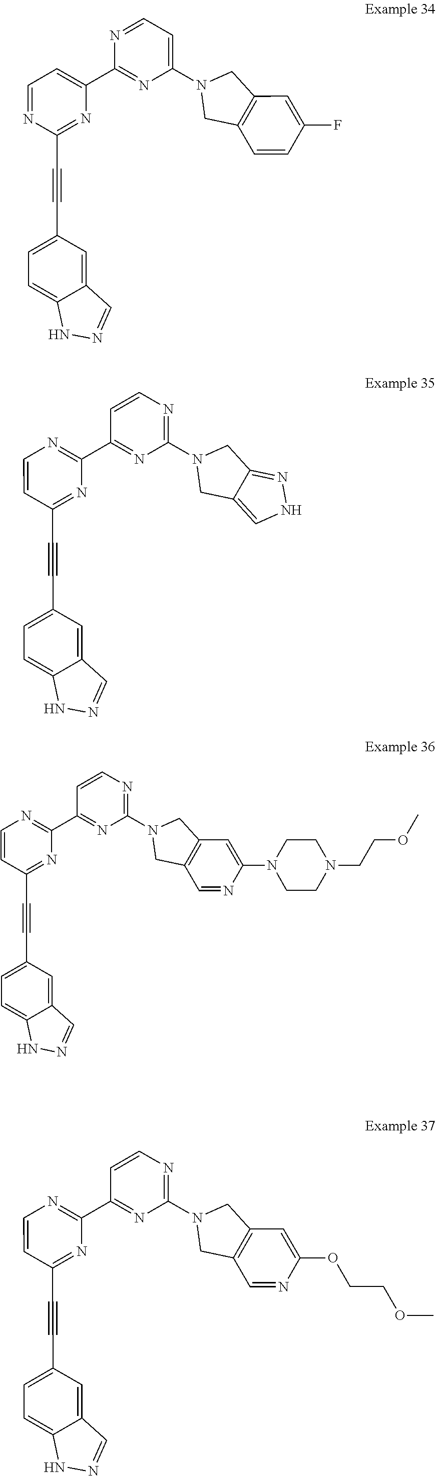 [SMILES: Fc1ccc2c(c1)CN(c1ccnc(-c3ccnc(C#Cc4ccc5[nH]ncc5c4)n3)n1)C2 ; C(#Cc1ccnc(-c2ccnc(N3Cc4c[nH]nc4C3)n2)n1)c1ccc2[nH]ncc2c1 ; COCCN1CCN(c2cc3c(cn2)CN(c2nccc(-c4nccc(C#Cc5ccc6[nH]ncc6c5)n4)n2)C3)CC1 ; COCCOc1cc2c(cn1)CN(c1nccc(-c3nccc(C#Cc4ccc5[nH]ncc5c4)n3)n1)C2]

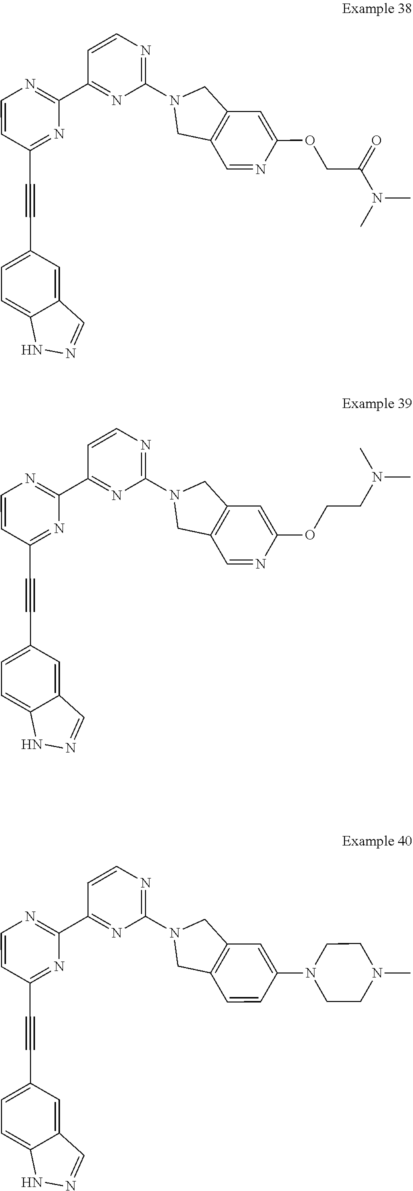 [SMILES: CN(C)C(=O)COc1cc2c(cn1)CN(c1nccc(-c3nccc(C#Cc4ccc5[nH]ncc5c4)n3)n1)C2 ; CN(C)CCOc1cc2c(cn1)CN(c1nccc(-c3nccc(C#Cc4ccc5[nH]ncc5c4)n3)n1)C2 ; CN1CCN(c2ccc3c(c2)CN(c2nccc(-c4nccc(C#Cc5ccc6[nH]ncc6c5)n4)n2)C3)CC1]

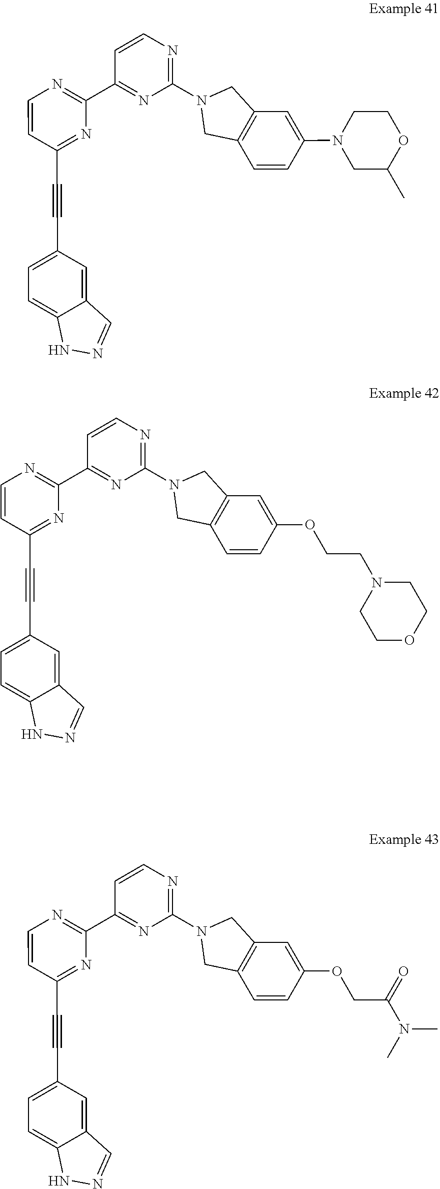 [SMILES: CC1CN(c2ccc3c(c2)CN(c2nccc(-c4nccc(C#Cc5ccc6[nH]ncc6c5)n4)n2)C3)CCO1 ; C(#Cc1ccnc(-c2ccnc(N3Cc4ccc(OCCN5CCOCC5)cc4C3)n2)n1)c1ccc2[nH]ncc2c1 ; CN(C)C(=O)COc1ccc2c(c1)CN(c1nccc(-c3nccc(C#Cc4ccc5[nH]ncc5c4)n3)n1)C2]

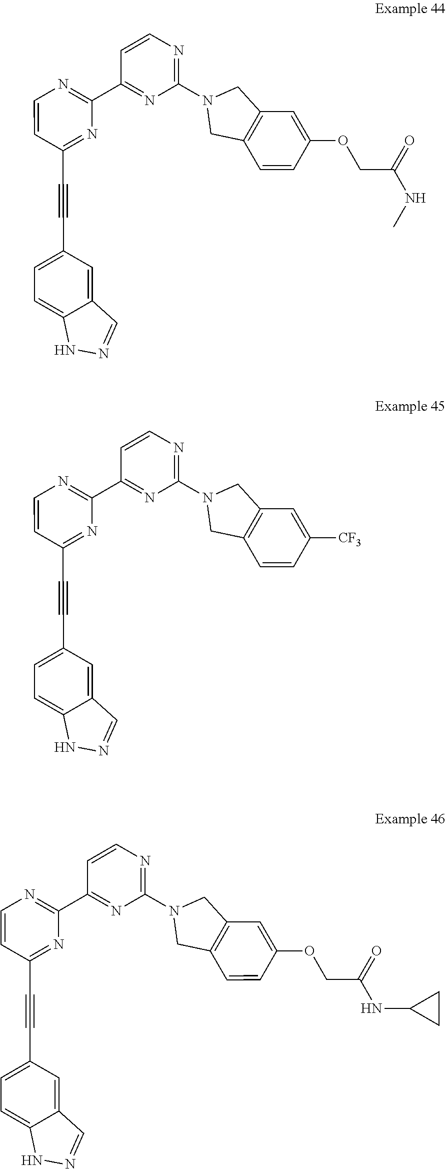 [SMILES: CNC(=O)COc1ccc2c(c1)CN(c1nccc(-c3nccc(C#Cc4ccc5[nH]ncc5c4)n3)n1)C2 ; FC(F)(F)c1ccc2c(c1)CN(c1nccc(-c3nccc(C#Cc4ccc5[nH]ncc5c4)n3)n1)C2 ; O=C(COc1ccc2c(c1)CN(c1nccc(-c3nccc(C#Cc4ccc5[nH]ncc5c4)n3)n1)C2)NC1CC1]

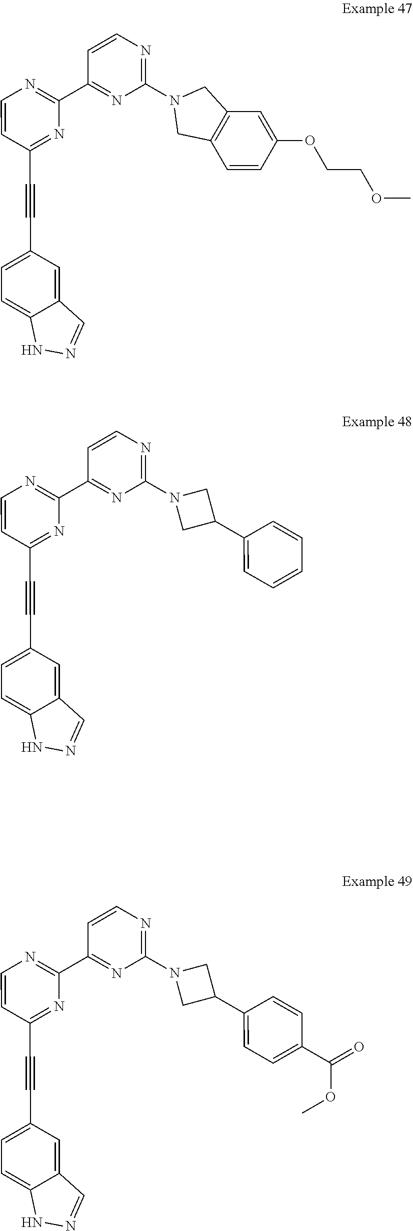 [SMILES: COCCOc1ccc2c(c1)CN(c1nccc(-c3nccc(C#Cc4ccc5[nH]ncc5c4)n3)n1)C2 ; C(#Cc1ccnc(-c2ccnc(N3CC(c4ccccc4)C3)n2)n1)c1ccc2[nH]ncc2c1 ; COC(=O)c1ccc(C2CN(c3nccc(-c4nccc(C#Cc5ccc6[nH]ncc6c5)n4)n3)C2)cc1]

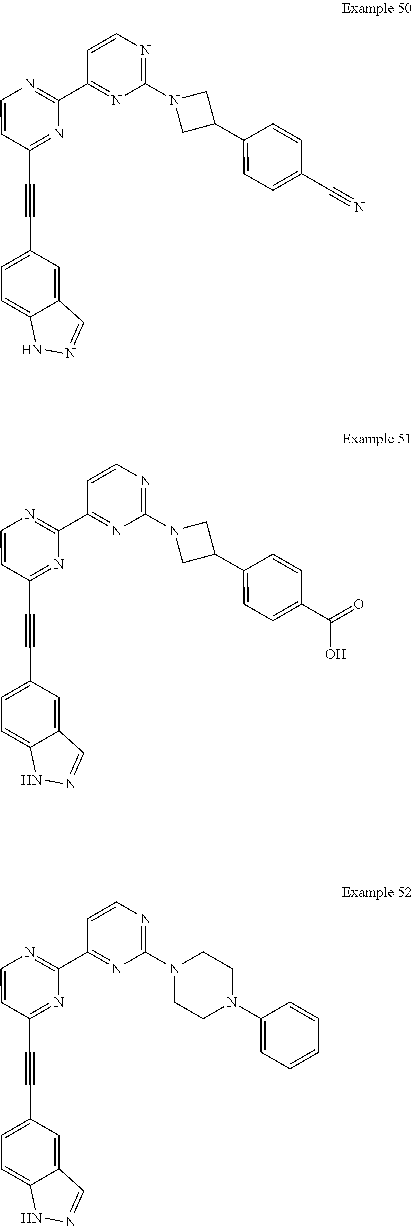 [SMILES: N#Cc1ccc(C2CN(c3nccc(-c4nccc(C#Cc5ccc6[nH]ncc6c5)n4)n3)C2)cc1 ; O=C(O)c1ccc(C2CN(c3nccc(-c4nccc(C#Cc5ccc6[nH]ncc6c5)n4)n3)C2)cc1 ; C(#Cc1ccnc(-c2ccnc(N3CCN(c4ccccc4)CC3)n2)n1)c1ccc2[nH]ncc2c1]

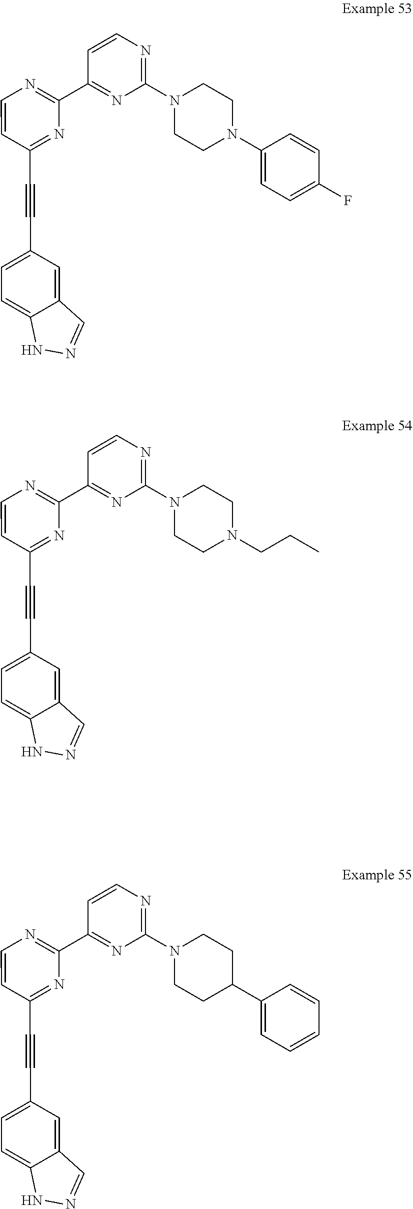 [SMILES: Fc1ccc(N2CCN(c3nccc(-c4nccc(C#Cc5ccc6[nH]ncc6c5)n4)n3)CC2)cc1 ; CCCN1CCN(c2nccc(-c3nccc(C#Cc4ccc5[nH]ncc5c4)n3)n2)CC1 ; C(#Cc1ccnc(-c2ccnc(N3CCC(c4ccccc4)CC3)n2)n1)c1ccc2[nH]ncc2c1]

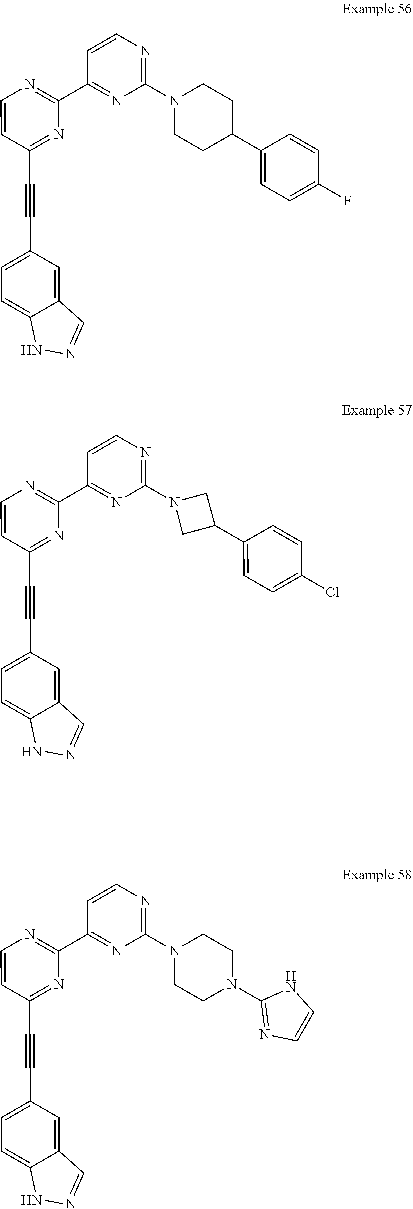 [SMILES: Fc1ccc(C2CCN(c3nccc(-c4nccc(C#Cc5ccc6[nH]ncc6c5)n4)n3)CC2)cc1 ; Clc1ccc(C2CN(c3nccc(-c4nccc(C#Cc5ccc6[nH]ncc6c5)n4)n3)C2)cc1 ; C(#Cc1ccnc(-c2ccnc(N3CCN(c4ncc[nH]4)CC3)n2)n1)c1ccc2[nH]ncc2c1]

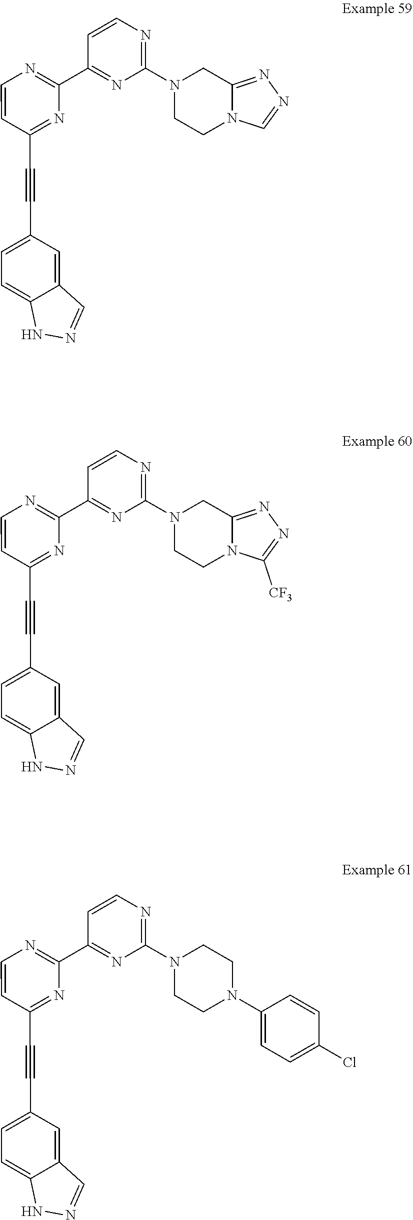 [SMILES: C(#Cc1ccnc(-c2ccnc(N3CCn4cnnc4C3)n2)n1)c1ccc2[nH]ncc2c1 ; FC(F)(F)c1nnc2n1CCN(c1nccc(-c3nccc(C#Cc4ccc5[nH]ncc5c4)n3)n1)C2 ; Clc1ccc(N2CCN(c3nccc(-c4nccc(C#Cc5ccc6[nH]ncc6c5)n4)n3)CC2)cc1]

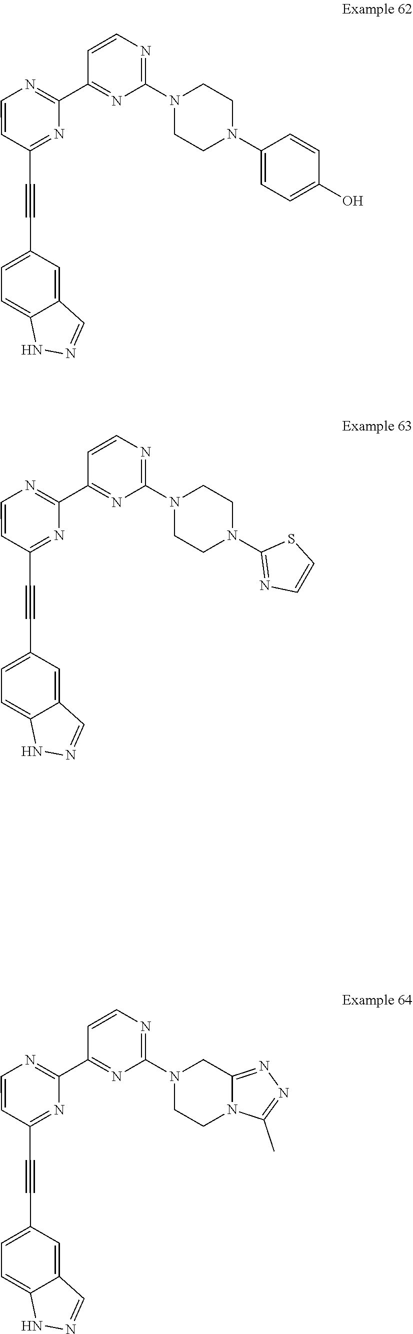 [SMILES: Oc1ccc(N2CCN(c3nccc(-c4nccc(C#Cc5ccc6[nH]ncc6c5)n4)n3)CC2)cc1 ; C(#Cc1ccnc(-c2ccnc(N3CCN(c4nccs4)CC3)n2)n1)c1ccc2[nH]ncc2c1 ; Cc1nnc2n1CCN(c1nccc(-c3nccc(C#Cc4ccc5[nH]ncc5c4)n3)n1)C2]

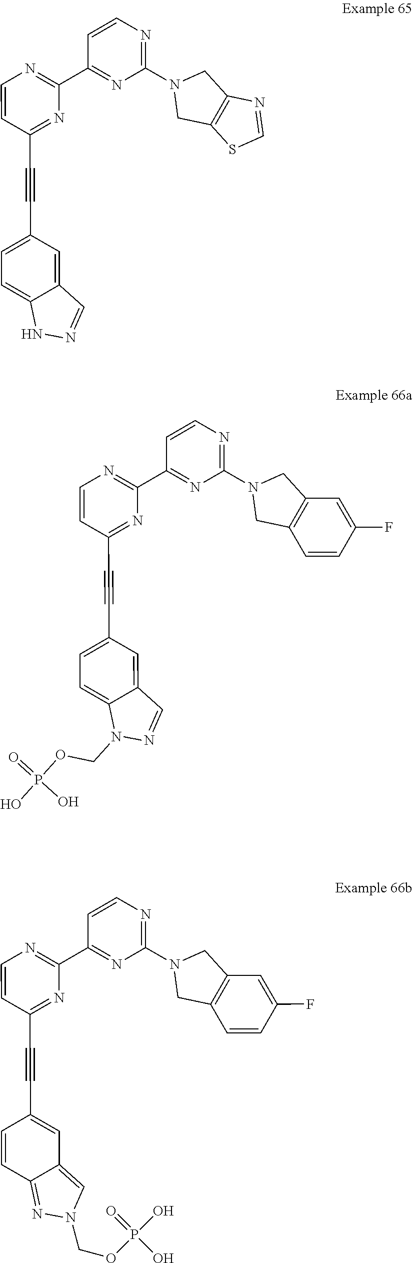 [SMILES: C(#Cc1ccnc(-c2ccnc(N3Cc4ncsc4C3)n2)n1)c1ccc2[nH]ncc2c1 ; O=P(O)(O)OCn1ncc2cc(C#Cc3ccnc(-c4ccnc(N5Cc6ccc(F)cc6C5)n4)n3)ccc21 ; O=P(O)(O)OCn1cc2cc(C#Cc3ccnc(-c4ccnc(N5Cc6ccc(F)cc6C5)n4)n3)ccc2n1]

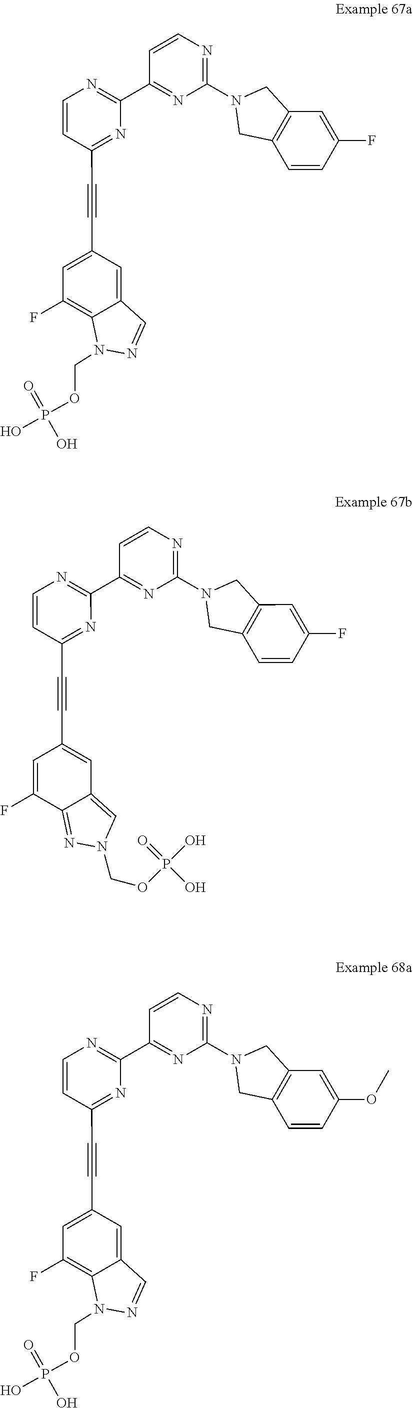 [SMILES: O=P(O)(O)OCn1ncc2cc(C#Cc3ccnc(-c4ccnc(N5Cc6ccc(F)cc6C5)n4)n3)cc(F)c21 ; O=P(O)(O)OCn1cc2cc(C#Cc3ccnc(-c4ccnc(N5Cc6ccc(F)cc6C5)n4)n3)cc(F)c2n1 ; COc1ccc2c(c1)CN(c1nccc(-c3nccc(C#Cc4cc(F)c5c(cnn5COP(=O)(O)O)c4)n3)n1)C2]

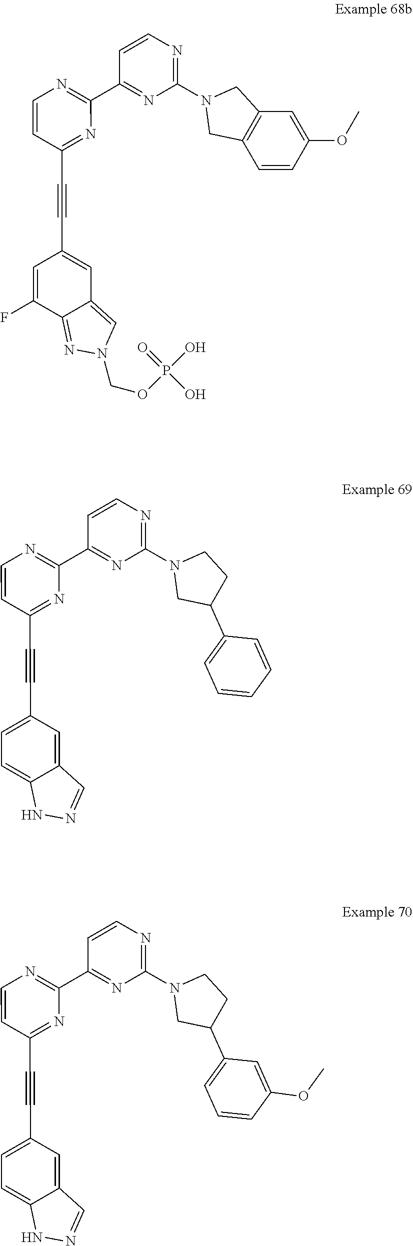 [SMILES: COc1ccc2c(c1)CN(c1nccc(-c3nccc(C#Cc4cc(F)c5nn(COP(=O)(O)O)cc5c4)n3)n1)C2 ; C(#Cc1ccnc(-c2ccnc(N3CCC(c4ccccc4)C3)n2)n1)c1ccc2[nH]ncc2c1 ; COc1cccc(C2CCN(c3nccc(-c4nccc(C#Cc5ccc6[nH]ncc6c5)n4)n3)C2)c1]

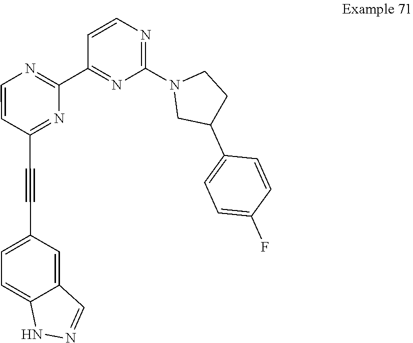 [SMILES: Fc1ccc(C2CCN(c3nccc(-c4nccc(C#Cc5ccc6[nH]ncc6c5)n4)n3)C2)cc1]

or a pharmaceutically acceptable salt thereof.

It will be appreciated that each of the compounds described herein and each of the subclasses of compounds described above may be substituted as described generally herein, or may be substituted according to any one or more of the subclasses described above and herein [e.g., i)-xx)].

Some of the foregoing compounds can comprise one or more asymmetric centers, and thus can exist in various isomeric forms, e.g., stereoisomers and/or diastereomers. Thus, provided compounds and pharmaceutical compositions thereof may be in the form of an individual enantiomer, diastereomer or geometric isomer, or may be in the form of a mixture of stereoisomers. In certain embodiments, the compounds described herein are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of stereoisomers. In addition to the above-mentioned compounds per se, the present disclosure also encompasses pharmaceutically acceptable derivatives of these compounds and compositions comprising one or more compounds described herein and one or more pharmaceutically acceptable excipients or additives. In some embodiments, a compound of Formula II or a subgenera thereof is provided as a pharmaceutically acceptable salt.

Provided compounds may be prepared by crystallization of a compound under different conditions and may exist as one or a combination of polymorphs. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses provided compounds, their derivatives, their tautomeric forms, their stereoisomers, their polymorphs, their pharmaceutically acceptable salts, their pharmaceutically acceptable solvates and pharmaceutically acceptable compositions containing them. Tautomeric forms of compounds of the present invention include, for example the substituted indazolyl compounds, in which the proton on the nitrogen can be attached to either of the two nitrogen atoms of any of the aforementioned disubstituted compounds of general Formula I and related formulas.

### Pharmaceutical Compositions

As discussed above the present disclosure provides novel compounds that have biological properties useful for the treatment of any of a number of conditions or diseases in which inhibiting ROCK1, ROCK2, and ROCK1/2 activities thereof have a therapeutically useful role.

Accordingly, in another aspect of the present disclosure, pharmaceutical compositions are provided, which comprise any one or more of the compounds of Formula I described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound described herein may be administered to a patient in need thereof in combination with the administration of one or more other therapeutic agents. For example, additional therapeutic agents for conjoint administration or inclusion in a pharmaceutical composition with a compound described herein may be an approved agent to treat the same or related indication, or it may be any one of a number of agents undergoing approval in the Food and Drug Administration that ultimately obtain approval for the treatment of any disorder described herein. It will also be appreciated that certain provided compounds can exist in free form for treatment, or where appropriate, as a pharmaceutically acceptable derivative thereof. According to the present disclosure, a pharmaceutically acceptable derivative includes, but is not limited to, pharmaceutically acceptable salts, esters, salts of such esters, or a pro-drug or other adduct or derivative of a compound of described herein which upon administration to a patient in need is capable of providing, directly or indirectly, a compound as otherwise described herein, or a metabolite or residue thereof.

As used herein with reference to compounds of Formula I and subgenera thereof, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of compounds of Formula I and subgenera thereof, or separately by reacting a free base or free acid function with a suitable reagent, as described generally below. For example, a free base function can be reacted with a suitable acid. Furthermore, where the compounds of Formula I and subgenera thereof carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of provided compounds which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood, or N-demethylation of a compound of the invention where R¹ is methyl. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present disclosure additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other nontoxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyethynylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present disclosure encompasses pharmaceutically acceptable topical formulations of provided compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations described herein may comprise excipients. Any pharmaceutically acceptable excipient known in the art may be used to prepare pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination with one or more provided compounds. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerin, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quatemary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations described herein comprise at least a compound of the invention and a penetration enhancing agent. The choice of topical formulation will depend or several factors, including the condition to be treated, the physicochemical characteristics of provided compounds and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g, aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions, sprays, inhalants or patches. In certain exemplary embodiments, formulations of the compositions described herein are creams, which may further contain saturated or unsaturated fatty acids such as stearic acid, palmitic acid, oleic acid, palmito-oleic acid, cetyl or oleyl alcohols, stearic acid being particularly preferred. Creams described herein may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this disclosure. Formulations for intraocular administration are also included. Additionally, the present disclosure contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions described herein can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a provided compound may be administered concurrently with another antiinflammatory agent), or they may achieve different effects (e.g., control of any adverse effects). In non-limiting examples, one or more compounds described herein may be formulated with at least one cytokine, growth factor or other biological, such as an interferon, e.g., alpha interferon, or with at least another small molecule compound. Non-limiting examples of pharmaceutical agents that may be combined therapeutically with compounds of the present disclosure include: antivirals and antifibrotics such as interferon alpha, combination of interferon alpha and ribavirin, Lamivudine, Adefovir dipivoxil and interferon gamma; anticoagulants such as heparin and warfarin; antiplatelets e.g., aspirin, ticlopidine and clopidogrel; other growth factors involved in regeneration, e.g., VEGF and FGF and mimetics of these growth factors; antiapoptotic agents; and motility and morphogenic agents.

In certain embodiments, the pharmaceutical compositions described herein further comprise one or more additional therapeutically active ingredients (e.g., anti-inflammatory and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs.

### Research Uses, Clinical Uses, Pharmaceutical Uses and Methods of Treatment

### Research Uses

According to the present invention, provided compounds may be assayed in any of the available assays known in the art for identifying compounds having the ability to modulate ROCK1, ROCK2, or ROCK1/2 activities and in particular to antagonize the activities of ROCK1, ROCK2, or ROCK1/2. For example, the assay may be cellular or non-cellular, in vivo or in vitro, high- or low-throughput format, etc.

Thus, in one aspect, preferred compounds disclosed herein include those which inhibit ROCK1, ROCK2, or ROCK1/2 activities.

Clinical Uses of Compounds with ROCK1, ROCK2, or ROCK1/2 Inhibitory Activities.

1. Fibrotic Liver Disease: Liver fibrosis is the scarring response of the liver to chronic liver injury; when fibrosis progresses to cirrhosis, morbid complications can develop. In fact, end-stage liver fibrosis or cirrhosis is the seventh leading cause of death in the United States, and afflicts hundreds of millions of people worldwide; deaths from end-stage liver disease in the United States are expected to triple over the next 10-15 years, mainly due to the hepatitis C epidemic. In addition to the hepatitis C virus, many other forms of chronic liver injury also lead to end-stage liver disease and cirrhosis, including other viruses such as hepatitis B and delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency).

Treatment of liver fibrosis has focused to date on eliminating the primary injury. For extrahepatic obstructions, biliary decompression is the recommended mode of treatment whereas patients with Wilson's disease are treated with zinc acetate. In chronic hepatitis C infection, interferon has been used as antiviral therapies with limited response: ˜20% when used alone or ˜50% response when used in combination with ribavirin. In addition to the low-level of response, treatment with interferon with or without ribavirin is associated with numerous severe side effects including neutropenia, thrombocytopenia, anemia, depression, generalized fatigue and flu-like symptoms, which are sufficiently significant to necessitate cessation of therapy. Treatments for other chronic liver diseases such as hepatitis B, autoimmune hepatitis and Wilson's disease are also associated with many side effects, while primary biliary cirrhosis, primary sclerosing cholangitis and non-alcoholic fatty liver disease have no effective treatment other than liver transplantation.

The advantage of treating fibrosis rather than only the underlying etiology, is that antifibrotic therapies should be broadly applicable across the full spectrum of chronic liver diseases. While transplantation is currently the most effective cure for liver fibrosis, mounting evidence indicates that not only fibrosis, but even cirrhosis is reversible. Unfortunately, patients often present with advanced stages of fibrosis and cirrhosis, when many therapies such as antivirals can no longer be safely used due to their side effect profile. Such patients would benefit enormously from effective antifibrotic therapy, because attenuating or reversing fibrosis may prevent many late stage complications such as infection, ascites, and loss of liver function and preclude the need for liver transplantation. The compounds disclosed herein are beneficial for the treatment of the foregoing conditions, and generally are antifibrotic and/or antiapoptotic agents for this and other organ or tissues.

2. Hepatic Ischemia-Reperfusion Injury: Currently, transplantation is the most effective therapeutic strategy for liver fibrosis. However, in spite of the significant improvement in clinical outcome during the last decade, liver dysfunction or failure is still a significant clinical problem after transplantation surgery. Ischemia-reperfusion (IR) injury to the liver is a major alloantigen-independent component affecting transplantation outcome, causing up to 10% of early organ failure, and leading to the higher incidence of both acute and chronic rejection. Furthermore, given the dramatic organ shortage for transplantation, surgeons are forced to consider cadaveric or steatotic grafts or other marginal livers, which have a higher susceptibility to reperfusion injury. In addition to transplantation surgery, liver IR injury is manifested in clinical situations such as tissue resections (Pringle maneuver), and hemorrhagic shock.

The damage to the postischemic liver represents a continuum of processes that culminate in hepatocellular injury. Ischemia activates Kupffer cells, which are the main sources of vascular reactive oxygen species (ROS) formation during the initial reperfusion period. In addition to Kupffer cell-induced oxidant stress, with increasing length of the ischemic episode, intracellular generation of ROS by xanthine oxidase and in particular mitochondria may also contribute to liver dysfunction and cell injury during reperfusion. Endogenous antioxidant compounds, such as superoxide dismutase, catalase, glutathione, alphatocopherol, and beta-carotene, may all limit the effects of oxidant injury but these systems can quickly become overwhelmed by large quantities of ROS. Work by Lemasters and colleagues, has indicated that in addition to formation of ROS, intracellular calcium dyshomeostasis is a key contributor to liver IR injury. Cell death of hepatocytes and endothelial cells in this setting is characterized by swelling of cells and their organelles, release of cell contents, eosinophilia, karyolysis, and induction of inflammation, characteristic of oncotic necrosis. More recent reports indicate that liver cells also die by apoptosis, which is morphologically characterized by cell shrinkage, formation of apoptotic bodies with intact cell organelles and absence of an inflammatory response.

Indeed, minimizing the adverse effects of IR injury could significantly increase the number of patients that may successfully undergo liver transplantation. Pharmacologic interventions that reduce cell death and/or enhance organ regeneration represent a therapeutic approach to improve clinical outcome in liver transplantation, liver surgery with vascular exclusion and trauma and can therefore reduce recipient/patient morbidity and mortality. The compounds disclosed herein are beneficial for the treatment of the foregoing conditions.

3. Cerebral Infarction. Stroke and cerebrovascular disease are a leading cause of morbidity and mortality in the US: at least 600,000 Americans develop strokes each year, and about 160,000 of these are fatal. Research on the pathophysiological basis of stroke has produced new paradigms for prevention and treatment, but translation of these approaches into improved clinical outcomes has proved to be painfully slow. Preventive strategies focus primarily on reducing or controlling risk factors such as diabetes, hypertension, cardiovascular disease, and lifestyle; in patients with severe stenosis, carotid endarterectomy may be indicated. Cerebral angioplasty is used investigationally, but the high restenosis rates observed following coronary angioplasty suggest this approach may pose unacceptable risk for many patients. Therapeutic strategies focus primarily on acute treatment to reduce injury in the ischemic penumbra, the region of reversibly damaged tissue surrounding an infarct. Thrombolytic therapy has been shown to improve perfusion to the ischemic penumbra, but it must be administered within three hours of the onset of infarction. Several neuroprotective agents that block specific tissue responses to ischemia are promising, but none have yet been approved for clinical use. While these therapeutic approaches limit damage in the ischemic penumbra, they do not address the underlying problem of inadequate blood supply due to occluded arteries. An alternative strategy is to induce formation of collateral blood vessels in the ischemic region; this occurs naturally in chronic ischemic conditions, but stimulation of vascularization via therapeutic angiogenesis has potential therapeutic benefit.

Recent advances in imaging have confirmed the pathophysiological basis of the clinical observations of evolving stroke. Analysis of impaired cerebral blood flow (CBF) in the region of an arterial occlusion supports the hypothesis that a central region of very low CBF, the ischemic core, is irreversibly damaged, but damage in surrounding or intermixed zones where CBF is of less severely reduced, the ischemic penumbra, can be limited by timely reperfusion. Plate recently reviewed the evidence suggesting that therapeutic angiogenesis may be useful for treatment or prevention of stroke. Analysis of cerebral vasculature in stroke patients showed a strong correlation between blood vessel density and survival and a higher density of microvessels in the ischemic hemisphere compared to the contralateral region. The compounds disclosed herein are beneficial for the treatment of the foregoing conditions.

4. Ischemic heart disease is a leading cause of morbidity and mortality in the US, afflicting millions of Americans each year at a cost expected to exceed $300 billion/year. Numerous pharmacological and interventional approaches are being developed to improve treatment of ischemic heart disease including reduction of modifiable risk factors, improved revascularization procedures, and therapies to halt progression and/or induce regression of atherosclerosis. One of the most exciting areas of research for the treatment of myocardial ischemia is therapeutic angiogenesis. Recent studies support the concept that administration of angiogenic growth factors, either by gene transfer or as a recombinant protein, augments nutrient perfusion through neovascularization. The newly developed, supplemental collateral blood vessels constitute endogenous bypass conduits around occluded native arteries, improving perfusion to ischemic tissue. The compounds disclosed herein are beneficial for the treatment of the foregoing conditions.

5. Renal Disease. Chronic renal dysfunction is a progressive, degenerative disorder that ultimately results in acute renal failure and requires dialysis as an intervention, and renal transplantation as the only potential cure. Initiating conditions of renal dysfunction include ischemia, diabetes, underlying cardiovascular disease, or renal toxicity associated with certain chemotherapeutics, antibiotics, and radiocontrast agents. Most end-stage pathological changes include extensive fibrinogenesis, epithelial atrophy, and inflammatory cell infiltration into the kidneys.

Acute renal failure is often a complication of diseases including diabetes or renal ischemia, procedures such as heminephrectomy, or as a side effect of therapeutics administered to treat disease. The widely prescribed anti-tumor drug cis-diamminedichloroplatinum (cisplatin), for example, has side effects that include a high incidence of nephrotoxicity and renal dysfunction, mainly in the form of renal tubular damage that leads to impaired glomerular filtration. Administration of gentamicin, an aminoglycoside antibiotic, or cyclosporin A, a potent immunosuppressive compound, causes similar nephrotoxicity. The serious side effects of these effective drugs restrict their use. The development of agents that protect renal function and enhance renal regeneration after administration of nephrotoxic drugs will be of substantial benefit to numerous patients, especially those with malignant tumors, and may allow the maximal therapeutic potentials of these drugs to be realized. The compounds disclosed herein are beneficial for the treatment of the renal diseases mentioned above.

6. Lung (Pulmonary) Fibrosis. Idiopathic pulmonary fibrosis (IPF) accounts for a majority of chronic interstitial lung diseases, and has an estimated incidence rate of 10.7 cases for 100,000 per year, with an estimated mortality of 50-70%. IPF is characterized by an abnormal deposition of collagen in the lung with an unknown etiology. Although the precise sequence of the pathogenic sequelae is unknown, disease progression involves epithelial injury and activation, formation of distinctive subepithelial fibroblast/myofibroblast foci, and excessive extracellular matrix accumulation. The development of this pathological process is preceded by an inflammatory response, often dominated by macrophages and lymphocytes, which is mediated by the local release of chemoattractant factors and upregulation of cell-surface adhesion molecules. Lung injury leads to vasodilatation and leakage of plasma proteins into interstitial and alveolar spaces, as well as activation of the coagulation cascade and deposition of fibrin. Fibroblasts migrate into this provisional fibrin matrix where they synthesize extracellular matrix molecules. In non-pathogenic conditions, excess fibrin is usually degraded by plasmin, a proteinase that also has a role in the activation of matrix metalloproteinases (MMPs). Activated MMPs degrade extracellular matrix and participate in fibrin removal, resulting in the clearance of the alveolar spaces and the ultimate restoration of injured tissues. In pathological conditions, however, these processes can lead to progressive and irreversible changes in lung architecture, resulting in progressive respiratory insufficiency and an almost universally terminal outcome in a relatively short period of time. Fibrosis is the final common pathway of a variety of lung disorders, and in this context, the diagnosis of pulmonary fibrosis implies the recognition of an advanced stage in the evolution of a complex process of abnormal repair. While many studies have focused on inflammatory mechanisms for initiating the fibrotic response, the synthesis and degradation the extracellular matrix represent the central event of the disease. It is this process that presents a very attractive site of therapeutic intervention.

The course of IPF is characterized by progressive respiratory insufficiency, leading to death within 3 to 8 years from the onset of symptoms. Management of interstitial lung disease in general, and in particular idiopathic pulmonary fibrosis, is difficult, unpredictable and unsatisfactory. Attempts have been made to use antiinflammatory therapy to reverse inflammation, relief, stop disease progression and prolong survival. Corticosteroids are the most frequently used antiinflammatory agents and have been the mainstay of therapy for IPF for more than four decades, but the efficacy of this approach is unproven, and toxicities are substantial. No studies have compared differing dosages or duration of corticosteroid treatment in matched patients. Interpretation of therapy efficacy is obscured by several factors including heterogeneous patient populations, inclusion of patients with histologic entities other than usual interstitial pneumonia, lack of objective, validated endpoints, and different criteria for “response.” Cytotoxic drugs such as Azathioprine and cyclophosphamide have also being used in combination with low dose oral corticosteroids. The results of such treatments vary from no improvement to significant prolongation of survival. Overall, currently available treatments for lung fibrosis are sub-optimal. Potential new therapies have emerged from the use of animal models of pulmonary fibrosis and recent advances in the cellular and molecular biology of inflammatory reactions. Such therapies involve the use of cytokines, oxidants and growth factors that are elaborated during the fibrotic reaction. Despite the use of newer strategies for treatment, the overall prognosis for patients with interstitial lung disease has had little quantifiable change, and the population survival remains unchanged for the last 30 years. Interferon gamma (IFN) may be effective in the treatment of IPF in some patients but its role is controversial. Literature indicated that IFN-gamma may be involved in small airway disease in silicotic lung. Others showed that IFN gamma mediates, bleomycin-induced pulmonary inflammation and fibrosis. The compounds disclosed herein are beneficial for the treatment of the foregoing condition, among other fibrotic diseases.

### Exemplary Assays

Efficacy of the compounds disclosed herein on the aforementioned disorders and diseases or the potential to be of benefit for the prophylaxis or treatment thereof may be demonstrated in various studies, ranging from biochemical effects evaluated in vitro and effects on cells in culture, to in-vivo models of disease, wherein direct clinical manifestations of the disease can be observed and measured, or wherein early structural and/or functional events occur that are established to be involved in the initiation or progression of the disease. The positive effects of the compounds disclosed herein have been demonstrated in a variety of such assays and models, for a number of diseases and disorders. One skilled in the art can readily determine following the guidance described herein whether a compound disclosed herein useful for the purposed herein described.

As detailed in the exemplification herein, in assays to determine the ability of compounds to inhibit the activities of ROCK1, ROCK2, or ROCK1/2 measured in vitro, certain provided compounds exhibited IC₅₀ values ≤50 μM. In certain other embodiments, provided compounds exhibit IC₅₀ values ≤40 μM. In certain other embodiments, provided compounds exhibit IC₅₀ values ≤30 μM. In certain other embodiments, provided compounds exhibit IC₅₀ values ≤20 μM. In certain other embodiments, provided compounds exhibit IC₅₀ values ≤10 μM. In certain other embodiments, provided compounds exhibit IC₅₀ values ≤7.5 μM. In certain embodiments, provided compounds exhibit IC₅₀ values ≤5 μM. In certain other embodiments, provided compounds exhibit IC₅₀ values ≤2.5 μM. In certain embodiments, provided compounds exhibit IC₅₀ values ≤1 μM. In certain other embodiments, provided compounds exhibit IC₅₀ values ≤750 nM. In certain other embodiments, provided compounds exhibit IC₅₀ values ≤500 nM. In certain other embodiments, provided compounds exhibit IC₅₀ values ≤250 nM. In certain other embodiments, provided compounds exhibit IC₅₀ values ≤100 nM. In other embodiments, exemplary compounds exhibited IC₅₀ values ≤75 nM. In other embodiments, exemplary compounds exhibited IC₅₀ values ≤50 nM. In other embodiments, exemplary compounds exhibited IC₅₀ values ≤40 nM. In other embodiments, exemplary compounds exhibited IC₅₀ values ≤30 nM. In other embodiments, exemplary compounds exhibited IC₅₀ values ≤20 nM. In other embodiments, exemplary compounds exhibited IC₅₀ values ≤10 nM. In other embodiments, exemplary compounds exhibited IC₅₀ values ≤5 nM.

As detailed in the exemplification herein, in assays to determine the affinity of compounds in binding to ROCK1, ROCK2, or ROCK1/2 measured in vitro, certain provided compounds exhibited equilibrium dissociation constant Kd values ≤50 μM. In certain other embodiments, provided compounds exhibit Kd values ≤40 μM. In certain other embodiments, provided compounds exhibit Kd values ≤30 μM. In certain other embodiments, provided compounds exhibit Kd values ≤20 μM. In certain other embodiments, provided compounds exhibit Kd values ≤10 μM. In certain other embodiments, provided compounds exhibit Kd values ≤7.5 μM. In certain embodiments, provided compounds exhibit Kd values ≤5 μM. In certain other embodiments, provided compounds exhibit Kd values ≤2.5 μM. In certain embodiments, provided compounds exhibit Kd values ≤1 μM. In certain other embodiments, provided compounds exhibit Kd values ≤750 nM. In certain other embodiments, provided compounds exhibit Kd values ≤500 nM. In certain other embodiments, provided compounds exhibit Kd values ≤250 nM. In certain other embodiments, provided compounds exhibit Kd values ≤100 nM. In other embodiments, exemplary compounds exhibited Kd values ≤75 nM. In other embodiments, exemplary compounds exhibited Kd values ≤50 nM. In other embodiments, exemplary compounds exhibited Kd values ≤40 nM. In other embodiments, exemplary compounds exhibited Kd values ≤30 nM. In other embodiments, exemplary compounds exhibited Kd values ≤20 nM. In other embodiments, exemplary compounds exhibited Kd values ≤10 nM. In other embodiments, exemplary compounds exhibited Kd values ≤5 nM.

In certain embodiments, the compounds disclosed herein are selective inhibitors of either ROCK1 or ROCK2. In some embodiments, compounds disclosed herein selectively inhibit ROCK2, and thus, in some embodiments, exhibit less of ability to cause hypotension. In some embodiments, compounds disclosed herein inhibit both ROCK1 and ROCK2 to achieve optimal efficacies.

As used herein, the term “selective inhibition” or “selectively inhibit(s)” means that a provided compound has greater inhibition of ROCK2 in at least one assay described herein (e.g., biochemical or cellular) as compared to ROCK1. In some embodiments, the term “selective inhibition” or “selectively inhibit(s)” means that a provided compound is at least 2 times, at least 3 times, at least 5 times, at 10 times, at least 15 times, at least 20 times, at least 25 times, at least 30 times, at least 40 times, at least 50 times, at least 60 times, at least 70 times, at least 80 times, at least 90 times, at least 100 times, at least 150 times, at least 200 times, at least 300 times, at least 400 times, at least 500 times, or at least 1000 times more potent as an inhibitor of ROCK2 as compared to inhibition of ROCK1. In some embodiments, the selectivity of a provided compound is determined based on an assay described herein. In some such embodiments, the selectivity of a provided compound is determined based on DiscoverX's KINOMEscan™ KdELECT technology.

### Pharmaceutical Uses and Methods of Treatment

As discussed above, certain of the compounds as described herein exhibit activity generally as modulators of ROCK1, ROCK2, or ROCK1/2 activities. More specifically, compounds disclosed herein demonstrate the ability to inhibit ROCK1, ROCK2, or ROCK1/2 activities. Thus, in certain embodiments, compounds disclosed herein are useful for the treatment of any of a number of conditions or diseases in which inhibiting ROCK1, ROCK2, or ROCK1/2 activities thereof have a therapeutically useful role, in particular antifibrotic. Thus, compounds disclosed herein are useful for the treatment of any condition, disease or disorder in which inhibiting ROCK1, ROCK2, or ROCK1/2 activities would have a beneficial role.

Accordingly, in another aspect, methods for the treatment of ROCK1, ROCK2, or ROCK1/2 related disorders are provided comprising administering a therapeutically effective amount of a compound of formula (I) as described herein, to a subject in need thereof. In certain embodiments, a method for the treatment of ROCK1, ROCK2, or ROCK1/2 activities related disorders is provided comprising administering a therapeutically effective amount of a provided compound, or a pharmaceutical composition comprising a provided compound to a subject in need thereof, in such amounts and for such time as is necessary to achieve the desired result.

In certain embodiments, the method involves the administration of a therapeutically effective amount of the compound or a pharmaceutically acceptable derivative(s) thereof to a subject (including, but not limited to a human or animal) in need of it. Subjects for which the benefits of the compounds disclosed herein are intended for administration include, in addition to humans, livestock, domesticated, zoo and companion animals.

Thus, as described above, in one aspect, a method for the treatment of disorders related to inhibiting ROCK1, ROCK2, or ROCK1/2 activities is provided comprising administering a therapeutically effective amount of a compound of Formula I or Formula II as described herein, to a subject in need thereof. In certain embodiments of special interest, the provided method is used for the treatment of, in the case of ROCK1, ROCK2, or ROCK1/2 hyperactivities, hepatic disease, stroke, myocardial infarction and other ischemic or fibrotic diseases. It will be appreciated that the compounds and compositions, according to the method disclosed herein, may be administered using any amount and any route of administration effective for the treatment of conditions or diseases in which inhibiting ROCK1, ROCK2, or ROCK1/2 activities thereof have a therapeutically useful role. Thus, the expression “effective amount” as used herein, refers to a sufficient amount of agent to inhibit ROCK1, ROCK2, or ROCK1/2 activities, and to exhibit a therapeutic effect. The exact amount required will vary from subject to subject, depending on the species, age, and general condition of the subject, the severity of the infection, the particular therapeutic agent, its mode and/or route of administration, and the like. The compounds disclosed herein are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions disclosed herein will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, the present disclosure provides a method of inhibiting ROCK1 and/or ROCK2 in a patient or in a biological sample. In some embodiments, the present disclosure provides a method of inhibiting ROCK1 and/or ROCK2, the method comprising contacting a biological sample with a compound of Formula I, or a compound of Formula II, or a pharmaceutically acceptable salt thereof.

In some embodiments, the present disclosure provides a method of inhibiting ROCK2 selectively as compared to ROCK1 in a biological sample or in a patient.

In some embodiments, the present disclosure provides a method of treating or lessening the severity of one or more diseases or disorders associated with or mediated by ROCK1 and/or ROCK2. In some embodiments, a disease or disorder associated with or mediated by ROCK1 and/or ROCK2 is a disease or disorder as described herein. In some embodiments, a method of treating or lessening the severity of one or more diseases or disorders associated with or mediated by ROCK1 and/or ROCK2 includes the step of administering to a patient in need thereof a compound of Formula I, or a compound of Formula II, or a pharmaceutically acceptable salt thereof. In some embodiments, a patient in need thereof comprises a subject, or a population of subjects, who is/are suffering from, has/have been diagnosed with, or is/are suspected of having a disease or disorder associated with or mediated by ROCK1 and/or ROCK2.

Furthermore, after formulation with an appropriate pharmaceutically acceptable carrier in a desired dosage, the pharmaceutical compositions disclosed herein can be administered to humans and other animals orally, rectally, parenterally, intracisternally, intravaginally, intraperitoneally, subcutaneously, intradermally, intra-ocularly, topically (as by powders, ointments, or drops), buccally, as an oral or nasal spray, or the like, depending on the severity of the disease or disorder being treated. In certain embodiments, the compounds disclosed herein may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Moreover, pharmaceutical compositions comprising one or more compounds disclosed herein may also contain other compounds or agents for which co-administration with the compound(s) disclosed herein is therapeutically advantageous. As many pharmaceutical agents are used in the treatment of the diseases and disorders for which the compounds disclosed herein are also beneficial, any may be formulated together for administration. Synergistic formulations are also embraced herein, where the combination of at least one compound disclosed herein and at least one other compound act more beneficially than when each is given alone.

### Treatment Kit

In other embodiments, the present disclosure relates to a kit for conveniently and effectively carrying out the methods in accordance with the present disclosure. In general, the pharmaceutical pack or kit comprises one or more containers filled with one or more of the ingredients of the pharmaceutical compositions described herein. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples that follow are intended to help illustrate the compounds, compositions, and methods described herein, and are not intended to, nor should they be construed to, limit the scope of the embodiments described. Indeed, various modifications of embodiments described herein and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

### 1) General Description of Synthetic Methods:

The practitioner has a well-established literature of small molecule chemistry to draw upon, in combination with the information contained herein, for guidance on synthetic strategies, protecting groups, and other materials and methods useful for the synthesis of the compounds of this invention.

The various references cited herein provide helpful background information on preparing compounds similar to the provided compounds described herein or relevant intermediates, as well as information on formulation, uses, and administration of such compounds which may be of interest.

Moreover, the practitioner is directed to the specific guidance and examples provided in this document relating to various exemplary compounds and intermediates thereof.

The compounds of this disclosure and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

According to the present disclosure, any available techniques can be used to make or prepare the provided compounds or compositions including them. For example, a variety of solution phase synthetic methods such as those discussed in detail below may be used. Alternatively or additionally, the provided compounds may be prepared using any of a variety combinatorial techniques, parallel synthesis and/or solid phase synthetic methods known in the art.

It will be appreciated as described below, that a variety of provided compounds can be synthesized according to the methods described herein. The starting materials and reagents used in preparing these compounds are either available from commercial suppliers such as Aldrich Chemical Company (Milwaukee, Wis.), Bachem (Torrance, Calif.), Sigma (St. Louis, Mo.), or are prepared by methods well known to a person of ordinary skill in the art following procedures described in such references as Fieser and Fieser 1991, “Reagents for Organic Synthesis”, vols 1-17, John Wiley and Sons, New York, N.Y., 1991; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; and Larock 1990, “Comprehensive Organic Transformations: A Guide to Functional Group Preparations”, 2^{nd} ed. VCH Publishers. These schemes are merely illustrative of some methods by which the compounds of this invention can be synthesized, and various modifications to these schemes can be made and will be suggested to a person of ordinary skill in the art having regard to this disclosure.

The starting materials, intermediates, and compounds of this disclosure may be isolated and purified using conventional techniques, including filtration, distillation, crystallization, chromatography, and the like. They may be characterized using conventional methods, including physical constants and spectral data.

### General Reaction Procedures:

Unless mentioned specifically, reaction mixtures were stirred using a magnetically driven stirrer bar. An inert atmosphere refers to either dry argon or dry nitrogen. Reactions were monitored either by thin layer chromatography, by proton nuclear magnetic resonance (NMR) or by high-pressure liquid chromatography (HPLC), of a suitably worked up sample of the reaction mixture.

### General Work Up Procedures:

Unless mentioned specifically, reaction mixtures were cooled to room temperature or below then quenched, when necessary, with either water or a saturated aqueous solution of ammonium chloride. Desired products were extracted by partitioning between water and a suitable water-immiscible solvent (e.g. ethyl acetate, dichloromethane, diethyl ether). The desired product-containing extracts were washed appropriately with water followed by a saturated solution of brine. On occasions where the product containing extract was deemed to contain residual oxidants, the extract was washed with a 10% solution of sodium sulphite in saturated aqueous sodium bicarbonate solution, prior to the aforementioned washing procedure. On occasions where the product containing extract was deemed to contain residual acids, the extract was washed with saturated aqueous sodium bicarbonate solution, prior to the aforementioned washing procedure (except in those cases where the desired product itself had acidic character). On occasions where the product containing extract was deemed to contain residual bases, the extract was washed with 10% aqueous citric acid solution, prior to the aforementioned washing procedure (except in those cases where the desired product itself had basic character). Post washing, the desired product containing extracts were dried over anhydrous magnesium sulphate, and then filtered. The crude products were then isolated by removal of solvent(s) by rotary evaporation under reduced pressure, at an appropriate temperature (generally less than 45° C.).

### General Purification Procedures:

Unless mentioned specifically, chromatographic purification refers to flash column chromatography on silica and/or preparative thin layer chromatography (TLC) plates, using a single solvent or mixed solvent as eluent. Suitably purified desired product containing elutes were combined and concentrated under reduced pressure at an appropriate temperature (generally less than 45° C.) to constant mass. Final compounds were dissolved in 50% aqueous acetonitrile, filtered and transferred to vials, then freeze-dried under high vacuum before submission for biological testing.

### 1) Synthesis of Exemplary Compounds:

In certain exemplary embodiments, compounds of formula I may be prepared as follows according to Scheme 1:

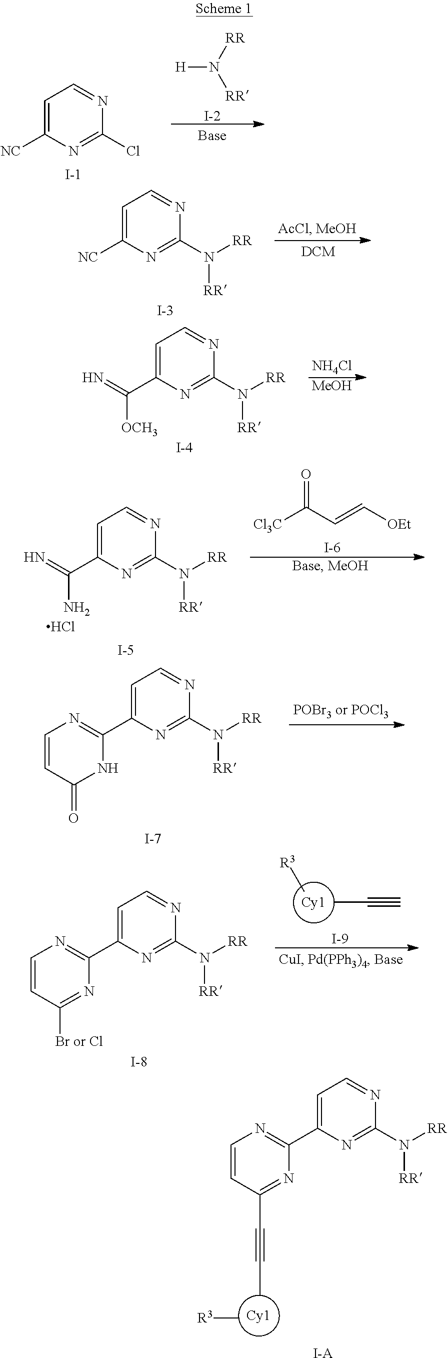

wherein

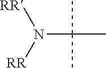

is R—, representing an optionally substituted heterocyclic, aromatic, or heteroaromatic; wherein, the optional substituents are selected from one or more independent hydrogen, deuterium, halo, —CN, —NO₂, aliphatic, alicyclic, heteroaliphatic, heterocyclic, aromatic, heteroaromatic, —OR^{a}, —NR^{b}R^{c}, —S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{k}CO₂R^{p}; R³ and Cy1 have the same meanings as those in the claims; “Base” refers to inorganic or organic bases. Some examples of organic bases include but are not limited to Me₃N, Et₃N, n-Pr₃N, i-Pr₃N, n-Bu₃N, s-Bu₃N, i-Bu₃N, t-Bu₃N, i-Pr₂NEt, pyridine, 1,8-diazabicyclo(5.4.0)undec-7-ene (DBU), 1,4-diazabicyclo[2.2.2]octane (DABCO), 1,1,2,3,3-pentamethylguanidine, 1,1,2,3,3-pentaethylguanidine, N-methylmorpholine, N-ethylmorpholine, N-isopropylmorpholine, N-methylpiperidine, N-ethylpiperidine, N-isopropylpiperidine, 1,4-dimethylpiperazine, 1,4-diethylpiperazine, 1,4-diisopropylpiperazine, N-methylpyrrolidine, N-ethylpyrrolidine, N-isopropylpyrrolidine, MeONa, MeOK, MeOLi, EtOLi, EtONa, EtOK, n-PrOLi, n-PrONa, n-PrOK, i-PrOLi, i-PrONa, i-PrOK, n-BuOLi, n-BuONa, n-BuOK, i-BuOLi, i-BuONa, i-BuOK, s-BuOLi, s-BuONa, s-BuOK, t-BuOLi, t-BuONa, t-BuOK, n-BuLi, s-BuLi, t-BuLi, NaN(SiMe₃)₂, LiN(SiMe₃)₂, and KN(SiMe₃)₂. Some examples of inorganic bases include but are not limited to LiOH, NaOH, KOH, RbOH, CsOH, Cs₂CO₃, Rb₂CO₃, Li₂CO₃, Na₂CO₃, K₂CO₃, NaHCO₃, LiF, NaF, KF, RbF, CsF, K₃PO₃, K₂HPO₄, KH₂PO₄, Na₃PO₃, Na₂HPO₄, NaH₂PO₄, Li₃PO₃, Li₂HPO₄, LiH₂PO₄, NaH, LuH, KH, RbH, CsH, CaO, Ca(OH)₂, Ca₂CO₃, MgO, Mg(OH)₂, or Mg₂CO₃.

Starting material I-1 is commercially available from multiple suppliers. The displacement reaction between I-1 and I-2 gave product I-3. Compound I-3 was converted into the amidine intermediate I-5 in one step. Compound I-5 reacted with I-6 to give the pyrimidonyl compound I-7. Treatment of I-7 with POCl₃ or POBr₃ neat or in a solvent or a mixture of solvents including but not limited to acetonitrile, dichloromethane, 1,2-dichloroethane, N,N′-dimethylformamide, and N,N′-dimethylacetamide afforded chloride or bromide I-8. Sonagoshira coupling of I-8 with alkyne I-9 gave the target compound I-A. Sonagoshira coupling is a name reaction and more information can be found in a paper (R. Chinchilla and C. Najera Chem. Soc. Rev. 2011, 40, 5084-5121).

It will be appreciated that the reaction sequence illustrated in Scheme 1 is general in nature, and one skilled in the art will recognize that the method could be used to prepare analogues in which Cy1, R³, RR, and RR′ represent virtually any type of substituents.

In certain exemplary embodiments, compounds of formula I may be prepared as follows according to Scheme 2:

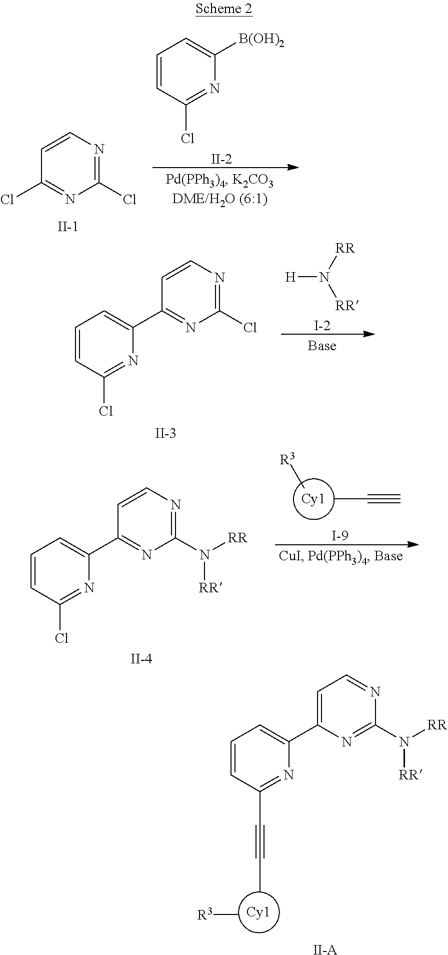

The definitions of

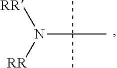

Cy1, and R³ are the same as those in Scheme 1. Suzuki Coupling of 2,4-dichloropyrimidine (II-1) with boronic acid II-2 under standard condition to give dichloro intermediate II-3. The displacement reaction between II-3 and amine I-2 give chloride II-4. Sonagoshira coupling of II-4 with alkyne I-9 give the target compound II-A. Suzuki coupling is a name reaction in organic chemistry. More detailed information about Suzuki Coupling reaction can be found in a publication (N. Miyaura and A. Suzuki Chem. Rev. 1995, 95, 2457-2483).

It will be appreciated that the reaction sequence illustrated in Scheme 2 is general in nature, and one skilled in the art will recognize that the method could be used to prepare analogues in which Cy1, R³, RR, and RR′ represent virtually any type of substituents.

In certain exemplary embodiments, compounds of formula I may be prepared as follows according to Scheme 3:

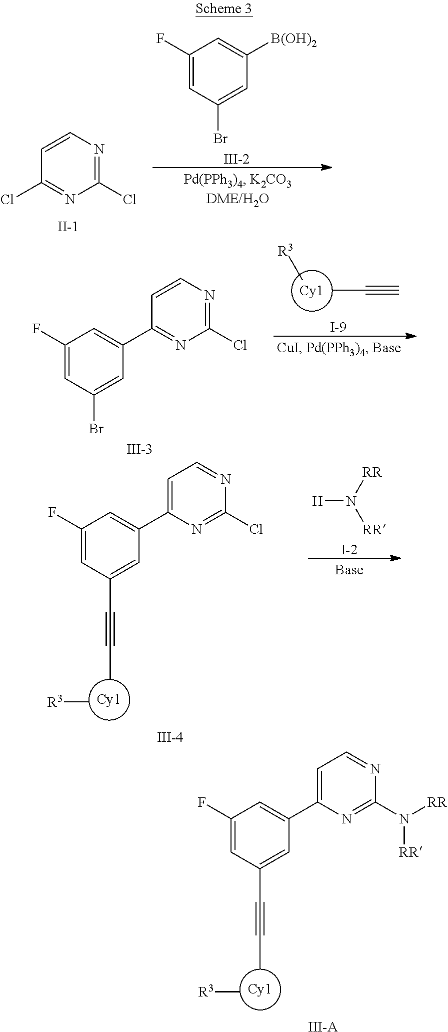

The definitions of

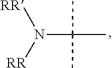

Cy1, and R³ are the same as those in Scheme 1. Suzuki Coupling of 2,4-dichloropyrimidine (II-1) with boronic acid III-2 under standard condition to give intermediate III-3. Sonagoshira coupling of III-4 with alkyne I-9 give the chloride III-4. The displacement reaction between III-4 and amine I-2 give the target molecules III-A.

It will be appreciated that the reaction sequence illustrated in Scheme 3 is general in nature, and one skilled in the art will recognize that the method could be used to prepare analogues in which Cy1, R³, RR, and RR′ represent virtually any type of substituents.

The following represent non-limiting examples of the synthetic methods.

### Example 1. 5-Methoxy-2-(4-(pyridin-4-ylethynyl)-[2,4′-bipyrimidin]-2′-yl)isoindoline (Ex. 1)

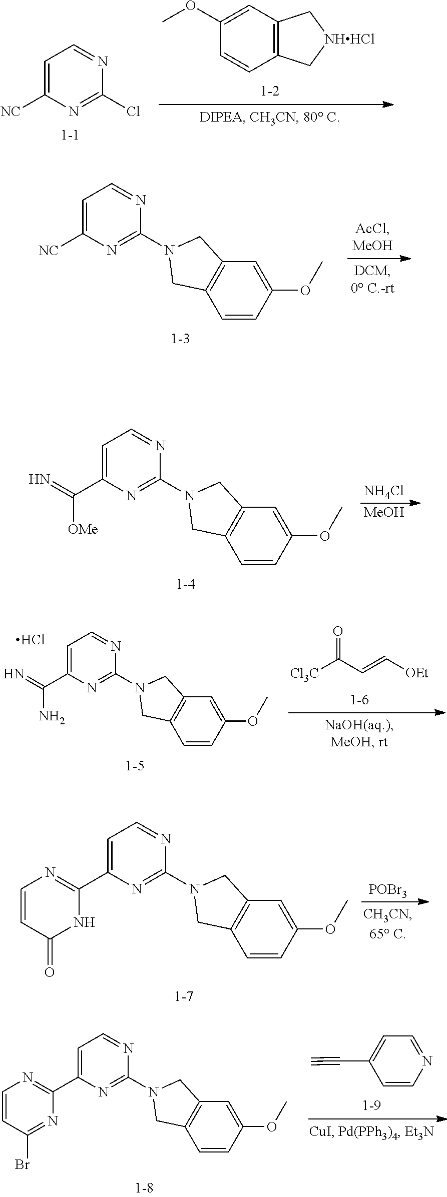

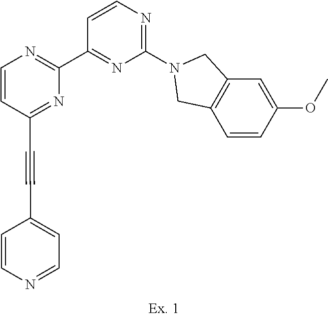 [SMILES: COc1ccc2c(c1)CN(c1nccc(-c3nccc(C#Cc4ccncc4)n3)n1)C2]

Step 1: 2-(5-Methoxyisoindolin-2-yl)pyrimidine-4-carbonitrile (1-3): To a stirred mixture of 2-chloropyrimidine (1-1, 1.5 g, 10.8 mmol) and 5-methoxyisoindoline hydrochloride (1-2, 2.0 g, 10.8 mmol) in anhydrous acetonitrile (40 mL) was dropwise added N,N-diisopropylethylamine (4.14 mL, 23.76 mmol). The reaction mixture was stirred for 3 h at 80° C. The resulting solution was concentrated under vacuum and then triturated with water, and filtered. The filter cake was thoroughly washed with water and dried under vacuum to give brownish product (1-3, 2.45 g, yield: 90%). MS (ESI⁺): m/z: 253.1 (M+H)⁺.

Step 2: Methyl 2-(5-methoxyisoindolin-2-yl)pyrimidine-4-carbimidate (1-4): To a stirred slurry of 1-3 (1.2 g, 4.8 mmol) in anhydrous methylene chloride (25 mL) was successively added acetyl chloride (3.4 mL, 47.6 mmol) and anhydrous methanol (2.9 mL, 71.4 mmol) at 0° C. The reaction mixture was slowly warmed up to rt and stirred for 12 h and then solvent was removed under vacuum to afford a yellowish solid (1-4). The solid was used for the next step without further purification.

Step 3: 2-(5-Methoxyisoindolin-2-yl)pyrimidin-4-carboximidamide hydrochloride (1-5): The yellowish solid 1-4 from the previous step was treated with ammonium chloride (565 mg, 10.56 mmol) in methanol at reflux for 8 h. After cooled down to room temperature, the reaction mixture was concentrated under vacuum. The residue was triturated with ethyl acetate, and filtered. The filter cake was used for the next step without further purification. MS (ESI⁺): m/z: 270.1 (M+H)⁺.

Step 4: 2′-(5-Methoxyisoindolin-2-yl)-[2,4′-bipyrimidin]-4-ol (1-7): A solution of (E)-1,1,1-trichloro-4-ethoxybut-3-en-2-one (1-6, 7.1 g, 32.7 mmol) in DCM (300 mL) was added to a vigorously stirred mixture of 2-(5-methoxyisoindolin-2-yl)pyrimidine-4-carboximidamide hydrochloride (1-5, 10 g, 32.7 mmol) in 2 M solution of NaOH (aq., 100 mL). The resulting mixture was stirred at room temperature for 30 min. The aqueous layer was separated and acidified with 2 N HCl (aq.). The precipitates were collected by filtration and dried under vacuum to give 2′-(5-methoxyisoindolin-2-yl)-[2,4′-bipyrimidin]-4-ol as a yellow solid (1-7, 8.2 g, yield: 78%). MS (ESI⁺): m/z: 322.2 (M+H)⁺.

Step 5: 2-(4-Bromo-[2,4′-bipyrimidin]-2′-yl)-5-methoxyisoindoline (1-8): A suspension of 2′-(5-methoxyisoindolin-2-yl)-[2,4′-bipyrimidin]-4-ol. (1-7, 8.2 g, 25.5 mmol) and POBr₃ (8.7 g, 30.6 mmol) in anhydrous acetonitrile (200 mL) was stirred at 65° C. for 1 h. After cooled down to room temperature, the resulting mixture was concentrated and poured into ice-water (200 mL) and extracted with ethyl acetate (3×300 mL). The organic layers were combined, washed with saturated NaHCO₃ (aq., 200 mL), dried over sodium sulfate, filtered, and concentrated to dryness to give 2-(4-bromo-[2,4′-bipyrimidin]-2′-yl)-5-methoxyisoindoline (1-8, 9.7 g, yield: quantitative). MS (ESI⁺): m/z: 384.2 (M+H, ⁷⁹Br)⁺, 386.2 (M+H, ⁸¹Br)⁺.

Step 6: 5-Methoxy-2-(4-(pyridin-4-ylethynyl)-[2,4′-bipyrimidin]-2′-yl)isoindoline (Ex. 1): A mixture of 2-(4-bromo-[2,4′-bipyrimidin]-2′-yl)-5-methoxyisoindoline (1-8, 20.6 mg, 0.0533 mmol), 4-ethynylpyridine (1-9, 10.9 mg, 0.106 mmol), CuI (1.01 mg, 0.0053 mmol), and Pd(PPh₃)₄ (12.3 mg, 0.0107 mmol) in Et₃N (4 mL) was purged with nitrogen at room temperature for 5 min. The resulting mixture was stirred 80° C. for 2 h. After cooled down to room temperature, the reaction mixture was concentrated and the crude product was purified by flash chromatography (ISCO, silica gel, eluted with DCM/MeOH=30/1) to afford 5-methoxy-2-(4-(pyridin-4-ylethynyl)-[2,4′-bipyrimidin]-2′-yl)isoindoline as a white solid (Ex. 1, 11 mg, yield: 51%). ¹H-NMR (300 MHz, CDCl₃): δ (ppm): 9.02 (d, J=5.1 Hz, 1H), 8.71 (d, J=5.4 Hz, 2H), 8.62 (d, J=4.8 Hz, 1H), 7.64 (d, J=5.1 Hz, 1H), 7.53 (m, 3H), 7.26 (d, J=8.1 Hz, 1H), 6.88 (m, 2H), 5.10 (m, 2H), 4.96 (m, 2H), 3.84 (s, 3H). MS (ESI⁺): m/z: 407.2 (M+H)⁺.

### Example 2. 2-(4-((1H-Pyrazol-4-yl)ethynyl)-[2,4′-bipyrimidin]-2′-yl)-5-methoxyisoindoline (Ex. 2)

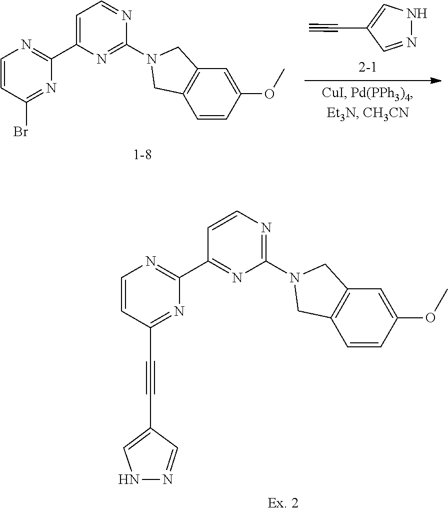

A mixture of 2-(4-bromo-[2,4′-bipyrimidin]-2′-yl)-5-methoxyisoindoline (1-8, 60 mg, 0.155 mmol), 4-ethynyl-1H-pyrazole (2-1, 29 mg, 0.311 mmol), CuI (2.95 mg, 0.0155 mmol), and Pd(PPh₃)₄ (35.8 mg, 0.031 mmol) in Et₃N (2 mL) and CH₃CN (5 mL) was purged with nitrogen at room temperature for 5 min. The resulting mixture was stirred at 75° C. for 1.5 h. After cooled down to room temperature, the reaction mixture was concentrated and the crude product was purified by flash chromatography (ISCO, silica gel, eluted with DCM/MeOH=20/1) to afford 2-(4-((1H-pyrazol-4-yl)ethynyl)-[2,4′-bipyrimidin]-2′-yl)-5-methoxyisoindoline as a pale yellow solid (Ex. 2, 20 mg, yield: 33%). ¹H-NMR (300 MHz, DMSO-d₆): δ (ppm): 13.47 (s, 1H), 9.02 (d, J=5.1 Hz, 1H), 8.65 (d, J=5.0 Hz, 1H), 8.39 (br, 1H), 7.96 (br, 1H), 7.72 (d, J=5.1 Hz, 1H), 7.54 (d, J=5.0 Hz, 1H), 7.35 (m, 1H), 7.06 (m, 1H), 6.90 (dd, J=8.2, 2.5 Hz, 1H), 4.87 (m, 4H), 3.78 (s, 3H). MS (ESI⁺): m/z: 396.2 (M+H)⁺.

### Example 3. 5-((2′-(5-Methoxyisoindolin-2-yl)-[2,4′-bipyrimidin]-4-yl)ethynyl)-1H-indazole (Ex. 3)

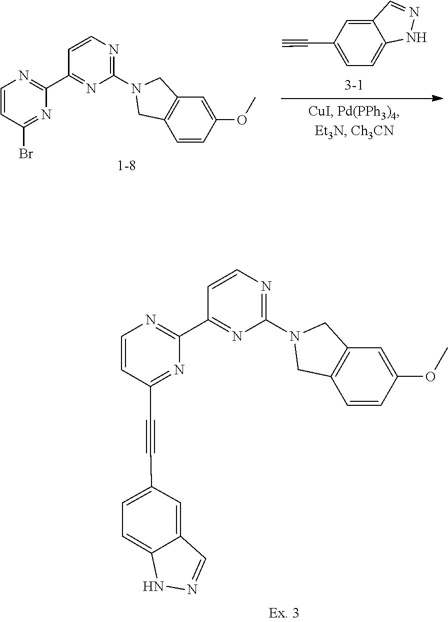 [SMILES: COc1ccc2c(c1)CN(c1nccc(-c3nccc(Br)n3)n1)C2 ; C#Cc1ccc2[nH]ncc2c1 ; COc1ccc2c(c1)CN(c1nccc(-c3nccc(C#Cc4ccc5[nH]ncc5c4)n3)n1)C2]

A mixture of 2-(4-bromo-[2,4′-bipyrimidin]-2′-yl)-5-methoxyisoindoline (I-8, 60 mg, 0.155 mmol), 5-ethynyl-1H-indazole (3-1, 44.2 mg, 0.311 mmol), CuI (2.95 mg, 0.0155 mmol), and Pd(PPh₃)₄ (35.8 mg, 0.031 mmol) in Et₃N (2 mL) and CH₃CN (5 mL) was purged with nitrogen at room temperature for 5 min. The resulting mixture was stirred at 75° C. for 1.5 h. After cooled down to room temperature, the reaction mixture was concentrated and the crude product was purified by flash chromatography (ISCO, silica gel, eluted with DCM/MeOH=20/1) to afford 5-((2′-(5-methoxyisoindolin-2-yl)-[2,4′-bipyrimidin]-4-yl)ethynyl)-1H-indazole as a pale yellow solid (Ex. 3, 22 mg, yield: 32%). ¹H-NMR (300 MHz, DMSO-d₆): δ (ppm): 13.44 (s, 1H), 9.07 (d, J=5.1 Hz, 1H), 8.67 (d, J=4.9 Hz, 1H), 8.25 (s, 1H), 8.21 (s, 1H), 7.84 (d, J=5.0 Hz, 1H), 7.66 (m, 2H), 7.57 (d, J=5.0 Hz, 1H), 7.35 (s, 1H), 7.05 (m, 1H), 6.9 (dd, J=8.5, 2.4 Hz, 1H), 4.90 (m, 4H), 3.8 (s, 3H). MS (ESI⁺): m/z: 446.2 (M+H)⁺.

### Example 4. 6-((2′-(5-Methoxyisoindolin-2-yl)-[2,4′-bipyrimidin]-4-yl)ethynyl)isoquinolin-1-amine (Ex. 4)

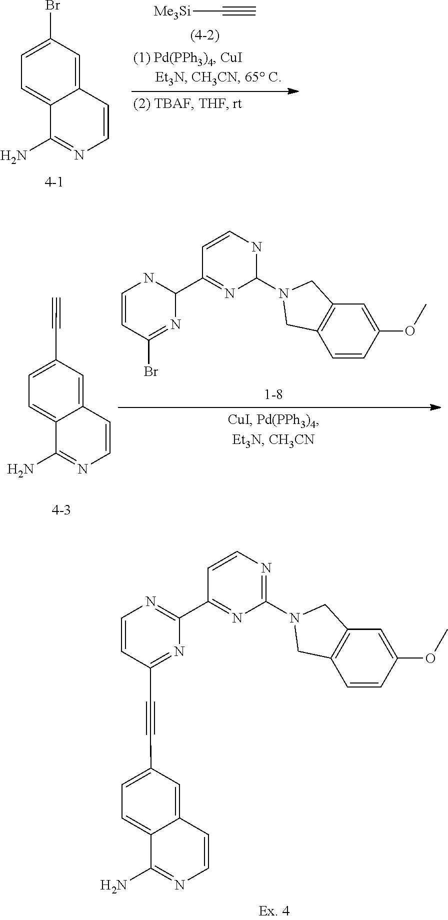

Step 1: 6-Ethynylisoquinolin-1-amine (4-3): A mixture of 6-bromoisoquinolin-1-amine (4-1, 1.0 g, 4.5 mmol), trimethylsilylacetylene (4-2, 1.8 mL, 13.5 mmol), Pd(PPh₃)₄ (100 mg, 0.09 mmol), CuI (17 mg, 0.09 mmol), and Et₃N (1.8 mL, 13.5 mmol) in acetonitrile (25 mL) was purged with nitrogen for 3 min. The resulting mixture was stirred at 65° C. for 2 h. After cooled down to room temperature, the reaction was filtered and the filtrate was concentrated in vacuo. The residue was dissolved in THF (20 mL) and TBAF (6.7 mmol) was added. The resulting mixture was stirred at room temperature for 20 min and concentrated in vacuo. The residue was purified by silica gel column chromatography to give 6-ethynylisoquinolin-1-amine (4-3, 225 mg, yield: 30%). MS (ESI⁺): m/z: 169.2 (M+H)⁺.

Step 2: 6-((2′-(5-Methoxyisoindolin-2-yl)-[2,4′-bipyrimidin]-4-yl)ethynyl)isoquinolin-1-amine (Ex. 4): A mixture of 2-(4-Bromo-[2,4′-bipyrimidin]-2′-yl)-5-methoxyisoindoline (1-8, 100 mg, 0.26 mmol), 6-ethynylisoquinolin-1-amine (4-3, 44 mg, 0.26 mmol), Pd(PPh₃)₄ (6 mg, 0.0052 mmol), CuI (1.0 mg, 0.0052 mL) and Et₃N (0.14 mL, 1.04 mmol) in acetonitrile (5 mL) was purged with nitrogen for 3 min. The resulting mixture was stirred at 65° C. for 2 h. After cooled down to room temperature, the reaction mixture was concentrated and the crude product was purified by flash chromatography (ISCO, silica gel, eluted with DCM/MeOH=10/1) to afford 6-((2′-(5-methoxyisoindolin-2-yl)-[2,4′-bipyrimidin]-4-yl)ethynyl)isoquinolin-1-amine as a brown solid (Ex. 4, 30 mg, yield: 26%). ¹H-NMR (300 MHz, CD₃OD-CDCl₃): δ (ppm): 8.93 (d, J=5.1 Hz, 1H), 8.53 (d, J=5.1 Hz, 1H), 7.99-7.92 (m, 2H), 7.81 (d, J 6 Hz, 1H), 7.67-7.56 (m, 3H), 7.21 (d, J=6.6 Hz, 1H), 6.96 (d, J=6 Hz, 1H), 6.86-6.81 (m, 2H), 5.07-4.87 (m, 4H), 3.79 (s. 3H). MS (ESI⁺): m/z: 472.3 (M+H)⁺.

### Example 5. 3-Fluoro-5-((2′-(5-methoxyisoindolin-2-yl)-[2,4′-bipyrimidin]-4-yl)ethynyl)-1H-indazole (Ex. 5)

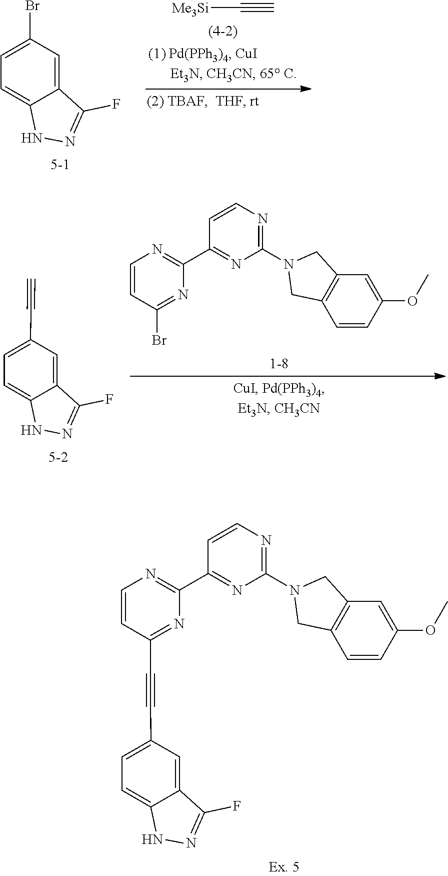

Step 1: 5-Ethynyl-3-fluoro-1H-indazole (5-2): Prepared according to the procedure for Intermediate 4-3. 320 mg obtained. Yield: 56%. MS (ESI⁺): m/z: 161.2 (M+H)⁺.

Step 2: 3-Fluoro-5-((2′-(5-methoxyisoindolin-2-yl)-[2,4′-bipyrimidin]-4-yl)ethynyl)-1H-indazole (Ex. 5): Prepared according to the procedure in Step 2 for Ex. 4. Yield: 17%. ¹H-NMR (300 MHz, CD₃OD-CDCl₃): δ (ppm): 8.93 (d, J=5.4 Hz, 1H), 8.55 (d, J=4.8 Hz, 1H), 8.07 (s, 1H), 7.67-7.57 (m, 3H), 7.48-7.44 (m, 1H), 7.25 (d, J=8.1 Hz, 1H), 6.91-6.84 (m, 2H), 5.05-4.90 (m, 4H), 3.81 (s. 3H). MS (ESI⁺): m/z: 464.3 (M+H)⁺.

### Example 6. 7-Fluoro-5-((2′-(5-methoxyisoindolin-2-yl)-[2,4′-bipyrimidin]-4-yl)ethynyl)-1H-indazole (Ex. 6)

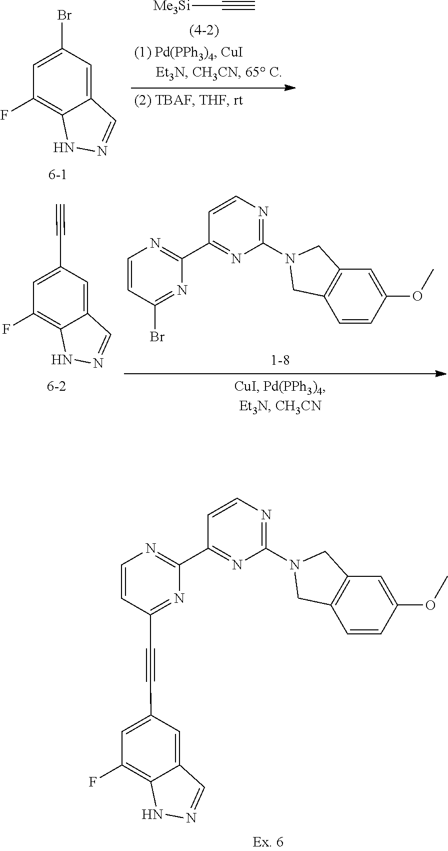

Step 1: 5-Ethynyl-7-fluoro-1H-indazole (6-2): Prepared according to the procedure for Intermediate 4-3. 510 mg obtained. Yield: 67%. MS (ESI⁺): m/z: 161.2 (M+H)⁺.

Step 2: 7-Fluoro-5-((2′-(5-methoxyisoindolin-2-yl)-[2,4′-bipyrimidin]-4-yl)ethynyl)-1H-indazole (Ex. 6): Prepared according to the procedure in Step 2 for Ex. 4. Yield: 23%. ¹H-NMR (300 MHz, CD₃OD-CDCl₃): δ (ppm): 8.92 (d, J=5.4 Hz, 1H), 8.55 (d, J=5.1 Hz, 1H), 8.13 (s, 1H), 7.94 (s, 1H), 7.64-7.58 (m, 2H), 7.34-7.7.23 (m, 2H), 6.90-6.86 (m, 2H), 5.06-4.92 (m, 4H), 3.81 (s, 3H). MS (ESI⁺): m/z: 464.3 (M+H)⁺.

### Example 7. 5-((2′-(5-Methoxyisoindolin-2-yl)-[2,4′-bipyrimidin]-4-yl)ethynyl)isoindolin-1-one (Ex. 7)

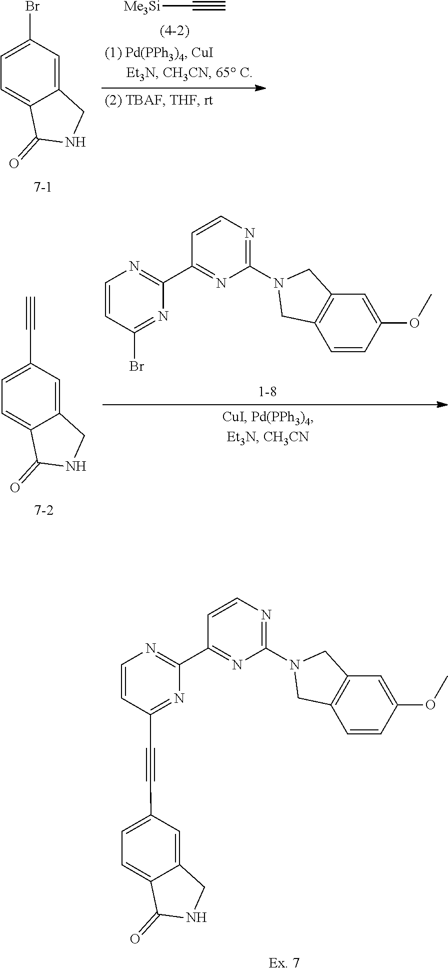

Step 1: 5-Ethynylisoindolin-1-one (7-2): Prepared according to the procedure for Intermediate 4-3. 232 mg obtained. Yield: 43%. MS (ESI⁺): m/z: 158.2 (M+H)⁺.

Step 2: 5-((2′-(5-Methoxyisoindolin-2-yl)-[2,4′-bipyrimidin]-4-yl)ethynyl)isoindolin-1-one (Ex. 7): Prepared according to the procedure in Step 2 for Ex. 4. Yield: 20%. ¹H-NMR (300 MHz, CD₃OD-CDCl₃): δ (ppm): 9.11 (d, J=4.8 Hz, 1H), 8.79 (s, 1H), 8.65 (d, J=4.8 Hz, 1H), 7.94-7.80 (m, 4H), 7.56 (s, 1H), 7.37-7.29 (m, 1H), 7.07-7.05 (m. 1H), 6.90-6.86 (m, 1H), 4.85 (t, J=12.3 Hz, 4H), 4.43 (s, 2H), 3.76 (s, 3H). MS (ESI⁺): m/z: 461.3 (M+H)⁺.

### Example 8. Methyl 4-((2′-(5-methoxyisoindolin-2-yl)-[2,4′-bipyrimidin]-4-yl)ethynyl)benzoate (Ex. 8)

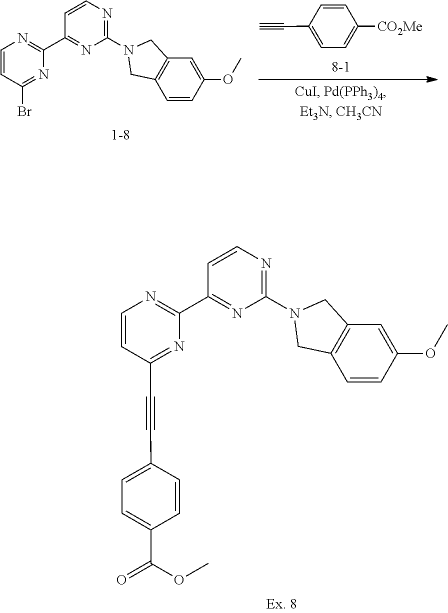

Prepared according to the procedure in Step 2 for Ex. 4. Yield: 64%. ¹H-NMR (300 MHz, CD₃OD-CDCl₃): δ (ppm): 8.94 (d, J=4.8 Hz, 1H), 8.53 (d, J=4.8 Hz, 1H), 8.04 (d, J=8.1 Hz. 2H), 7.72-7.57 (m, 4H), 7.22 (d, J=8.1 Hz, 1H), 6.87-6.82 (m, 2H), 5.04 (s, 2H), 4.90 (s, 2H), 3.79 (s, 3H). MS (ESI⁺): m/z: 464.3 (M+H)⁺.

### Example 9. 4-((2′-(5-Methoxyisoindolin-2-yl)-[2,4′-bipyrimidin]-4-yl)ethynyl)benzonitrile (Ex. 9)

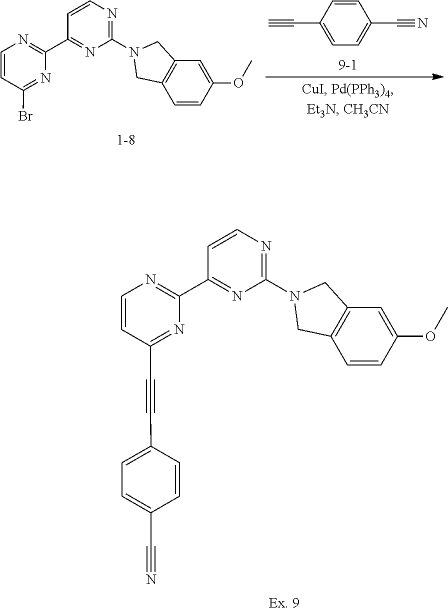

Prepared according to the procedure in Step 2 for Ex. 4. Yield: 67%. ¹H-NMR (300 MHz, CDCl₃): δ (ppm): 9.10 (d, J=4.8 Hz, 1H), 8.98 (t, J=5.4 Hz, 1H), 8.01 (d, J=6.3 Hz, 1H), 7.77-7.64 (m, 6H), 7.01-6.89 (m, 2H), 5.27 (d, J=9.9 Hz, 2H), 5.17 (d, J=11.0 Hz, 2H), 3.84 (s, 3H). MS (ESI⁺): m/z: 431.3 (M+H)⁺.

### Example 10. 4-((2′-(5-Methoxyisoindolin-2-yl)-[2,4′-bipyrimidin]-4-yl)ethynyl)benzoic acid (Ex. 10)

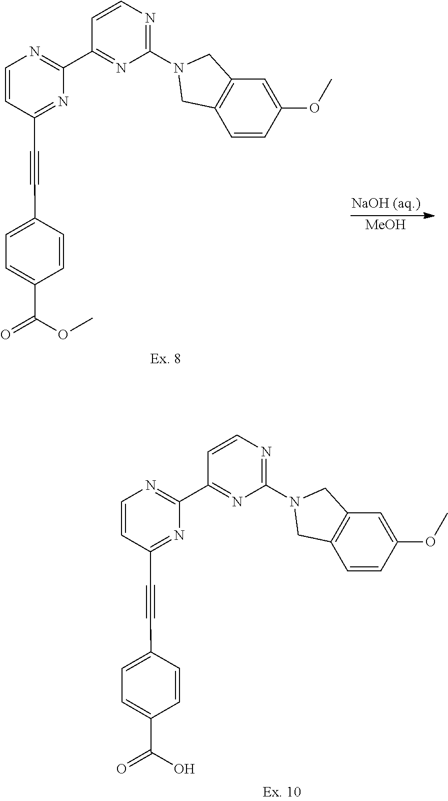 [SMILES: "MeOH" ; COC(=O)c1ccc(C#Cc2ccnc(-c3ccnc(N4Cc5ccc(OC)cc5C4)n3)n2)cc1 ; COc1ccc2c(c1)CN(c1nccc(-c3nccc(C#Cc4ccc(C(=O)O)cc4)n3)n1)C2]

A mixture of methyl 4-((2′-(5-methoxyisoindolin-2-yl)-[2,4′-bipyrimidin]-4-yl)ethynyl)benzoate (Ex. 8, 20 mg, 0.04 mmol) and 10% NaOH (aq., 1.0 mL) in MeOH (1.0 mL) was stirred at 65° C. for 1 h. After cooled down to room temperature, the reaction mixture was acidified with 1 M HCl (aq.) to pH≈2 and evaporated to dryness. The crude product was triturated with MeOH/DCM (1:1) and filtered. The filtrate was evaporated to dryness to give 4-((2′-(5-methoxyisoindolin-2-yl)-[2,4′-bipyrimidin]-4-yl)ethynyl)benzoic acid as a brown solid (Ex. 10, 12 mg, yield: 62%). ¹H-NMR (300 MHz, CDCl₃): δ (ppm): 8.95 (d, J=5.1 Hz, 1H), 8.60 (d, J=5.1 Hz, 1H), 8.1-8.08 (m, 3H), 7.68-7.65 (m, 2H), 7.50 (d, J=4.8 Hz, 1H), 6.89-6.88 (m, 2H), 5.12-4.92 (m, 4H), 3.83 (s, 3H). MS (ESI⁺): m/z: 450.3 (M+H)⁺.

### Example 11. 4-((2′-(5-Methoxyisoindolin-2-yl)-[2,4′-bipyrimidin]-4-yl)ethynyl)-N-methylbenzamide (Ex. 11)

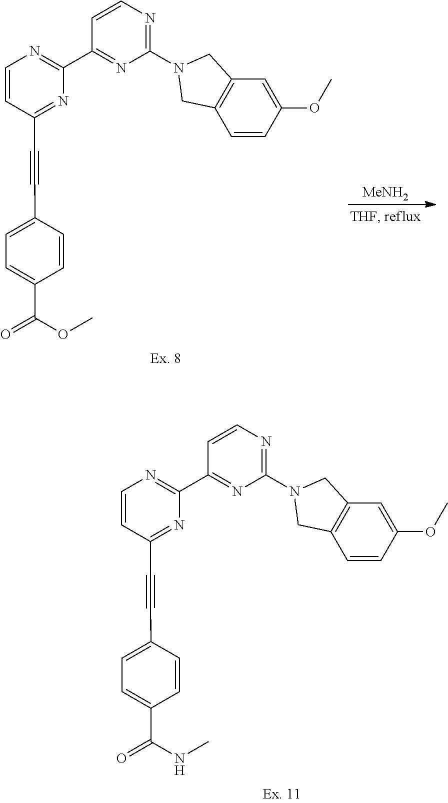 [SMILES: "MeNH2" ; COC(=O)c1ccc(C#Cc2ccnc(-c3ccnc(N4Cc5ccc(OC)cc5C4)n3)n2)cc1 ; CNC(=O)c1ccc(C#Cc2ccnc(-c3ccnc(N4Cc5ccc(OC)cc5C4)n3)n2)cc1]

A mixture of methyl 4-((2′-(5-methoxyisoindolin-2-yl)-[2,4′-bipyrimidin]-4-yl)ethynyl)benzoate (Ex. 8, 30 mg. 0.064 mmol) and 1.0 M solution of methylamine in THF (5 mL) was refluxed overnight. The solvent was evaporated and the crude product was purified by silica gel column chromatography to give 4-((2′-(5-methoxyisoindolin-2-yl)-[2,4′-bipyrimidin]-4-yl)ethynyl)-N-methylbenzamide as a light yellow solid (Ex. 11, 3 mg, yield: 10%). ¹H-NMR (300 MHz, CD₃OD-CDCl₃): δ (ppm): 8.93 (d, J=5.1 Hz, 1H), 8.12-8.08 (m, 1H), 7.86-7.82 (m, 2H), 7.69 (d, J=8.1 Hz, 1H), 7.61-7.57 (m, 1H), 7.23 (d, J 8.1 Hz, 1H), 6.88-6.82 (m, 2H), 5.02 (s, 2H), 4.88 (s, 2H), 3.80 (s, 3H), 2.93 (d, J=4.5 Hz, 3H). MS (ESI⁺): m/z: 463.3 (M+H)⁺.

### Example 12. 5-((2′-(Isoindolin-2-yl)-[2,4′-bipyrimidin]-4-yl)ethynyl)-1H-indazole (Ex. 12)

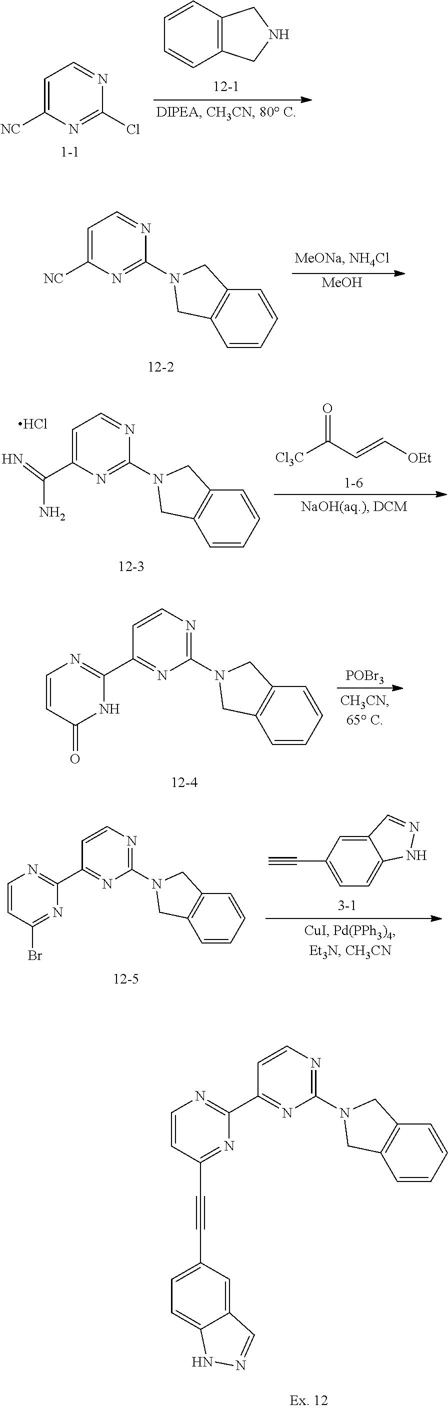

Step 1: 2-(Isoindolin-2-yl)pyrimidine-4-carbonitrile (1-3): To a stirred mixture of 2-chloropyrimidine (1-1. 583 mg, 4.18 mmol) and isoindoline (12-1, 498 mg, 4.18 mmol) in anhydrous acetonitrile (25 mL) was dropwise added N,N-diisopropylethylamine (1.6 mL, 9.19 mmol). The reaction mixture was stirred for 1 h at 80° C. The resulting solution was concentrated in vacuo and to the residue was added water. The solid product was collected by filtration, washed with water (3×5 mL), and then hexane (3×5 mL) to 2-(isoindolin-2-yl)pyrimidine-4-carbonitrile as a greyish solid (680 mg, yield: 65%). MS (ESI⁺): m/z: 223.1 (M+H)⁺.

Step 2: 2-(Isoindolin-2-yl)pyrimidine-4-carboximidamide hydrochloride (12-3): To a solution of 2-(isoindolin-2-yl)pyrimidine-4-carbonitrile (1-3, 1.3 g, 5.85 mmol) in MeOH (120 mL) was added NaOCH₃ (349 mg, 6.14 mmol) at room temperature. The resulting reaction mixture was stirred at room temperature for 3 days. Ammonium chloride (690 mg, 12.9 mmol) was added and the reaction was stirred at reflux overnight. After cooled down to room temperature, the reaction mixture was concentrated and to the residue was added anhydrous ethanol (150 mL). The reaction mixture was refluxed for 3 h, then cooled down to room temperature, and filtered. The solid product was washed with ethanol (3×5 mL) followed by hexane (3×5 mL) to give 2-(isoindolin-2-yl)pyrimidine-4-carboximidamide hydrochloride as an ivory-colored solid (12-3, 1.57 g, yield: 98%). MS (ESI⁺): m/z: 240.1 (M+H)⁺.

Step 3: 2′-(Isoindolin-2-yl)-[2,4′-bipyrimidin]-4(3H)-one (12-4): A solution of (E)-1,1,1-trichloro-4-ethoxybut-3-en-2-one (1-6, 940 mg, 3.41 mmol) in DCM (60 mL) was added to a vigorously stirred mixture of 2-(isoindolin-2-yl)pyrimidine-4-carboximidamide hydrochloride (12-3, 740 mg, 3.41 mmol) in 2 M solution of NaOH (aq., 10 mL). The resulting mixture was stirred at room temperature for 2 days. The aqueous layer was separated and acidified with 2 N HCl (aq.). The precipitates were collected by filtration and dried in vacuo to give 2′-(isoindolin-2-yl)-[2,4′-bipyrimidin]-4(3H)-one as a yellow solid (12-4, 742 mg, yield: 75%). MS (ESI⁺): m/z: 292.2 (M+H)⁺.

Step 4: 2-(4-Bromo-[2,4′-bipyrimidin]-2′-yl)isoindoline (12-5): A suspension of 2′-(isoindolin-2-yl)-[2,4′-bipyrimidin]-4(3H)-one (12-4, 562 mg, 1.93 mmol) and POBr₃ (1.11 g, 3.86 mmol) in anhydrous acetonitrile (20 mL) was stirred at 65° C. for 2.5 h. After cooled down to room temperature, the resulting mixture was concentrated and poured into ice-water (50 mL) and extracted with ethyl acetate (3×50 mL). The organic layers were combined, washed with saturated NaHCO₃ (aq., 50 mL), dried over sodium sulfate, filtered, and concentrated to dryness. The crude product was purified by silica gel flash chromatography (ISCO) to afford 2-(4-bromo-[2,4′-bipyrimidin]-2′-yl)isoindoline as a yellow solid (12-5, 148 mg, yield: 22%). MS (ESI⁺): m/z: 354.0 (M+H, ⁷⁹Br)⁺, 356.0 (M+H, ⁸¹Br)⁺.

Step 6: 5-((2′-(Isoindolin-2-yl)-[2,4′-bipyrimidin]-4-yl)ethynyl)-1H-indazole (Ex. 12): A mixture of 2-(4-bromo-[2,4′-bipyrimidin]-2′-yl)isoindoline (12-5, 45 mg, 0.127 mmol), 4-ethynylpyridine (1-9, 36.1 mg, 0.254 mmol), CuI (2.42 mg, 0.0127 mmol), and Pd(PPh₃)₄ (29.4 mg, 0.0254 mmol) in Et₃N (2 mL) and acetonitrile (5 mL) was purged with nitrogen at room temperature for 5 min. The resulting mixture was stirred 75° C. for 1.5 h. After cooled down to room temperature, the reaction mixture was concentrated and the crude product was purified by flash chromatography (ISCO, silica gel, eluted with DCM/MeOH=30/1) to afford 5-((2′-(isoindolin-2-yl)-[2,4′-bipyrimidin]-4-yl)ethynyl)-1H-indazole as a yellowish solid (Ex. 12, 38 mg, yield: 71%). ¹H-NMR (300 MHz, DMSO-d₆): δ (ppm): 13.43 (s, 1H), 9.08 (d, J=5.1 Hz, 1H), 8.67 (d, J=5.0 Hz, 1H), 8.26 (s, 1H), 8.21 (s, 1H), 7.84 (d, J=5.0 Hz, 1H), 7.66 (m, 2H), 7.58 (d, J=5.0 Hz, 1H), 7.46 (br, 2H), 7.35 (m, 2H), 4.90 (m, 4H). MS (ESI⁺): m/z: 416.2 (M+H)⁺.

### Example 13. 5-((2′-(5-Fluoroisoindolin-2-yl)-[2,4′-bipyrimidin]-4-yl)ethynyl)-1H-indazole (Ex. 13)

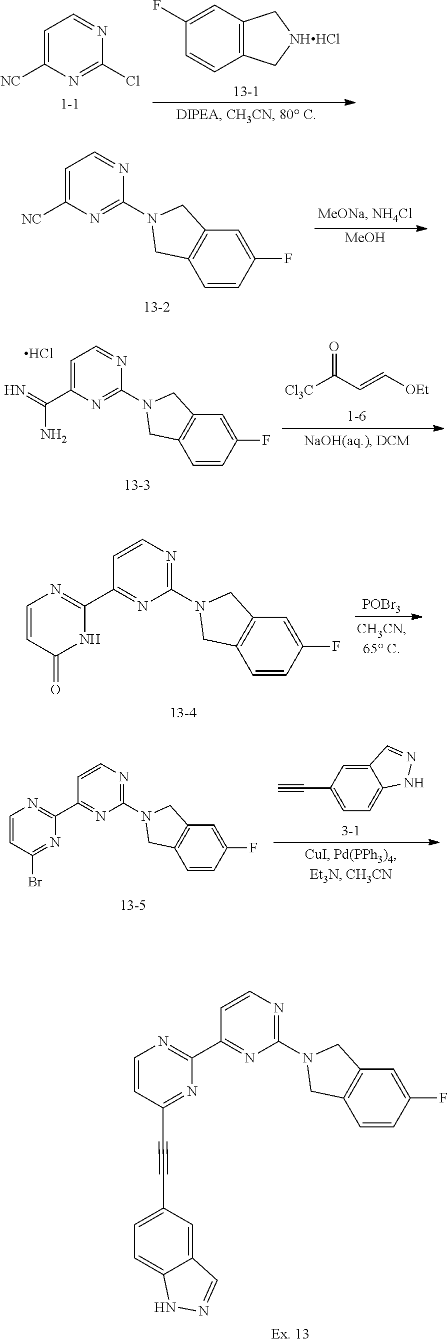

Step 1: 2-(5-Fluoroisoindolin-2-yl)pyrimidine-4-carbonitrile (13-2): To a stirred mixture of 2-chloropyrimidine-4-carbonitrile (1-1, 19.3 g, 138.3 mmol) and 5-fluoroisoindoline hydrochloride (13-1, 24.0 g, 138.3 mmol) in anhydrous acetonitrile (500 mL) was added dropwise N,N-diisopropylethylamine (53.0 mL, 304 mmol) at room temperature. The reaction mixture was stirred at 80° C. for 3 h. The resulting solution was concentrated in vacuo and then triturated with water (500 mL), and filtered. The filter cake was thoroughly washed with water (2×20 mL), followed by hexanes (3×20 mL), and dried in vacuo to give 2-(5-fluoroisoindolin-2-yl)pyrimidine-4-carbonitrile as a grey solid (13-2, 31.5 g, yield: 95%). MS (ESI⁺): m/z: 254.04 (M+H)⁺.

Step 2: 2-(5-Fluoroisoindolin-2-yl)pyrimidine-4-carboximidamide hydrochloride (13-3): To a stirred suspension of 2-(5-fluoroisoindolin-2-yl)pyrimidine-4-carbonitrile (13-2, 20.0 g, 83.2 mmol) in anhydrous methanol (500 mL) was added sodium methoxide (4.72 g, 87.4 mmol) slowly portion-wise at room temperature. The reaction mixture was stirred at 50° C. for 8 h, then to it was added ammonium chloride (9.8 g, 183.1 mmol). The resulting mixture was stirred at reflux for 8 h. After completion, the solvent was evaporated and the residue was triturated with ether (50 mL). The solid product was collected by filtration, washed with water (2×100 mL), followed by hexanes (2×100 mL), and dried in vacuo to give 2-(5-fluoroisoindolin-2-yl)pyrimidine-4-carboximidamide hydrochloride as a grey solid (13-3, 22.1 g, yield: 91%). MS (ESI⁺): m/z: 258.04 (M+H)⁺.

Step 3: 2′-(5-Fluoroisoindolin-2-yl)-[2,4′-bipyrimidin]-4(3H)-one (13-4): A suspension of 2-(5-fluoroisoindolin-2-yl)pyrimidine-4-carboximidamide hydrochloride (13-3, 3.0 g, 10.21 mmol) in 2 M NaOH (aq., 30.6 mL) and DCM (20 mL) was stirred vigorously for 10 min. (E)-1,1,1-trichloro-4-ethoxybut-3-en-2-one (1-6, 2.814 g, 12.94 mmol) and tetra-n-butylammonium bromide (100 mg) were added. The resulting mixture was stirred at 50° C. for 2 h. After completion, the reaction was diluted with water (10 mL) and the pH was adjusted to ˜1-2 by slow addition of 2 M HCl (aq.). The solid product was collected by filtration, washed with water (25 mL) followed by DCM (25 mL), and dried in vacuo to afford 2′-(5-fluoroisoindolin-2-yl)-[2,4′-bipyrimidin]-4(3H)-one as a yellow solid (13-4, 2.75 g, yield: 87%). MS (ESI⁺): m/z: 310.1 (M+H)⁺.

Step 4: 2-(4-Bromo-[2,4′-bipyrimidin]-2′-yl)-5-fluoroisoindoline (13-5): A suspension of 2′-(5-fluoroisoindolin-2-yl)-[2,4′-bipyrimidin]-4(3H)-one (13-4, 104 mg, 0.336 mmol) and POBr₃ (193 mg, 0.672 mmol) in anhydrous acetonitrile (5 mL) was stirred at 65° C. for 2.5 h. After cooled down to room temperature, the resulting mixture was concentrated and poured into ice-water (20 mL) and extracted with ethyl acetate (3×30 mL). The organic layers were combined, washed with saturated NaHCO₃ (aq., 30 mL), dried over sodium sulfate, filtered, and concentrated to dryness. The crude product was purified by silica gel flash chromatography (ISCO) to afford 2-(4-bromo-[2,4′-bipyrimidin]-2′-yl)-5-fluoroisoindoline as a yellow solid (13-5, 43 mg, yield: 34%). MS (ESI⁺): m/z: 372.2 (M+H, ⁷⁹Br)⁺, 374.2 (M+H, ⁸¹Br)⁺.

Step 5: 5-((2′-(5-Fluoroisoindolin-2-yl)-[2,4′-bipyrimidin]-4-yl)ethynyl)-1H-indazole (Ex. 13): A mixture of 2-(4-bromo-[2,4′-bipyrimidin]-2′-yl)-5-fluoroisoindoline (13-5, 24 mg, 0.0645 mmol), 4-ethynylpyridine (1-9, 18.3 mg, 0.129 mmol), CuI (1.23 mg, 0.00645 mmol), and Pd(PPh₃)₄ (14.9 mg, 0.0129 mmol) in Et₃N (1.5 mL) and acetonitrile (3.75 mL) was purged with nitrogen at room temperature for 5 min. The resulting mixture was stirred 75° C. for 1.5 h. After cooled down to room temperature, the reaction mixture was concentrated and the crude product was purified by flash chromatography (ISCO, silica gel, eluted with DCM/MeOH=30/1) to afford 5-((2′-(5-fluoroisoindolin-2-yl)-[2,4′-bipyrimidin]-4-yl)ethynyl)-1H-indazole as an ivory-colored solid (Ex. 13, 11.5 mg, yield: 41%). ¹H-NMR (300 MHz, DMSO-d₆): δ (ppm): 13.44 (s, 1H), 9.07 (d, J=5.1 Hz, 1H), 8.68 (d, J=5.0 Hz, 1H), 8.25 (s, 1H), 8.21 (s, 1H), 7.84 (d, J=5.1 Hz, 1H), 7.66 (m, 2H), 7.59 (d, J=5.0 Hz, 1H), 7.48 (br, 1H), 7.33 (br, 1H), 7.17 (m, 2H), 4.92 (m, 4H). MS (ESI⁺): m/z: 434.2 (M+H)⁺.

### Example 14. 7-Fluoro-5-((2′-(5-fluoroisoindolin-2-yl)-[2,4′-bipyrimidin]-4-yl)ethynyl)-1H-indazole (Ex. 14)

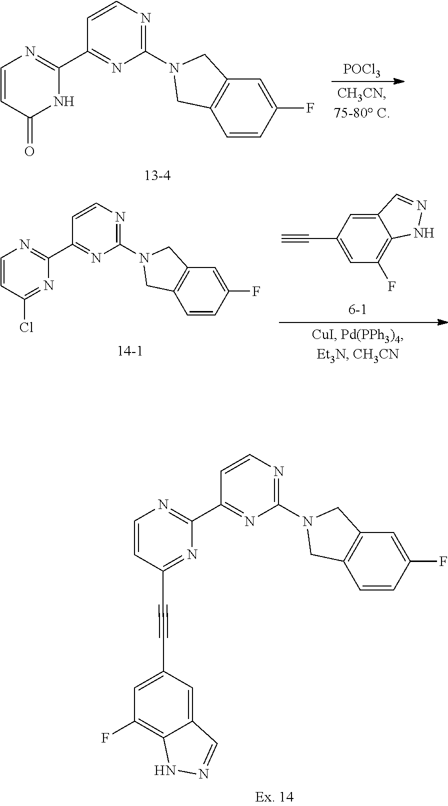 [SMILES: O=c1ccnc(-c2ccnc(N3Cc4ccc(F)cc4C3)n2)[nH]1 ; "POCl3" ; Fc1ccc2c(c1)CN(c1nccc(-c3nccc(Cl)n3)n1)C2 ; C#Cc1cc(F)c2[nH]ncc2c1 ; Fc1ccc2c(c1)CN(c1nccc(-c3nccc(C#Cc4cc(F)c5[nH]ncc5c4)n3)n1)C2]

Step 1: 2-(4-Chloro-[2,4′-bipyrimidin]-2′-yl)-5-fluoroisoindoline (14-1): A suspension of 2′-(5-fluoroisoindolin-2-yl)-[2,4′-bipyrimidin]-4(3H)-one (13-4, 1.0 g, 3.23 mmol) in acetonitrile (10 mL) and POCl₃ (1.0 mL, excess) was stirred at 80° C. for 2 h. After completion, POCl₃ and acetonitrile were removed completely under reduced pressure and the residue was quenched with saturated NaHCO₃ (aq.). The precipitated product was collected by filtration, washed with water (10 mL), followed by hexanes (50 mL), and dried in vacuo to give 2-(4-chloro-[2,4′-bipyrimidin]-2′-yl)-5-fluoroisoindoline as a pale yellow solid (14-1, 1.03 g, yield: 97%). MS (ESI⁺): m/z: 328.0 (M+H, ³⁵Cl)⁺, 330.0 (M+H, ³⁷Cl)⁺.

Step 2: 7-Fluoro-5-((2′-(5-fluoroisoindolin-2-yl)-[2,4′-bipyrimidin]-4-yl)ethynyl)-1H-indazole (Ex. 14): A suspension of 2-(4-chloro-[2,4′-bipyrimidin]-2′-yl)-5-fluoroisoindoline (14-1, 100 mg, 0.305 mmol), 5-ethynyl-7-fluoro-1H-indazole (6-1, 97.7 mg, 0.610 mmol), and CuI (5.81 mg, 0.031 mmol) in Et₃N (1.0 mL) and acetonitrile (2.0 mL) was purged with nitrogen for 10 min. Pd(PPh₃)₄ (70.5 mg, 0.061 mmol) was added and the mixture was stirred 75° C. for 1 h. After cooled down to room temperature, the reaction mixture was concentrated and the crude product was purified by silica gel flash chromatography (ISCO, eluted with 3% MeOH (contains 7 N ammonia) in DCM) to afford 7-fluoro-5-((2′-(5-fluoroisoindolin-2-yl)-[2,4′-bipyrimidin]-4-yl)ethynyl)-1H-indazole as a pale yellow solid (Ex. 14, 42.0 mg, yield: 30%). ¹H-NMR (300 MHz, DMSO-d₆): δ (ppm): 14.06 (s, 1H), 9.09 (d, J=5.0 Hz, 1H), 8.68 (d, J=5.0 Hz, 1H), 8.33 (s, 1H), 8.11 (s, 1H), 7.86 (d, J=5.1 Hz, 1H), 7.60-7.14 (m, 5H), 4.92 (s, 4H). MS (ESI⁺): m/z: 452.5 (M+H)⁺.

### Example 15. 5-((2′-(6-Methoxy-1,3-dihydro-2H-pyrrolo[3,4-c]pyridin-2-yl)-[2,4′-bipyrimidin]-4-yl)ethynyl)-1H-indazole (Ex. 15)

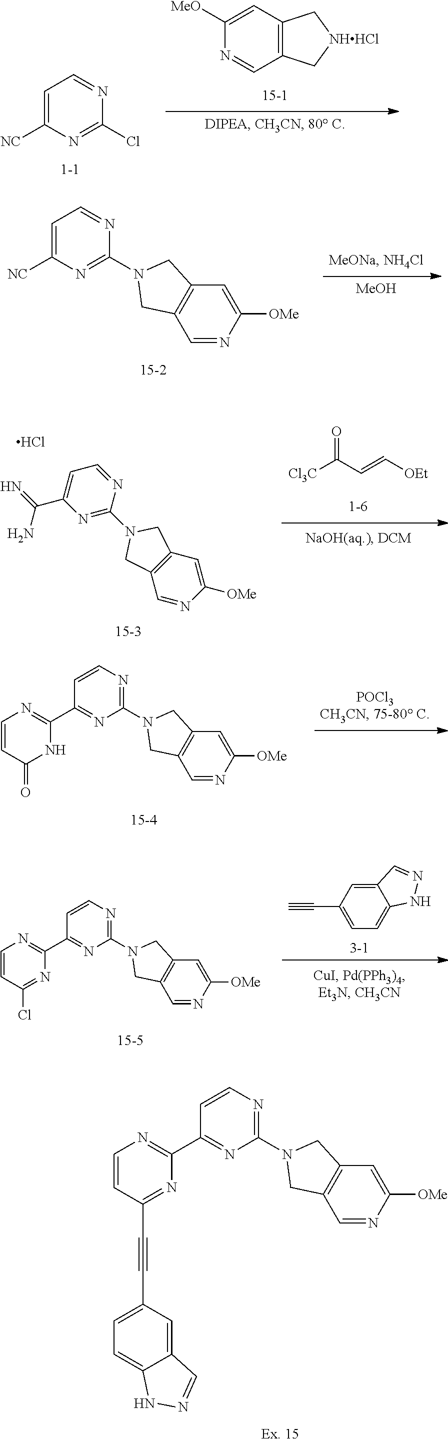

Step 1: 2-(6-Methoxy-1,3-dihydro-2H-pyrrolo[3,4-c]pyridin-2-yl)pyrimidine-4-carbonitrile (15-2): Prepared according to the procedure for Intermediate 13-2. 201 mg obtained. Yield: 99%. MS (ESI⁺): m/z: 254.05 (M+H)⁺.

Step 2: 2-(6-Methoxy-1,3-dihydro-2H-pyrrolo[3,4-c]pyridin-2-yl)pyrimidine-4-carboximidamide hydrochloride (15-3): Prepared according to the procedure for Intermediate 13-3. 220 mg obtained. Yield: 91%. MS (ESI*): m/z: 271.04 (M+H)⁺.

Step 3: 2′-(6-Methoxy-1,3-dihydro-2H-pyrrolo[3,4-c]pyridin-2-yl)-[2,4′-bipyrimidin]-4(3H)-one (15-4): Prepared according to the procedure for Intermediate 13-4. 2.0 g obtained. Yield: 95%. MS (ESI⁺): m/z: 323.05 (M+H)*.

Step 4: 2-(4-Chloro-[2,4′-bipyrimidin]-2′-yl)-6-methoxy-2,3-dihydro-1H-pyrrolo[3,4-c]pyridine (15-5): Prepared according to the procedure for Intermediate 14-1. 110 mg obtained. Yield: 87%. MS (ESI⁺): m/z: 341.07 (M+H, ³⁵Cl)⁺, 343.07 (M+H, ³⁷Cl)⁺.

Step 5: 5-((2′-(6-Methoxy-1,3-dihydro-2H-pyrrolo[3,4-c]pyridin-2-yl)-[2,4′-bipyrimidin]-4-yl)ethynyl)-1H-indazole (Ex. 15): Prepared according to the procedure in Step 2 for synthesizing Ex. 14. 101 mg obtained as a brown solid. Yield: 77%. ¹H-NMR (300 MHz, DMSO-d₆): δ (ppm): 9.07 (d, J=4.8 Hz, 1H), 8.68 (d, J=5.0 Hz, 1H), 8.25 (s, 1H), 8.21 (s, 1H), 7.84 (s, J=5.2 Hz, 1H), 7.70-7.59 (m, 3H), 7.41-7.38 (m, 1H), 6.97-6.89 (m, 1H), 4.91 (br, 2H), 4.87 (br, 2H), 3.87 (s, 3H). MS (ESI⁺): m/z: 447.13 (M+H)⁺.

### Example 16. 2-(4-((1H-Indazol-5-yl)ethynyl)-[2,4′-bipyrimidin]-2′-yl)-2,3-dihydro-1H-pyrrolo[3,4-c]pyridin-6-ol (Ex. 16)

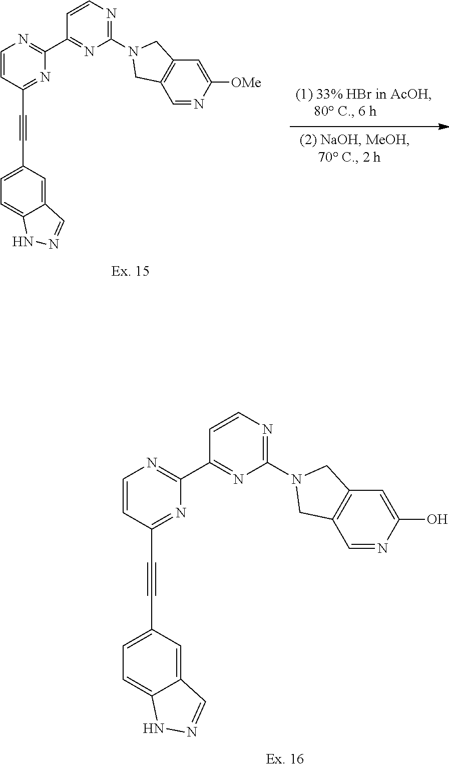 [SMILES: COc1cc2c(cn1)CN(c1nccc(-c3nccc(C#Cc4ccc5[nH]ncc5c4)n3)n1)C2 ; "(2) NaOH, MeOH,     70° C., 2 h" ; Oc1cc2c(cn1)CN(c1nccc(-c3nccc(C#Cc4ccc5[nH]ncc5c4)n3)n1)C2]

A suspension of 5-((2′-(6-methoxy-1,3-dihydro-2H-pyrrolo[3,4-c]pyridin-2-yl)-[2,4′-bipyrimidin]-4-yl)ethynyl)-1H-indazole (Ex. 15, 300 mg, 0.672 mmol) in 33% HBr in acetic acid (9 mL) was stirred at 80° C. for 6 h. LC-MS showed Ex. 15 was completely consumed. After cooled down to room temperature, the reaction mixture was concentrated under reduced pressure to dryness and the residue was suspended in MeOH (5.0 mL). NaOH (134.4 mg, 3.36 mmol) was added and the resulting mixture was refluxed for 2 h. LC-MS showed the reaction was complete. The reaction mixture was concentrated under reduced pressure and the residue was triturated with water (5 mL). The solid product was collected by filtration, washed with DCM, dried in vacuo to give the desired product 2-(4-((1H-indazol-5-yl)ethynyl)-[2,4′-bipyrimidin]-2′-yl)-2,3-dihydro-1H-pyrrolo[3,4-c]pyridin-6-ol (Ex. 16, 252.0 mg, yield: 86%). ¹H-NMR (300 MHz, DMSO-d₆): δ (ppm): 13.47 (s, 1H), 11.5 (s, 1H), 9.06 (d, J=4.4 Hz, 1H), 8.67 (d, J=4.9 Hz, 1H), 8.22 (s, 1H), 8.18 (s, 1H), 7.84 (d, J=4.9 Hz, 1H), 7.69-7.44 (m, 4H). 6.39 (s, 1H), 4.70 (br, 4H). MS (ESI⁺): m/z: 433.08 (M+H)⁺.

### Example 17. 5-((2′-(6-Chloro-1,3-dihydro-2H-pyrrolo[3,4-c]pyridin-2-yl)-[2,4′-bipyrimidin]-4-yl)ethynyl)-1H-indazole (Ex. 17)

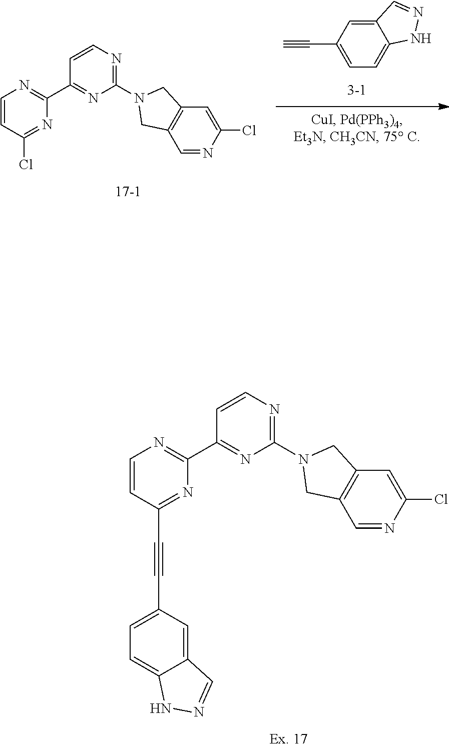 [SMILES: Clc1cc2c(cn1)CN(c1nccc(-c3nccc(Cl)n3)n1)C2 ; C#Cc1ccc2[nH]ncc2c1 ; "CuI, Pd(PPh3)4,Et3N, CH3CN, 75° C." ; Clc1cc2c(cn1)CN(c1nccc(-c3nccc(C#Cc4ccc5[nH]ncc5c4)n3)n1)C2]

Prepared by following the same procedure for synthesizing compound Ex. 15. 25 mg product obtained as a yellow solid. Yield: 20%. ¹H-NMR (300 MHz, DMSO-d₆): δ (ppm): 13.45 (s, 1H), 9.08 (d, J=5.1 Hz, 1H), 8.70 (d, J=4.9 Hz, 1H), 8.51 (s, 1H), 8.23 (s, 1H), 8.19 (s, 1H), 7.85 (d, J=5.2 Hz, 1H), 7.70-7.62 (m, 4H), 4.99 (br, 2H), 4.94 (br, 2H). MS (ESI⁺): m/z: 451.05 (M+H, ³⁵Cl)⁺, 453.05 (M+H, ³⁷Cl)⁺.

### Example 18. 5-((6-(2-(5-Methoxyisoindolin-2-yl)pyrimidin-4-yl)pyridin-2-yl)ethynyl)-1H-indazole (Ex. 18)

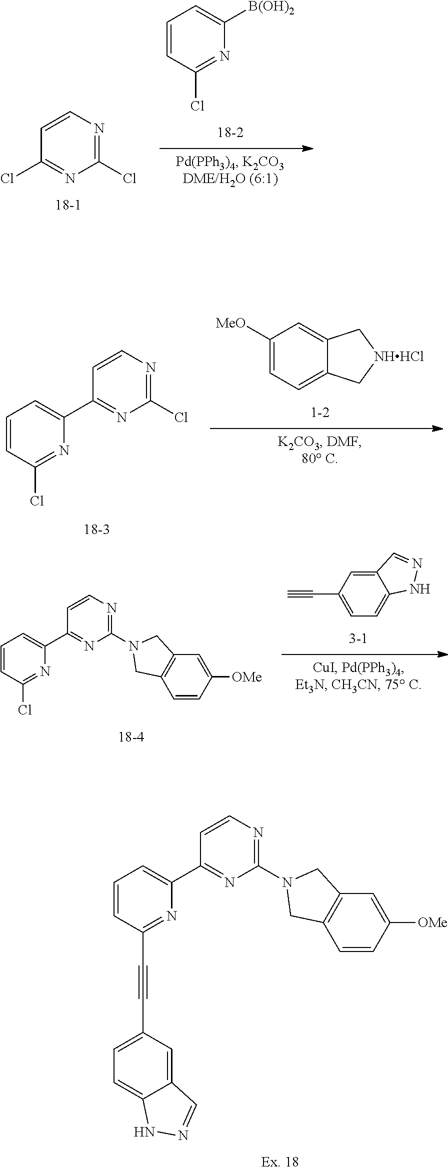

Step 1: 2-Chloro-4-(6-chloropyridin-2-yl)pyrimidine (18-3): A mixture of (6-chloropyridin-2-yl)boronic acid (18-2, 460 mg, 2.92 mmol), 2,4-dichloropyrimidine (18-1, 443 mg. 2.98 mmol), Pd(PPh₃)₄ (337 mg, 0.292 mmol), and K₂CO₃ (1.21 g, 8.76 mmol) in DME (18 mL) and water (3 mL) was purged with nitrogen at room temperature for 5 min. The resulting mixture was stirred at 80° C. for 2 days. After cooled down to room temperature, the reaction mixture was quenched with water (5 mL) and extracted with DCM (3×10 mL). The organic layers were combines, dried over MgSO₄, filtered, and then concentrated. The crude product was purified by silica gel flash chromatography (ISCO, eluted with DCM) to afford 2-chloro-4-(6-chloropyridin-2-yl)pyrimidine as a white solid (18-3, 247 mg, yield: 37%). MS (ESI⁺): m/z: 226.1 (M+H, ³⁵Cl, ³⁵Cl)⁺, 228.1 (M+H, ³⁵Cl, ³⁷Cl)⁺.

Step 2: 2-(4-(6-Chloropyridin-2-yl)pyrimidin-2-yl)-5-methoxyisoindoline (18-4): A mixture of 2-chloro-4-(6-chloropyridin-2-yl)pyrimidine (18-3, 100 mg, 0.442 mmol), 5-methoxyisoindoline hydrochloride (1-2, 82 mg, 0.442 mmol), and K₂CO₃ (184 mg, 1.33 mmol) in DMF (4 mL) was stirred at room temperature for 30 min, and then at 80° C. for 7 h. After cooled down to room temperature, the reaction mixture was quenched with water (5 mL) and extracted with ethyl acetate (3×10 mL). The organic layers were combines, dried over MgSO₄, filtered, and then concentrated. The crude product was purified by silica gel flash chromatography (ISCO, eluted with DCM) to afford 2-(4-(6-chloropyridin-2-yl)pyrimidin-2-yl)-5-methoxyisoindoline as a white solid (18-4, 44.4 mg, yield: 30%). MS (ESI): m/z: 339.2 (M+H, ³⁵Cl)⁺, 341.2 (M+H, ³⁷Cl)⁺.

Step 3: 5-((6-(2-(5-Methoxyisoindolin-2-yl)pyrimidin-4-yl)pyridin-2-yl)ethynyl)-1H-indazole (Ex. 18): A mixture of 2-(4-(6-chloropyridin-2-yl)pyrimidin-2-yl)-5-methoxyisoindoline (18-4, 20 mg, 0.059 mmol), 5-ethynyl-1H-indazole (3-1, 8.39 mg, 0.059 mmol), CuI (1.12 mg, 0.0059 mmol), and Pd(PPh₃)₄ (13.6 mg, 0.0118 mmol) in Et₃N (1.0 mL) and MeCN (2.5 mL) was purged with nitrogen at room temperature for 5 min. The reaction mixture was stirred at 75° C. for 4 h, then cooled down to room temperature, and concentrated. The residue was purified by silica gel flash chromatography to afford 5-((6-(2-(5-methoxyisoindolin-2-yl)pyrimidin-4-yl)pyridin-2-yl)ethynyl)-1H-indazole as a yellow solid (Ex. 18, 3.3 mg, yield: 13%). ¹H-NMR (300 MHz, CDCl₃-CD₃OD): δ (ppm): 8.48 (d, J=5.0 Hz, 1H), 8.43 (d, J=7.7 Hz, 1H), 8.03 (m. 2H), 7.84 (t, J=8.1 Hz, 1H), 7.65 (m, 4H), 7.22 (m. 1H), 6.86 (m, 2H), 4.91 (m, 4H). MS (ESI⁺): m/z: 445.6 (M+H)⁺.

### Example 19. 7-Fluoro-5-((6-(2-(5-methoxyisoindolin-2-yl)pyrimidin-4-yl)pyridin-2-yl)ethynyl)-1H-indazole (Ex. 19)

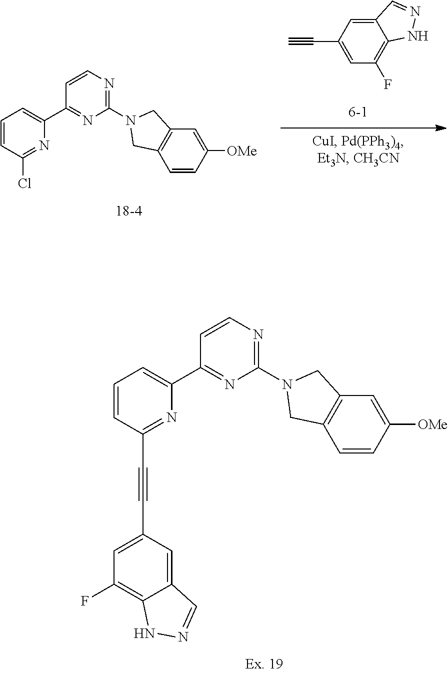

A mixture of 2-(4-(6-chloropyridin-2-yl)pyrimidin-2-yl)-5-methoxyisoindoline (18-4, 21.6 mg, 0.0638 mmol), 5-ethynyl-7-fluoro-1H-indazole (6-1, 9.1 mg, 0.0368 mmol), CuI (1.22 mg, 0.00645 mmol), and Pd(PPh₃)₄ (14.9 mg, 0.00638 mmol) in Et₃N (1.0 mL) and MeCN (3.0 mL) was purged with nitrogen at room temperature for 5 min. The reaction mixture was stirred at 75° C. for 4 h, then cooled down to room temperature, and concentrated. The residue was purified by silica gel flash chromatography (ISCO, eluted with DCM/MeOH=30:1) to afford 7-fluoro-5-((6-(2-(5-methoxyisoindolin-2-yl)pyrimidin-4-yl)pyridin-2-yl)ethynyl)-1H-indazole as an ivory-colored solid (Ex. 19, 5.6 mg, yield: 19%). MS (ESI⁺): m/z: 463.3 (M+H)*.

### Example 20. 5-((6-(2-(5-Fluoroisoindolin-2-yl)pyrimidin-4-yl)pyridin-2-yl)ethynyl)-1H-indazole (Ex. 20)

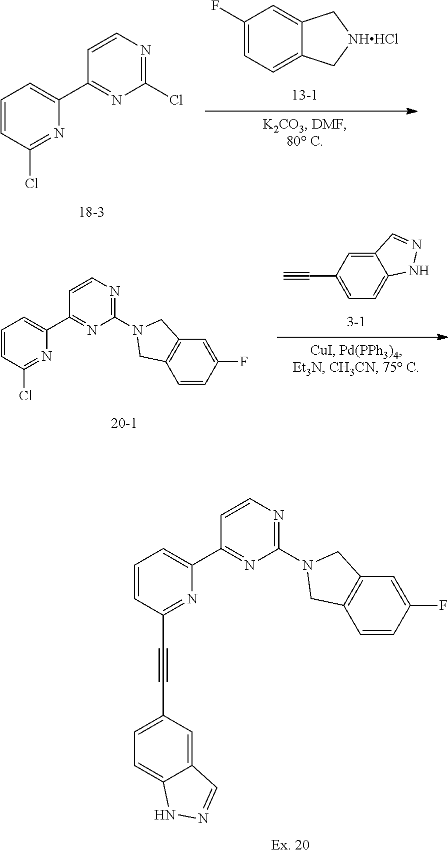

Step 1: 2-(4-(6-Chloropyridin-2-yl)pyrimidin-2-yl)-5-fluoroisoindoline (20-1): A mixture of 2-chloro-4-(6-chloropyridin-2-yl)pyrimidine (18-3, 167 mg, 0.739 mmol), 5-fluoroisoindoline hydrochloride (13-1, 138 mg, 0.739 mmol), and K₂CO₃ (307 mg, 2.22 mmol) in DMF (5 mL) was stirred at room temperature for 30 min, and then at 80° C. for 7 h. After cooled down to room temperature, the reaction mixture was quenched with water (5 mL). The solid product were collected by filtration, washed with water (3×5 mL) and hexanes (3×5 mL), and dried to afford 2-(4-(6-chloropyridin-2-yl)pyrimidin-2-yl)-5-fluoroisoindoline as a grey solid (20-1, 190 mg, yield: 79%). MS (ESI⁺): m/z: 327.2 (M+H, ³⁵Cl)⁺, 329.2 (M+H, ³⁷Cl)⁺.

Step 2: 5-((6-(2-(5-Fluoroisoindolin-2-yl)pyrimidin-4-yl)pyridin-2-yl)ethynyl)-1H-indazole (Ex. 20): A suspension of 2-(4-(6-chloropyridin-2-yl)pyrimidin-2-yl)-5-fluoroisoindoline (20-1, 70 mg, 0.214 mmol), 5-ethynyl-1H-indazole (3-1, 30.1 mg, 0.214 mmol), Pd(PPh₃)₄ (49.5 mg, 0.0428 mmol), and CuI (4.08 mg, 0.0214 mmol) in Et₃N (1.5 mL) and acetonitrile (4.5 mL) was purged with nitrogen for 5 min. The resulting mixture was stirred 75° C. for 4 h. After cooled down to room temperature, the reaction mixture was concentrated and the crude product was purified by washing with ethyl acetate and DCM to give 5-((6-(2-(5-fluoroisoindolin-2-yl)pyrimidin-4-yl)pyridin-2-yl)ethynyl)-1H-indazole as an ivory-colored solid (Ex. 20, 57.5 mg, yield: 62%). ¹H-NMR (300 MHz, DMSO-d₆): δ (ppm): 13.47 (s, 1H), 8.63 (d, J=5.0 Hz, 1H), 8.49 (d, J=7.7 Hz, 1H), 8.16 (m, 2H), 8.09 (t, J=7.7 Hz, 1H), 7.82 (d, J=7.7 Hz, 1H), 7.62 (m, 3H), 7.48 (m, 1H), 7.31 (m, 1H), 7.17 (m, 1H), 4.97 (m, 2H), 4.88 (m, 2H). MS (ESI⁺): m/z: 433.2 (M+H)⁺.

### Example 21. 7-Fluoro-5-((6-(2-(5-fluoroisoindolin-2-yl)pyrimidin-4-yl)pyridin-2-yl)ethynyl)-1H-indazole (Ex. 21)

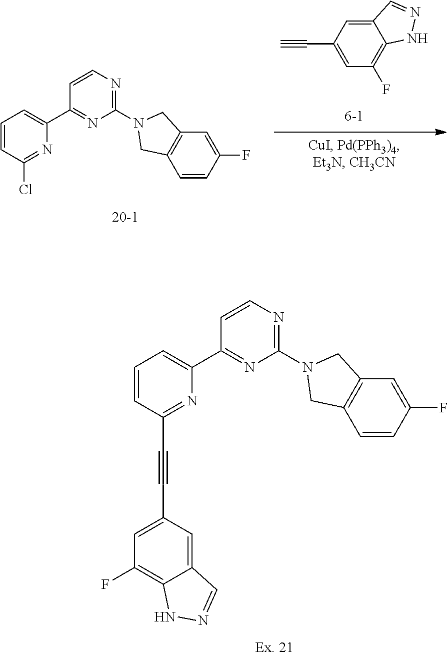

A suspension of 2-(4-(6-chloropyridin-2-yl)pyrimidin-2-yl)-5-fluoroisoindoline (20-1, 70 mg, 0.214 mmol), 5-ethynyl-7-fluoro-1H-indazole (6-1, 34.3 mg, 0.214 mmol), Pd(PPh₃)₄ (49.5 mg, 0.0428 mmol), and CuI (4.08 mg, 0.0214 mmol) in Et₃N (1.5 mL) and acetonitrile (4.5 mL) was purged with nitrogen for 5 min. The resulting mixture was stirred 75° C. for 4 h. After cooled down to room temperature, the reaction mixture was concentrated and the crude product was purified by washing with ethyl acetate and DCM to give 7-fluoro-5-((6-(2-(5-fluoroisoindolin-2-yl)pyrimidin-4-yl)pyridin-2-yl)ethynyl)-1H-indazole as a white solid (Ex. 21, 10 mg, yield: 10%). ¹H-NMR (300 MHz, DMSO-d₆): δ (ppm): 13.98 (s, 1H), 8.63 (d, J=5.1 Hz, 1H), 8.50 (d, J=7.8 Hz, 1H), 8.29 (s, 1H), 8.10 (t, J=7.8 Hz, 1H), 8.02 (s, 1H), 7.83 (d, J=7.7 Hz, 1H), 7.60 (d, J=5.1 Hz, 1H), 7.48 (m, 2H), 7.26 (m, 1H), 7.17 (m, 1H), 4.97 (m, 2H), 4.88 (m, 2H). MS (ESI⁺): m/z: 451.2 (M+H)⁺.

### Example 22. 2-((2-(4-(6-((1H-Indazol-5-yl)ethynyl)pyridin-2-yl)pyrimidin-2-yl)isoindolin-5-yl)oxy)-N,N-dimethylethanamine (Ex. 22)

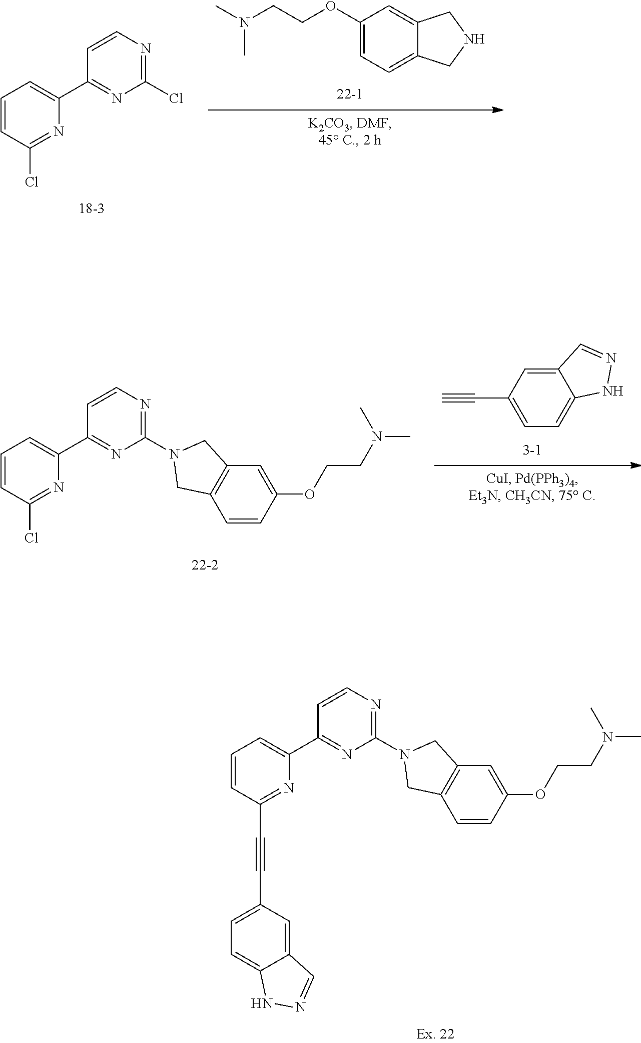

Step 1: 2-((2-(4-(6-Chloropyridin-2-yl)pyrimidin-2-yl)isoindolin-5-yl)oxy)-N,N-dimethylethanamine (22-2): A mixture of 2-chloro-4-(6-chloropyridin-2-yl)pyrimidine (18-3, 200 mg, 1.3 mmol), 2-(isoindolin-5-yloxy)-N,N-dimethylethanamine (22-1, prepared according to WO2008005565, 202 mg, 1.0 mmol), and K₂CO₃ (517 mg, 4.0 mmol) in DMF (2 mL) was stirred at 45° C. for 2 h. After cooled down to room temperature, the reaction mixture was concentrated in vacuo and the residue was purified by silica gel flash chromatography (ISCO, eluted with 0-5% MeOH (contains 7 M NH₃) in DCM) to afford 2-((2-(4-(6-chloropyridin-2-yl)pyrimidin-2-yl)isoindolin-5-yl)oxy)-N,N-dimethylethanamine as a yellowish solid (22-2, 100.2 mg, yield: 38%). MS (ESI⁺): m/z: 396.1 (M+H, ³⁵Cl)⁺, 398.1 (M+H, ³⁷Cl)⁺.

Step 2: 2-((2-(4-(6-((1H-Indazol-5-yl)ethynyl)pyridin-2-yl)pyrimidin-2-yl)isoindolin-5-yl)oxy)-N,N-dimethylethanamine (Ex. 22): A suspension of 2-((2-(4-(6-chloropyridin-2-yl)pyrimidin-2-yl)isoindolin-5-yl)oxy)-N,N-dimethylethanamine (22-2, 100 mg, 0.25 mmol), 5-ethynyl-1H-indazole (3-1, 43.6 mg, 0.3 mmol), Pd(PPh₃)₄ (57.8 mg, 0.05 mmol), and CuI (5.7 mg, 0.03 mmol) in Et₃N (1.0 mL) and acetonitrile (3.0 mL) was purged with nitrogen for 10 min. The resulting mixture was stirred 75° C. for 3.5 h. After cooled down to room temperature, the reaction mixture was concentrated and the crude product was purified by silica gel flash chromatography (ISCO, eluted with 0-5% MeOH (contains 7 M NH₃) in DCM) to give 2-((2-(4-(6-((1H-indazol-5-yl)ethynyl)pyridin-2-yl)pyrimidin-2-yl)isoindolin-5-yl)oxy)-N,N-dimethylethanamine as a white solid (Ex. 22). ¹H-NMR (300 MHz, CD₃OD): δ (ppm): 8.45 (t, J=3.1 Hz, 2H), 8.03 (d, J=3.9 Hz, 2H), 7.85 (m, 1H), 7.65 (d, J=2.3 Hz, 1H), 7.64-7.61 (m, 1H), 7.59 (d, J=4.5 Hz, 1H), 7.49 (d, J=4.2 Hz, 1H), 7.21 (d, J=3.3 Hz, 1H), 6.85 (m, 2H), 4.94-4.86 (br, 4H), 4.06 (m, 2H), 2.76 (m, 2H), 2.33 (s, 6H). MS (ESI⁺): m/z: 502.3 (M+H)⁺.

### Example 23. 5-((6-(2-(5-(4-Methylpiperazin-1-yl)isoindolin-2-yl)pyrimidin-4-yl)pyridin-2-yl)ethynyl)-1H-indazole (Ex. 23)

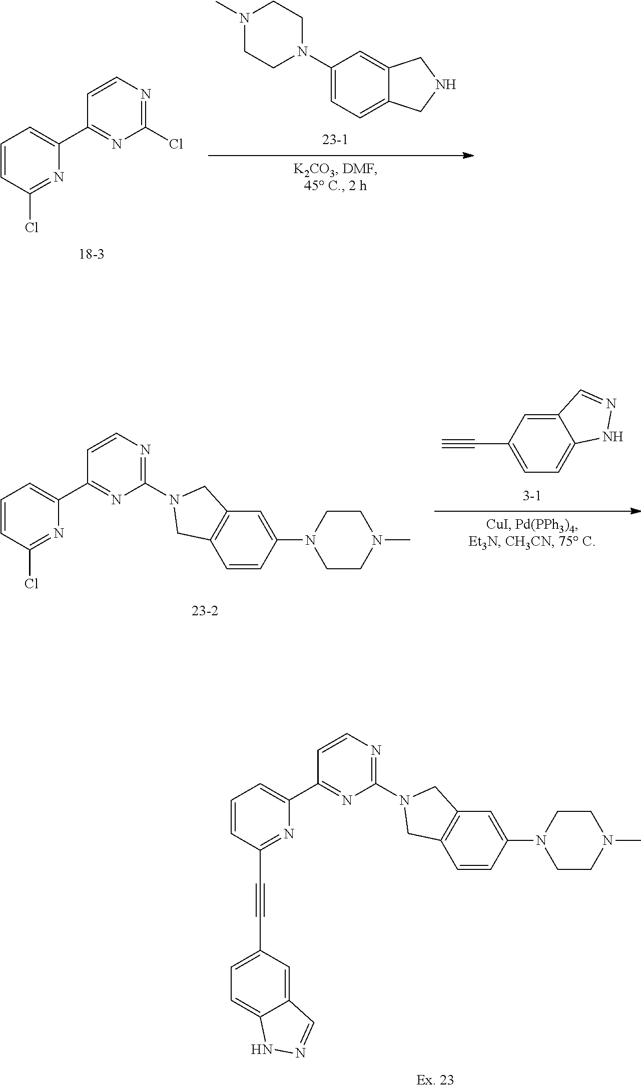

Step 1: 2-(4-(6-Chloropyridin-2-yl)pyrimidin-2-yl)-5-(4-methylpiperazin-1-yl)isoindoline (23-2): A mixture of 2-chloro-4-(6-chloropyridin-2-yl)pyrimidine (18-3, 80.6 mg, 0.51 mmol), 5-(4-methylpiperazin-1-yl)isoindoline (23-1, prepared according to WO2017007756, 100 mg, 0.39 mmol), and K₂CO₃ (504 mg, 3.9 mmol) in DMF (2 mL) was stirred at 55° C. for 2 h. After cooled down to room temperature, the reaction mixture was concentrated in vacuo and the residue was purified by silica gel flash chromatography (ISCO, eluted with 0-5% MeOH (contains 7 M NH₃) in DCM) to afford 2-(4-(6-chloropyridin-2-yl)pyrimidin-2-yl)-5-(4-methylpiperazin-1-yl)isoindoline as a yellowish solid (23-2, 41 mg, yield: 20%). MS (ESI⁺): m/z: 407.2 (M+H, ³⁵Cl)⁺, 409.2 (M+H, ³⁷Cl)⁺.

Step 2: 5-((6-(2-(5-(4-Methylpiperazin-1-yl)isoindolin-2-yl)pyrimidin-4-yl)pyridin-2-yl)ethynyl)-1H-indazole (Ex. 23): A suspension of 2-(4-(6-chloropyridin-2-yl)pyrimidin-2-yl)-5-(4-methylpiperazin-1-yl)isoindoline (23-2, 41 mg, 0.10 mmol), 5-ethynyl-1H-indazole (3-1, 17.7 mg, 0.12 mmol), Pd(PPh₃)₄ (23 mg, 0.02 mmol), and CuI (1.9 mg, 0.01 mmol) in Et₃N (1.0 mL) and acetonitrile (3.0 mL) was purged with nitrogen for 10 min. The resulting mixture was stirred 75° C. for 7 h. After cooled down to room temperature, the reaction mixture was concentrated and the crude product was purified by silica gel flash chromatography (ISCO, eluted with 0-5% MeOH (contains 7 M NH₃) in DCM) to give 5-((6-(2-(5-(4-methylpiperazin-1-yl)isoindolin-2-yl)pyrimidin-4-yl)pyridin-2-yl)ethynyl)-1H-indazole as a white solid (Ex. 23). ¹H-NMR (300 MHz, DMSO-d₆): δ (ppm): 8.60 (s, 1H), 8.46 (d, J=3.3 Hz, 1H), 8.15 (s, 1H), 8.06 (m, 1H), 7.78 (d, J=3.9 Hz, 1H), 7.57 (m, 3H), 7.25 (s, 1H), 6.99 (s, 1H), 6.91 (d, J=3.3 Hz, 1H), 4.87-4.80 (m, 4H), 3.13 (s, 4H), 2.20 (s, 4H), 0.82 (s, 3H). MS (ESI⁺): m/z: 513.3 (M+H)⁺.

### Example 24. 5-((3-Fluoro-5-(2-(5-methoxyisoindolin-2-yl)pyrimidin-4-yl)phenyl)ethynyl)-1H-indazole (Ex. 24)

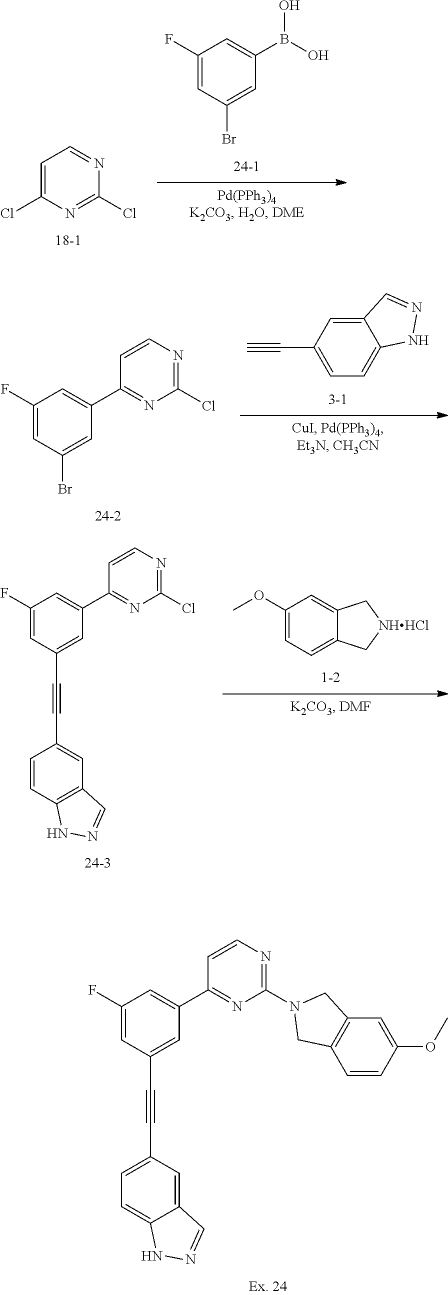

Step 1: 4-(3-Bromo-5-fluorophenyl)-2-chloropyrimidine (24-2): A mixture of 3-bromo-5-fluorobenzenebronic acid (24-1, 2.188 g, 10 mmol), 2,4-dichloropyrimidine (18-1, 1.634 g, 11 mmol), and Pd(PPh₃)₄ (577.8 mg, 0.5 mmol) in a mixture of 2.0 M K₂CO₃ (aq., 15.0 mL, 30 mmol) and dimethoxyethane (30.0 mL) was purged with nitrogen at room temperature for 10 min. The resulting mixture was stirred at 90° C. overnight. After cooled down to room temperature, the reaction mixture was extracted with ethyl acetate (3×30 mL). The organic layers were combines, dried over MgSO₄, filtered, and then concentrated. The crude product was purified by silica gel flash chromatography (ISCO, eluted with DCM) to afford 4-(3-bromo-5-fluorophenyl)-2-chloropyrimidine as a white solid (24-2, 1.995, g, yield: 69%). MS (ESI⁺): m/z: 287.2 (M+H, ³⁵Cl, ⁷⁹Br)⁺, 289.2 (M+H, ³⁵Cl, ⁸¹Br or ³⁷Cl, ⁷⁹Br)⁺, 291.2 (M+H, ³⁷Cl, ⁸¹Br)⁺.

Step 2: 5-((3-(2-Chloropyrimidin-4-yl)-5-fluorophenyl)ethynyl)-1H-indazole (24-3): A mixture of 4-(3-bromo-5-fluorophenyl)-2-chloropyrimidine (24-2, 854.7 mg, 2.93 mmol), 5-ethynyl-1H-indazole (3-1, 500.3 mg, 3.52 mmol), CuI (55.9 mg, 0.29 mmol), and Pd(PPh₃)₄ (677.9 mg, 0.59 mmol) in Et₃N (19.5 mL) and MeCN (48.8 mL) was purged with nitrogen at room temperature for 10 min. The reaction mixture was stirred at 75° C. for 2 h, then cooled down to room temperature. The reaction mixture was quenched with water (50 mL) and extracted with ethyl acetate (3×80 mL). The organic layers were combined, washed with brine, and dried over MgSO₄, filtered, and concentrated in vacuo to give a brown solid, which was purified by silica gel flash chromatography (ISCO, eluted with 0-10% MeOH in DCM) to afford 5-((3-(2-chloropyrimidin-4-yl)-5-fluorophenyl)ethynyl)-1H-indazole as a pale yellow solid (24-3, 582 mg, yield: 57%). MS (ESI⁺): m/z: 349.2 (M+H, ³⁵Cl)⁺, 351.2 (M+H, ³⁷Cl)⁺.

Step 3: 5-((3-Fluoro-5-(2-(5-methoxyisoindolin-2-yl)pyrimidin-4-yl)phenyl) ethynyl)-1H-indazole (Ex. 24): A mixture of 5-((3-(2-chloropyrimidin-4-yl)-5-fluorophenyl)ethynyl)-1H-indazole (24-3, 47 mg, 0.13 mmol), 5-methoxyisoindoline hydrochloride (1-2, 37.5 mg, 0.2 mmol), and DIPEA (28.4 mg, 0.22 mmol) in DMSO (1 mL) was purged with nitrogen for 5 min and then stirred at 100° C. for 5 h. After cooled down to room temperature, the reaction mixture was diluted with 2 mL of aqueous HCl solution (pH: ˜5-6). The aqueous layer was extracted with DCM (3×15 mL). The organic layers were combined, washed by brine, dried over MgSO₄, filtered, and concentrated. The residue was purified by silica gel flash chromatography (ISCO, eluted with 0-4% MeOH (contains 7 M NH₃) in DCM) to afford 5-((3-fluoro-5-(2-(5-methoxyisoindolin-2-yl)pyrimidin-4-yl)phenyl)ethynyl)-1H-indazole as a pale yellow solid (Ex. 24, 25 mg, yield: 42%). ¹H-NMR (300 MHz, CDCl₃): δ (ppm): 8.72 (m, 1H), 8.50 (s, 1H), 8.12 (s, 1H), 8.05 (m, 3H), 7.88 (d, J=4.5 Hz, 1H), 7.67 (d, J=4.0 Hz, 1H), 7.58 (t, J=3.4 Hz, 2H), 7.52 (d, J=4.2 Hz, 1H), 7.00 (d, J=2.8 Hz, 1H), 6.89 (t, J=3.6 Hz), 4.85-4.97 (br, 4H). 3.83 (s, 3H). MS (ESI⁺): m/z: 462.3 (M+H)⁺.

### Example 25. 5-((3-Fluoro-5-(2-(5-fluoroisoindolin-2-yl)pyrimidin-4-yl)phenyl)ethynyl)-1H-indazole (Ex. 25)

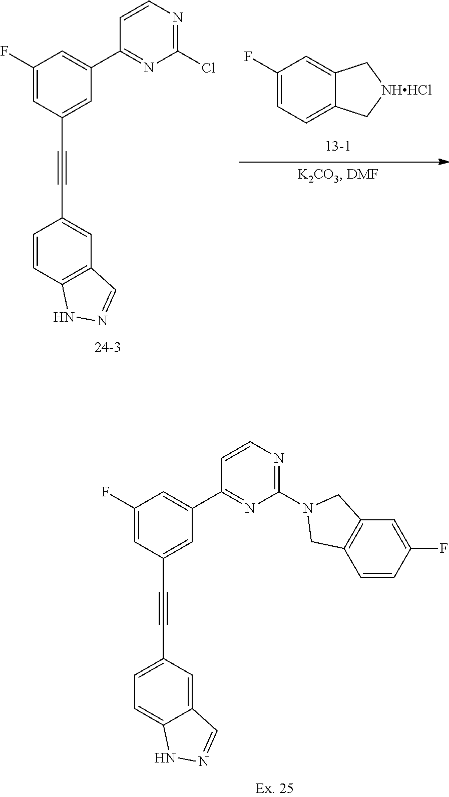

A mixture of 5-((3-(2-chloropyrimidin-4-yl)-5-fluorophenyl)ethynyl)-1H-indazole (24-3, 77 mg, 0.22 mmol), 5-fluoroisoindoline hydrochloride (13-1, 57.5 mg, 0.33 mmol), and K₂CO₃ (113.7 mg, 0.88 mmol) in DMF (2 mL) was purged with nitrogen for 5 min and then stirred at 80° C. for 3 h. After cooled down to room temperature, the reaction mixture was concentrated in vacuo and the solids was collected and washed with 50 mL of 5% MeOH (contains 7.0 M NH₃) in DCM to afford 5-((3-fluoro-5-(2-(5-fluoroisoindolin-2-yl)pyrimidin-4-yl)phenyl)ethynyl)-1H-indazole as a white solid (Ex. 25, 52 mg, yield: 54%). ¹H-NMR (300 MHz, DMSO-d₆): δ (ppm): 13.23 (s, 1H), 8.56 (m, 1H), 8.20 (s, 1H), 8.11 (s, 1H), 8.05 (m. 2H), 7.71-7.68 (br, 1H), 7.59 (d, J=4.0 Hz, 1H), 7.49 (d, J=3.8 Hz, 2H), 7.42 (s, 1H), 7.14 (t, J=12.4 Hz, 1H), 4.85-4.97 (br, 4H). MS (ESI⁺): m/z: 450.2 (M+H)⁺.

### Example 26. 5-((3-(2-(5-Chloroisoindolin-2-yl)pyrimidin-4-yl)-5-fluorophenyl)ethynyl)-1H-indazole (Ex. 26)

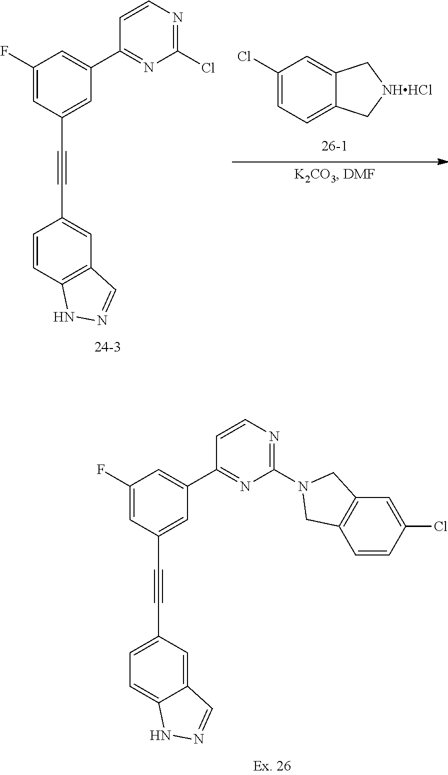

A mixture of 5-((3-(2-chloropyrimidin-4-yl)-5-fluorophenyl)ethynyl)-1H-indazole (24-3, 60 mg, 0.17 mmol), 5-chloroisoindoline hydrochloride (26-1, 49 mg, 0.26 mmol), and K₂CO₃ (88.9 mg, 0.69 mmol) in DMF (2 mL) was purged with nitrogen for 5 min and then stirred at 80° C. for 2 h. After cooled down to room temperature, the reaction mixture was concentrated in vacuo and the solids was collected and washed with 50 mL of 5% MeOH (contains 7.0 M NH₃) in DCM to give 5-((3-(2-(5-chloroisoindolin-2-yl)pyrimidin-4-yl)-5-fluorophenyl)ethynyl)-1H-indazole as a white solid (Ex. 26, 48 mg, yield: 61%). ¹H-NMR (300 MHz, DMSO-d₆): δ (ppm): 13.32 (s, 1H), 8.56 (d, J=6.0 Hz, 1H). 8.20 (s, 1H), 8.13 (s, 1H), 8.07 (m, 2H), 7.61-7.58 (m, 3H), 7.53 (m, 1H), 7.50 (s, 1H), 7.40 (m, 2H), 7.34 (m, 1H), 4.84-4.98 (br, 4H). MS (ESI⁺): m/z: 466.3 (M+H, ³⁵Cl)⁺, 468.3 (M+H, ³⁷Cl)⁺.

### Example 27. 5-((3-(2-(5-Bromoisoindolin-2-yl)pyrimidin-4-yl)-5-fluorophenyl)ethynyl)-1H-indazole (Ex. 27)

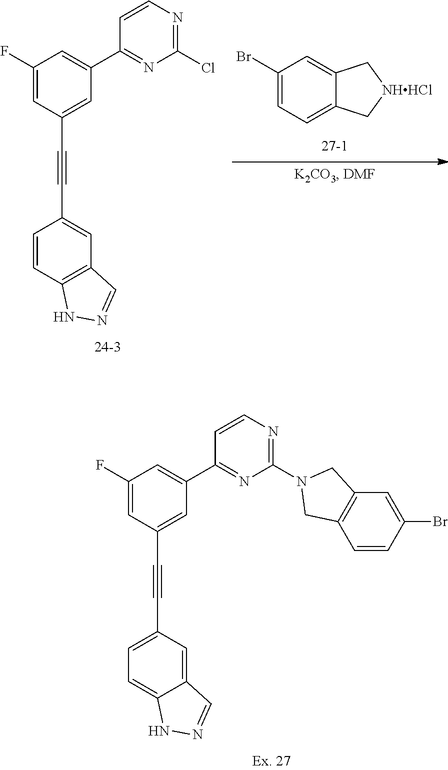

A mixture of 5-((3-(2-chloropyrimidin-4-yl)-5-fluorophenyl)ethynyl)-1H-indazole (24-3, 58 mg. 0.17 mmol), 5-bromoisoindoline hydrochloride (27-1. 58.5 mg, 0.25 mmol), and K₂CO₃ (85.8 mg, 0.68 mmol) in DMF (2 mL) was purged with nitrogen for 5 min and then stirred at 80° C. for 2 h. After cooled down to room temperature, the reaction mixture was concentrated in vacuo and the solids was collected and washed with 50 mL of 5% MeOH (contains 7.0 M NH₃) in DCM to give 5-((3-(2-(5-bromoisoindolin-2-yl)pyrimidin-4-yl)-5-fluorophenyl)ethynyl)-1H-indazole as a white solid (Ex. 27, 41 mg, yield: 47%). ¹H-NMR (300 MHz, DMSO-d₆): δ (ppm): 13.34 (s, 1H), 8.57 (d, J=8.0 Hz, 1H), 8.21 (s, 1H), 8.15 (s, 1H), 8.08 (m, 2H), 7.70-7.66 (m, 3H), 7.61 (d, J=4.5 Hz, 2H), 7.55 (s, 1H), 7.50 (d, J=4.5 Hz, 1H), 7.43 (m, 1H), 4.87-5.00 (br, 4H). MS (ESI): m/z: 510.3 (M+H, ⁷⁹Br)⁺, 512.3 (M+H, ⁷⁹Br)⁺.

### Example 28. 2-((2-(4-(3-((1H-Indazol-5-yl)ethynyl)-5-fluorophenyl)pyrimidin-2-yl)isoindolin-5-yl)oxy)-N,N-dimethylethanamine (Ex. 28)

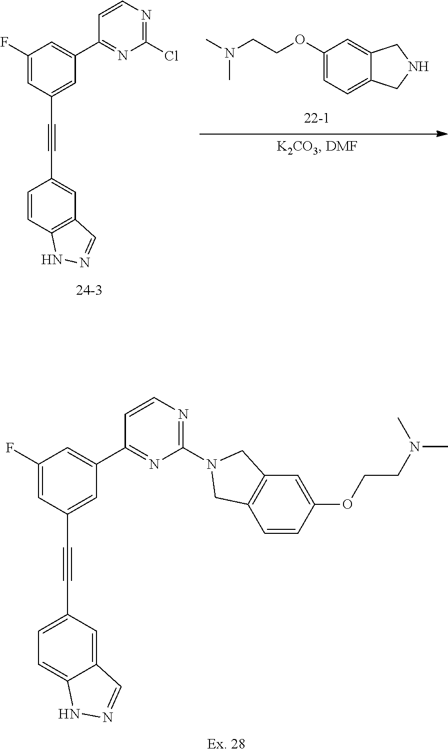

A mixture of 5-((3-(2-chloropyrimidin-4-yl)-5-fluorophenyl)ethynyl)-1H-indazole (24-3, 129 mg, 0.37 mmol), 2-(isoindolin-5-yloxy)-N,N-dimethylethanamine (22-1, 152.4 mg, 0.74 mmol), and K₂CO₃ (191.3 mg, 1.48 mmol) in DMF (2 mL) was purged with nitrogen for 5 min and then stirred at 80° C. for 2 h. After cooled down to room temperature, the reaction mixture was concentrated in vacuo and the residue was purified by silica gel flash chromatography (ISCO, eluted with 0-4% MeOH (contains 7 M NH₃) in DCM) to afford 2-((2-(4-(3-((1H-indazol-5-yl)ethynyl)-5-fluorophenyl)pyrimidin-2-yl)isoindolin-5-yl)oxy)-N,N-dimethylethanamine as a pale yellow solid (Ex. 28, 136 mg, yield: 71%). ¹H-NMR (300 MHz, DMSO-d₆): δ (ppm): 8.52 (d, J=6.0 Hz, 1H), 8.18 (s, 1H), 8.13 (s, 1H), 8.09 (m, 2H), 7.63-7.52 (m, 3H), 7.37 (d, J=2.55 Hz, 1H), 7.25 (br, 1H), 7.02 (d, J=7.5 Hz, 1H), 6.86 (d, J=4.1 Hz, 1H), 4.91-4.79 (m, 4H), 4.02 (t, J=3.3 Hz, 2H), 2.60 (t, J=2.4 Hz, 2H), 2.18 (s, 6H). MS (ESI⁺): m/z: 519.3 (M+H)⁺.

### Example 29. 5-((3-Fluoro-5-(2-(5-(4-methylpiperazin-1-yl)isoindolin-2-yl)pyrimidin-4-yl)phenyl)ethynyl)-1H-indazole (Ex. 29)

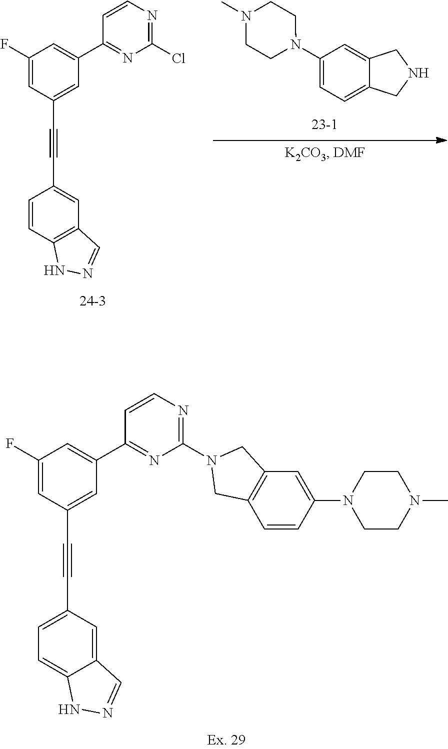

A mixture of 5-((3-(2-chloropyrimidin-4-yl)-5-fluorophenyl)ethynyl)-1H-indazole (24-3, 111.6 mg, 0.32 mmol), 5-(4-methylpiperazin-1-yl)isoindoline (23-1, 66 mg, 0.32 mmol), and K₂CO₃ (330.9 mg, 2.56 mmol) in DMF (2 mL) was purged with nitrogen for 10 min and then stirred at 80° C. for 2 h. After cooled down to room temperature, the reaction mixture was concentrated in vacuo and the residue was purified by silica gel flash chromatography (ISCO, eluted with 0-4% MeOH (contains 7 M NH₃) in DCM) to afford 5-((3-fluoro-5-(2-(5-(4-methylpiperazin-1-yl)isoindolin-2-yl)pyrimidin-4-yl)phenyl)ethynyl)-1H-indazole as a pale yellow solid (Ex. 29, 80 mg, yield: 41%). ¹H-NMR (300 MHz, CDCl₃): δ (ppm): 10.37 (br, 1H), 8.48 (d, J=2.6 Hz, 1H), 8.11 (s, 1H), 8.03 (s, 1H), 7.86 (d, J=3.0 Hz, 1H), 7.55 (dd, J=2.5, 8.7 Hz, 2H), 7.34 (d, J=4.5 Hz, 2H), 6.99 (d, J=4.5 Hz, 1H), 6.92 (d, J=2.9 Hz, 1H), 5.01-4.92 (m, 4H), 3.24 (m, 4H), 2.62 (m, 4H), 2.37 (s, 3H). MS (ESI⁺): m/z: 530.3 (M+H)⁺.

### Example 30. 5-((2′-(5-Bromoisoindolin-2-yl)-[2,4′-bipyrimidin]-4-yl)ethynyl)-1H-indazole (Ex. 30)

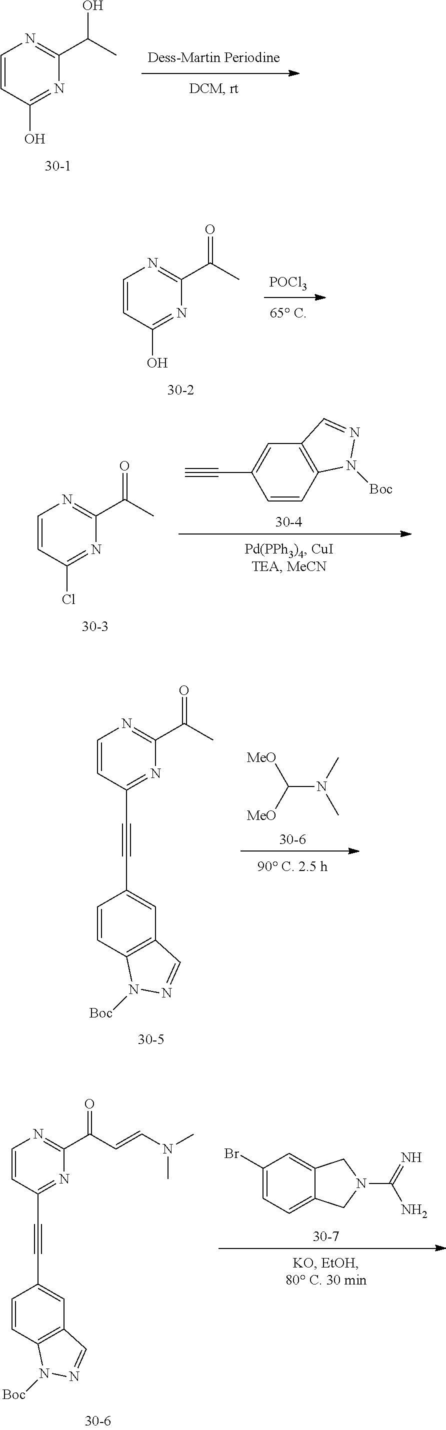

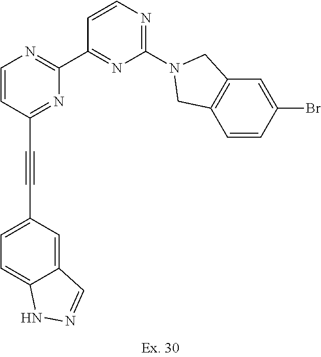 [SMILES: Brc1ccc2c(c1)CN(c1nccc(-c3nccc(C#Cc4ccc5[nH]ncc5c4)n3)n1)C2]

Step 1: 1-(4-Hydroxypyrimidin-2-yl)ethenone (30-2): To a stirred solution of 2-(1-hydroxyethyl)pyrimidin-4-ol (30-1, prepared according to B. L. Mylari et al. J. Med. Chem. 2001, 44(17), 2695-2700, 1.00 g, 7.0 mmol) in DCM (40 mL) was added Dess-Martin periodinane (4.5 g, 10.7 mmol) in portions. The resulting mixture was stirred at room temperature overnight. TLC showed the reaction was complete. 20 mL of a 1:1 mixture of 10% Na₂S₂O₃ (aq.) and saturated NaHCO₃ (aq.) was added to quench the reaction. The mixture was extracted with DCM (3×50 mL). The organic layers were combined, dried over MgSO₄, filtered, and concentrated in vacuo. The residue was purified by silica gel flash chromatography (ISCO, eluted with 0-5% MeOH in DCM) to afford 1-(4-hydroxypyrimidin-2-yl)ethenone as an off-white solid (30-2, ˜1.0 g, yield: quantitative). MS (ESI⁺): m/z: 139.07 (M+H)⁺.

Step 2: 1-(4-Chloropyrimidin-2-yl)ethenone (30-3): A mixture of 1-(4-hydroxypyrimidin-2-yl)ethenone (30-2, ˜1.0 g) POCl₃ (20 mL) was stirred at 65° C. for 3 h. LC-MS showed the reaction was complete. After the reaction mixture was cooled down to room temperature, excess amount of POCl₃ was removed in vacuo and the residue was used directly in the next step without purification. MS (ESI⁺): m/z: 157.01 (M+H)⁺.

Step 3: tert-Butyl 5-((2-acetylpyrimidin-4-yl)ethynyl)-1H-indazole-1-carboxylate (30-5): A mixture of 1-(4-chloropyrimidin-2-yl)ethenone (30-3, 177 mg, 1.13 mmol), tert-butyl 5-ethynyl-1H-indazole-1-carboxylate (30-4, 357 mg, 1.47 mmol), Pd(PPh₃)₄ (254.2 mg, 0.22 mmol) and CuI (20.9 mg, 0.11 mmol) in TEA (7.5 mL) and MeCN (11.3 mL) was purged with nitrogen for 15 min. The resulting mixture was then stirred at 65° C. for 0.5 h. LC-MS showed the reaction was complete. After cooled down to room temperature, solids were removed by filtration and the filtrate was concentrated in vacuo. The residue was purified by silica gel flash chromatography (ISCO, eluted with 0-50% ethyl acetate in hexanes) to afford tert-butyl 5-((2-acetylpyrimidin-4-yl)ethynyl)-1H-indazole-1-carboxylate as an orange solid (30-5), which was used in the next step without further purification. MS (ESI⁺): m/z: 363.15 (M+H)⁺.

Step 4: tert-Butyl 5-((2-(3-(dimethylamino)acryloyl)pyrimidin-4-yl)ethynyl)-1H-indazole-1-carboxylate (30-6): A mixture of tert-butyl 5-((2-acetylpyrimidin-4-yl)ethynyl)-1H-indazole-1-carboxylate (30-5, 50 mg, 0.14 mmol) and N,N-dimethylformamide dimethyl acetal (DMF-DMA, 0.5 mL) was stirred at 90° C. for 2 h. LC-MS showed the reaction was complete. After cooled down to room temperature, the reaction mixture was filtered and filtrate was concentrated in vacuo. The remaining solvent was removed azeotropically with toluene to give tert-butyl 5-((2-(3-(dimethylamino)acryloyl)pyrimidin-4-yl)ethynyl)-1H-indazole-1-carboxylate (30-6), which was used in the next step without purification. MS (ESI⁺): m/z: 418.20 (M+H)⁺.

Step 5: 5-((2′-(5-Bromoisoindolin-2-yl)-[2,4′-bipyrimidin]-4-yl)ethynyl)-1H-indazole (Ex. 20): A mixture of tert-butyl 5-((2-(3-(dimethylamino)acryloyl)pyrimidin-4-yl)ethynyl)-1H-indazole-1-carboxylate (30-6, 65 mg, crude), 5-bromoisoindoline-2-carboximidamide (30-7, 65 mg, 0.28 mmol) and KOH (31 mg, 0.56 mmol) in anhydrous EtOH (1 mL) was stirred at 80° C. for 30 min. LC-MS showed the reaction was complete. After cooled down to room temperature, the reaction mixture was concentrated and the residue was purified by silica gel flash chromatography (ISCO, eluted with 0-5% MeOH in DCM) to afford 5-((2′-(5-bromoisoindolin-2-yl)-[2,4′-bipyrimidin]-4-yl)ethynyl)-1H-indazole (Ex. 20) as a yellow solid. ¹H-NMR (300 MHz, CDCl₃): δ (ppm): 8.96 (d, J=2.6 Hz, 1H), 8.62 (d, J=2.6 Hz), 8.13 (d, J=2.6 Hz), 7.67 (t, J=2.4 Hz), 7.63 (m, 1H), 7.56 (d, J=0.3 Hz, 1H) 7.53-7.51 (m, 1H), 7.49 (br, 1H), 7.43 (d, J=3.54 Hz), 7.22 (d, J=4.1 Hz), 4.93-5.10 (m, 4H). MS (ESI⁺): m/z: 494.00 (M+H, ⁷⁹Br)⁺, 496.00 (M+H, ⁸¹Br)⁺.

The foregoing are merely exemplary of synthetic routes to the compound of the invention. The foregoing compounds, compositions and methods of the invention are illustrated by the following examples, which are merely exemplary of aspects of the invention and are not limiting.

### Example 31. 3-Fluoro-5-((2′-(5-fluoroisoindolin-2-yl)-[2,4′-bipyrimidin]-4-yl)ethynyl)-1H-indazole (Ex. 31)

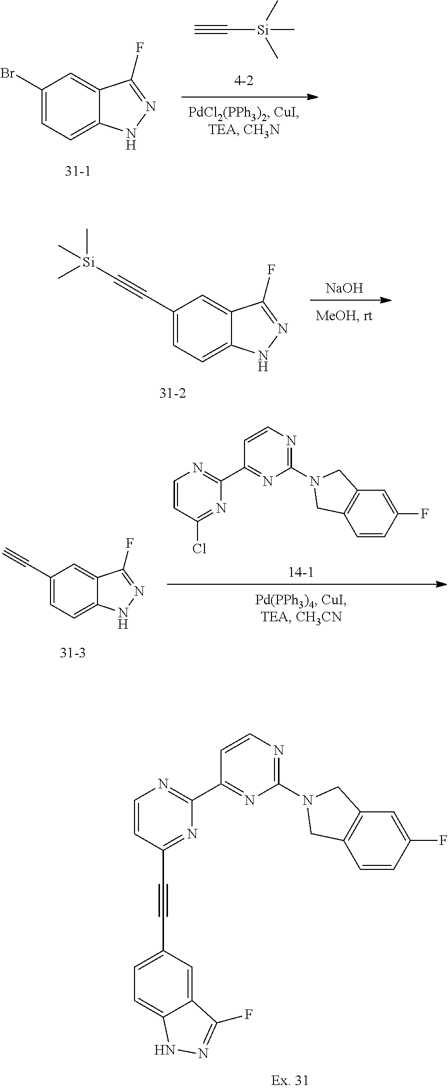

Step 1: 3-Fluoro-5-((trimethylsilyl)ethynyl)-1H-indazole (31-2): Under nitrogen, to a mixture of 5-bromo-3-fluoro-1H-indazole (31-1 (CAS #1211537-09-5, commercially available or can be easily prepared according to WO 2019/225552) (6.0 g, 27.9 mmol), trimethylsilylacetylene (4-2, 5.48 g. 55.8 mmol), Cu(I) iodide (57 mg, 0.3 mmol), PdCl₂(PPh₃)₂ (210.6 mg, 0.3 mmol), and Et₃N (8.0 mL) were added acetonitrile (30 mL). The resulting mixture was stirred at 70° C. for 2 h. LC-MS showed the reaction was complete. After cooling to room temperature, the reaction mixture was filtered, filtrate concentrated and washed with water (3×30 mL). The solids were used directly in the next step without purification.

Step 2: 5-Ethynyl-3-fluoro-1H-indazole (31-3): To a solution of the residue from Step 1 in methanol (50 mL) was added NaOH (2.232 g, 55.8 mmol). The reaction mixture was stirred at room temperature for 2 h. LC-MS showed the reaction was complete. The reaction mixture was diluted with water (50 mL) and filtered. The aqueous layer was collected and extracted with DCM (3×100 mL). The organic layers were combined, dried over MgSO₄, filtered, and concentrated in vacuo to give 5-ethynyl-3-fluoro-1H-indazole (31-3) as an off white solid, which was used directly in the next step without purification.

Step 3: 3-Fluoro-5-((2′-(5-fluoroisoindolin-2-yl)-[2,4′-bipyrimidin]-4-yl)ethynyl)-1H-indazole (Ex. 31): A suspension of 2-(4-chloro-[2,4′-bipyrimidin]-2′-yl)-5-fluoroisoindoline (14-1, 250 mg, 0.762 mmol), 5-ethynyl-3-fluoro-1H-indazole (31-3, 183.0 mg, 1.144 mmol) and CuI (14.5 mg, 0.0762 mmol) in Et₃N (1.5 mL) and acetonitrile (5.0 mL) was taken in a glass vial and purged with nitrogen gas for 10 min, then Pd(Ph₃)₄ (88.01 mg, 0.0762 mmol) was added, and the mixture was stirred at 75° C. for 1 h. After completion, the reaction mixture was cooled to room temperature and concentrated under reduced pressure. The crude product was purified by trituration in 10% MeOH in DCM (5 mL, twice) to afford 3-fluoro-5-((2′-(5-fluoroisoindolin-2-yl)-[2,4′-bipyrimidin]-4-yl)ethynyl)-1H-indazole (Ex. 31, 250.0 mg, yield: 73%) as pale brown solid. ¹H-NMR (300 MHz, DMSO-d₆): δ (ppm): 12.99 (s, 1H), 9.12 (d, J=5.1 Hz, 1H), 8.71 (d, J=4.8 Hz, 1H), 8.24 (s, 1H), 7.88 (d, J=5.1 Hz, 1H), 7.76-7.61 (m, 3H), 7.54-7.45 (m, 1H), 7.40-7.41 (m, 1H), 7.22-7.16 (m, 1H), 4.94 (brs, 4H). MS (ESI⁺): m/z: 452.4 (M+H)⁺.

### Example 32. 5-((2′-(5-Fluoroisoindolin-2-yl)-[2,4′-bipyrimidin]-4-yl)ethynyl)-1H-pyrazolo[3,4-b]pyridine (Ex. 32)

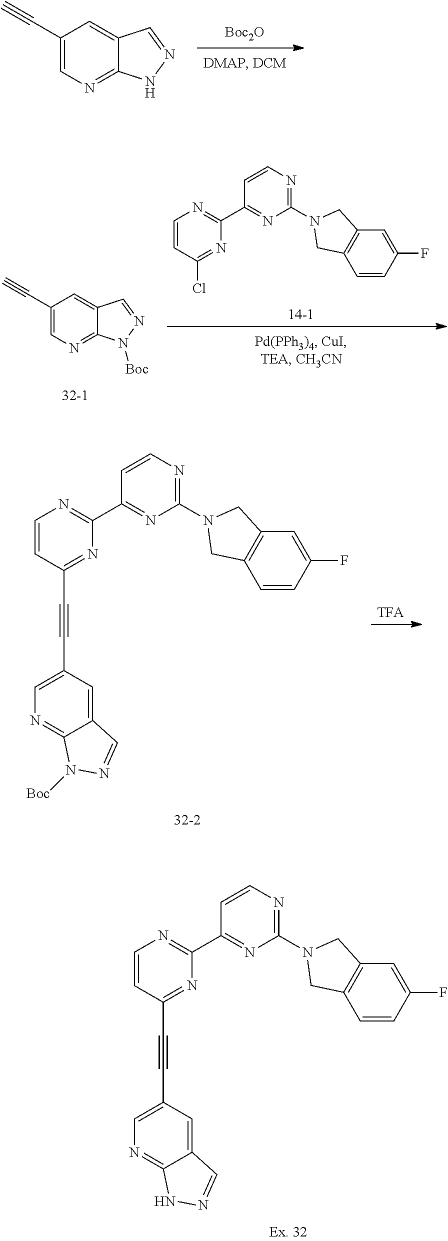

Step 1: tert-Butyl-5-ethynyl-1H-pyrazolo[3,4-b]pyridine-1-carboxylate (32-1): To a suspension of the compound 5-ethynyl-1H-pyrazolo[3,4-b]pyridine (CAS #1207351-15-2, commercially available or can be easily prepared according to X. Ren et al. J. Med. Chem. 2013, 56, 879-894) (0.4 g, 2.03 mmol) in DCM (25.0 mL) was added 4-dimethylaminopyridine (DMAP, 0.25 g, 2.03 mmol) and di-tert-butyl dicarbonate (Boc₂O, 0.58 g, 2.64 mmol). The resulting mixture was stirred at room temperature for 1 h. LC-MS showed the reaction was complete. The reaction mixture was partitioned between DCM (25 mL) and water (25 mL). The organic layer was collected and the aqueous layer was extracted with DCM (50 mL). The combined organic layers were washed with brine, dried over Na₂SO₄, filtered, and concentrated under reduced pressure. The residue was purified by column chromatography (ISCO) on neutral alumina to afford tert-butyl-5-ethynyl-1H-pyrazolo[3,4-b]pyridine-1-carboxylate (32-1, 250 mg, yield: 51%) as a pale yellow gum. MS (ESI): m/z: 244.2 (M+H)⁺.

Step 2: tert-Butyl-5-((2′-(5-fluoroisoindolin-2-yl)-[2,4′-bipyrimidin]-4-yl)ethynyl)-1H-pyrazolo[3,4-b]pyridine-1-carboxylate (32-2): A suspension of 2-(4-chloro-[2,4′-bipyrimidin]-2′-yl)-5-fluoroisoindoline (14-1, 50 mg, 0.152 mmol), tert-butyl-5-ethynyl-1H-pyrazolo[3,4-b]pyridine-1-carboxylate (32-1, 44.5 mg, 0.183 mmol) and CuI (2.9 mg, 0.0152 mmol) in Et₃N (0.25 mL) and acetonitrile (2.0 mL) was taken in a glass vial and purged with nitrogen gas for 10 min. Pd(PPh₃)₄ (17.6 mg, 0.0152 mmol) was added and the resulting mixture was stirred at 75° C. for 22 h until LC-MS showed the reaction was complete. After cooling to room temperature, the reaction mixture was concentrated under reduced pressure, and the residue was purified by column chromatography (ISCO) on neutral alumina (eluting with 20% DCM in hexanes, 50% DCM in hexanes followed by 2% MeOH in DCM) to give tert-butyl-5-((2′-(5-fluoroisoindolin-2-yl)-[2,4′-bipyrimidin]-4-yl)ethynyl)-1H-pyrazolo[3,4-b]pyridine-1-carboxylate (32-2, 50 mg, yield: 61%) as pale brown solid. MS (ESI): m/z: 535.3 (M+H)⁺.

Step 3: 5-((2′-(5-Fluoroisoindolin-2-yl)-[2,4′-bipyrimidin]-4-yl)ethynyl)-1H-pyrazolo[3,4-b]pyridine (Ex. 32): A mixture of tert-butyl-5-((2′-(5-fluoroisoindolin-2-yl)-[2,4′-bipyrimidin]-4-yl)ethynyl)-1H-pyrazolo[3,4-b]pyridine-1-carboxylate (32-2, 48.0 mg, 0.0897 mmol) in 30% TFA in DCM (1 mL) was stirred at room temperature for 1 h. LC-MS showed the reaction was complete. The reaction mixture was concentrated under reduced pressure to dryness and the residue was neutralized with aq. NaHCO₃ (minimum amount was used). The precipitated product was centrifuged and collected. The trace water in the solid product was azeotropically removed by co-distilling with toluene to afford 5-((2′-(5-fluoroisoindolin-2-yl)-[2,4′-bipyrimidin]-4-yl)ethynyl)-1H-pyrazolo[3,4-b]pyridine (Ex. 32, 31 mg, yield: 79%) as a brown solid. ¹H-NMR (300 MHz, DMSO-d₆): δ (ppm): 14.05 (s, 1H), 9.09 (d, J=4.8 Hz, 1H), 8.82 (d, J=1.8 Hz, 1H), 8.68 (d, J=1.8 Hz, 1H), 8.66 (d, J=5.1 Hz, 2H), 7.86 (d, J=5.1 Hz, 1H), 7.57 (d, J=5.1 Hz, 1H), 7.52-7.38 (m, 1H), 7.34-7.24 (m, 1H), 7.18-7.08 (m, 1H), 4.89 (brs, 4H). MS (ESI⁺): m/z: 435.2 (M+H)⁺.

### Example 33. 5-((2′-(5-Methoxyisoindolin-2-yl)-[2,4′-bipyrimidin]-4-yl)ethynyl)-1H-pyrazolo[3,4-b]pyridine (Ex. 33)

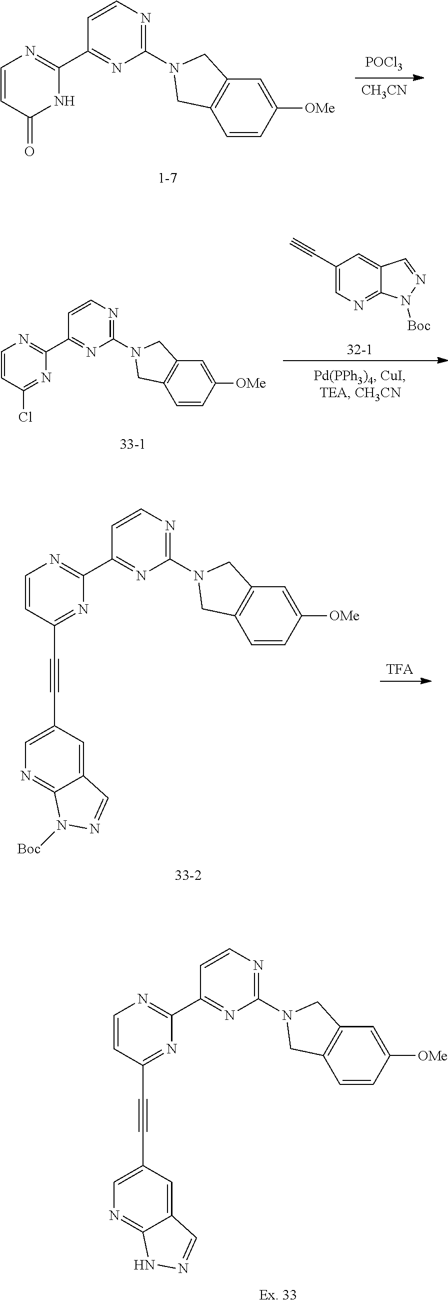

Step 1: 2-(4-Chloro-[2,4′-bipyrimidin]-2′-yl)-5-methoxyisoindoline (33-1): Prepared by following the procedure for 14-1 by treating 1-7 with POCl₃. MS (ESI⁺): m/z: 340.0 (M+H, ³⁵Cl)⁺, 342.0 (M+H, ³⁷Cl)⁺.

Step 2: tert-Butyl-5-((2′-(5-methoxyisoindolin-2-yl)-[2,4′-bipyrimidin]-4-yl)ethynyl)-1H-pyrazolo[3,4-b]pyridine-1-carboxylate (33-2): A suspension of 2-(4-chloro-[2,4′-bipyrimidin]-2′-yl)-5-methoxyisoindoline (33-1, 50 mg, 0.147 mmol), tert-butyl-5-ethynyl-1H-pyrazolo[3,4-b]pyridine-1-carboxylate (32-1, 43.0 mg, 0.176 mmol) and CuI (2.8 mg. 0.0147 mmol) in Et₃N (0.25 mL) and acetonitrile (2.0 mL) was taken in a glass vial and purged with nitrogen gas for 10 min. Pd(PPh₃)₄ (16.9 mg, 0.0147 mmol) was then added. The resulting mixture was stirred at 75° C. for 22 h. LC-MS showed the reaction was complete. After cooling to room temperature, the reaction mixture was concentrated under reduced pressure, and the crude mixture was purified by column chromatography (ISCO) on neutral alumina (eluting with 20% DCM in hexanes, 50% DCM in hexanes followed by 2% MeOH in DCM) to give tert-butyl-5-((2′-(5-methoxyisoindolin-2-yl)-[2,4′-bipyrimidin]-4-yl)ethynyl)-1H-pyrazolo[3,4-b]pyridine-1-carboxylate (33-2, 47 mg, yield: 58%) as a pale brown solid. MS (ESI⁺): m/z: 547.3 (M+H)⁺.

Step 3: 5-((2′-(5-Methoxyisoindolin-2-yl)-[2,4′-bipyrimidin]-4-yl)ethynyl)-1H-pyrazolo[3,4-b]pyridine (Ex. 33): A mixture of tert-butyl-5-((2′-(5-methoxyisoindolin-2-yl)-[2,4′-bipyrimidin]-4-yl)ethynyl)-1H-pyrazolo[3,4-b]pyridine-1-carboxylate (33-2, 45.0 mg, 0.0823 mmol) in 30% TFA in DCM (1.0 mL) was stirred at rt for 1 h. LC-MS showed the reaction was complete. The solvent was evaporated completely under reduced pressure and the residue was neutralized with aq. NaHCO₃ (minimum amount was used). The precipitated product was centrifuged and collected. The trace water in the solid product was azeotropically removed by co-distilling with toluene to afford 5-((2′-(5-methoxyisoindolin-2-yl)-[2,4′-bipyrimidin]-4-yl)ethynyl)-1H-pyrazolo[3,4-b]pyridine (Ex. 33, 30 mg, yield: 82%) as a brown solid. ¹H-NMR (300 MHz, DMSO-d₆): δ (ppm): 14.1 (s, 1H), 9.14 (d, J=4.8 Hz, 1H), 8.87 (s, 1H), 8.73 (s, 1H), 8.70 (d, J=4.8 Hz, 1H), 8.32 (s, 1H), 7.91 (d, J=4.8 Hz, 1H), 7.60 (d, J=4.8 Hz, 1H), 7.42-7.30 (m, 1H), 7.14-7.06 (m, 1H), 6.93 (d, J=8.4 Hz, 1H), 4.91 (t, J=12.9 Hz, 4H), 3.81 (s, 3H). MS (ESI⁺): m/z: 447.2 (M+H)⁺.

### Example 34. 5-((4-(5-Fluoroisoindolin-2-yl)-[2,4′-bipyrimidin]-2′-yl)ethynyl)-1H-indazole (Ex. 34)

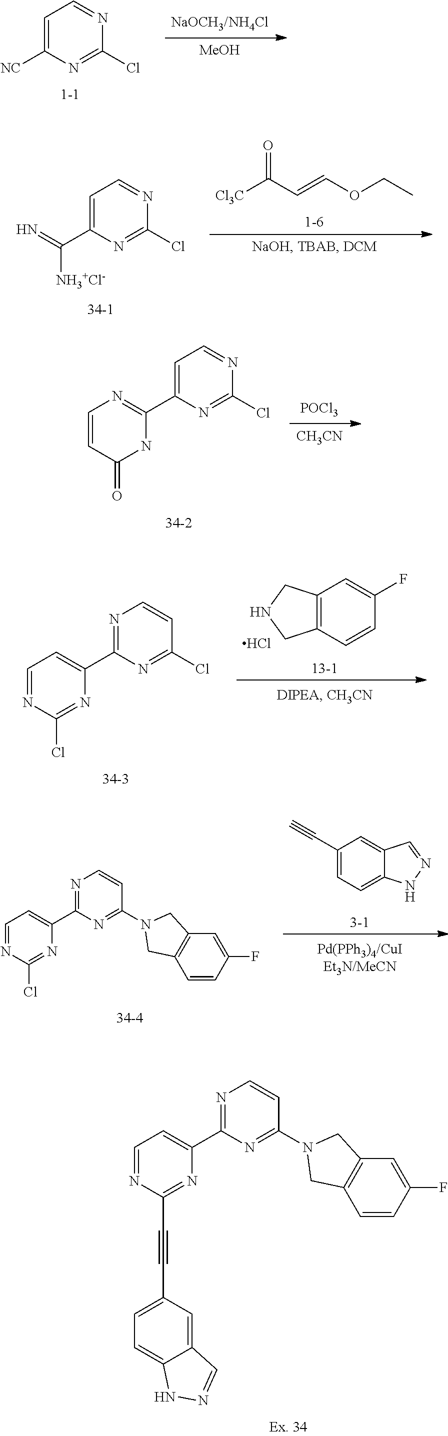

Step 1: 2-Chloropyrimidine-4-carboximidamide HCl salt (34-1): To a solution of 2-chloropyrimidine-4-carbonitrile (1-1, 20 g, 143.3 mmol) in MeOH (200 mL) was added NaOCH₃ (5.42 g, 100.3 mmol) at room temperature. The resulting mixture was stirred at rt for 40 min. NH₄Cl (15.3 g, 286.6 mmol) was added and the reaction mixture was stirred at 50° C. for 2.5 h. After cooling to rt, the solvent was removed in vacuo to give 2-chloropyrimidine-4-carboximidamide HCl salt (34-1) as a brownish solid, which was used directly in the nest step without further purification.

Step 2: 2′-Chloro-[2,4′-bipyrimidin]-4(3H)-one (34-2): A solution of (E)-1,1,1-trichloro-4-ethoxybut-3-en-2-one (1-6, 31.2 g, 143 mmol) in DCM (300 mL) was added to a vigorously stirred mixture of 2-chloropyrimidine-4-carboximidamide HCl salt (34-1, 27.6 g, 143 mmol) in aq. 2M NaOH (286 mL) and tetrabutylammonium bromide (TBAB, cat. 0.6 g). The resulting mixture was stirred at rt for 7 h. The aqueous layer was collected, acidified with conc. HCl to pH≈2, and extracted with DCM (3×100 mL). The combined organic layer was dried over MgSO₄, filtered and concentrated to dryness to give 2′-chloro-[2,4′-bipyrimidin]-4(3H)-one (34-2, 12.08 g) as a yellow solid, which was used directly in the next step without further purification.

Step 3: 2′,4-Dichloro-2,4′-bipyrimidine (34-3): Under N₂, to a suspension of 2′-chloro-[2,4′-bipyrimidin]-4(3H)-one (34-2) from Step 2 in anhydrous acetonitrile was added POCl₃ dropwise. The resulting mixture was stirred at 65° C. for 40 min. LC-MS showed the reaction was complete. Excess POCl₃ was removed completely under reduced pressure and the residue was partitioned between sat. NaHCO₃ and DCM (pH≥8). The product was extracted with DCM (3×100 mL). The combined organic layer was dried over MgSO₄, filtered and concentrated. The crude product was purified by column chromatography (ISCO) (DCM:EA=10:1) to afford the desired product 2′,4-dichloro-2,4′-bipyrimidine (34-3) as a white solid (yield: 68%). ¹H-NMR (300 MHz, DMSO-d₆): δ (ppm): 9.04 (d, J=5.34 Hz, 1H), 9.03 (d, J=5.1 Hz. 1H), 8.34 (d, J=5.05 Hz, 1H), 7.94 (d, J=5.35 Hz, 1H). MS (ESI): m/z 226.97 (M+H)⁺.

Step 4: 2-(2′-Chloro-[2,4′-bipyrimidin]-4-yl)-5-fluoroisoindoline (34-4): To a stirred mixture of 2′,4-dichloro-2,4′-bipyrimidine (34-3, 0.2 g, 0.881 mmol) and 5-fluoroisoindoline hydrochloride (13-1, 160.6 mg, 0.924 mmol) in anhydrous acetonitrile (2 mL) was added N,N-diisopropylethylamine (DIPEA, 0.61 mL, 3.52 mmol) at room temperature. The resulting mixture was stirred at 80° C. for 2 h. LC-MS showed the reaction was complete. The reaction mixture was concentrated in vacuo and the crude product was purified by column chromatography on silica gel (ISCO) (eluting with 100% DCM, 1% MeOH in DCM followed by 5% MeOH in DCM) to afford 2-(2′-chloro-[2,4′-bipyrimidin]-4-yl)-5-fluoroisoindoline (34-4, 110 mg, yield: 38%) as a brown colored fluffy solid. MS (ESI): m/z: 328.3 (M+H)⁺.

Step 5: 5-((4-(5-Fluoroisoindolin-2-yl)-[2,4′-bipyrimidin]-2′-yl)ethynyl)-1H-indazole (Ex. 34): A suspension of 2-(2′-chloro-[2,4′-bipyrimidin]-4-yl)-5-fluoroisoindoline (34-4, 50 mg, 0.15 mmol), 5-ethynyl-1H-indazole (3-1, 32.5 mg, 0.23 mmol) and CuI (2.86 mg, 0.015 mmol) in Et₃N (0.5 mL) and acetonitrile (2.0 mL) was added to a glass vial and purged with nitrogen gas for 10 min. Pd(PPh₃)₄ (17.3 mg, 0.015 mmol) was added and the mixture was stirred at 75° C. for 1 h. LC-MS showed the reaction was complete. After cooling to room temperature, the reaction mixture was concentrated under reduced pressure. The crude product was purified by flash chromatography on ISCO (mobile phase: 3% Methanol (contains 7N ammonia) in DCM) to afford the desired compound 5-((4-(5-fluoroisoindolin-2-yl)-[2,4′-bipyrimidin]-2′-yl)ethynyl)-1H-indazole (Ex. 34, 5.0 mg, yield: 8%) as a pale brown solid. ¹H-NMR (300 MHz, DMSO-d₆): δ (ppm): 13.38 (s, 1H). 9.01 (d, J=5.1 Hz, 1H), 8.48 (d, J=6.0 Hz, 1H), 8.30 (d, J=5.1 Hz, 1H), 8.19 (d, J=7.5 Hz, 1H), 7.68-7.52 (m, 3H), 7.51-7.40 (m, 1H), 7.38-7.25 (m, 1H), 7.27-7.18 (m, 1H), 6.78 (d, J=6.3 Hz, 1H), 4.98 (d, J=9.9 Hz, 2H), 4.98 (d, J=10.8 Hz, 2H). MS (ESI⁺): m/z: 434.4 (M+H)⁺.

### Example 35. 5-((2′-(2,6-Dihydropyrrolo[3,4-c]pyrazol-5(4H)-yl)-[2,4′-bipyrimidin]-4-yl)ethynyl)-1H-indazole (Ex. 35)

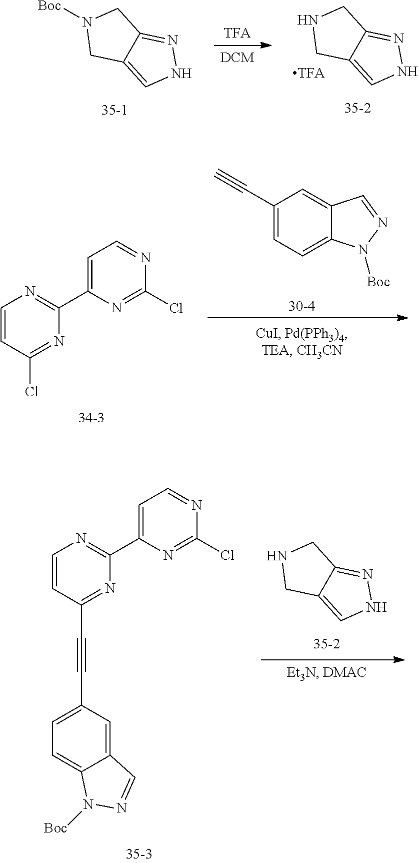

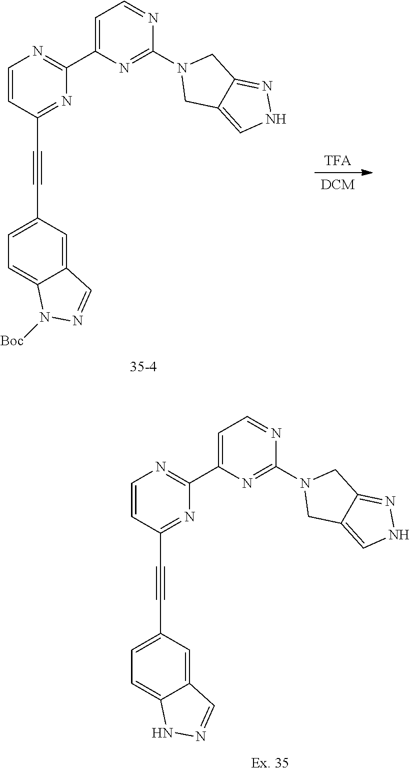

Step 1: 2,4,5,6-Tetrahydropyrrolo[3,4-c]pyrazole TFA salt (35-2): To a stirred solution of tert-butyl-2,6-dihydropyrrolo[3,4-c]pyrazol-5(4H)-carboxylate (35-1, 200 mg, 0.956 mmol) in DCM (4 mL) was added dropwise trifluoroacetic acid (2 mL) at room temperature. The reaction mixture was stirred at room temperature for 1 h. LC-MS showed the reaction was complete. The reaction mixture was concentrated in vacuo to give 2,4,5,6-tetrahydropyrrolo[3,4-c]pyrazole TFA salt (35-2, 52 mg, yield: 50%), which was used directly in the next step without purification.

Step 2: tert-Butyl 5-((2′-chloro-[2,4′-bipyrimidin]-4-yl)ethynyl)-1H-indazole-1-carboxylate (35-3): Under N₂, to a mixture of 2′,4-dichloro-2,4′-bipyrimidine (34-3, 1.0 g, 4.44 mmol), tert-butyl 5-ethynyl-1H-indazole-1-carboxylate (30-4, 1.18 g, 4.88 mmol), CuI (85.5 mg, 0.45 mmol), and Pd(PPh₃)₄ (1.025 g, 0.9 mmol) was added NEt₃ (2.4 mL) followed by MeCN (30 mL). The resulting mixture was degassed for 10 min with N₂ and then stirred at 70-72° C. for 6 h. After cooling to rt, the reaction mixture was left at rt overnight. The precipitates were collected by filtration and washed with diethyl ether to afford the desired product tert-butyl 5-((2′-chloro-[2,4′-bipyrimidin]-4-yl)ethynyl)-1H-indazole-1-carboxylate (35-3, yield, 70%). ¹H NMR (300 MHz, CDCl₃) δ 8.97 (d, J=5.1 Hz, 1H), 8.85 (d, J=5.1 Hz, 1H), 8.45 (d, J=4.8 Hz 1H), 8.26-8.21 (m, 2H), 8.08 (s, 1H), 8.79 (m, 1H), 8.56 (m, 1H), 1.73 (s, 9H), MS (ESI⁺): m/z: 533.18 (M+H)⁺.

Step 3: tert-Butyl 5-((2′-(pyrrolo[3,4-c]pyrazol-5(2H,4H,6H)-yl)-[2,4′-bipyrimidin]-4-yl)ethynyl)-1H-indazole-1-carboxylate (35-4): To a stirred mixture of tert-butyl 5-((2′-chloro-[2,4′-bipyrimidin]-4-yl)ethynyl)-1H-indazole-1-carboxylate (35-3. 50 mg, 0.116 mmol) and 2,4,5,6-tetrahydropyrrolo[3,4-c]pyrazole TFA salt (35-2, 25.3 mg, 0.232 mmol) in anhydrous dimethylacetamide (DMAC, 1.0 mL) was added triethylamine (0.1 mL, 0.717 mmol) dropwise at room temperature. The reaction mixture was stirred at 70° C. for 20 h. The resulting solution was cooled to rt and then diluted with H₂O (5 mL). The aqueous phase was extracted with DCM (3×5 mL). The combined organic phases were dried over Na₂SO₄, filtered, and concentrated in vacuo to afford tert-butyl 5-((2′-(pyrrolo[3,4-c]pyrazol-5(2H,4H,6H)-yl)-[2,4′-bipyrimidin]-4-yl)ethynyl)-1H-indazole-1-carboxylate (35-4), which was used directly in the next step without further purification. MS (ESI⁺): m/z: 506.23 (M+H)⁺.

Step 4: 5-((2′-(2,6-Dihydropyrrolo[3,4-c]pyrazol-5(4H)-yl)-[2,4′-bipyrirmidin]-4-yl)ethynyl)-1H-indazole (Ex. 35): To a stirred solution of tert-butyl 5-((2′-(2,6-dihydropyrrolo[3,4-c]pyrazol-5(4H)-yl)-[2,4′-bipyrimidin]-4-yl)ethynyl)-1H-indazole-1-carboxylate (35-4) in DCM (1.0 mL) was added trifluoroacetic acid (0.5 mL) dropwise at room temperature. The reaction mixture was stirred at room temperature for 1 h. LC-MS showed the reaction was complete. The resulting mixture was then concentrated in vacuo and neutralized with sat. NaHCO₃ solution, then extracted with ethyl acetate. The organic layer was dried over sodium sulfate, filtered, and concentrated to dryness. The crude product was purified by silica gel flash chromatography (ISCO) to afford 5-((2′-(2,6-dihydropyrrolo[3,4-c]pyrazol-5(4H)-yl)-[2,4′-bipyrimidin]-4-yl)ethynyl)-1H-indazole (Ex. 35, 20 mg, yield: 43% for 2 steps) as a light yellow solid. ¹H-NMR (300 MHz, DMSO-d₆): δ (ppm): 9.07 (d, J=5.1 Hz, 1H), 8.66 (d, J=5.0 Hz, 1H), 8.25 (m, 1H), 8.21 (m, 1H), 7.83 (d, J=5.1 Hz, 1H), 7.69-7.64 (m, 2H), 7.60 (m, 1H), 7.57 (d, J=5.0 Hz, 1H), 4.69 (m, 4H). MS (ESI+): m/z: 406.28 (M+H)⁺.

### Example 36. 5-((2′-(6-(4-(2-Methoxyethyl)piperazin-1-yl)-1,3-dihydro-2H-pyrrolo[3,4-c]pyridin-2-yl)-[2,4′-bipyrimidin]-4-yl)ethynyl)-1H-indazole trifluoroacetate salt (Ex. 36)

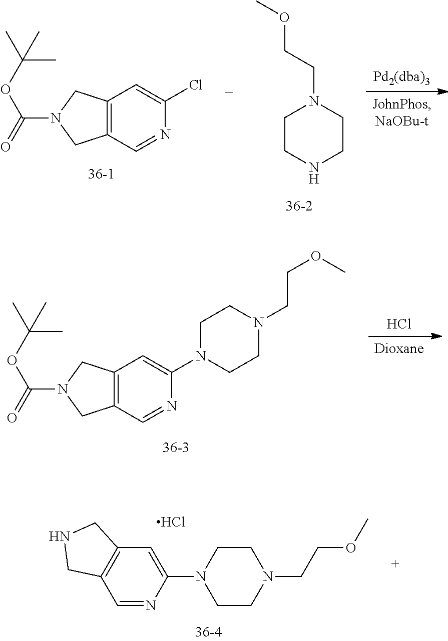

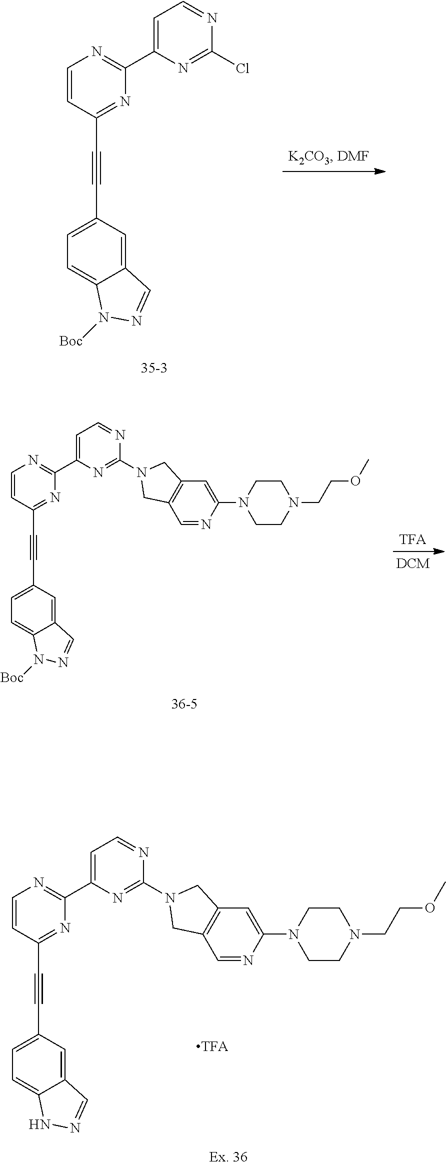

Step 1: tert-Butyl 6-(4-(2-methoxyethyl)piperazin-1-yl)-1,3-dihydro-2H-pyrrolo[3,4-c]pyridine-2-carboxylate (36-3): In a 20 mL glass vial, a mixture of tert-butyl 6-chloro-1H-pyrrolo[3,4-c]pyridine-2(3H)-carboxylate (36-1, 300 mg, 1.178 mmol). 1-(2-methoxyethyl)piperazine (36-2, 203.8 mg, 1.41 mmol), sodium tert-butoxide (565.5 mg, 5.89 mmol), and (2-biphenyl)di-tert-butylphosphine (JohnPhos, 17.6 mg, 0.0588 mmol) in toluene was purged with nitrogen gas at rt for 3 min. Pd₂(dba)₃ (27.2 mg, 0.03 mmol) was then added. The resulting mixture was stirred under N₂ at 80° C. overnight. LC-MS showed the reaction was complete. After cooling to rt, the solvent was evaporated under reduced pressure and the crude product was purified by column chromatography (ISCO) (eluting with 50% DCM in hexanes to 100% DCM followed by 2% MeOH in DCM) to give tert-butyl 6-(4-(2-methoxyethyl)piperazin-1-yl)-1H-pyrrolo[3,4-c]pyridine-2(3H)-carboxylate (36-3, 50 mg, yield: 12%) as an off-white solid. MS (ESI⁺): m/z: 363.2 (M+H)⁺.

Steps 2 and 3: 6-(4-(2-methoxyethyl)piperazin-1-yl)-2,3-dihydro-1H-pyrrolo[3,4-c]pyridine HCl salt (36-4) and tert-butyl 5-((2′-(6-(4-(2-methoxyethyl)piperazin-1-yl)-1H-pyrrolo[3,4-c]pyridin-2(3H)-yl)-[2,4′-bipyrimidin]-4-yl)ethynyl)-1H-indazole-1-carboxylate (36-5): In a 20 mL glass vial, a mixture of tert-butyl 6-(4-(2-methoxyethyl)piperazin-1-yl)-1H-pyrrolo[3,4-c]pyridine-2(3H)-carboxylate (36-3, 40 mg, 0.11 mmol) in 4N HCl in dioxane (3.0 mL) was stirred at rt for 1 h. LC-MS showed the reaction was complete. The solvent was removed completely in vacuo and to the crude product (36-4) was added tert-butyl 5-((2′-chloro-[2,4′-bipyrimidin]-4-yl)ethynyl)-1H-indazole-1-carboxylate (35-3, 47.7 mg. 0.11 mmol), K₂CO₃ (60.8 mg, 0.44 mmol), and DMF (1.0 mL). The resulting mixture was stirred at 70° C. for 4 h. LC-MS showed the reaction was compete. After cooling to rt, the solvent was removed under reduced pressure and the residue was purified by column chromatography (ISCO) on neutral alumina (eluting with 20% DCM in hexanes, 50% DCM in hexanes followed by 100% DCM) to afford tert-butyl 5-((2′-(6-(4-(2-methoxyethyl)piperazin-1-yl)-1H-pyrrolo[3,4-c]pyridin-2(3H)-yl)-[2,4′-bipyrimidin]-4-yl)ethynyl)-1H-indazole-1-carboxylate (36-5, 15 mg, yield: 21%) as an off-white solid. MS (ESI⁺): m/z: 659.4 (M+H)⁺.

Step 4: 5-((2′-(6-(4-(2-Methoxyethyl)piperazin-1-yl)-1,3-dihydro-2H-pyrrolo[3,4-c]pyridin-2-yl)-[2,4′-bipyrimidin]-4-yl)ethynyl)-1H-indazole trifluoroacetate salt (Ex. 36): A mixture of tert-butyl 5-((2′-(6-(4-(2-methoxyethyl)piperazin-1-yl)-1H-pyrrolo[3,4-c]pyridin-2(3H)-yl)-[2,4′-bipyrimidin]-4-yl)ethynyl)-1H-indazole-1-carboxylate (36-5, 14.0 mg, 0.02125 mmol) in 30% TFA in DCM was stirred at rt for 1 h. LC-MS showed the reaction was complete. The solvent was evaporated under reduced pressure to give the crude product, which was purified by trituration with ether (2×5 mL) to afford 5-((2′-(6-(4-(2-methoxyethyl)piperazin-1-yl)-1,3-dihydro-2H-pyrrolo[3,4-c]pyridin-2-yl)-[2,4′-bipyrimidin]-4-yl)ethynyl)-1H-indazole trifluoroacetate salt (Ex. 36, 5.0 mg, yield: 42%) as a brown solid. ¹H-NMR (300 MHz, DMSO-d₆): δ (ppm): 13.44 (s, 1H), 9.75 (brs, 1H), 9.05 (d, J=5.1 Hz, 1H), 8.67 (d, J=4.8 Hz, 1H), 8.27-8.19 (m, 3H), 7.83 (d, J=5.1 Hz, 1H), 7.68-7.57 (m, 3H), 7.08-7.06 (m, 1H), 4.86 (brs, 2H), 4.37 (brs, 2H), 3.67-3.66 (m, 2H), 3.58-3.49 (m, 3H), 3.44-3.30 (m, 5H), 3.18 (s, 3H), 2.34-2.20 (m, 2H). MS (ESI): m/z: 559.4 (M+H)⁺.

### Example 37. 5-((2′-(6-(2-Methoxyethoxy)-1,3-dihydro-2H-pyrrolo[3,4-c]pyridin-2-yl)-[2,4′-bipyrimidin]-4-yl)ethynyl)-1H-indazole (Ex. 37)

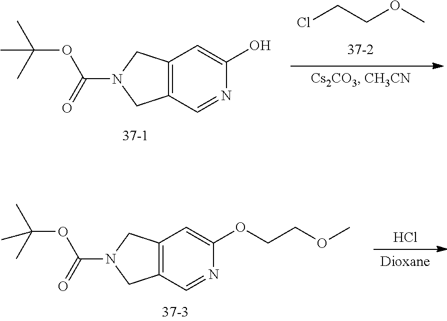

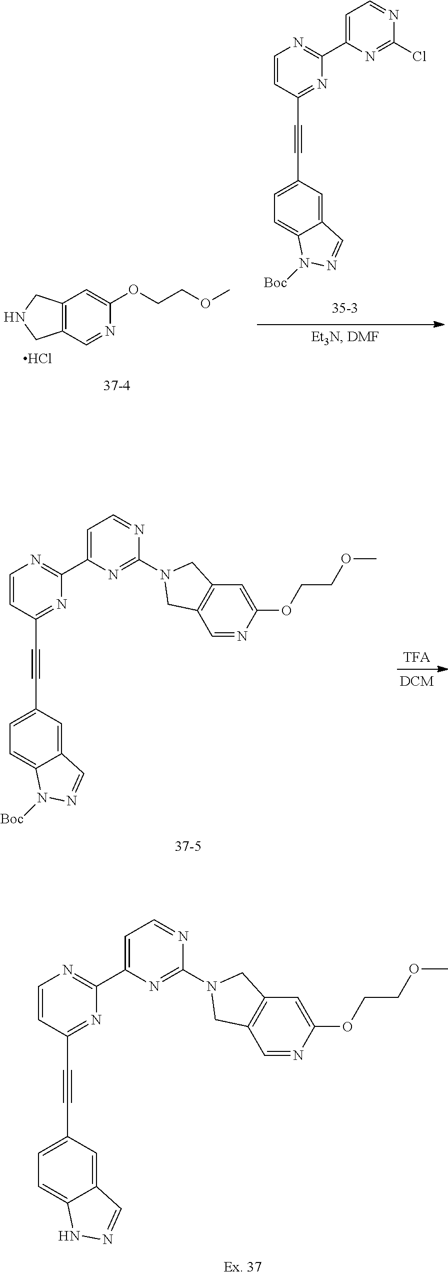

Step 1: tert-Butyl 6-(2-Methoxyethoxy)-1,3-dihydro-2H-pyrrolo[3,4-c]pyridine-2-carboxylate (37-3): To a suspension of tert-butyl 6-hydroxy-1H-pyrrolo[3,4-c]pyridine-2(3H)-carboxylate (37-1, 50 mg, 0.2116 mmol) in acetonitrile (5 mL) was added 2-chloroethyl-methylether (37-2, 48.3 μL, 0.529 mmol) followed by Cs₂CO₃ (344.7 mg, 1.058 mmol). The resulting mixture was stirred at 70° C. for 6 h. LC-MS showed the reaction was complete. After cooling to rt, the solvent was removed completely under reduced pressure and the residue was purified by column chromatography (ISCO) on neutral alumina by sequential elution with 20% EtOAc in hexanes, 50% EtOAc in hexanes, followed by 100% EtOAc to give tert-butyl 6-(2-methoxyethoxy)-1,3-dihydro-2H-pyrrolo[3,4-c]pyridine-2-carboxylate (37-3, 30.1 mg, yield: 48%) as a gummy solid. MS (ESI⁺): m/z: 295.3 (M+H)⁺.

Steps 2 and 3: 6-(2-Methoxyethoxy)-2,3-dihydro-1H-pyrrolo[3,4-c]pyridine HCl salt (37-4) and tert-Butyl-5-((2′-(6-(2-methoxyethoxy)-1,3-dihydro-2H-pyrrolo[3,4-c]pyridin-2-yl)-[2,4′-bipyrimidin]-4-yl)ethynyl)-1H-indazole-1-carboxylate (37-5): In a 20 mL glass vial, a mixture of tert-butyl 6-(2-methoxyethoxy)-1,3-dihydro-2H-pyrrolo[3,4-c]pyridine-2-carboxylate (37-3, 18.7 mg, 0.063 mmol) in 4N HCl in dioxane (1.0 mL) was stirred at rt for 1 h. LC-MS showed the reaction was complete. The solvent was removed completely under reduced pressure to give crude product 37-4. To this crude product was added tert-butyl 5-((2′-chloro-[2,4′-bipyrimidin]-4-yl)ethynyl)-1H-indazole-1-carboxylate (35-3, 25.0 mg, 0.0577 mmol) and Et₃N (32.2 μL, 0.231 mmol) in DMF (1.0 mL). The resulting mixture was stirred at rt for 4 h. LC-MS showed the reaction was complete. The solvent was evaporated under reduced pressure and the residue was purified by column chromatography (ISCO) on neutral alumina (eluting with 20% DCM in hexanes, 50% DCM in hexanes followed by 100% DCM) to afford tert-butyl-5-((2′-(6-(2-methoxyethoxy)-1,3-dihydro-2H-pyrrolo[3,4-c]pyridin-2-yl)-[2,4′-bipyrimidin]-4-yl)ethynyl)-1H-indazole-1-carboxylate (37-5, 9.0 mg, yield: 26%) as a pale yellow solid. MS (ESI⁺): m/z: 591.4 (M+H)⁺.

Step 4: 5-((2′-(6-(2-Methoxyethoxy)-1,3-dihydro-2H-pyrrolo[3,4-c]pyridin-2-yl)-[2,4′-bipyrimidin]-4-yl)ethynyl)-1H-indazole (Ex. 37): A mixture of tert-butyl-5-((2′-(6-(2-methoxyethoxy)-1,3-dihydro-2H-pyrrolo[3,4-c]pyridin-2-yl)-[2,4′-bipyrimidin]-4-yl)ethynyl)-1H-indazole-1-carboxylate (37-5, 8.0 mg, 0.0135 mmol) in 30% TFA in DCM (1 mL) was stirred at rt for 1 h. LC-MS showed the reaction was complete. The solvent was evaporated completely under reduced pressure and the residue was neutralized with aq. NaHCO₃ (minimum amount used). The precipitated product was centrifuged, collected, and dried to give 5-((2′-(6-(2-methoxyethoxy)-1,3-dihydro-2H-pyrrolo[3,4-c]pyridin-2-yl)-[2,4′-bipyrimidin]-4-yl)ethynyl)-1H-indazole (Ex. 37, 6.0 mg, yield: 91%) as a gray solid. ¹H-NMR (300 MHz, DMSO-d₆): δ (ppm): 9.09 (d, J=5.1 Hz, 1H), 8.71 (d, J=4.8 Hz, 1H), 8.25 (d, J=10.8 Hz, 2H), 7.87 (d, J=5.1 Hz, 1H), 7.78 (s, 1H), 7.74-7.61 (m, 3H), 6.53 (brs, 1H), 4.78 (brs, 4H), 4.09 (t, J=5.1 Hz, 2H), 3.61 (t, J=5.1 Hz, 2H), 3.28 (s, 3H). MS (ESI⁺): m/z: 491.3 (M+H)⁺.

### Example 38. 2-((2-(4-((1H-indazol-5-yl)ethynyl)-[2,4′-bipyrimidin]-2′-yl)-2,3-dihydro-1H-pyrrolo[3,4-c]pyridin-6-yl)oxy)-N,N-dimethylacetamide (Ex. 38)

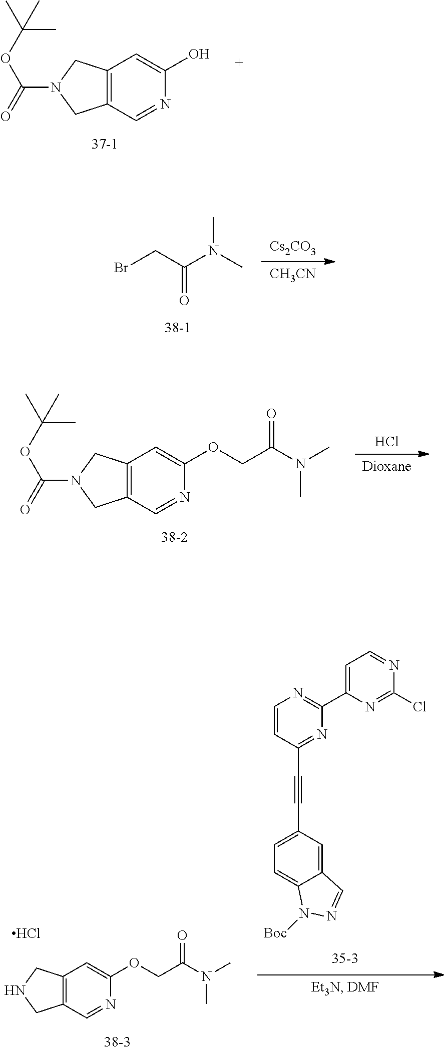

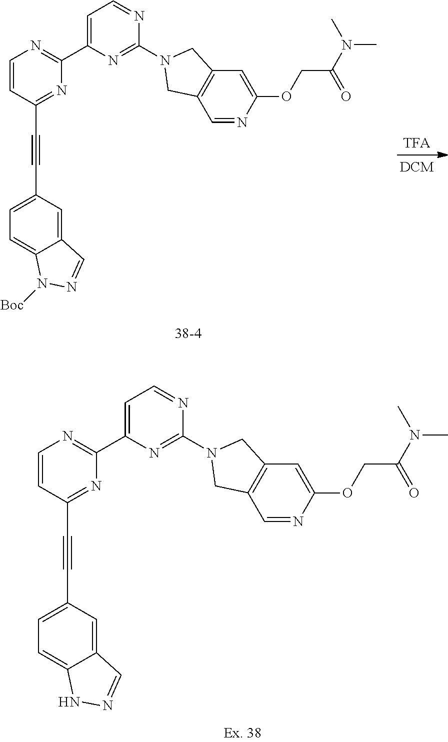

Step 1: tert-Butyl-6-(2-(dimethylamino)-2-oxoethoxy)-1,3-dihydro-2H-pyrrolo[3,4-c]pyridine-2-carboxylate (38-2): To a suspension of tert-butyl 6-hydroxy-1H-pyrrolo[3,4-c]pyridine-2(3H)-carboxylate (37-1, 50 mg, 0.2116 mmol) in acetonitrile (5 mL) was added 2-bromo-N,N-dimethylacetamide (38-1, 52.7 mg, 0.3174 mmol) followed by Cs₂CO₃ (172.3 mg, 0.529 mmol). The resulting mixture was stirred at 70° C. for 6 h. LC-MS showed the reaction was complete. After cooling to rt, the solvent was removed completely under reduced pressure and the residue was purified by column chromatography (ISCO) on neutral alumina by sequential elution with 20% EtOAc in hexanes, 50% EtOAc in hexanes, followed by 100% EtOAc to afford tert-butyl-6-(2-(dimethylamino)-2-oxoethoxy)-1,3-dihydro-2H-pyrrolo[3,4-c]pyridine-2-carboxylate (38-2, 32.0 mg, yield: 47%) as an off-white solid. MS (ESI⁺): m/z: 322.1 (M+H)⁺.

Steps 2 and 3: 2-((2,3-Dihydro-1H-pyrrolo[3,4-c]pyridin-6-yl)oxy)-N,N-dimethylacetamide HCl salt (38-3) and tert-Butyl-5-((2′-(6-(2-(dimethylamino)-2-oxoethoxy)-1,3-dihydro-2H-pyrrolo[3,4-c]pyridin-2-yl)-[2,4′-bipyrimidin]-4-yl)ethynyl)-1H-indazole-1-carboxylate (38-4): In a 20 mL glass vial, a mixture of tert-butyl-6-(2-(dimethylamino)-2-oxoethoxy)-1,3-dihydro-2H-pyrrolo[3,4-c]pyridine-2-carboxylate (38-2, 20.4 mg, 0.063 mmol) in 4N HCl in dioxane (1.0 mL) was stirred at rt for 1 h. LC-MS showed the reaction was complete. The solvent was removed completely in vacuo to give the crude product 38-3. To this crude compound was added tert-butyl 5-((2′-chloro-[2,4′-bipyrimidin]-4-yl)ethynyl)-1H-indazole-1-carboxylate (35-3, 25.0 mg, 0.0577 mmol), Et₃N (32.2 μL, 0.231 mmol), and DMF (1.0 mL). The resulting mixture was stirred at rt for 4 h. The solvent was evaporated under reduced pressure and the residue was purified by column chromatography (ISCO) on neutral alumina (eluting with 20% DCM in hexanes, 50% DCM in hexanes followed by 100% DCM) to afford tert-butyl-5-((2′-(6-(2-(dimethylamino)-2-oxoethoxy)-1,3-dihydro-2H-pyrrolo[3,4-c]pyridin-2-yl)-[2,4′-bipyrimidin]-4-yl)ethynyl)-1H-indazole-1-carboxylate (38-4, 12.0 mg, yield: 34%) as a brownish solid. MS (ESI⁺): m/z: 618.2 (M+H)⁺.

Step 4: 2-((2-(4-((1H-indazol-5-yl)ethynyl)-[2,4′-bipyrimidin]-2′-yl)-2,3-dihydro-1H-pyrrolo[3,4-c]pyridin-6-yl)oxy)-N,N-dimethylacetamide (Ex. 38): A mixture of tert-butyl-5-((2′-(6-(2-(dimethylamino)-2-oxoethoxy)-1,3-dihydro-2H-pyrrolo[3,4-c]pyridin-2-yl)-[2,4′-bipyrimidin]-4-yl)ethynyl)-1H-indazole-1-carboxylate (38-4, 12.0 mg, 0.0194 mmol) in 30% TFA in DCM (1 mL) was stirred at rt for 1 h. LC-MS showed the reaction was complete. The solvent was evaporated completely under reduced pressure to give the crude product, which was then neutralized with aq. NaHCO₃ (minimum amount used). The precipitated product was centrifuged and collected. The trace water in the solid product was azeotropically removed by co-distilling with toluene to afford 2-((2-(4-((1H-indazol-5-yl)ethynyl)-[2,4′-bipyrimidin]-2′-yl)-2,3-dihydro-1H-pyrrolo[3,4-c]pyridin-6-yl)oxy)-N,N-dimethylacetamide (Ex. 38, 8.0 mg, yield: 80%) as a gray solid. ¹H-NMR (300 MHz, DMSO-d₆): δ (ppm): 13.5 (s, 1H), 9.09 (d, J=5.1 Hz, 1H), 8.71 (d, J=4.8 Hz, 1H), 8.25 (d, J=11.4 Hz, 2H), 7.87 (d, J=5.1 Hz, 1H), 7.74-7.61 (m, 3H), 6.51 (brs, 1H), 4.83 (s, 2H), 4.75 (brs, 4H), 3.09 (s, 3H), 2.89 (s, 3H). MS (ESI⁺): m/z: 518.1 (M+H)⁺.

### Example 39. 2-((2-(4-((1H-Indazol-5-yl)ethynyl)-[2,4′-bipyrimidin]-2′-yl)isoindolin-5-yl)oxy)-N,N-dimethylethanamine (Ex. 39)

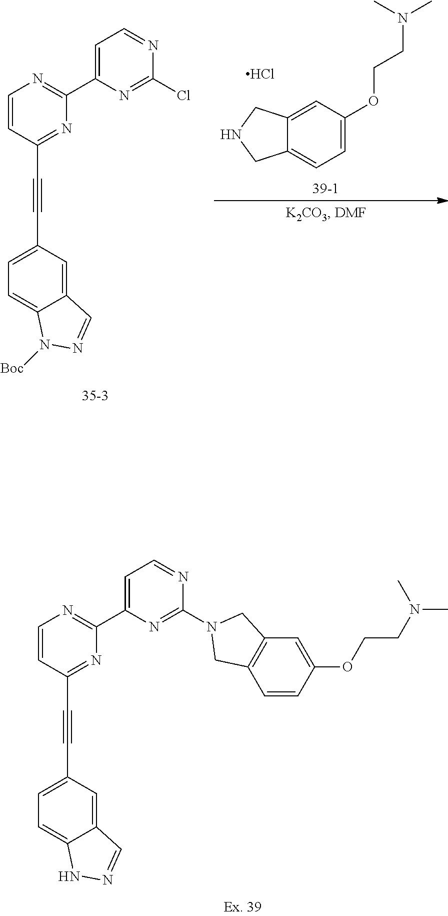

Under N₂, a mixture of 2-(isoindolin-5-yloxy)-N,N-dimethylethan-1-amine HCl salt (CAS #1093293-90-3. Can be easily prepared according to WO 2008/155001) (39-1, 53 mg, 0.19 mmol), tert-butyl 5-((2′-chloro-[2,4′-bipyrimidin]-4-yl)ethynyl)-1H-indazole-1-carboxylate (35-3, 56.5 mg, 0.13 mmol) and K₂CO₃ (200 mg, 1.45 mmol) in DMF (2 mL) was stirred at 70° C. for 3 h. LC-MS showed the reaction was complete. After cooling to rt, the reaction was filtered, and the filtrate concentrated to dryness. The residue was purified by flash chromatography on silica (eluted with 7N methanolic ammonia/DCM, 0-3%) to give 2-((2-(4-((TH-indazol-5-yl)ethynyl)-[2,4′-bipyrimidin]-2′-yl)isoindolin-5-yl)oxy)-N,N-dimethylethanamine (Ex. 39, 15.0 mg, yield: 23%) as a brown solid. ¹H-NMR (300 MHz, DMSO-d₆): δ (ppm): 9.05 (d, J=6.0 Hz, 1H), 8.65 (d, J=4.8 Hz, 1H), 8.21 (d, J=10.8 Hz, 2H), 7.82 (d, J=5.1 Hz, 1H), 7.63 (d, J=3.3 Hz, 2H), 7.55 (d, J=4.8 Hz, 2H), 7.31 (br, 1H), 7.03 (d, J=14.4 Hz, 1H), 6.88 (d, J=9.0 Hz, 1H), 4.85 (br, 4H), 4.04 (t, J=5.1 Hz, 2H), 2.61 (t, J=5.7 Hz, 2H), 2.20 (s, 6H). MS (ESI⁺): m/z: 503.19 (M+H)⁺.

### Example 40. 5-((2′-(5-(4-Methylpiperazin-1-yl)isoindolin-2-yl)-[2,4′-bipyrimidin]-4-yl)ethynyl)-1H-indazole (Ex. 40)

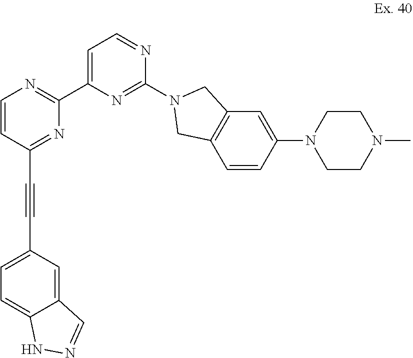 [SMILES: CN1CCN(c2ccc3c(c2)CN(c2nccc(-c4nccc(C#Cc5ccc6[nH]ncc6c5)n4)n2)C3)CC1]

Ex. 40 was prepared from 5-(4-methylpiperazin-1-yl)isoindoline and tert-butyl 5-((2′-chloro-[2,4′-bipyrimidin]-4-yl)ethynyl)-1H-indazole-1-carboxylate (35-3) in a manner analogous to Example 39 to provide the compound in 16.5% yield as a brown solid. ¹H-NMR (300 MHz, DMSO-d₆): δ (ppm): 9.05 (d, J=4.5 Hz, 1H), 8.63 (dd, J₁=4.8 Hz, J₂=2.1 Hz, 1H), 8.21 (m, 2H), 7.81 (dd, J₁=5.1 Hz, J₂=2.1 Hz, 1H), 7.63 (m, 2H), 7.53 (dd, J₁=4.5 Hz, J₂=1.5 Hz, 1H), 7.26 (t, J=9.3 Hz, 1H), 7.01 (d, J=15.6 Hz, 1H), 6.91 (d, J=8.1 Hz, 1H), 4.82 (t, J=17.4 Hz, 4H), 3.12 (br, 4H), 2.42 (s, 3H), 2.20 (s, 4H). MS (ESI⁺): m/z: 514.21 (M+H)⁺.

### Example 41. 4-(2-(4-((1H-Indazol-5-yl)ethynyl)-[2,4′-bipyrimidin]-2′-yl)isoindolin-5-yl)-2-methylmorpholine (Ex. 41)

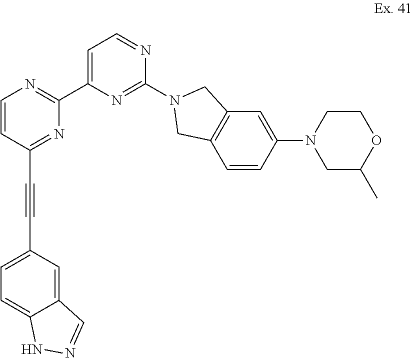 [SMILES: CC1CN(c2ccc3c(c2)CN(c2nccc(-c4nccc(C#Cc5ccc6[nH]ncc6c5)n4)n2)C3)CCO1]

Ex. 41 was prepared from 4-(isoindolin-5-yl)-2-methylmorpholine and tert-butyl 5-((2′-chloro-[2,4′-bipyrimidin]-4-yl)ethynyl)-1H-indazole-1-carboxylate (35-3) in a manner analogous to Example 39 to provide the compound in 18.5% yield as a brownish solid. ¹H-NMR (300 MHz, DMSO-d₆): δ (ppm): 9.06 (d, J=2.4 Hz, 1H), 8.65 (d, J=3.0 Hz, 1H), 8.23 (d, J=14.7 Hz, 2H), 7.82 (m, 1H), 7.64 (s, 2H), 7.55 (d, J=7.8 Hz, 1H), 7.28 (s, 1H), 7.00 (br, 2H), 4.83 (s, 4H), 4.56 (br, 1H), 3.85 (d, J=21 Hz, 2H), 3.61 (br, 4H), 1.65 (d, J=9.3 Hz, 3H). MS (ESI⁺): m/z: 515.24 (M+H)⁺.

### Example 42. 4-(2-((2-(4-((1H-Indazol-5-yl)ethynyl)-[2,4′-bipyrimidin]-2′-yl)isoindolin-5-yl)oxy)ethyl)morpholine (Ex. 42)

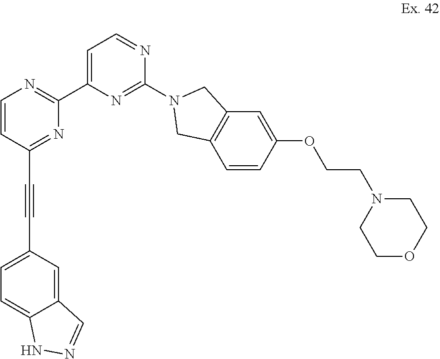 [SMILES: C(#Cc1ccnc(-c2ccnc(N3Cc4ccc(OCCN5CCOCC5)cc4C3)n2)n1)c1ccc2[nH]ncc2c1]

Ex. 42 was prepared from 4-(2-(isoindolin-5-yloxy)ethyl)morpholine and tert-butyl 5-((2′-chloro-[2,4′-bipyrimidin]-4-yl)ethynyl)-1H-indazole-1-carboxylate (35-3) in a manner analogous to Example 39 to provide the compound in 22% yield as a yellow solid. ¹H-NMR (300 MHz, DMSO-d₆): δ (ppm): 13.4 (s, 1H), 9.05 (d, J=6.0 Hz, 1H), 8.65 (d, J=6.3 Hz, 1H), 8.20 (d, J=16.8 Hz, 2H), 7.82 (d, J=6.3 Hz, 1H), 7.65 (m, 2H), 7.55 (d, J=5.4 Hz, 1H), 7.33 (s, 1H), 7.07 (s, 1H), 6.90 (s, 1H), 4.87 (s, 4H), 4.08 (s, 2H), 3.57 (s, 4H), 2.68 (s, 2H). MS (ESI⁺): m/z: 545.30 (M+H)⁺.

### Example 43. 2-((2-(4-((1H-Indazol-5-yl)ethynyl)-[2,4′-bipyrimidin]-2′-yl)isoindolin-5-yl)oxy)-N,N-dimethylacetamide (Ex. 43)

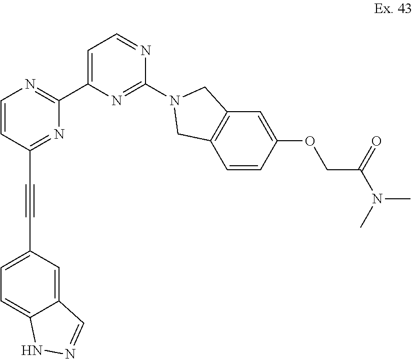 [SMILES: CN(C)C(=O)COc1ccc2c(c1)CN(c1nccc(-c3nccc(C#Cc4ccc5[nH]ncc5c4)n3)n1)C2]

Ex. 43 was prepared from 2-(isoindolin-5-yloxy)-N,N-dimethylacetamide and tert-butyl 5-((2′-chloro-[2,4′-bipyrimidin]-4-yl)ethynyl)-1H-indazole-1-carboxylate (35-3) in a manner analogous to Example 39 to provide the compound in 25.9% yield as a yellow solid. ¹H-NMR (300 MHz, DMSO-d₆): δ (ppm): 13.4 (s, 1H), 9.04 (d, J=6.9 Hz, 1H), 8.64 (d, J=5.4 Hz, 1H), 8.22 (d, J=14.7 Hz, 2H), 7.81 (d, J=4.8 Hz, 1H), 7.63 (m, 3H), 7.31 (s, 1H), 7.02 (s, 1H), 6.88 (s, 1H), 4.79 (m, 6H), 3.00 (s, 3H), 2.83 (s, 3H). MS (ESI⁺): m/z: 517.34 (M+H)⁺.

### Example 44. 2-((2-(4-((1H-Indazol-5-yl)ethynyl)-[2,4′-bipyrimidin]-2′-yl)isoindolin-5-yl)oxy)-N-methylacetamide (Ex. 44)

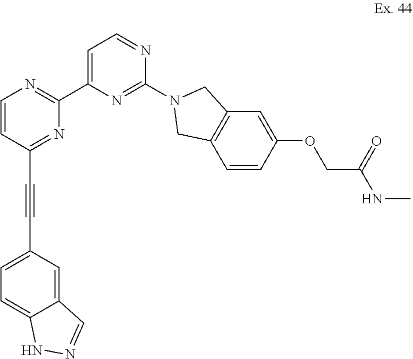 [SMILES: CNC(=O)COc1ccc2c(c1)CN(c1nccc(-c3nccc(C#Cc4ccc5[nH]ncc5c4)n3)n1)C2]

Ex. 44 was prepared from 2-(isoindolin-5-yloxy)-N-methylacetamide and tert-butyl 5-((2′-chloro-[2,4′-bipyrimidin]-4-yl)ethynyl)-1H-indazole-1-carboxylate (35-3) in a manner analogous to Example 39 to provide the compound in 20.3% yield as a yellowish solid. ¹H-NMR (300 MHz, CD₃OD): δ (ppm): 8.91 (d, J=5.1 Hz, 1H), 8.56 (d, J=6.0 Hz, 1H), 8.14 (s, 1H), 8.08 (s, 1H), 7.61 (m, 3H), 7.56 (d, J=7.8 Hz, 1H), 7.28 (d, J=9.0 Hz, 1H), 6.96 (s, 1H), 6.90 (dd, J₁=8.4 Hz, J₂=2.1 Hz, 1H), 4.96 (d, J=24 Hz, 4H), 4.48 (s, 2H), 2.85 (d, J=4.5 Hz, 3H). MS (ESI⁺): m/z: 503.19 (M+H)⁺.

### Example 45. 5-((2′-(5-(Trifluoromethyl)isoindolin-2-yl)-[2,4′-bipyrimidin]-4-yl)ethynyl)-1H-indazole trifluoroacetate (Ex. 45)

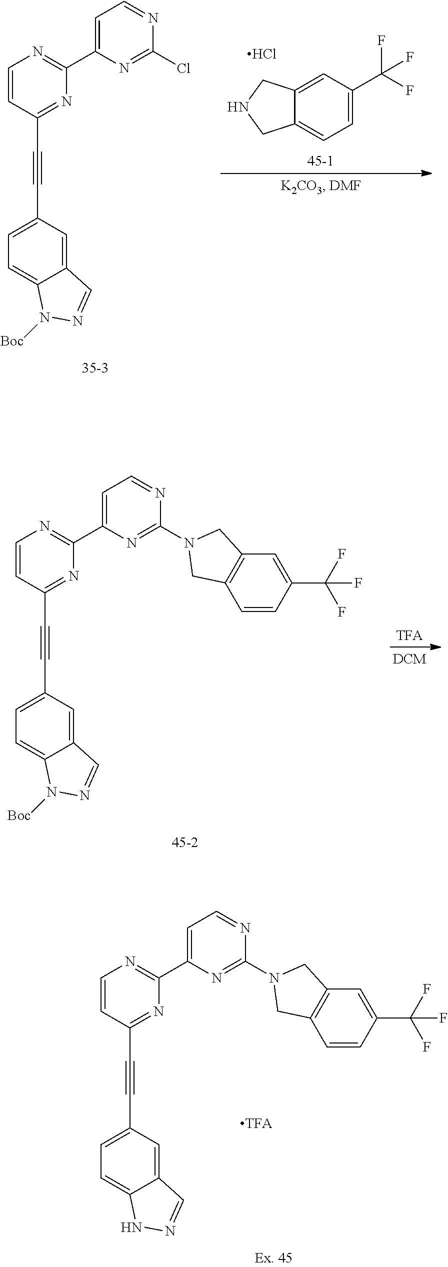

Step 1: tert-Butyl 5-((2′-(5-(trifluoromethyl)isoindolin-2-yl)-[2,4′-bipyrimidin]-4-yl)ethynyl)-1H-indazole-1-carboxylate (45-2). Under N₂, a mixture of 5-(trifluoromethyl)isoindoline HCl salt (45-1, 20.0 mg, 0.09 mmol), tert-butyl 5-((2′-chloro-[2,4′-bipyrimidin]-4-yl)ethynyl)-1H-indazole-1-carboxylate (35-3, 10.0 mg, 0.02 mmol) and K₂CO₃ (50 mg, 0.362 mmol) in DMF (1 mL) was stirred at 75° C. for 2 h. LC-MS showed the reaction was complete. The reaction mixture was filtered, filtrate concentrated using rotovap. The residue was purified by ISCO flash chromatography on alumina gel column (eluated with 0-60% EA in Hexane) to afford tert-butyl 5-((2′-(5-(trifluoromethyl)isoindolin-2-yl)-[2,4′-bipyrimidin]-4-yl)ethynyl)-1H-indazole-1-carboxylate (45-2, 5.7 mg, yield: 10.8%) as a bright yellow solid. MS (ESI⁺): m/z: 584.64 (M+H)⁺.

Step 2: 5-((2′-(5-(Trifluoromethyl)isoindolin-2-yl)-[2,4′-bipyrimidin]-4-yl)ethynyl)-1H-indazole trifluoroacetate (Ex. 45): A mixture of tert-butyl 5-((2′-(5-(trifluoromethyl)isoindolin-2-yl)-[2,4′-bipyrimidin]-4-yl)ethynyl)-1H-indazole-1-carboxylate (45-2, 5.7 mg) in TFA (0.2 mL and DCM (2.0 mL) was stirred at rt for 2 h. LC-MS showed the reaction was complete. The reaction mixture was concentrated using rotovap and the crude product was dissolved in minimum amount of ethyl acetate. The product precipitated out by adding hexanes to afford 5-((2′-(5-(trifluoromethyl)isoindolin-2-yl)-[2,4′-bipyrimidin]-4-yl)ethynyl)-1H-indazole trifluoroacetate (Ex. 45, 5.3 mg, yield: quantitative) as a brown solid. (5.3 mg, quant.). ¹H-NMR (300 MHz, DMSO-d₆): δ (ppm): 13.46 (s, 1H), 9.06 (d, J=5.1 Hz, 1H), 8.68 (d, J=4.8 Hz, 1H), 8.21 (d, J=10.5 Hz, 2H), 7.82 (m, 2H), 7.68 (s, 2H), 7.65 (s, 1H), 7.62 (d, J=1.5 Hz, 1H), 7.59 (d, J=5.1 Hz, 1H), 4.99 (d, J=9.3 Hz, 4H). MS (ESI⁺): m/z: 484.24 (M+H)⁺.

### Example 46. 2-((2-(4-((1H-Indazol-5-yl)ethynyl)-[2,4′-bipyrimidin]-2′-yl)isoindolin-5-yl)oxy)-N-cyclopropylacetamide trifluoroacetate (Ex. 46)

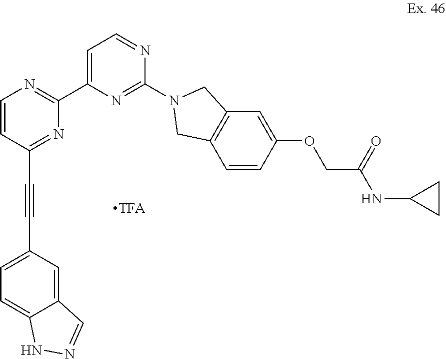 [SMILES: O=C(COc1ccc2c(c1)CN(c1nccc(-c3nccc(C#Cc4ccc5[nH]ncc5c4)n3)n1)C2)NC1CC1]

Ex. 46 was prepared from N-cyclopropyl-2-(isoindolin-5-yloxy)acetamide and tert-butyl 5-((2′-chloro-[2,4′-bipyrimidin]-4-yl)ethynyl)-1H-indazole-1-carboxylate (35-3) in a manner analogous to Example 45 to provide the compound in 16.3% yield as a brown solid. ¹H-NMR (300 MHz, DMSO-d₆): δ (ppm): 13.4 (s, 2H), 9.05 (d, J=5.1 Hz, 1H), 8.65 (d, J=4.8 Hz, 1H), 8.21 (d, J=12 Hz, 2H), 8.12 (s, 1H), 7.82 (d, J=5.1 Hz, 1H), 7.63 (d, J=4.8 Hz, 1H) 7.56 (d, J=5.1 Hz, 1H), 7.34 (s, 1H), 7.05 (d, J=12.0 Hz, 1H), 6.90 (d, J=9.6 Hz, 1H), 4.85 (s, 4H), 4.43 (s, 2H), 0.60 (m, 2H), 0.48 (M, 2H). MS (ESI⁺): m/z: 529.29 (M+H)⁺.

### Example 47. 5-((2′-(5-(2-Methoxyethoxy)isoindolin-2-yl)-[2,4′-bipyrimidin]-4-yl)ethynyl)-1H-indazole (Ex. 47)

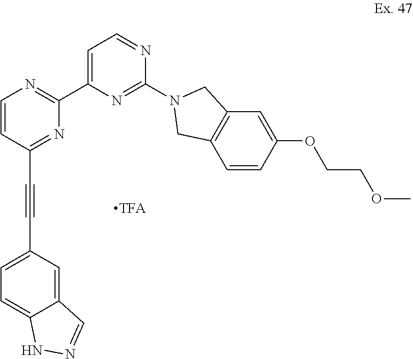 [SMILES: COCCOc1ccc2c(c1)CN(c1nccc(-c3nccc(C#Cc4ccc5[nH]ncc5c4)n3)n1)C2]

Ex. 47 was prepared from 5-(2-methoxyethoxy)isoindoline and tert-butyl 5-((2′-chloro-[2,4′-bipyrimidin]-4-yl)ethynyl)-1H-indazole-1-carboxylate (35-3) in a manner analogous to Example 45 to provide the compound in 10.1% yield as a brown solid. ¹H-NMR (300 MHz, DMSO-d₆): δ (ppm): 13.42 (s, 2H), 9.05 (d, J=5.1 Hz, 1H), 8.65 (d, J=5.1 Hz, 1H), 8.23 (d, J=11.7 Hz, 2H), 7.82 (d, J=5.1 Hz, 1H), 7.63 (q, J=6.0 Hz, 2H), 7.55 (d, J=4.9 Hz, 1H), 7.33 (s, 1H), 7.06 (s, 1H), 6.90 (dd, J₁=8.4 Hz, J₂=2.4 Hz, 1H), 4.85 (s, 4H), 4.09 (m, 2H), 3.49 (m, 2H), 3.30 (s, 3H). MS (ESI⁺): m/z: 490.26 (M+H)⁺.

### Example 48. 5-((2′-(3-Phenylazetidin-1-yl)-[2,4′-bipyrimidin]-4-yl)ethynyl)-1H-indazole trifluoroacetate (Ex. 48)

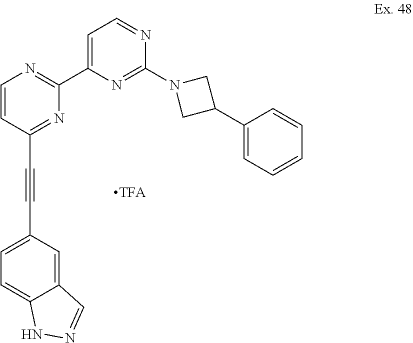 [SMILES: C(#Cc1ccnc(-c2ccnc(N3CC(c4ccccc4)C3)n2)n1)c1ccc2[nH]ncc2c1]

Ex. 48 was prepared from 3-phenylazetidine and tert-butyl 5-((2′-chloro-[2,4′-bipyrimidin]-4-yl)ethynyl)-1H-indazole-1-carboxylate (35-3) in a manner analogous to Example 45 to provide the compound as a pale orange solid. ¹H-NMR (300 MHz, CDCl₃.CD3OD) δ (ppm): 8.92 (d, J=5.1, 1H), 8.53 (d, J=5.1, 1H), 8.16 (s, 1H), 8.11 (s, 1H), 7.67 (m, 2H), 7.61 (m, 2H), 7.37 (m, 4H), 7.25 (m, 1H), 4.69 (m, 2H), 4.29 (m, 2H), 3.99 (m, 1H). MS (ESI⁺): m/z 430.3 (M+H)⁺.

### Example 49. Methyl 4-(1-(4-((1H-indazol-5-yl)ethynyl)-[2,4′-bipyrimidin]-2′-yl)azetidin-3-yl)benzoate trifluoroacetate (Ex. 49)

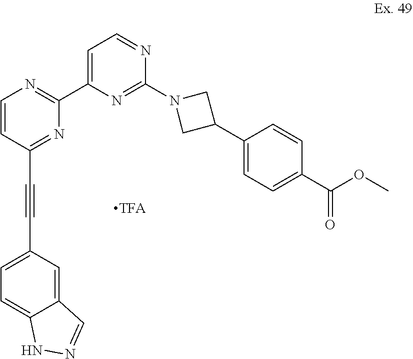 [SMILES: COC(=O)c1ccc(C2CN(c3nccc(-c4nccc(C#Cc5ccc6[nH]ncc6c5)n4)n3)C2)cc1]

Ex. 49 was prepared from methyl 4-(azetidin-3-yl)benzoate and tert-butyl 5-((2′-chloro-[2,4′-bipyrimidin]-4-yl)ethynyl)-1H-indazole-1-carboxylate (35-3) in a manner analogous to Example 45 to provide the compound as a pale orange solid. ¹H-NMR (300 MHz, CDCl₃-CD3OD) δ (ppm): 8.90 (d, J=5.1, 1H), 8.57 (d, J=5.1, 1H), 8.10 (m, 4H), 7.66 (d, J=5.2, 1H), 7.50 (m, 5H), 4.71 (m, 2H), 4.32 (m, 2H), 3.99 (m, 11H). MS ESI: m/z 488.22 (M+1)⁺.

### Example 50. 4-(1-(4-((1H-Indazol-5-yl)ethynyl)-[2,4′-bipyrimidin]-2′-yl)azetidin-3-yl)benzonitrile trifluoroacetate (Ex. 50)

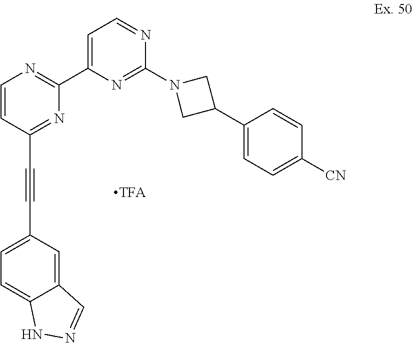 [SMILES: N#Cc1ccc(C2CN(c3nccc(-c4nccc(C#Cc5ccc6[nH]ncc6c5)n4)n3)C2)cc1]

Ex. 50 was prepared from 4-(azetidin-3-yl)benzonitrile and tert-butyl 5-((2′-chloro-[2,4′-bipyrimidin]-4-yl)ethynyl)-1H-indazole-1-carboxylate (35-3) in a manner analogous to Example 45 to provide the compound as a pale orange solid. ¹H-NMR (300 MHz, CDCl₃) δ (ppm): 8.90 (d, J=5.1, 1H), 8.57 (d, J=5.1, 1H), 8.10 (d, J=0.9, 1H), 8.05 (m, 1H), 7.72 (d, J=5.1, 1H), 7.63 (m, 2H), 7.51 (m, 5H), 4.70 (m, 2H), 4.32 (m, 2H), 3.91 (m, 1H). MS ESI: m/z 455.21 (M+1)⁺.

### Example 51. 4-(1-(4-((1H-Indazol-5-yl)ethynyl)-[2,4′-bipyrimidin]-2′-yl)azetidin-3-yl)benzoic acid (Ex. 51)

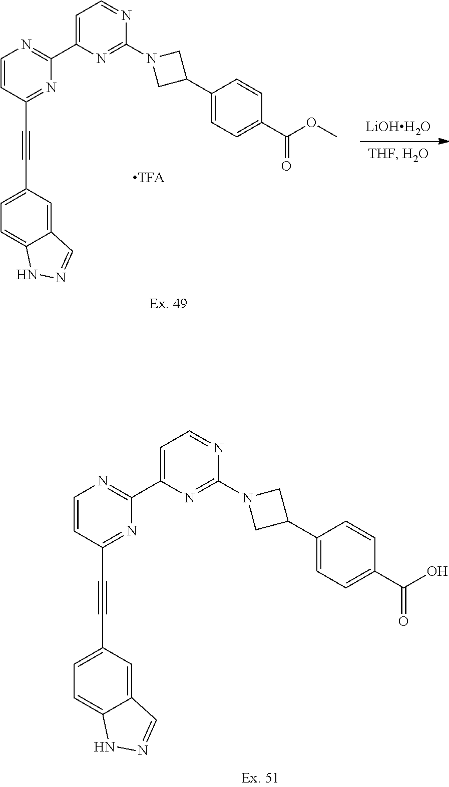

A mixture of methyl 4-(1-(4-((1H-indazol-5-yl)ethynyl)-[2,4′-bipyrimidin]-2′-yl)azetidin-3-yl)benzoate trifluoroacetate (Ex. 49, 18.2 mg, 0.0373 mmol) and LiOH.H₂O (7.83 mg, 0.187 mmol) in dioxane (3 mL) and water (1.5 mL) was stirred at rt for 6 h. LC-MS showed the reaction was complete. To the reaction mixture was added diluted HCl (aq.) to adjust the pH≈4.0. The solids that precipitated out were collected by filtration, washed with water and hexane, and dried under vacuum to give 4-(1-(4-((1H-indazol-5-yl)ethynyl)-[2,4′-bipyrimidin]-2′-yl)azetidin-3-yl)benzoic acid (Ex. 51, yield: 68%) as an ivory-colored solid. ¹H-NMR (300 MHz, DMSO-d₆) δ (ppm): 13.4 (bs, 1H), 12.9 (bs, 1H), 9.03 (d, J=5.1, 1H), 8.60 (d, J=5.0, 1H), 8.60 (d, J=5.0, 1H), 8.21 (m, 1H), 8.18 (s, 1H), 7.93 (d, J=8.1, 1H), 7.80 (d, J=5.1, 1H), 7.60 (m, 5H), 4.58 (m, 2H), 4.11 (m, 3H). MS ESI: 474.21 m z (M+1)⁺.

### Example 52. 5-((2′-(4-Phenylpiperazin-1-yl)-[2,4′-bipyrimidin]-4-yl)ethynyl)-1H-indazole (Ex. 52)

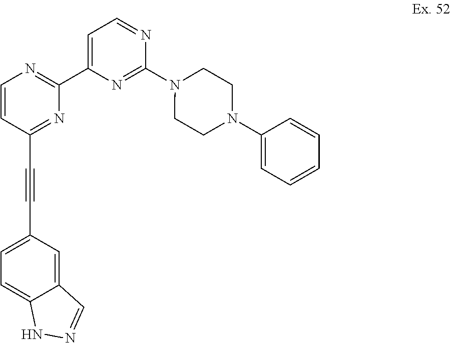 [SMILES: C(#Cc1ccnc(-c2ccnc(N3CCN(c4ccccc4)CC3)n2)n1)c1ccc2[nH]ncc2c1]

Ex. 52 was prepared from 1-phenylpiperazine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 41% yield. ¹H-NMR (400 MHz, CDCl₃) δ 8.94 (d, J=6.8 Hz, 1H), 8.58 (d, J=6.4 Hz, 1H), 8.12 (m, 2H), 7.63 (m, 2H), 7.53 (m, 2H), 7.31 (m, 2H), 7.12 (m, 2H), 6.98 (m, 1H), 4.22 (m, 4H), 3.35 (m, 4H). MS (ESI⁺): m/z: 459.31 (M+H)⁺.

### Example 53. 5-((2′-(4-(4-Fluorophenyl)piperazin-1-yl)-[2,4′-bipyrimidin]-4-yl)ethynyl)-1H-indazole (Ex. 53)

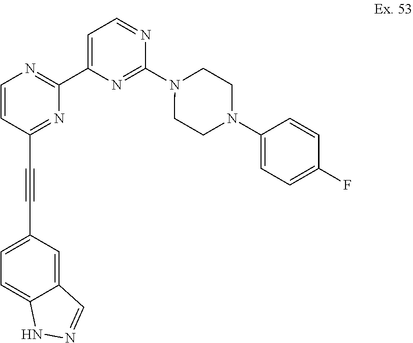 [SMILES: Fc1ccc(N2CCN(c3nccc(-c4nccc(C#Cc5ccc6[nH]ncc6c5)n4)n3)CC2)cc1]

Ex. 53 was prepared from 1-(4-fluorophenyl)piperazine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 36% yield. ¹H-NMR (400 MHz, CDCl₃) δ 8.96 (d, J=6.8 Hz, 1H), 8.59 (d, J=6.8 Hz, 1H), 8.13 (m, 2H), 7.68 (m, 2H), 7.55 (m, 2H), 7.20 (m, 2H), 7.06 (m, 2H), 4.29-4.27 (m, 4H), 3.32-3.30 (m, 4H). MS (ESI⁺): m/z: 477.29 (M+H)⁺.

### Example 54. 5-((2′-(4-Propylpiperazin-1-yl)-[2,4′-bipyrimidin]-4-yl)ethynyl)-1H-indazole trifluoroacetate (Ex. 54)

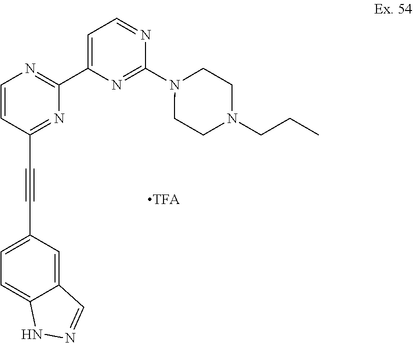 [SMILES: CCCN1CCN(c2nccc(-c3nccc(C#Cc4ccc5[nH]ncc5c4)n3)n2)CC1]

Ex. 54 was prepared from 1-propylpiperazine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 39% yield. ¹H-NMR (400 MHz, CDCl₃) δ 9.05 (d, J=6.8 Hz, 1H), 8.60 (d, J=6.8 Hz, 1H), 8.16 (m, 2H), 7.75 (m, 2H), 7.60 (m, 2H), 5.12-5.06 (m, 2H), 3.74-3.61 (m, 4H), 2.84-2.79 (m, 4H), 1.86-1.82 (m, 2H), 1.09 (t, 3H). MS (ESI⁺): m/z: 425.33 (M+H)⁺.

### Example 55. 5-((2′-(4-Phenylpiperidin-1-yl)-[2,4′-bipyrimidin]-4-yl)ethynyl)-1H-indazole (Ex. 55)

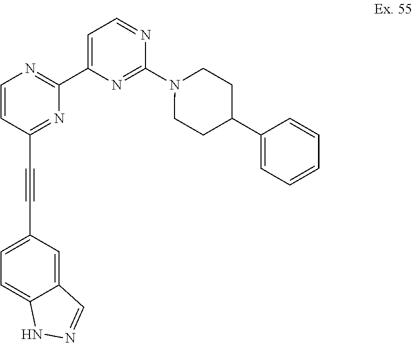 [SMILES: C(#Cc1ccnc(-c2ccnc(N3CCC(c4ccccc4)CC3)n2)n1)c1ccc2[nH]ncc2c1]

Ex. 55 was prepared from 4-phenylpiperidine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 33% yield. ¹H-NMR (400 MHz, CDCl₃) δ 8.93 (d, J=6.8 Hz, 1H), 8.56 (d, J=6.8 Hz, 1H), 8.12 (m, 2H), 7.61-7.50 (m, 4H), 7.31-7.21 (m, 5H), 5.16-5.10 (m, 2H), 3.11-3.02 (m, 2H), 2.81 (m, 1H), 2.0-1.96 (m, 2H), 1.83-1.74 (m, 2H). MS (ESI⁺): m/z: 458.29 (M+H)⁺.

### Example 56. 5-((2′-(4-(4-Fluorophenyl)piperidin-1-yl)-[2,4′-bipyrimidin]-4-yl)ethynyl)-1H-indazole (Ex. 56)

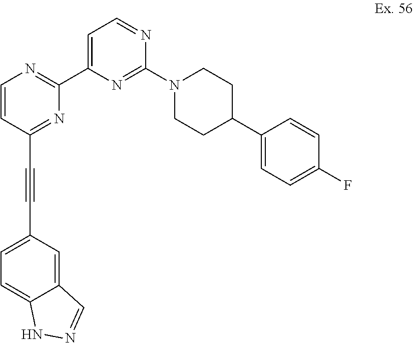 [SMILES: Fc1ccc(C2CCN(c3nccc(-c4nccc(C#Cc5ccc6[nH]ncc6c5)n4)n3)CC2)cc1]

Ex. 56 was prepared from 4-(4-fluorophenyl)piperidine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 40% yield. ¹H-NMR (400 MHz, CDCl₃) δ 8.92 (d, J=6.8 Hz, 1H), 8.55 (d, J=6.8 Hz, 1H), 8.12 (m, 2H), 7.58-7.49 (m, 4H), 7.14 (m, 2H), 7.01-6.95 (m, 2H), 5.12-5.07 (M, 2H), 3.0-2.99 (m, 2H), 2.76 (m, 1H), 1.88-1.68 (m, 2H), 1.67-1.64 (m, 2H). MS (ESI⁺): m/z: 476.28 (M+H)⁺.

### Example 57. 5-((2′-(3-(4-Chlorophenyl)azetidin-1-yl)-[2,4′-bipyrimidin]-4-yl)ethynyl)-1H-indazole (Ex. 57)

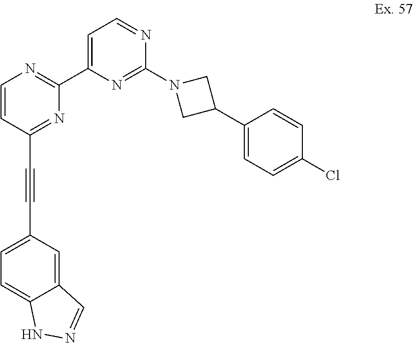 [SMILES: Clc1ccc(C2CN(c3nccc(-c4nccc(C#Cc5ccc6[nH]ncc6c5)n4)n3)C2)cc1]

Ex. 57 was prepared from 3-(4-chlorophenyl)azetidine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 40% yield. ¹H-NMR (400 MHz, CDCl₃) δ 8.91 (d, J=6.8 Hz, 1H), 8.57 (d, J=6.8 Hz, 1H), 8.10 (m, 2H), 7.70 (d, J=6.8 Hz, 1H), 7.53-7.48 (m, 3H), 7.29 (m, 4H), 4.69-4.63 (m, 2H), 4.26-4.21 (m, 2H), 3.89-3.85 (m, 1H). MS (ESI⁺): m/z: 464.22 (M+H)⁺.

### Example 58. 5-((2′-(4-(1H-Imidazol-2-yl)piperazin-1-yl)-[2,4′-bipyrimidin]-4-yl)ethynyl)-1H-indazole (Ex. 58)

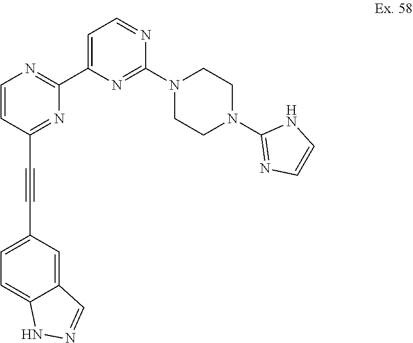 [SMILES: C(#Cc1ccnc(-c2ccnc(N3CCN(c4ncc[nH]4)CC3)n2)n1)c1ccc2[nH]ncc2c1]

Ex. 58 was prepared from 1-(1H-imidazol-2-yl)piperazine and tert-butyl 5-((2′-chloro-[2,4′-bipyrimidin]-4-yl)ethynyl)-1H-indazole-1-carboxylate (35-3) in a manner analogous to Steps 3 and 4 of Example 35. MS (ESI⁺): m/z: 449.0 (M+H)⁺.

### Example 59. 7-(4-((1H-Indazol-5-yl)ethynyl)-[2,4′-bipyrimidin]-2′-yl)-5,6,7,8-tetrahydro-[1,2,4]triazolo[4,3-a]pyrazine (Ex. 59)

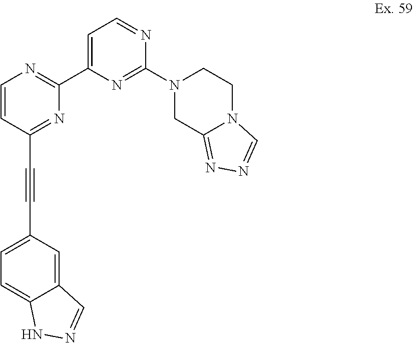 [SMILES: C(#Cc1ccnc(-c2ccnc(N3CCn4cnnc4C3)n2)n1)c1ccc2[nH]ncc2c1]

Ex. 59 was prepared from 5,6,7,8-tetrahydro-[1,2,4]triazolo[4,3-a]pyrazine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 42% yield. ¹H-NMR (400 MHz, CDCl₃) δ 8.90 (d, J=6.8 Hz, 1H), 8.62 (d, J=6.8 Hz, 1H), 8.20 (s, 1H), 8.12 (d, J=6.4 Hz, 2H), 7.76 (d, J=6.8 Hz, 1H), 7.57-7.52 (m, 3H), 5.39 (s, 2H), 4.48-4.44 (m, 2H), 4.21-4.17 (m, 2H). MS (ESI⁺): m/z: 421.27 (M+H)⁺.

### Example 60. 7-(4-((1H-Indazol-5-yl)ethynyl)-[2,4′-bipyrimidin]-2′-yl)-3-(trifluoromethyl)-5,6,7,8-tetrahydro-[1,2,4]triazolo[4,3-a]pyrazine (Ex. 60)

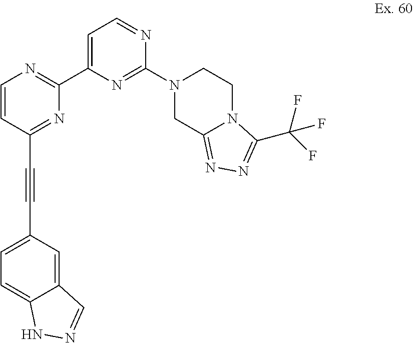 [SMILES: FC(F)(F)c1nnc2n1CCN(c1nccc(-c3nccc(C#Cc4ccc5[nH]ncc5c4)n3)n1)C2]

Ex. 60 was prepared from 3-(trifluoromethyl)-5,6,7,8-tetrahydro-[1,2,4]triazolo[4,3-a]pyrazine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 42% yield. ¹H-NMR (400 MHz, CDCl₃) δ 8.92 (d, J=6.8 Hz, 1H), 8.63 (d, J=6.8 Hz, 1H), 8.13 (d, 2H), 7.79 (d, J=6.4 Hz, 1H), 7.57-7.52 (m, 3H), 5.43 (s, 2H), 4.50-4.46 (m, 2H), 4.24-4.21 (m, 2H). MS (ESI⁺): m/z: 489.17 (M+H)⁺.

### Example 61. 5-((2′-(4-(4-Chlorophenyl)piperazin-1-yl)-[2,4′-bipyrimidin]-4-yl)ethynyl)-1H-indazole (Ex. 61)

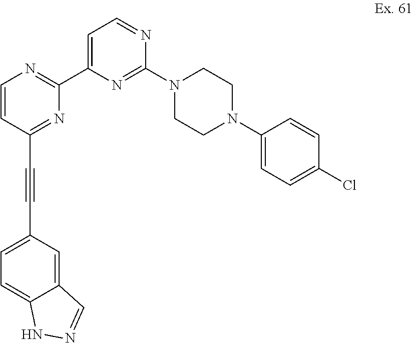 [SMILES: Clc1ccc(N2CCN(c3nccc(-c4nccc(C#Cc5ccc6[nH]ncc6c5)n4)n3)CC2)cc1]

Ex. 61 was prepared from 1-(4-chlorophenyl)piperazine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 35% yield. ¹H-NMR (400 MHz, CDCl₃) δ 8.92 (d, J=6.8 Hz, 1H), 8.56 (d, J=6.8 Hz, 1H), 8.13 (m, 2H), 7.64 (m, 2H), 7.52 (m, 2H), 7.24 (m, 2H), 7.93-6.90 (m, 2H), 4.15-4.10 (m, 4H), 3.26-3.23 (m, 4H). MS (ESI⁺): m/z: 493.16 (M+H)⁺.

### Example 62. 4-(4-(4-((1H-Indazol-5-yl)ethynyl)-[2,4′-bipyrimidin]-2′-yl)piperazin-1-yl)phenol (Ex. 62)

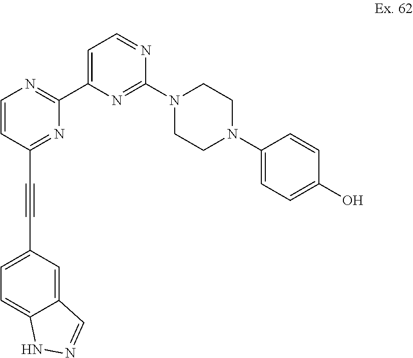 [SMILES: Oc1ccc(N2CCN(c3nccc(-c4nccc(C#Cc5ccc6[nH]ncc6c5)n4)n3)CC2)cc1]

Ex. 62 was prepared from 4-(piperazin-1-yl)phenol and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 32% yield. ¹H-NMR (400 MHz, CD₃OD) δ 8.95 (d, J=6.8 Hz, 1H), 8.59 (d, J=6.8 Hz, 1H), 8.19 (m, 2H), 7.72-7.63 (m, 4H), 7.10-7.07 (m, 2H), 680-6.77 (m, 2H), 4.20-4.19 (m, 4H), 3.30-3.29 (m, 4H). MS (ESI⁺): m/z: 475.26 (M+H)⁺.

### Example 63. 2-(4-(4-((1H-Indazol-5-yl)ethynyl)-[2,4′-bipyrimidin]-2′-yl)piperazin-1-yl)thiazole (Ex. 63)

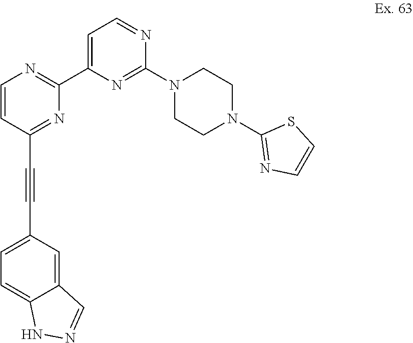 [SMILES: C(#Cc1ccnc(-c2ccnc(N3CCN(c4nccs4)CC3)n2)n1)c1ccc2[nH]ncc2c1]

Ex. 63 was prepared from 2-(piperazin-1-yl)thiazole and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 38% yield. ¹H-NMR (400 MHz, CDCl₃) δ 8.95 (d, J=6.8 Hz, 1H), 8.51 (d, J=6.8 Hz, 1H), 8.05 (m, 2H), 7.61-7.7.48 (m, 4H), 7.17 (d, J=4.8 Hz, 1H), 6.57 (d, J=4.8 Hz, 1H), 4.10-4.08 (m, 4H), 3.65-3.58 (m, 4H). MS (ESI⁺): m/z: 466.18 (M+H)⁺.

### Example 64. 7-(4-((1H-Indazol-5-yl)ethynyl)-[2,4′-bipyrimidin]-2′-yl)-3-methyl-5,6,7,8-tetrahydro-[1,2,4]triazolo[4,3-a]pyrazine (Ex. 64)

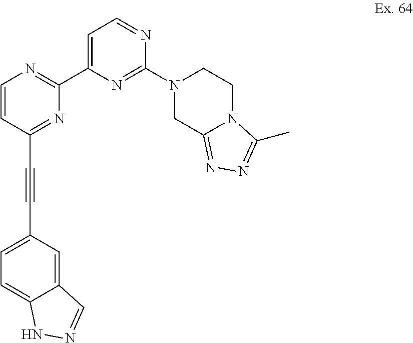 [SMILES: Cc1nnc2n1CCN(c1nccc(-c3nccc(C#Cc4ccc5[nH]ncc5c4)n3)n1)C2]

Ex. 64 was prepared from 3-methyl-5,6,7,8-tetrahydro-[1,2,4]triazolo[4,3-a]pyrazine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 31% yield. ¹H-NMR (400 MHz, CDCl₃) δ 8.90 (d, J=6.8 Hz, 1H), 8.75 (d, J=6.4 Hz, 1H), 8.09 (d, 2H), 7.74 (d, J=6.4 Hz, 1H), 7.65-7.58 (m, 3H), 5.27 (s, 2H), 4.49-4.47 (m, 2H), 4.10-4.08 (m, 2H), 2.42 (s, 3H). MS (ESI⁺): m/z: 435.23 (M+H)⁺.

### Example 65. 5-(4-((1H-Indazol-5-yl)ethynyl)-[2,4′-bipyrimidin]-2′-yl)-5,6-dihydro-4H-pyrrolo[3,4-d]thiazole (Ex. 65)

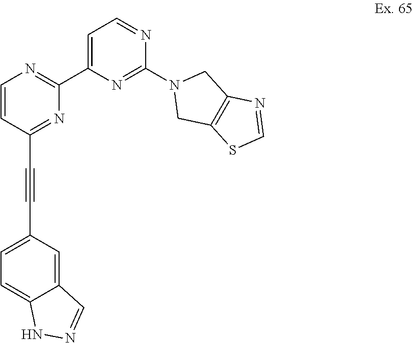 [SMILES: C(#Cc1ccnc(-c2ccnc(N3Cc4ncsc4C3)n2)n1)c1ccc2[nH]ncc2c1]

Ex. 65 was prepared from 5,6-dihydro-4H-pyrrolo[3,4-d]thiazole and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 48% yield. ¹H-NMR (400 MHz, CDCl₃) δ 9.15 (m, 2H), 8.77 (s, 111), 8.31 (s, 111), 8.26 (s, 111), 7.87-7.45 (m, 4H), 5.06 (m, 4H). MS (ESI⁺): m/z: 423.12 (M+H)⁺.

### Examples 66a and 66b. (5-((2′-(5-Fluoroisoindolin-2-yl)-[2,4′-bipyrimidin]-4-yl)ethynyl)-1H-indazol-1-yl)methyl dihydrogen phosphate (Ex. 66a) and (5-((2′-(5-Fluoroisoindolin-2-yl)-[2,4′-bipyrimidin]-4-yl)ethynyl)-2H-indazol-2-yl)methyl dihydrogen phosphate (Ex. 66b)

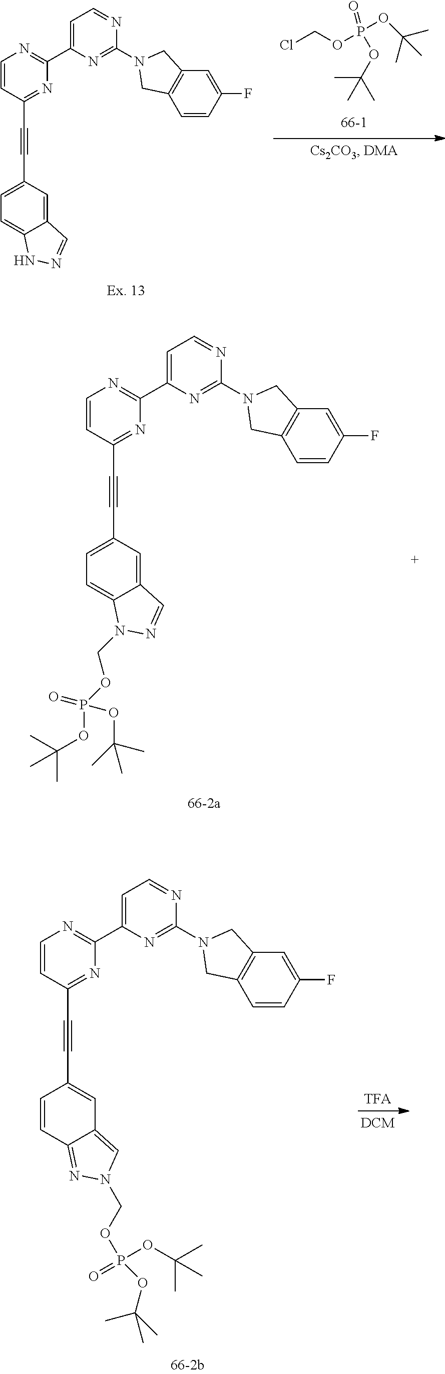

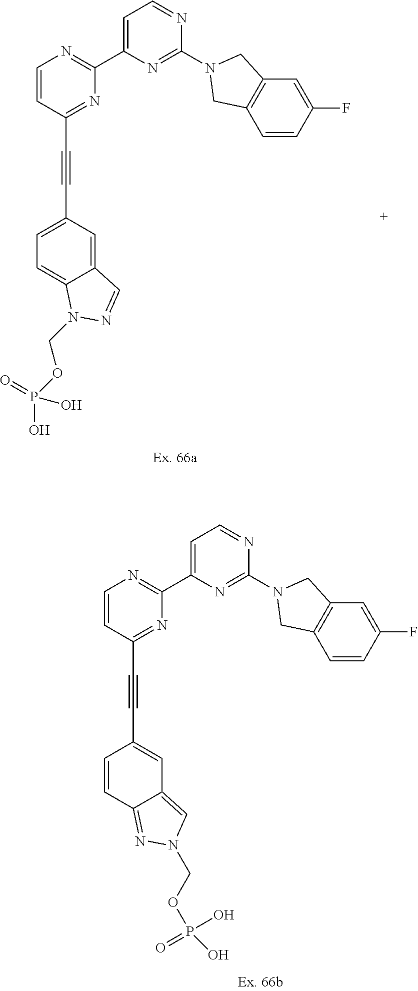 [SMILES: O=P(O)(O)OCn1ncc2cc(C#Cc3ccnc(-c4ccnc(N5Cc6ccc(F)cc6C5)n4)n3)ccc21 ; O=P(O)(O)OCn1cc2cc(C#Cc3ccnc(-c4ccnc(N5Cc6ccc(F)cc6C5)n4)n3)ccc2n1]

Step 1: Di-tert-butyl ((5-((2′-(5-fluoroisoindolin-2-yl)-[2,4′-bipyrimidin]-4-yl)ethynyl)-1H-indazol-1-yl)methyl) phosphate (66-2a) and di-tert-butyl ((5-((2′-(5-fluoroisoindolin-2-yl)-[2,4′-bipyrimidin]-4-yl)ethynyl)-2H-indazol-2-yl)methyl) phosphate (66-2b). To a suspension of 5-((2′-(5-fluoroisoindolin-2-yl)-[2,4′-bipyrimidin]-4-yl)ethynyl)-1H-indazole (Ex. 13, 100 mg, 0.231 mmol) in N,N-dimethylacetamide (DMA, 5 mL) was added Cs₂CO₃ (150.5 mg, 0.462 mmol). The resulting mixture was stirred at rt for 20 min, and then di-tert-butyl (chloromethyl) phosphate (66-1, 82 μL, 0.346 mmol, as a solution in CH₃CN) was added. The reaction mixture was stirred at room temperature for 24 h. LC-MS showed the reaction was complete. Water (20 mL) was added and the mixture was extracted with DCM (2×25 mL). The organic layers were combined, washed with brine (10 mL), dried over anhydrous Na₂SO₄, and filtered. The filtrate was concentrated to dryness and the residue was purified by flash chromatography on ISCO (sequential elution with 50% EtOAc in Hexanes, 100% EtOAc then, 5% Methanol in EtOAc) to give a mixture of di-tert-butyl ((5-((2′-(5-fluoroisoindolin-2-yl)-[2,4′-bipyrimidin]-4-yl)ethynyl)-1H-indazol-1-yl)methyl) phosphate (66-2a) and di-tert-butyl ((5-((2′-(5-fluoroisoindolin-2-yl)-[2,4′-bipyrimidin]-4-yl)ethynyl)-2H-indazol-2-yl)methyl) phosphate (66-2b) (101 mg, yield: 67%) as a pale yellow solid. MS (ESI⁺): m/z: 656.3 (M+H)⁺.

Step 2: (5-((2′-(5-Fluoroisoindolin-2-yl)-[2,4′-bipyrimidin]-4-yl)ethynyl)-1H-indazol-1-yl)methyl dihydrogen phosphate (Ex. 66a) and (5-((2′-(5-Fluoroisoindolin-2-yl)-[2,4′-bipyrimidin]-4-yl)ethynyl)-2H-indazol-2-yl)methyl dihydrogen phosphate (Ex. 66b): To a mixture of 66-2a and 66-2b (40 mg, 0.061 mmol) in DCM (0.4 mL) was added 90% TFA in DCM (3.6 mL). The resulting mixture was stirred at room temperature for 2 h. LC-MS showed the reaction was complete. The reaction mixture was concentrated and co-evaporated with DCM multiple times to remove trace amounts of TFA. The resulting solid was triturated with ether (3 mL) to further remove remaining TFA. A mixture of Ex. 66a and Ex. 66b (26.5 mg, yield: 80%) was obtained as a yellow solid. For the major isomer: ¹H-NMR (300 MHz, DMSO-d₆): δ (ppm): 9.13 (d, J=4.8 Hz, 1H), 8.74 (d, J=5.1 Hz, 1H), 8.31 (s, 1H), 8.27 (s, 1H), 7.90 (d, J=5.1, 1H), 7.72-7.70 (m, 2H), 7.65 (d, J=5.1 Hz, 1H), 7.56-7.50 (m, 1H), 7.44-7.33 (m, 111), 7.26-7.19 (m, 1H), 5.82 (d, J=11.2 Hz, 2H), 5.01-4.90 (m, 411). MS (ESI⁺): m/z: 544.3 (M+H⁺).

### Examples 67a and 67b. (7-Fluoro-5-((2′-(5-fluoroisoindolin-2-yl)-[2,4′-bipyrimidin]-4-yl)ethynyl)-1H-indazol-1-yl)methyl dihydrogen phosphate (Ex. 67a) and (7-fluoro-5-((2′-(5-fluoroisoindolin-2-yl)-[2,4′-bipyrimidin]-4-yl)ethynyl)-2H-indazol-2-yl)methyl dihydrogen phosphate (Ex. 67b)

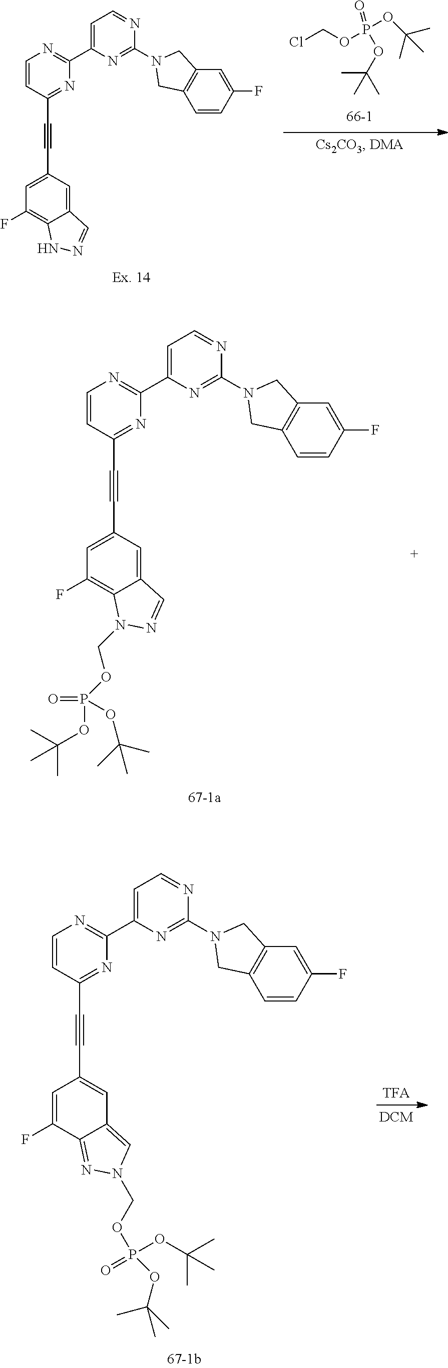

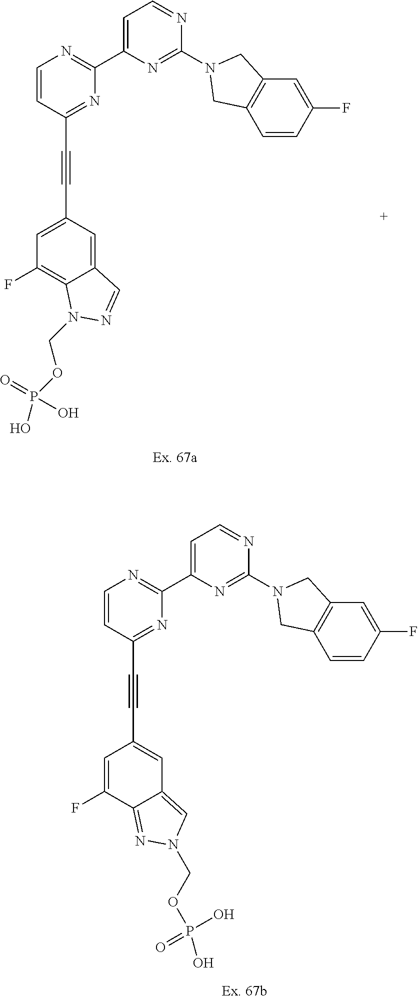 [SMILES: O=P(O)(O)OCn1ncc2cc(C#Cc3ccnc(-c4ccnc(N5Cc6ccc(F)cc6C5)n4)n3)cc(F)c21 ; O=P(O)(O)OCn1cc2cc(C#Cc3ccnc(-c4ccnc(N5Cc6ccc(F)cc6C5)n4)n3)cc(F)c2n1]

Ex. 67a and Ex. 67b were prepared from 7-fluoro-5-((2′-(5-fluoroisoindolin-2-yl)-[2,4′-bipyrimidin]-4-yl)ethynyl)-1H-indazole (Ex. 14) and di-tert-butyl (chloromethyl) phosphate in a manner analogous to Examples 66a and 66b to provide the compounds in 13.4% overall yield in 2 steps. For the major isomer: ¹H-NMR (300 MHz, DMSO-d₆): δ (ppm): 9.15 (d, J=5.1 Hz, 1H), 8.88 (d, J=2.7 Hz, 1H), 8.73 (d, J=5.1 Hz, 1H), 8.22 (s, 1H), 7.91 (d, J=5.1 Hz, 1H), 7.64 (d, J=4.8, 1H), 7.58-7.30 (m, 3H), 7.24-7.18 (m, 1H), 6.19 (d, J=11.1 Hz, 2H), 5.11-4.85 (m, 4H). MS (ESI⁺): m/z: 562.3 (M+H)⁺.

### Examples 68a and 68b. (7-Fluoro-5-((2′-(5-methoxyisoindolin-2-yl)-[2,4′-bipyrimidin]-4-yl)ethynyl)-1H-indazol-1-yl)methyl dihydrogen phosphate (Ex. 68a) and (7-fluoro-5-((2′-(5-methoxyisoindolin-2-yl)-[2,4′-bipyrimidin]-4-yl)ethynyl)-2H-indazol-2-yl)methyl dihydrogen phosphate (Ex. 68b)

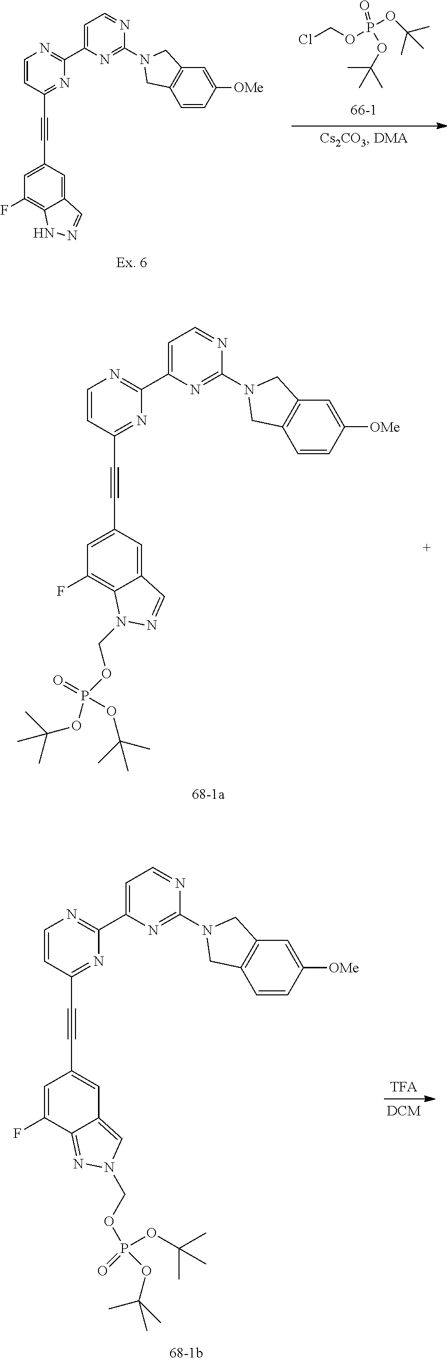

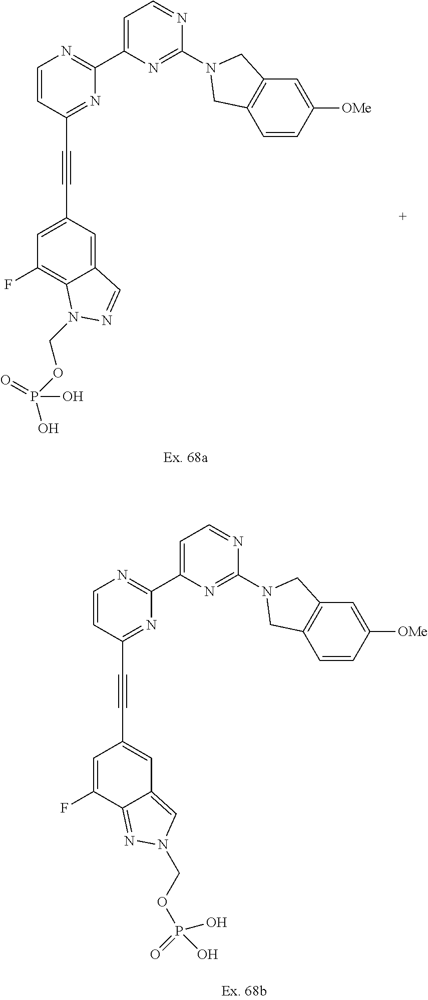 [SMILES: COc1ccc2c(c1)CN(c1nccc(-c3nccc(C#Cc4cc(F)c5c(cnn5COP(=O)(O)O)c4)n3)n1)C2 ; COc1ccc2c(c1)CN(c1nccc(-c3nccc(C#Cc4cc(F)c5nn(COP(=O)(O)O)cc5c4)n3)n1)C2]

Ex. 68a and Ex. 68b were prepared from 7-fluoro-5-((2′-(5-methoxyisoindolin-2-yl)-[2,4′-bipyrimidin]-4-yl)ethynyl)-1H-indazole (Ex. 6) and di-tert-butyl (chloromethyl) phosphate in a manner analogous to Examples 66a and 66b to provide the compound as a brown solid in 28.9% overall yield in 2 steps. For the major isomer: ¹H-NMR (300 MHz, DMSO-d₄): δ (ppm): 9.13 (d, J=4.8 Hz, 1H), 8.87 (s, 1H), 8.71 (d, J=4.8 Hz, 1H), 8.19 (s, 1H), 7.89 (d, J=4.5 Hz, 1H), 7.61 (d, J=4.8, 1H), 7.46-7.37 (m, 2H), 7.13-7.07 (m, 1H), 6.97-6.90 (m, 1H), 6.16 (d, J=11.1 Hz, 2H), 4.95-4.87 (m, 4H), 3.82 (s, 3H). MS (ESI⁺): m/z: 574.3 (M+H)⁺.

### Example 69. 5-((2′-(3-Phenylpyrrolidin-1-yl)-[2,4′-bipyrimidin]-4-yl)ethynyl)-1H-indazole (Ex. 69)

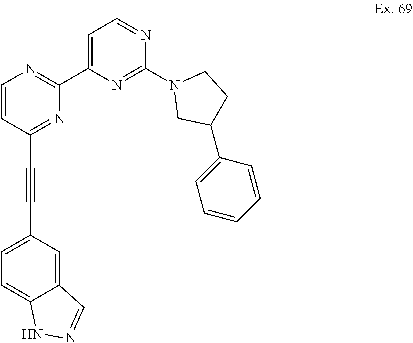 [SMILES: C(#Cc1ccnc(-c2ccnc(N3CCC(c4ccccc4)C3)n2)n1)c1ccc2[nH]ncc2c1]

Ex. 69 was prepared from 3-phenylpyrrolidine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 42% yield over two steps. ¹H-NMR (400 MHz, CDCl₃) δ 8.93 (d, J=6.8 Hz, 1H), 8.56 (d, J=6.4 Hz, 1H), 8.20 (m, 2H), 8.05 (m, 1H), 7.77 (m, 1H), 7.56-7.48 (m, 2H), 7.30 (m, 4H), 7.25 (m, 1H), 3.74-3.70 (m, 3H), 2.94 (m, 1H), 2.86 (m, 1H), 2.44 (m, 1H), 2.16 (m, 1H). MS (ESI⁺): m/z: 444.23 (M+H)⁺.

### Example 70. 5-((2′-(3-(3-Methoxyphenyl)pyrrolidin-1-yl)-[2,4′-bipyrimidin]-4-yl)ethynyl)-1H-indazole (Ex. 70)

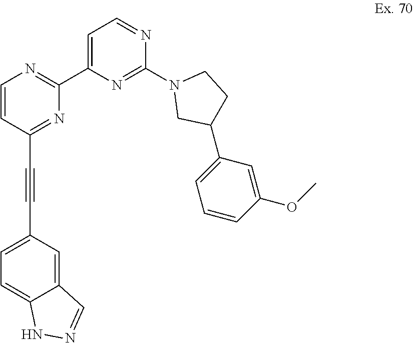 [SMILES: COc1cccc(C2CCN(c3nccc(-c4nccc(C#Cc5ccc6[nH]ncc6c5)n4)n3)C2)c1]

Ex. 70 was prepared from 3-(3-methoxyphenyl)pyrrolidine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 38% yield over two steps. ¹H-NMR (400 MHz, CDCl₃) δ 9.01 (d, J=6.8 Hz, 1H), 8.71 (d, J=6.4 Hz, 1H), 8.18 (m, 2H), 7.87 (m, 1H). 768-7.55 (m, 2H), 7.26 (m, 1H), 6.89-6.80 (m, 3H), 4.46 (m, 1H), 4.17-4.15 (m, 3H), 3.81 (s, 3H), 3.48 (m, 2H), 2.52 (m. 1H), 2.23 (m, 1H). MS (ESI⁺): m/z: 474.18 (M+H)⁺.

### Example 71. 5-((2′-(3-(4-Fluorophenyl)pyrrolidin-1-yl)-[2,4′-bipyrimidin]-4-yl)ethynyl)-1H-indazole (Ex. 71)

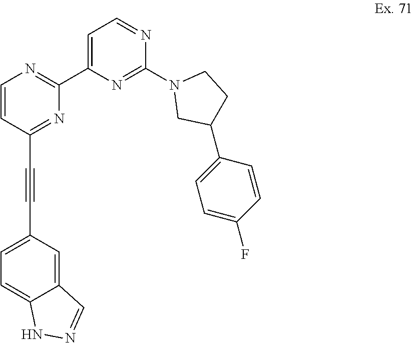 [SMILES: Fc1ccc(C2CCN(c3nccc(-c4nccc(C#Cc5ccc6[nH]ncc6c5)n4)n3)C2)cc1]

Ex. 71 was prepared from 3-(4-fluorophenyl)pyrrolidine and tert-butyl 5-((2′-chloro-[2,4′-bipyrimidin]-4-yl)ethynyl)-1H-indazole-1-carboxylate (35-3) in a manner analogous to Steps 3 and 4 of Example 35 to provide the compound in 35% yield over two steps. ¹H-NMR (400 MHz, CDCl₃) δ 9.02 (d, J=6.8 Hz, 1H), 8.67 (d, J=6.4 Hz, 1H), 8.16 (m, 2H), 7.91 (m, 1H), 7.66-7.59 (m, 3H), 7.27-7.24 (m, 2H), 7.07-7.021 (m, 2H), 4.51 (m, 1H), 4.27 (m, 1H), 3.97 (m, 1H), 3.83-3.82 (m, 2H), 2.55 (m, 1H), 2.21 (m, 1H). MS (ESI⁺): m/z: 462.10 (M+H)⁺.

### 2) Biological Activity:

1. ROCK1 and ROCK2 kinase assays: The ROCK1 and ROCK2 kinase binding affinities of compounds in this invention were determined by DiscoverX's KINOMEscan™ KdELECT technology (https://www.discoverx.com/kinomescan-elect-kinase-screening-and-profiling-services): Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111X stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

The testing results of the equilibrium dissociation constant (Kd) of selected compounds of this invention are shown in the following table. The data for the reference compound KD025 were generated in the same assays for comparison.

| | Kd (μM) | | ROCK 2 |
|---|---|---|---|
| Compound | ROCK1 | ROCK2 | Selectivity* |
| KD025 | 13.0 | 0.12 | 108 |
| Ex. 1 | >30 | >30 | N/C |
| Ex. 2 | >30 | >30 | N/C |
| Ex. 3 | 0.82 | 0.0069 | 119 |
| Ex. 4 | >30 | 8.2 | >3.66 |
| Ex. 5 | >30 | 0.020 | >1500 |
| Ex. 6 | 29.0 | 0.012 | 2417 |
| Ex. 7 | >30 | >30 | N/C |
| Ex. 8 | >30 | >30 | N/C |
| Ex. 9 | >30 | >30 | N/C |
| Ex. 10 | >30 | >30 | N/C |
| Ex. 11 | >30 | >30 | N/C |
| Ex. 12 | 0.25 | 0.0018 | 139 |
| Ex. 13 | 9.2 | 0.0079 | 1164 |
| Ex. 14 | >30 | 0.028 | >1071 |
| Ex. 15 | >6.0 | 0.17 | >35 |
| Ex. 16 | >6.0 | 0.26 | >23 |
| Ex. 17 | >6.0 | 0.049 | >122 |
| Ex. 18 | 25 | 0.120 | 100 |
| Ex. 19 | >30 | 2.80 | >10.7 |
| Ex. 20 | >30 | 5.5 | >5.4 |
| Ex. 21 | >30 | 6.8 | >4.4 |
| Ex. 22 | >30 | 1.5 | >20 |
| Ex. 23 | >30 | 0.66 | >45 |
| Ex. 24 | >30 | >30 | N/C |
| Ex. 25 | >30 | >30 | N/C |
| Ex. 26 | >30 | 4.5 | >6.67 |
| Ex. 27 | >30 | >30 | N/C |
| Ex. 28 | >30 | >30 | N/C |
| Ex. 29 | >30 | >30 | N/C |
| Ex. 30 | 0.77 | 0.0058 | 132.8 |
| Ex. 31 | >30 | 0.073 | >411 |
| Ex. 32 | >30 | 0.29 | >103 |
| Ex. 33 | >30 | 0.68 | >44.1 |
| Ex. 34 | 0.089 | 0.031 | 2.87 |
| Ex. 35 | >30 | 0.072 | >417 |
| Ex. 36 | 0.069 | 0.0095 | 7.26 |
| Ex. 37 | 5.7 | 0.11 | 51.8 |
| Ex. 38 | 2.8 | 0.64 | 4.37 |
| Ex. 39 | 0.67 | 0.026 | 25.8 |
| Ex. 40 | 12 | 0.083 | 144 |
| Ex. 41 | 1.1 | 0.024 | 45.8 |
| Ex. 42 | 0.64 | 0.083 | 7.71 |
| Ex. 43 | >30 | 0.11 | >273 |
| Ex. 44 | 0.075 | 0.0023 | 32.6 |
| Ex. 45 | 0.1 | 0.0011 | 90.9 |
| Ex. 46 | 0.13 | 0.0023 | 56.5 |
| Ex. 47 | 0.16 | 0.0031 | 51.6 |
| Ex. 48 | 5.5 | 0.15 | 36.7 |
| Ex. 49 | >30 | 0.25 | >120 |
| Ex. 50 | 5.4 | 0.12 | 45 |
| Ex. 51 | 11 | 0.15 | 73 |
| Ex. 52 | 9 | 0.34 | 26.5 |
| Ex. 53 | 4.7 | 0.08 | 58.8 |
| Ex. 54 | 4.6 | 0.45 | 10.2 |
| Ex. 55 | 26 | 0.9 | 28.9 |
| Ex. 56 | 26 | 0.73 | 35.6 |
| Ex. 57 | 6.7 | 0.22 | 30.5 |
| Ex. 58 | 1.6 | 0.32 | 5 |
| Ex. 59 | 16 | 0.75 | 21.3 |
| Ex. 60 | 9.9 | 0.21 | 47.1 |
| Ex. 61 | 22 | 0.49 | 44.9 |
| Ex. 62 | 13 | 0.11 | 118.2 |
| Ex. 63 | >30 | 0.33 | >90.9 |
| Ex. 64 | 3.4 | 0.33 | 10.3 |
| Ex. 65 | 3.4 | 0.024 | 142 |
| Ex. 69 | 9.4 | 0.26 | 36.2 |
| Ex. 70 | 6.1 | 0.22 | 27.7 |
| Ex. 71 | 13 | 0.36 | 36.1 |

*N/C = not calculated

The data show compounds of this invention bind to both ROCK1 and ROCK2, especially the latter.

What is claimed is:

1. A compound of formula I: 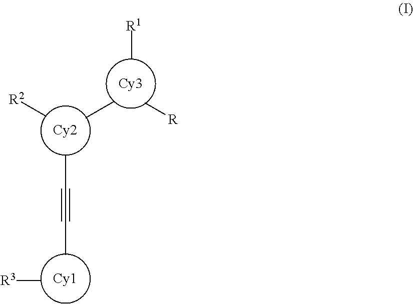
or a pharmaceutically acceptable salt thereof, wherein,
Cy1, Cy2, and Cy3 each independently represents an aryl, heteroaryl, or heterocyclic, which is optionally fused with a 3-8 membered cycloalkyl, 3-8 membered heterocycloalkyl, 6-membered aryl, or 5-6 membered heteroaryl;
R¹, R², and R³ each independently represent one, two, three, or four same or different substituents selected from hydrogen, deuterium, halo, —CN, —NO₂, or an optionally substituted aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R^{g}, —CO₂R^{h}, —CONRⁱR^{j}, —NR^{k}CONR^{l}R^{m}, —OCONRⁿR^{o}, or —NR^{p}CO₂R^{q};
R is a heterocyclic, aromatic, or heteroaromatic; optionally substituted with one or more independent hydrogen, deuterium, halo, —CN, —NO₂, aliphatic, alicyclic, heteroaliphatic, heterocyclic, aromatic, heteroaromatic, —OR^{a}, —NR^{b}R^{c}, —S(═O)_{w}R^{d}, —O—S(═O)_{w}R^{d}, —S(═O)_{w}NR^{e}R^{f}, —C(═O)R, —CO₂R^{h}, —CONRⁱR^{j}, —NR^{k}CONR^{l}R^{m}, —OCONRⁿR^{o}, or —NR^{k}CO₂R^{p};
R^{a}, R^{b}, R^{c}, R^{d}, R^{e}, R^{f}, R^{g}, R^{h}, Rⁱ, R^{j}, R^{k}, R^{l}, R^{m}, Rⁿ, R^{o}, R^{p} and R^{q}, for each occurrence, is independently selected from hydrogen, deuterium, halo, —CN, —NO₂, an optionally substituted aliphatic, alicyclic, heteroaliphatic, heterocyclic, aromatic, or heteroaromatic; wherein each optional substituent is independently selected from one or more independent hydrogen, deuterium, halo, —CN, —NO₂, aliphatic, alicyclic, heteroaliphatic, heterocyclic, aromatic, heteroaromatic, —OR^{aa}, —NR^{bb}R^{cc}, —S(═O)_{w}R^{dd}, —S(═O)_{w}NR^{ee}R^{ff}, —C(═O)R^{gg}, —CO₂R^{hh}, —CONRⁱⁱR^{jj}, —NR^{kk}CONR^{ll}R^{mm}, —OCONRⁿⁿR^{oo}, or —NR^{kk}CO₂R^{pp}; or R^{b} and R^{c}, R^{e} and R^{f}, Rⁱ and R^{j}, R^{l} and R^{m}, or Rⁿ and R^{o}, when attached to the same nitrogen, may optionally form a heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, aliphatic, alicyclic, heteroaliphatic, heterocyclic, aromatic, or heteroaromatic;
R^{aa}, R^{bb}, R^{cc}, R^{dd}, R^{ee}, R^{ff}, R^{gg}, R^{hh}, Rⁱⁱ, R^{jj}, R^{kk}, R^{ll}, R^{mm}, Rⁿⁿ, R^{oo}, and R^{pp}, for each occurrence, is independently selected from hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic; and
w is 0, 1, or 2.

2. The compound of claim 1, wherein Cy1 is a monocyclic or bicyclic or tricyclic aryl, heteroaryl, or heterocyclic.

3. The compound of claim 2, wherein Cy1 is selected from phenyl, pyridinyl, pyridonyl, pyrimidinyl, pyrazinyl, pyridazinyl, triazinyl, tetrazinyl, quinolinyl, quinazolinyl, quinoxalinyl, cinnolinyl, isoquinolinyl, indolyl, aza-indolyl, indolinonyl, indolinyl, oxoindolinyl, tetrahydro-indazolyl, pyrazolyl, imidazolyl, triazolyl, tetrazolyl, oxazolyl, thiazolyl, benzimidazolyl, indazolyl, aza-indazolyl, benzoxazolyl, or benzothiazolyl.

4. The compound of claim 1, wherein Cy2 and Cy3 each independently represents a monocyclic or bicyclic aromatic, a monocyclic or bicyclic heteroaromatic, or a monocyclic or bicyclic heterocyclic.

5. The compound of claim 4, wherein Cy2 and Cy3 is each independently selected from phenyl, naphthyl, pyridinyl, pyridonyl, pyrimidinyl, pyrazinyl, pyridazinyl, triazinyl, tetrazinyl, quinolinyl, quinazolinyl, quinoxalinyl, cinnolinyl, indolyl, aza-indolyl, pyrazolyl, imidazolyl, triazolyl, tetrazolyl, oxazolyl, thiazolyl, benzimidazolyl, indazolyl, benzoxazolyl, or benzothiazolyl.

6. The compound of claim 1, wherein R is a heterocyclic group.

7. The compound of claim 6, wherein R is selected from azetidinyl, pyrrolidinyl, piperidinyl, piperazinyl, 5,6,7,8-tetrahydro-[1,2,4]triazolo[4,3-a]pyrazinyl, 4,5,6,7-tetrahydro-1H-pyrazolo[4,3-c]pyridinyl, indolinyl, isoindolinyl, aza-indolinyl, aza-isoindolinyl, dihydroindazolyl, tetrahydroquinolinyl, tetrahydroisoquinolinyl, aza-tetrahydroquinolinyl or aza-tetrahydroisoquinolinyl.

8. The compound of claim 1, wherein the structure of the compound is formula Ia: 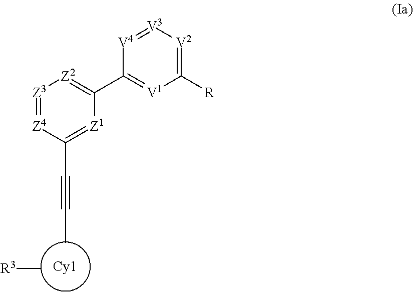
wherein V1, V², V³ and V⁴ are each independently N or C—R¹, wherein two R¹ groups on adjacent carbon atoms together with the carbons to which they are attached may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂;
Z¹, Z², Z³ and Z⁴ is each independently N or C—R², wherein two R² groups on adjacent carbon atoms together with the carbons to which they are attached may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O), CH₃, or an aliphatic, alicyclic, heteroaliphatic, heterocyclic, aromatic, or heteroaromatic, which may be optionally substituted with one or more independent deuterium, halo, —CN, —OH, —NO₂, —SH, —CO₂H, or —NH₂;
wherein the definitions of R, R¹, R², Cy1, and R³ are the same with those in claim 1.

9. The compound of claim 8, wherein the structure of the compound is formula Ib: 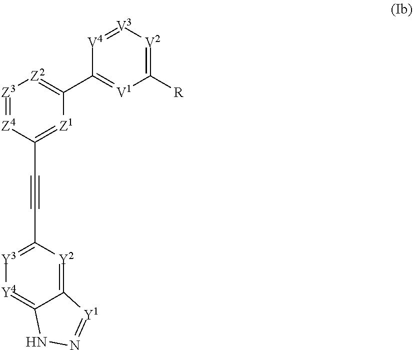
wherein Y¹, Y², Y³ and Y⁴ is each independently N or C—R³, wherein two R³ groups on adjacent carbon atoms together with the carbons they are attached to may optionally form a 5-7 membered aromatic, heteroaromatic, or heterocyclic ring, optionally containing 1-5 additional heteroatoms selected from O, S(O)_{w}, or N as the ring atoms, and may be optionally substituted with one or more independent hydrogen, deuterium, halo, —CN, —NO₂, —OH, —CH₂F, —CHF₂, —CF₃, —OCH₃, —OCH₂F, —OCHF₂, —OCF₃, —NH₂, —NHCH₃, —N(CH₃)₂, —CO₂H, —SH, —S(O)_{w}CH₃, or an aliphatic, alicyclic, heteroaliphatic, heterocyclic, aromatic, or heteroaromatic;
wherein definitions of V¹, V¹, V², V⁴, Z¹, Z², Z³, and Z⁴ are the same with those in claim 8; and R and R³ have the same meaning with those in claim 1.

10. The compound of claim 9, wherein the structure of the compound is formula Ic or Id: 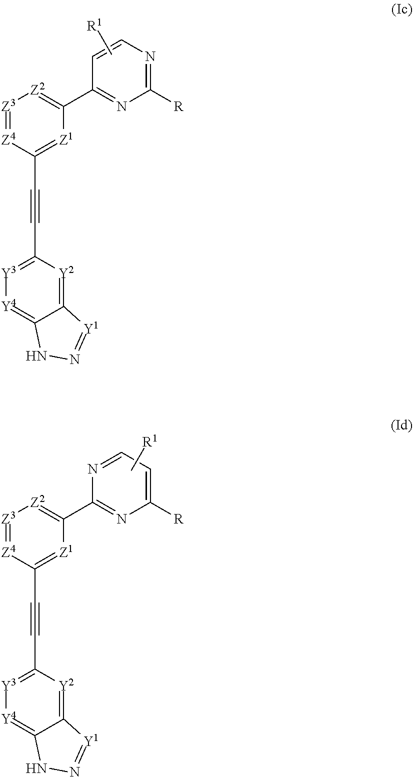
wherein the definitions of Z¹, Z², Z³, and Z⁴ are the same with those in claim 8; the definitions of Y¹, Y², Y³, and Y⁴ are the same with those in claim 9; and R and R¹ have the same meaning with those in claim 1.

11. The compound of claim 10, wherein the structure of the compound is formula Ie, If, Ig, Ih, Ii, or Ij: 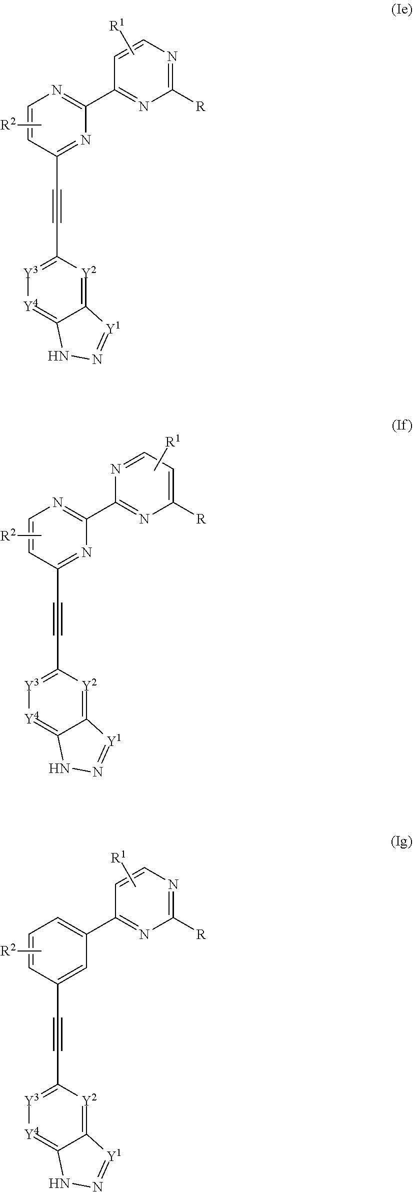 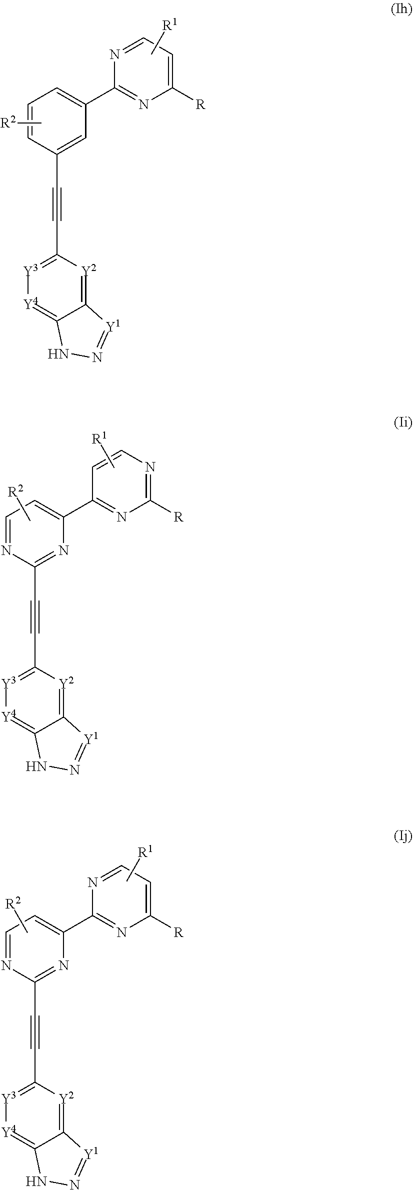
wherein the definitions of Y¹, Y², Y³, and Y⁴ are the same with those in claim 9; and the definitions of R, R¹, and R² have the same meaning with those in claim 1.

12. The compound of claim 11, wherein the structure of the compound is formula Ik, Il, Im, In, Io, or Ip: 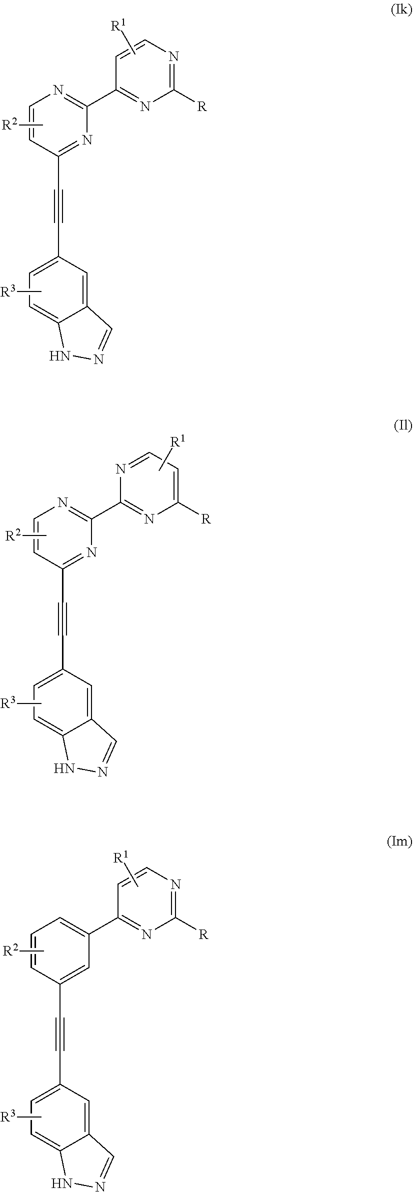 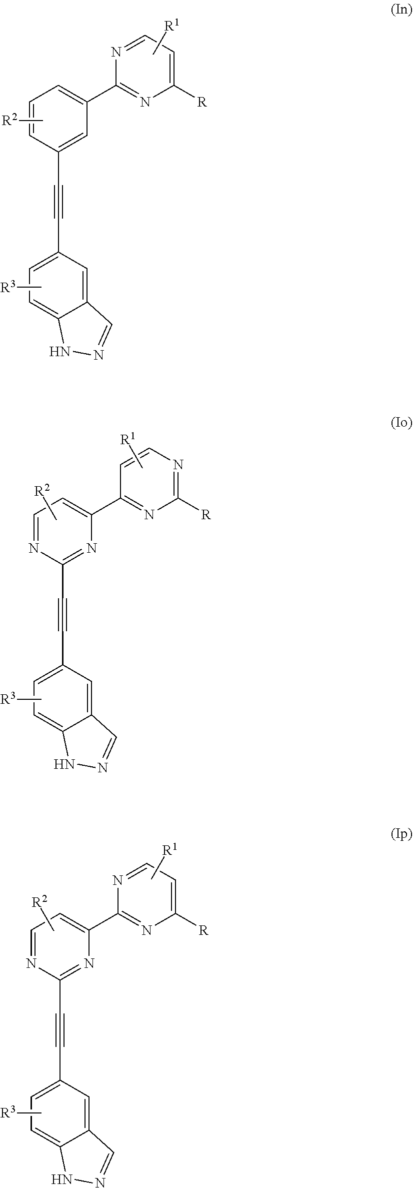
wherein R, R¹, R², and R³ have the same meaning with those in claim 1; and wherein the R³ group can be connected to any carbon atom in the indazolyl ring.

13. The compound of claim 1 or a pharmaceutically acceptable salt thereof, wherein the compound is selected from the following:
5-Methoxy-2-(4-(pyridin-4-ylethynyl)-[2,4′-bipyrimidin]-2′-yl)isoindoline;
2-(4-((1H-pyrazol-4-yl)ethynyl)-[2,4′-bipyrimidin]-2′-yl)-5-methoxyisoindoline;
5-((2′-(5-methoxyisoindolin-2-yl)-[2,4′-bipyrimidin]-4-yl)ethynyl)-1H-indazole;
6-((2′-(5-methoxyisoindolin-2-yl)-[2,4′-bipyrimidin]-4-yl)ethynyl)isoquinolin-1-amine;
3-fluoro-5-((2′-(5-methoxyisoindolin-2-yl)-[2,4′-bipyrimidin]-4-yl)ethynyl)-1H-indazole;
7-fluoro-5-((2′-(5-methoxyisoindolin-2-yl)-[2,4′-bipyrimidin]-4-yl)ethynyl)-1H-indazole;
5-((2′-(5-methoxyisoindolin-2-yl)-[2,4′-bipyrimidin]-4-yl)ethynyl)isoindolin-1-one;
methyl 4-((2′-(5-methoxyisoindolin-2-yl)-[2,4′-bipyrimidin]-4-yl)ethynyl)benzoate;
4-((2′-(5-methoxyisoindolin-2-yl)-[2,4′-bipyrimidin]-4-yl)ethynyl)benzonitrile;
4-((2′-(5-methoxyisoindolin-2-yl)-[2,4′-bipyrimidin]-4-yl)ethynyl)benzoic acid;
4-((2′-(5-methoxyisoindolin-2-yl)-[2,4′-bipyrimidin]-4-yl)ethynyl)-N-methylbenzamide;
5-((2′-(isoindolin-2-yl)-[2,4′-bipyrimidin]-4-yl)ethynyl)-1H-indazole;
5-((2′-(5-fluoroisoindolin-2-yl)-[2,4′-bipyrimidin]-4-yl)ethynyl)-1H-indazole;
7-fluoro-5-((2′-(5-fluoroisoindolin-2-yl)-[2,4′-bipyrimidin]-4-yl)ethynyl)-1H-indazole;
5-((2′-(6-methoxy-1,3-dihydro-2H-pyrrolo[3,4-c]pyridin-2-yl)-[2,4′-bipyrimidin]-4-yl)ethynyl)-1H-indazole;
2-(4-((1H-indazol-5-yl)ethynyl)-[2,4′-bipyrimidin]-2′-yl)-2,3-dihydro-1H-pyrrolo[3,4-c]pyridin-6-ol;
5-((2′-(6-chloro-1,3-dihydro-2H-pyrrolo[3,4-c]pyridin-2-yl)-[2,4′-bipyrimidin]-4-yl)ethynyl)-1H-indazole;
5-((6-(2-(5-methoxyisoindolin-2-yl)pyrimidin-4-yl)pyridin-2-yl)ethynyl)-1H-indazole;
7-fluoro-5-((6-(2-(5-methoxyisoindolin-2-yl)pyrimidin-4-yl)pyridin-2-yl)ethynyl)-1H-indazole;
5-((6-(2-(5-fluoroisoindolin-2-yl)pyrimidin-4-yl)pyridin-2-yl)ethynyl)-1H-indazole;
7-fluoro-5-((6-(2-(5-fluoroisoindolin-2-yl)pyrimidin-4-yl)pyridin-2-yl)ethynyl)-1H-indazole;
2-((2-(4-(6-((1H-indazol-5-yl)ethynyl)pyridin-2-yl)pyrimidin-2-yl)isoindolin-5-yl)oxy)-N,N-dimethylethanamine;
5-((6-(2-(5-(4-methylpiperazin-1-yl)isoindolin-2-yl)pyrimidin-4-yl)pyridin-2-yl)ethynyl)-1H-indazole;
5-((3-fluoro-5-(2-(5-methoxyisoindolin-2-yl)pyrimidin-4-yl)phenyl)ethynyl)-1H-indazole;
5-((3-fluoro-5-(2-(5-fluoroisoindolin-2-yl)pyrimidin-4-yl)phenyl)ethynyl)-1H-indazole;
5-((3-(2-(5-chloroisoindolin-2-yl)pyrimidin-4-yl)-5-fluorophenyl)ethynyl)-1H-indazole;
5-((3-(2-(5-bromoisoindolin-2-yl)pyrimidin-4-yl)-5-fluorophenyl)ethynyl)-1H-indazole;
2-((2-(4-(3-((1H-indazol-5-yl)ethynyl)-5-fluorophenyl)pyrimidin-2-yl)isoindolin-5-yl)oxy)-N,N-dimethylethanamine;
5-((3-fluoro-5-(2-(5-(4-methylpiperazin-1-yl)isoindolin-2-yl)pyrimidin-4-yl)phenyl)ethynyl)-1H-indazole;
5-((2′-(5-bromoisoindolin-2-yl)-[2,4′-bipyrimidin]-4-yl)ethynyl)-1H-indazole;
5-((2′-(5-chloroisoindolin-2-yl)-[2,4′-bipyrimidin]-4-yl)ethynyl)-1H-indazole;
5-((2′-(5-cyanoisoindolin-2-yl)-[2,4′-bipyrimidin]-4-yl)ethynyl)-1H-indazole;
5-((2′-(5-(fluoromethyl)isoindolin-2-yl)-[2,4′-bipyrimidin]-4-yl)ethynyl)-1H-indazole;
5-((2′-(5-(difluoromethyl)isoindolin-2-yl)-[2,4′-bipyrimidin]-4-yl)ethynyl)-1H-indazole;
5-((2′-(5-(trifluoromethyl)isoindolin-2-yl)-[2,4′-bipyrimidin]-4-yl)ethynyl)-1H-indazole;
5-((2′-(5-(difluoromethoxyl)isoindolin-2-yl)-[2,4′-bipyrimidin]-4-yl)ethynyl)-1H-indazole;
5-((2′-(5-(triifluoromethoxyl)isoindolin-2-yl)-[2,4′-bipyrimidin]-4-yl)ethynyl)-1H-indazole;
7-fluoro-5-((2′-(isoindolin-2-yl)-[2,4′-bipyrimidin]-4-yl)ethynyl)-1H-indazole;
7-fluoro-5-((2′-(5-chloroisoindolin-2-yl)-[2,4′-bipyrimidin]-4-yl)ethynyl)-1H-indazole;
7-fluoro-5-((2′-(5-bromoisoindolin-2-yl)-[2,4′-bipyrimidin]-4-yl)ethynyl)-1H-indazole;
7-fluoro-5-((2′-(5-cyanoisoindolin-2-yl)-[2,4′-bipyrimidin]-4-yl)ethynyl)-1H-indazole;
7-fluoro-5-((2′-(5-difluoromethoxyisoindolin-2-yl)-[2,4′-bipyrimidin]-4-yl)ethynyl)-1H-indazole;
7-fluoro-5-((2′-(5-trifluoromethoxyisoindolin-2-yl)-[2,4′-bipyrimidin]-4-yl)ethynyl)-1H-indazole;
7-fluoro-5-((2′-(5-(fluoromethyl)isoindolin-2-yl)-[2,4′-bipyrimidin]-4-yl)ethynyl)-1H-indazole;
7-fluoro-5-((2′-(5-(difluoromethyl)isoindolin-2-yl)-[2,4′-bipyrimidin]-4-yl)ethynyl)-1H-indazole;
7-fluoro-5-((2′-(5-(trifluoromethyl)isoindolin-2-yl)-[2,4′-bipyrimidin]-4-yl)ethynyl)-1H-indazole;
3-fluoro-5-((2′-(isoindolin-2-yl)-[2,4′-bipyrimidin]-4-yl)ethynyl)-1H-indazole;
3-fluoro-5-((2′-(5-chloroisoindolin-2-yl)-[2,4′-bipyrimidin]-4-yl)ethynyl)-1H-indazole;
3-fluoro-5-((2′-(5-bromoisoindolin-2-yl)-[2,4′-bipyrimidin]-4-yl)ethynyl)-1H-indazole;
3-fluoro-5-((2′-(5-cyanoisoindolin-2-yl)-[2,4′-bipyrimidin]-4-yl)ethynyl)-1H-indazole;
3-fluoro-5-((2′-(5-difluoromethoxyisoindolin-2-yl)-[2,4′-bipyrimidin]-4-yl)ethynyl)-1H-indazole;
3-fluoro-5-((2′-(5-trifluoromethoxyisoindolin-2-yl)-[2,4′-bipyrimidin]-4-yl)ethynyl)-1H-indazole;
3-fluoro-5-((2′-(5-(fluoromethyl)isoindolin-2-yl)-[2,4′-bipyrimidin]-4-yl)ethynyl)-1H-indazole;
3-fluoro-5-((2′-(5-(difluoromethyl)isoindolin-2-yl)-[2,4′-bipyrimidin]-4-yl)ethynyl)-1H-indazole; and
3-fluoro-5-((2′-(5-(trifluoromethyl)isoindolin-2-yl)-[2,4′-bipyrimidin]-4-yl)ethynyl)-1H-indazole.

14. The compound of claim 13 or a pharmaceutically acceptable salt thereof, wherein the compound is selected from the following:
5-Methoxy-2-(4-(pyridin-4-ylethynyl)-[2,4′-bipyrimidin]-2′-yl)isoindoline;
2-(4-((1H-pyrazol-4-yl)ethynyl)-[2,4′-bipyrimidin]-2′-yl)-5-methoxyisoindoline;
5-((2′-(5-methoxyisoindolin-2-yl)-[2,4′-bipyrimidin]-4-yl)ethynyl)-1H-indazole;
6-((2′-(5-methoxyisoindolin-2-yl)-[2,4′-bipyrimidin]-4-yl)ethynyl)isoquinolin-1-amine;
3-fluoro-5-((2′-(5-methoxyisoindolin-2-yl)-[2,4′-bipyrimidin]-4-yl)ethynyl)-1H-indazole;
7-fluoro-5-((2′-(5-methoxyisoindolin-2-yl)-[2,4′-bipyrimidin]-4-yl)ethynyl)-1H-indazole;
5-((2′-(5-methoxyisoindolin-2-yl)-[2,4′-bipyrimidin]-4-yl)ethynyl)isoindolin-1-one;
methyl 4-((2′-(5-methoxyisoindolin-2-yl)-[2,4′-bipyrimidin]-4-yl)ethynyl)benzoate;
4-((2′-(5-methoxyisoindolin-2-yl)-[2,4′-bipyrimidin]-4-yl)ethynyl)benzonitrile;
4-((2′-(5-methoxyisoindolin-2-yl)-[2,4′-bipyrimidin]-4-yl)ethynyl)benzoic acid;
4-((2′-(5-methoxyisoindolin-2-yl)-[2,4′-bipyrimidin]-4-yl)ethynyl)-N-methylbenzamide;
5-((2′-(isoindolin-2-yl)-[2,4′-bipyrimidin]-4-yl)ethynyl)-1H-indazole;
5-((2′-(5-fluoroisoindolin-2-yl)-[2,4′-bipyrimidin]-4-yl)ethynyl)-1H-indazole;
7-fluoro-5-((2′-(5-fluoroisoindolin-2-yl)-[2,4′-bipyrimidin]-4-yl)ethynyl)-1H-indazole;
5-((2′-(6-methoxy-1,3-dihydro-2H-pyrrolo[3,4-c]pyridin-2-yl)-[2,4′-bipyrimidin]-4-yl)ethynyl)-1H-indazole;
2-(4-((1H-indazol-5-yl)ethynyl)-[2,4′-bipyrimidin]-2′-yl)-2,3-dihydro-1H-pyrrolo[3,4-c]pyridin-6-ol;
5-((2′-(6-chloro-1,3-dihydro-2H-pyrrolo[3,4-c]pyridin-2-yl)-[2,4′-bipyrimidin]-4-yl)ethynyl)-1H-indazole;
5-((6-(2-(5-methoxyisoindolin-2-yl)pyrimidin-4-yl)pyridin-2-yl)ethynyl)-1H-indazole;
7-fluoro-5-((6-(2-(5-methoxyisoindolin-2-yl)pyrimidin-4-yl)pyridin-2-yl)ethynyl)-1H-indazole;
5-((6-(2-(5-fluoroisoindolin-2-yl)pyrimidin-4-yl)pyridin-2-yl)ethynyl)-1H-indazole;
7-fluoro-5-((6-(2-(5-fluoroisoindolin-2-yl)pyrimidin-4-yl)pyridin-2-yl)ethynyl)-1H-indazole;
2-((2-(4-(6-((1H-indazol-5-yl)ethynyl)pyridin-2-yl)pyrimidin-2-yl)isoindolin-5-yl)oxy)-N,N-dimethylethanamine;
5-((6-(2-(5-(4-methylpiperazin-1-yl)isoindolin-2-yl)pyrimidin-4-yl)pyridin-2-yl)ethynyl)-1H-indazole;
5-((3-fluoro-5-(2-(5-methoxyisoindolin-2-yl)pyrimidin-4-yl)phenyl)ethynyl)-1H-indazole;
5-((3-fluoro-5-(2-(5-fluoroisoindolin-2-yl)pyrimidin-4-yl)phenyl)ethynyl)-1H-indazole;
5-((3-(2-(5-chloroisoindolin-2-yl)pyrimidin-4-yl)-5-fluorophenyl)ethynyl)-1H-indazole;
5-((3-(2-(5-bromoisoindolin-2-yl)pyrimidin-4-yl)-5-fluorophenyl)ethynyl)-1H-indazole;
2-((2-(4-(3-((1H-indazol-5-yl)ethynyl)-5-fluorophenyl)pyrimidin-2-yl)isoindolin-5-yl)oxy)-N,N-dimethylethanamine;
5-((3-fluoro-5-(2-(5-(4-methylpiperazin-1-yl)isoindolin-2-yl)pyrimidin-4-yl)phenyl)ethynyl)-1H-indazole; and
5-((2′-(5-bromoisoindolin-2-yl)-[2,4′-bipyrimidin]-4-yl)ethynyl)-1H-indazole.

15. The compound of any one of claims 1-14 wherein the compound has ROCK1, ROCK2, or ROCK1/2 inhibitory activities.

16. The compound of any one of claims 1-14 wherein the compound has ROCK2 or ROCK1/2 inhibitory activities.

17. A pharmaceutical composition comprising one or more compound of any one of claims 1-14 and a pharmaceutically acceptable carrier or diluent.

18. The composition of claim 17 wherein the compound has ROCK1, ROCK2, or ROCK1/2 inhibitory activities.

19. The composition of claim 18 wherein the compound has antifibrotic activity.

20. A method of modulating ROCK1, ROCK2, or ROCK1/2 activities in:
(a) a patient; or
(b) a biological sample;
which method comprises administering to said patient, or contacting said biological sample with:
a) a composition according to claim 17; or
b) a compound of any one of claims 1-14.

21. The method of claim 20 wherein the method is for treating a condition, disease or disorder in which ROCK1, ROCK2, or ROCK1/2 plays a role.

22. The method of claim 20 or 21 wherein the method is for treating or lessening the severity of a disease or condition selected from renal fibrosis, fibrotic liver disease, hepatic ischemia-reperfusion injury, cerebral infarction, ischemic heart disease, renal disease or lung (pulmonary) fibrosis.

23. The method of claim 22 wherein the method is for treating or lessening the severity of a disease or condition selected from liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemia/reperfusion injury; stroke; cerebrovascular disease; myocardial ischemia; atherosclerosis; renal failure; an ophthalmic disease, renal fibrosis and idiopathic pulmonary fibrosis.

24. The method of claim 22 wherein the method is for the treatment of wounds for acceleration of healing; vascularization of a damaged and/or ischemic organ, transplant or graft; amelioration of ischemia/reperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; acute or chronic heart failure, renal failure secondary to chronic diabetes and/or hypertension; amyotrophic lateral sclerosis, muscular dystrophy, glaucoma, corneal scarring, macular degeneration, diabetic retinopathy, and/or diabetes mellitus.

25. A compound of Formula II: 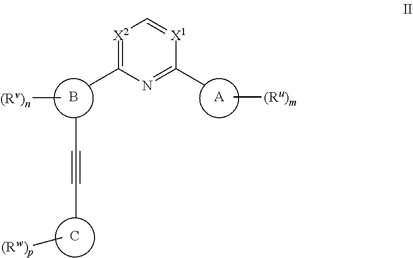
or a pharmaceutically acceptable salt thereof wherein:
each of X¹ and X² is selected from CH and N, wherein only one of X¹ and X² is N;
Ring A is selected from a 4- to 7-membered saturated or partially unsaturated heterocyclic ring comprising 1-2 heteroatoms independently selected from nitrogen, oxygen, and sulfur, or a 5- to 6-membered saturated heterocyclic ring comprising 1-2 heteroatoms independently selected from nitrogen, oxygen and sulfur fused to a group independently selected from phenyl and a 5- or 6-membered heteroaryl ring comprising 1-3 heteroatoms independently selected from nitrogen, oxygen, and sulfur;
Ring B is selected from phenyl and a 6-membered heteroaryl ring comprising 1-2 nitrogen atoms;
Ring C is selected from phenyl, a 5- to 6-membered heteroaryl ring comprising 1-3 heteroatoms independently selected from nitrogen, oxygen, and sulfur, and a 9- to 10-membered heteroaryl ring comprising 1-3 heteroatoms independently selected from nitrogen, oxygen, and sulfur;
each R^{u} is independently selected from halogen, OR″, and an optionally substituted group selected from C₁₋₆ aliphatic, phenyl, a 3- to 7-membered saturated or partially unsaturated heterocyclic ring comprising 1-3 heteroatoms independently selected from nitrogen, oxygen, and sulfur, and a 5- to 6-membered heteroaryl ring comprising 1-3 heteroatoms independently selected from nitrogen, oxygen, and sulfur;
each R^{v} is independently selected from halogen, CN, CO₂R″, C(O)NR″₂, NR″₂, OR″, SR″, and optionally substituted C₁₋₆ aliphatic;
each R^{w} is independently selected from halogen, CN, CO₂R″, C(O)NR″₂, NR″₂, OR″, SR″, and optionally substituted C₁₋₆ aliphatic, or
two independent occurrences of R^{w}, taken together with their intervening atom(s), form an optionally substituted 5-membered saturated or partially unsaturated heterocyclic ring comprising 1-2 heteroatoms independently selected from nitrogen, oxygen, and sulfur;
each R″ is independently selected from hydrogen or an optionally substituted group selected from C₁₋₆ aliphatic, phenyl, and a 3- to 7-membered saturated or partially unsaturated heterocyclic ring comprising 1-3 heteroatoms independently selected from nitrogen, oxygen, and sulfur; and
each of m, n, and p is independently 0-4.

26. The compound according to claim 25, wherein the compound is of Formula II-a: 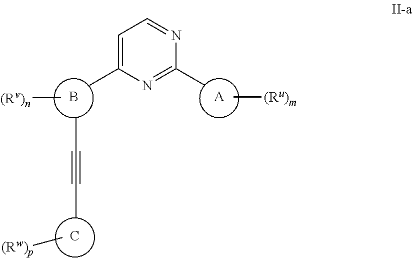
or a pharmaceutically acceptable salt thereof.

27. The compound according to claim 25 or claim 26, wherein Ring A is

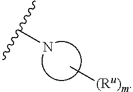

28. The compound according to any one of claims 25-27, wherein Ring A is selected from:

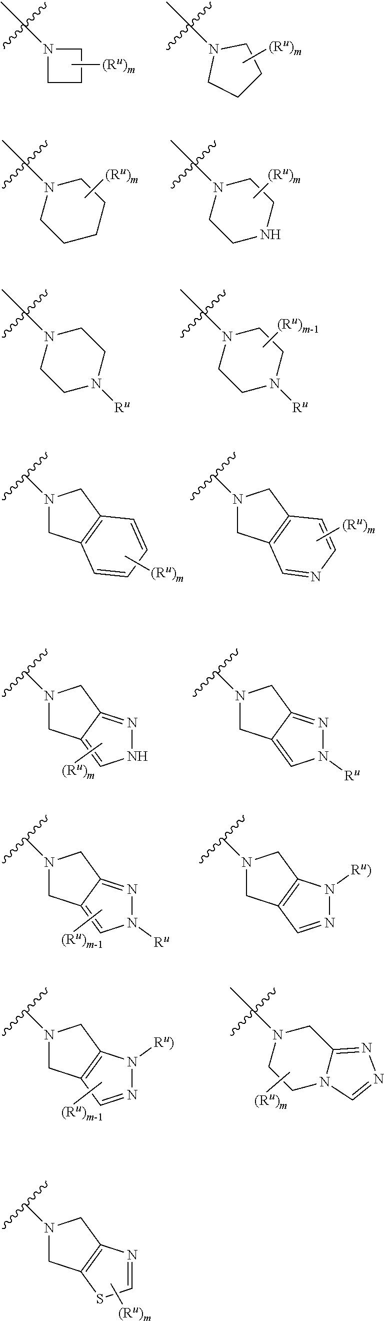

29. The compound according to any one of claims 1-28, wherein Ring B is selected from

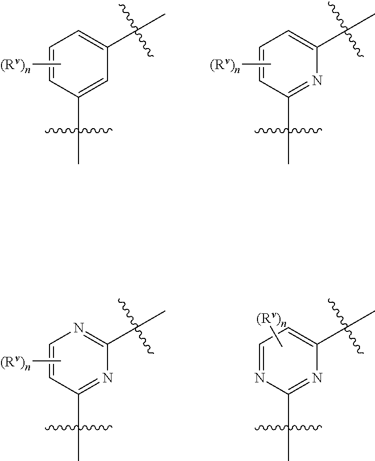

30. The compound according to any one of claims 25-29, wherein Ring C is phenyl.

31. The compound according to any one of claims 25-29, wherein Ring C is a 5- to 6-membered heteroaryl ring comprising 1-3 heteroatoms independently selected from nitrogen, oxygen, and sulfur.

32. The compound according to any one of claims 25-29, wherein Ring C is a 9- to 10-membered heteroaryl ring comprising 1-3 heteroatoms independently selected from nitrogen, oxygen, and sulfur.

33. The compound according to any one of claims 25-29, wherein Ring C is selected from

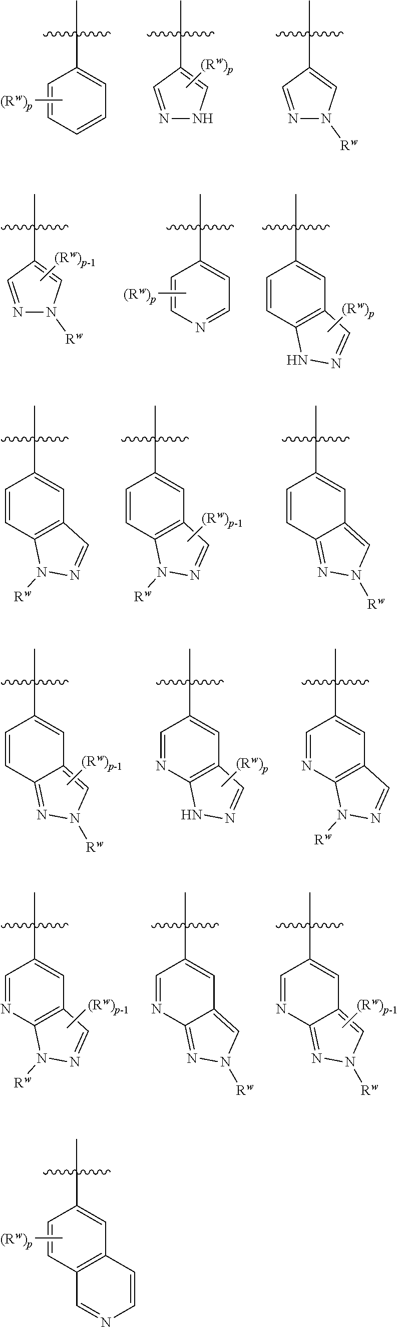

34. The compound according to claim 25, wherein the compound is of Formula II-b: 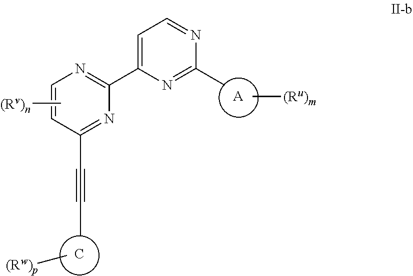
or a pharmaceutically acceptable salt thereof.

35. The compound according to claim 25, wherein the compound is of Formula II-c: 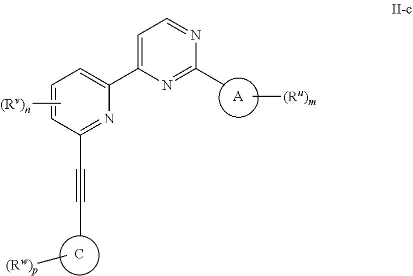
or a pharmaceutically acceptable salt thereof.

36. The compound according to claim 25, wherein the compound is of Formula II-d: 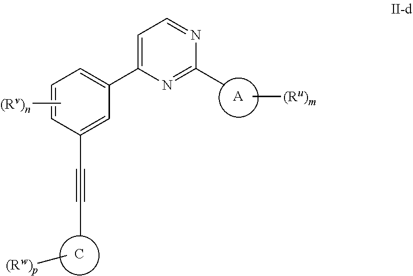
or a pharmaceutically acceptable salt thereof.

37. The compound according to claim 25, wherein the compound is of Formula II-e: 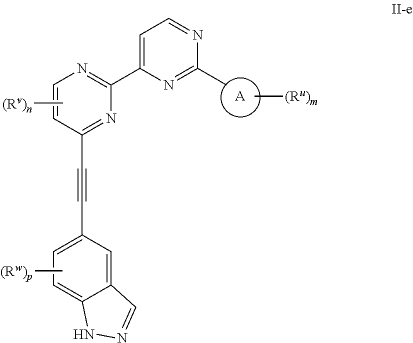
or a pharmaceutically acceptable salt thereof.

38. The compound according to claim 25, wherein the compound is of Formula II-f: 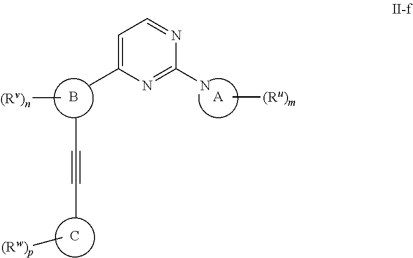
or a pharmaceutically acceptable salt thereof.

39. The compound according to claim 25, wherein the compound is of Formula II-g: 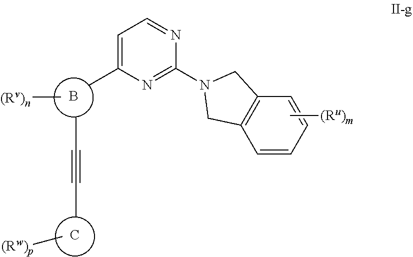
or a pharmaceutically acceptable salt thereof.

40. The compound according to claim 25, wherein the compound is of Formula II-h: 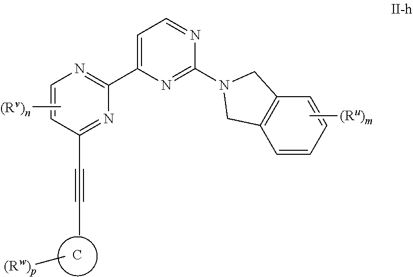
or a pharmaceutically acceptable salt thereof.

41. The compound according to claim 25, wherein the compound is selected from the group consisting of 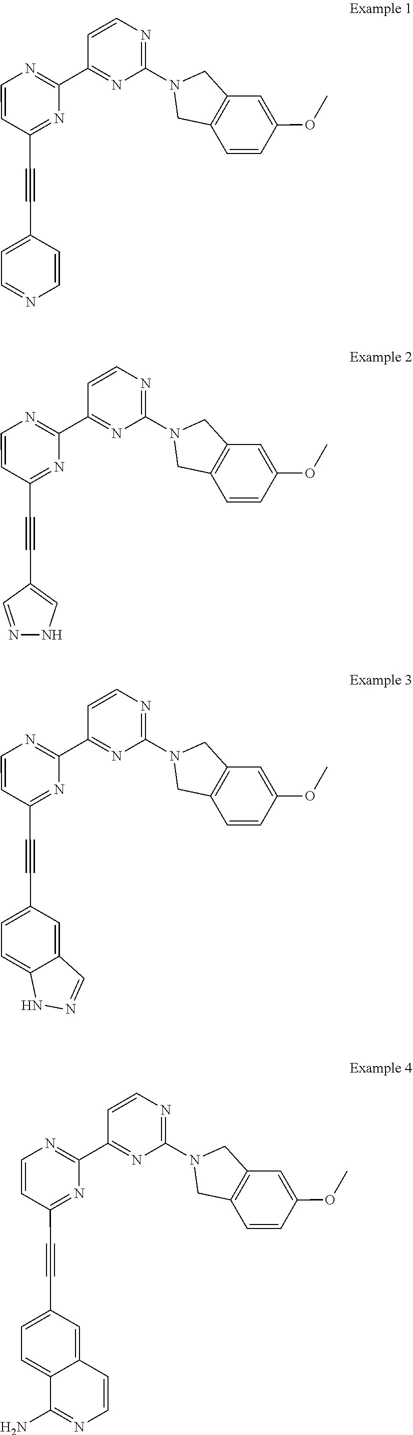 [SMILES: COc1ccc2c(c1)CN(c1nccc(-c3nccc(C#Cc4ccncc4)n3)n1)C2 ; COc1ccc2c(c1)CN(c1nccc(-c3nccc(C#Cc4cn[nH]c4)n3)n1)C2 ; COc1ccc2c(c1)CN(c1nccc(-c3nccc(C#Cc4ccc5[nH]ncc5c4)n3)n1)C2 ; COc1ccc2c(c1)CN(c1nccc(-c3nccc(C#Cc4ccc5c(N)nccc5c4)n3)n1)C2] 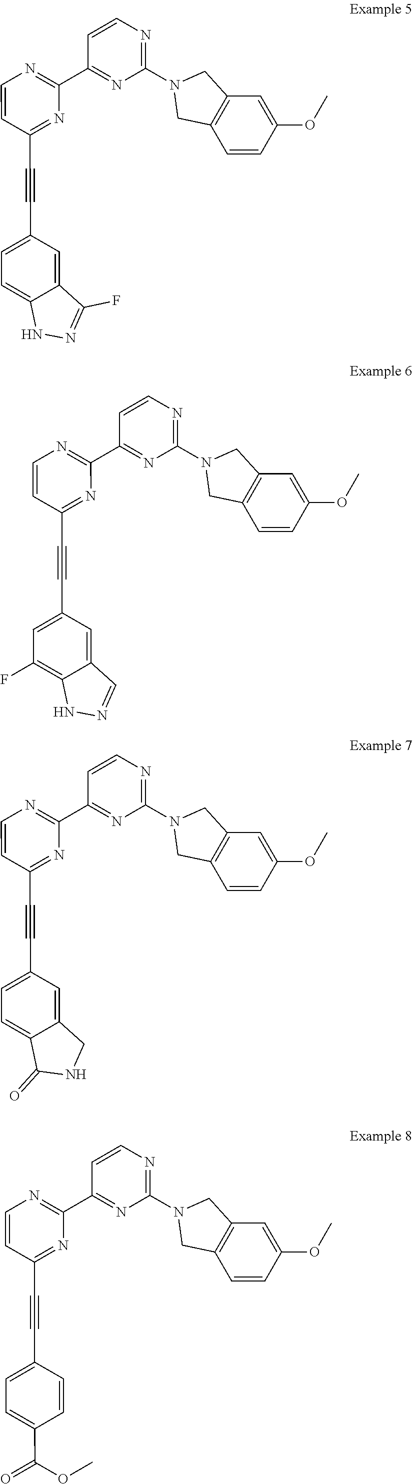 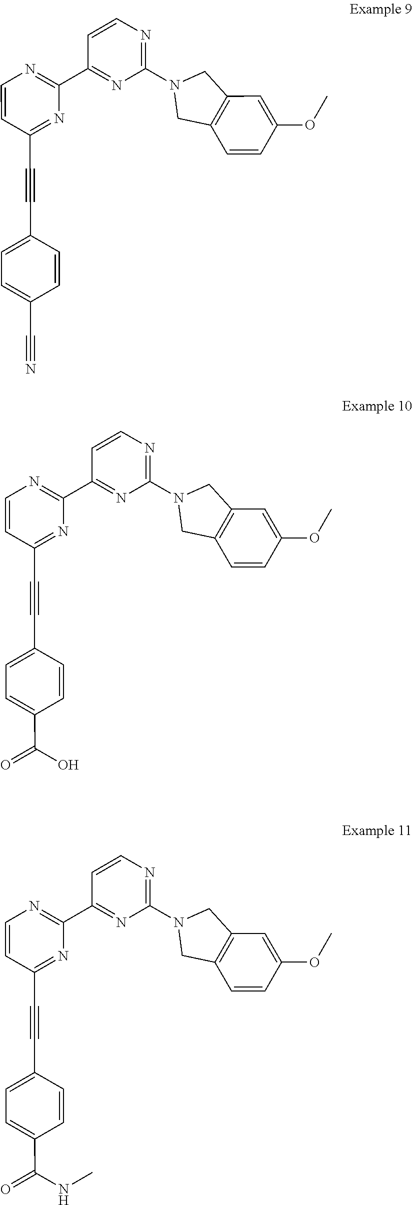 [SMILES: COc1ccc2c(c1)CN(c1nccc(-c3nccc(C#Cc4ccc(C#N)cc4)n3)n1)C2 ; COc1ccc2c(c1)CN(c1nccc(-c3nccc(C#Cc4ccc(C(=O)O)cc4)n3)n1)C2 ; CNC(=O)c1ccc(C#Cc2ccnc(-c3ccnc(N4Cc5ccc(OC)cc5C4)n3)n2)cc1] 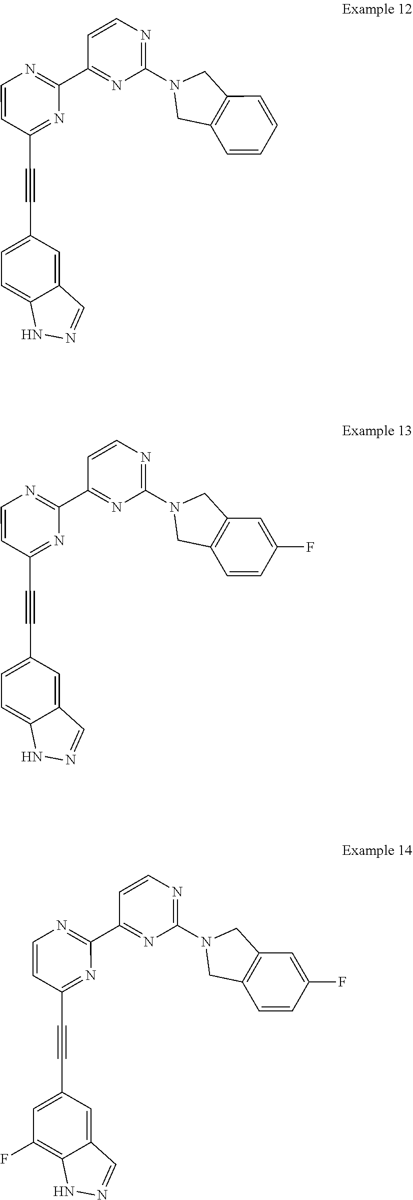 [SMILES: C(#Cc1ccnc(-c2ccnc(N3Cc4ccccc4C3)n2)n1)c1ccc2[nH]ncc2c1 ; Fc1ccc2c(c1)CN(c1nccc(-c3nccc(C#Cc4ccc5[nH]ncc5c4)n3)n1)C2 ; Fc1ccc2c(c1)CN(c1nccc(-c3nccc(C#Cc4cc(F)c5[nH]ncc5c4)n3)n1)C2] 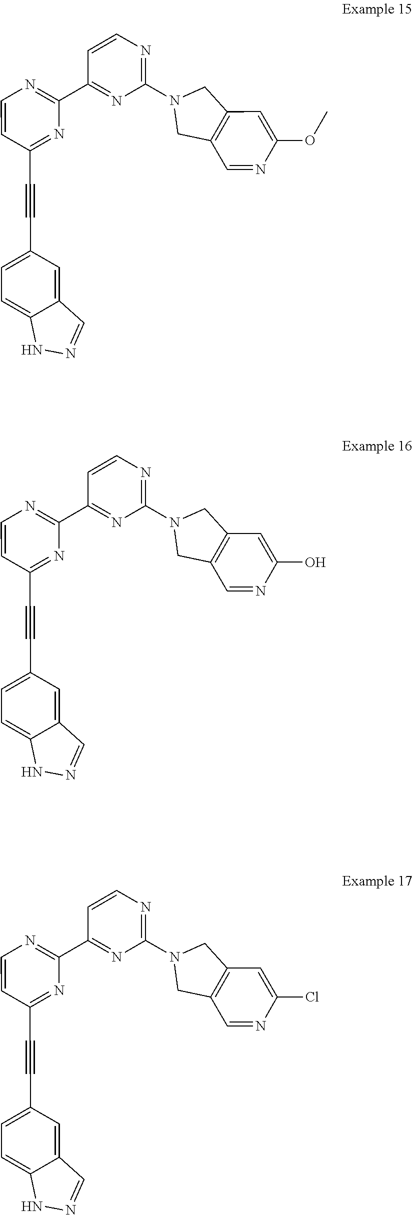 [SMILES: COc1cc2c(cn1)CN(c1nccc(-c3nccc(C#Cc4ccc5[nH]ncc5c4)n3)n1)C2 ; Oc1cc2c(cn1)CN(c1nccc(-c3nccc(C#Cc4ccc5[nH]ncc5c4)n3)n1)C2 ; Clc1cc2c(cn1)CN(c1nccc(-c3nccc(C#Cc4ccc5[nH]ncc5c4)n3)n1)C2] 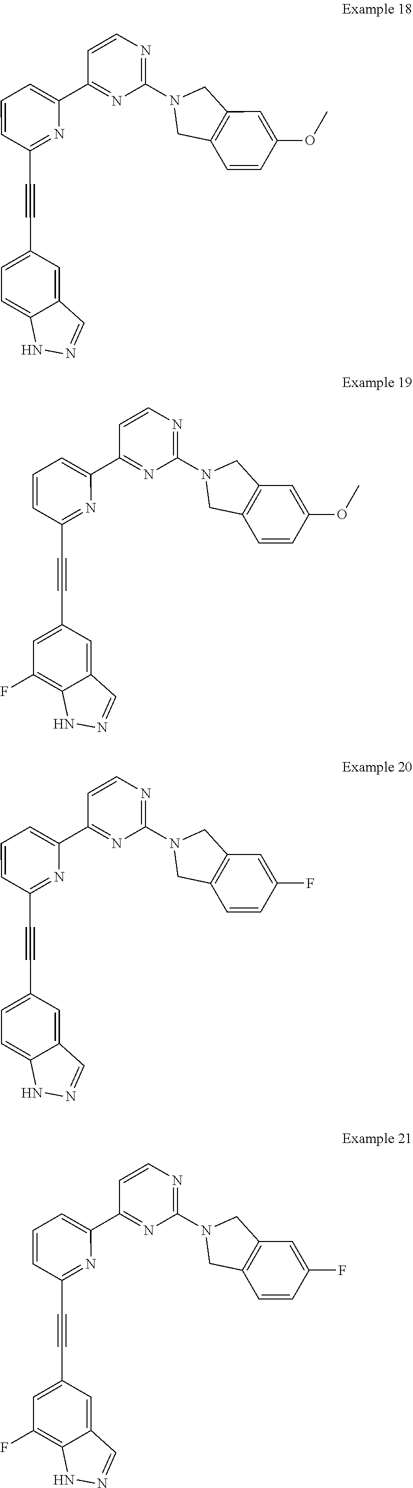 [SMILES: COc1ccc2c(c1)CN(c1nccc(-c3cccc(C#Cc4ccc5[nH]ncc5c4)n3)n1)C2 ; COc1ccc2c(c1)CN(c1nccc(-c3cccc(C#Cc4cc(F)c5[nH]ncc5c4)n3)n1)C2 ; Fc1ccc2c(c1)CN(c1nccc(-c3cccc(C#Cc4ccc5[nH]ncc5c4)n3)n1)C2 ; Fc1ccc2c(c1)CN(c1nccc(-c3cccc(C#Cc4cc(F)c5[nH]ncc5c4)n3)n1)C2] 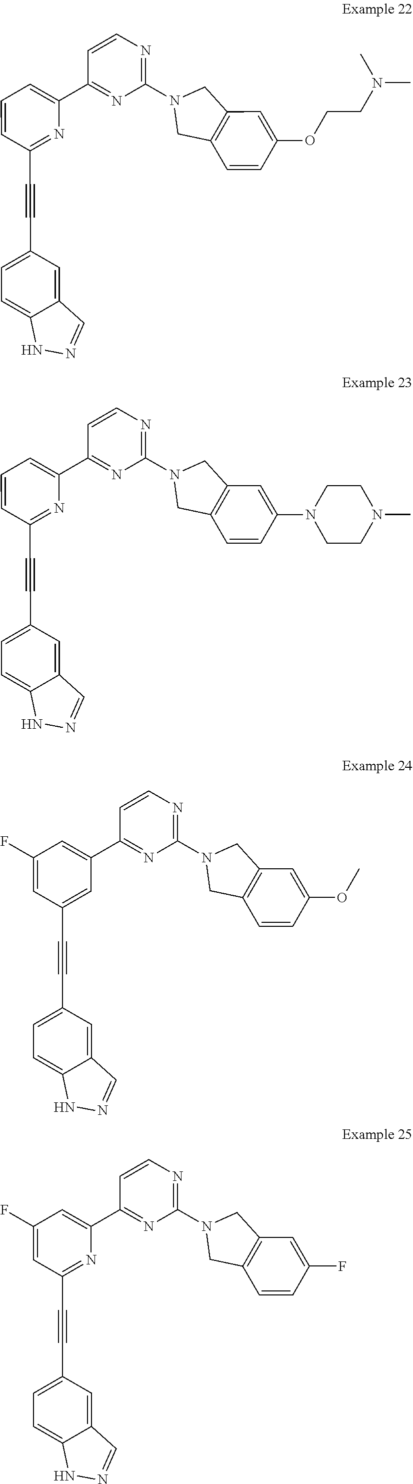 [SMILES: CN(C)CCOc1ccc2c(c1)CN(c1nccc(-c3cccc(C#Cc4ccc5[nH]ncc5c4)n3)n1)C2 ; CN1CCN(c2ccc3c(c2)CN(c2nccc(-c4cccc(C#Cc5ccc6[nH]ncc6c5)n4)n2)C3)CC1 ; COc1ccc2c(c1)CN(c1nccc(-c3cc(F)cc(C#Cc4ccc5[nH]ncc5c4)c3)n1)C2 ; Fc1cc(C#Cc2ccc3[nH]ncc3c2)nc(-c2ccnc(N3Cc4ccc(F)cc4C3)n2)c1] 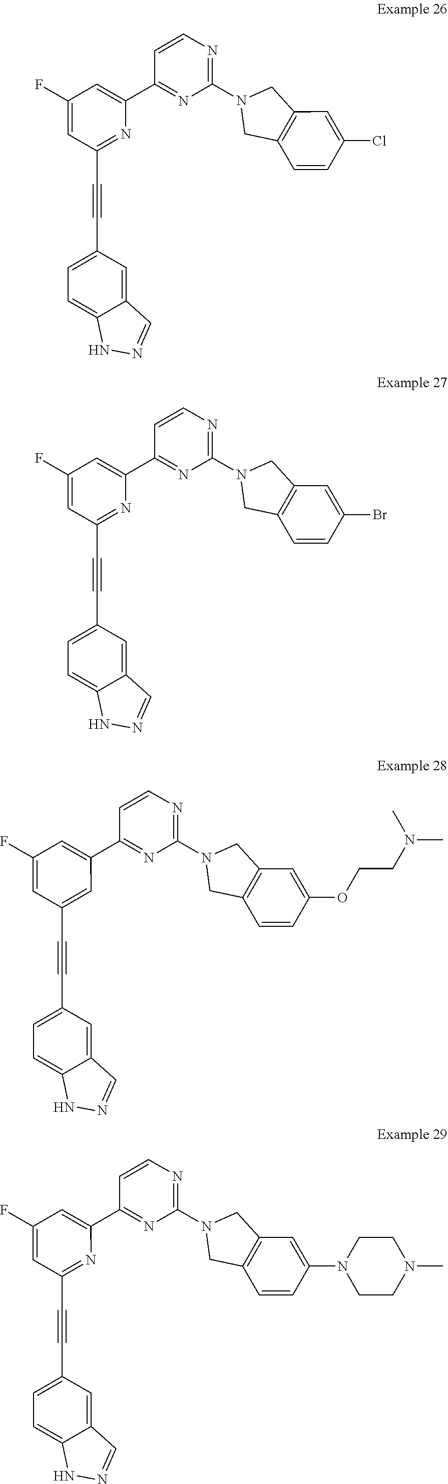 [SMILES: Fc1cc(C#Cc2ccc3[nH]ncc3c2)nc(-c2ccnc(N3Cc4ccc(Cl)cc4C3)n2)c1 ; Fc1cc(C#Cc2ccc3[nH]ncc3c2)nc(-c2ccnc(N3Cc4ccc(Br)cc4C3)n2)c1 ; CN(C)CCOc1ccc2c(c1)CN(c1nccc(-c3cc(F)cc(C#Cc4ccc5[nH]ncc5c4)c3)n1)C2 ; CN1CCN(c2ccc3c(c2)CN(c2nccc(-c4cc(F)cc(C#Cc5ccc6[nH]ncc6c5)n4)n2)C3)CC1] 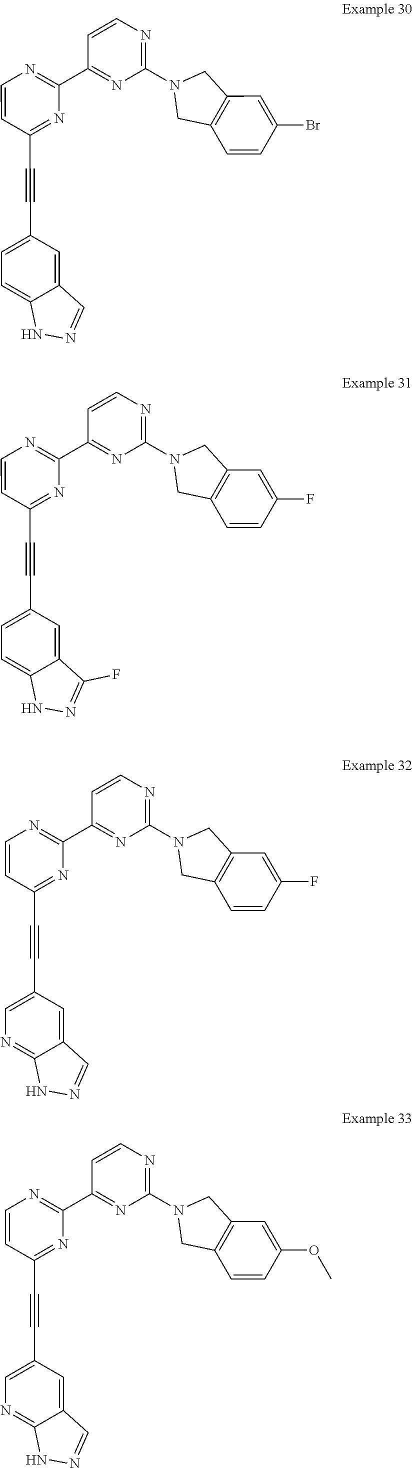 [SMILES: Brc1ccc2c(c1)CN(c1nccc(-c3nccc(C#Cc4ccc5[nH]ncc5c4)n3)n1)C2 ; Fc1ccc2c(c1)CN(c1nccc(-c3nccc(C#Cc4ccc5[nH]nc(F)c5c4)n3)n1)C2 ; Fc1ccc2c(c1)CN(c1nccc(-c3nccc(C#Cc4cnc5[nH]ncc5c4)n3)n1)C2 ; COc1ccc2c(c1)CN(c1nccc(-c3nccc(C#Cc4cnc5[nH]ncc5c4)n3)n1)C2] 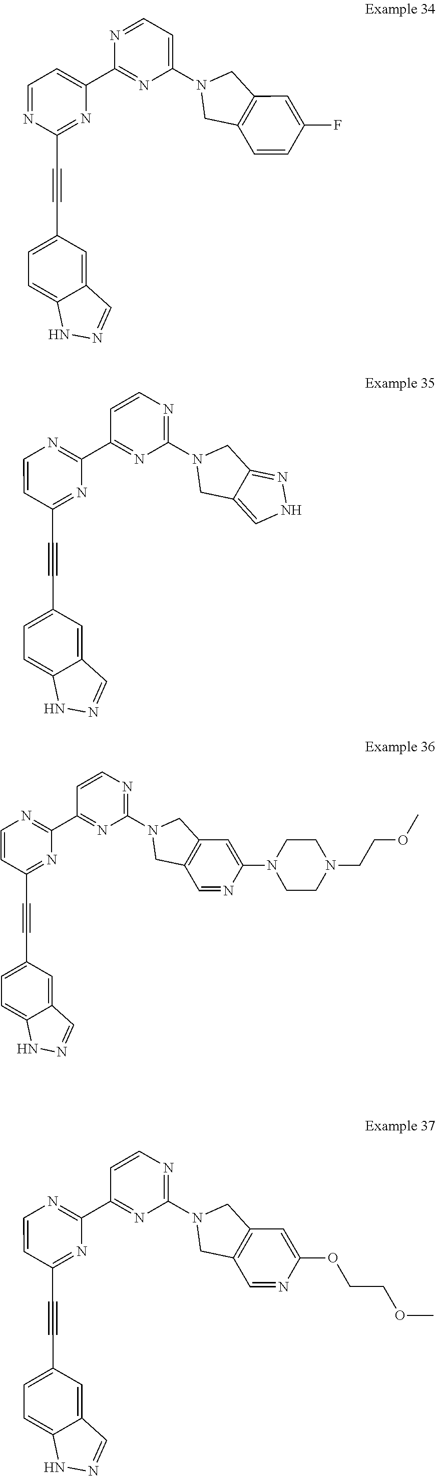 [SMILES: Fc1ccc2c(c1)CN(c1ccnc(-c3ccnc(C#Cc4ccc5[nH]ncc5c4)n3)n1)C2 ; C(#Cc1ccnc(-c2ccnc(N3Cc4c[nH]nc4C3)n2)n1)c1ccc2[nH]ncc2c1 ; COCCN1CCN(c2cc3c(cn2)CN(c2nccc(-c4nccc(C#Cc5ccc6[nH]ncc6c5)n4)n2)C3)CC1 ; COCCOc1cc2c(cn1)CN(c1nccc(-c3nccc(C#Cc4ccc5[nH]ncc5c4)n3)n1)C2] 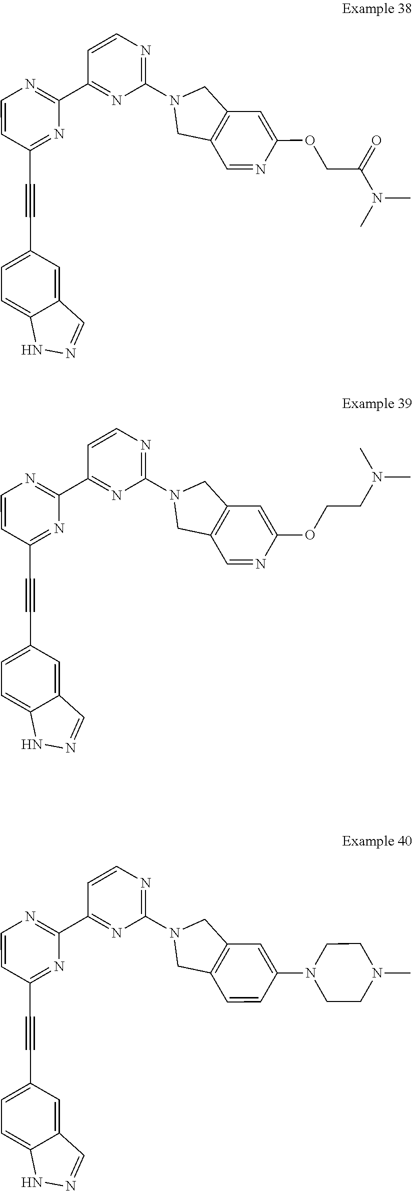 [SMILES: CN(C)C(=O)COc1cc2c(cn1)CN(c1nccc(-c3nccc(C#Cc4ccc5[nH]ncc5c4)n3)n1)C2 ; CN(C)CCOc1cc2c(cn1)CN(c1nccc(-c3nccc(C#Cc4ccc5[nH]ncc5c4)n3)n1)C2 ; CN1CCN(c2ccc3c(c2)CN(c2nccc(-c4nccc(C#Cc5ccc6[nH]ncc6c5)n4)n2)C3)CC1] 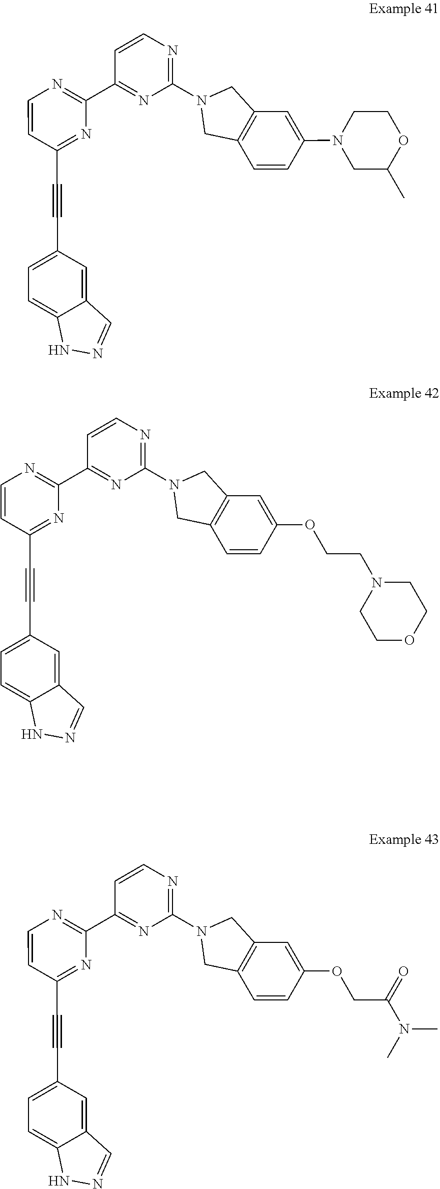 [SMILES: CC1CN(c2ccc3c(c2)CN(c2nccc(-c4nccc(C#Cc5ccc6[nH]ncc6c5)n4)n2)C3)CCO1 ; C(#Cc1ccnc(-c2ccnc(N3Cc4ccc(OCCN5CCOCC5)cc4C3)n2)n1)c1ccc2[nH]ncc2c1 ; CN(C)C(=O)COc1ccc2c(c1)CN(c1nccc(-c3nccc(C#Cc4ccc5[nH]ncc5c4)n3)n1)C2] 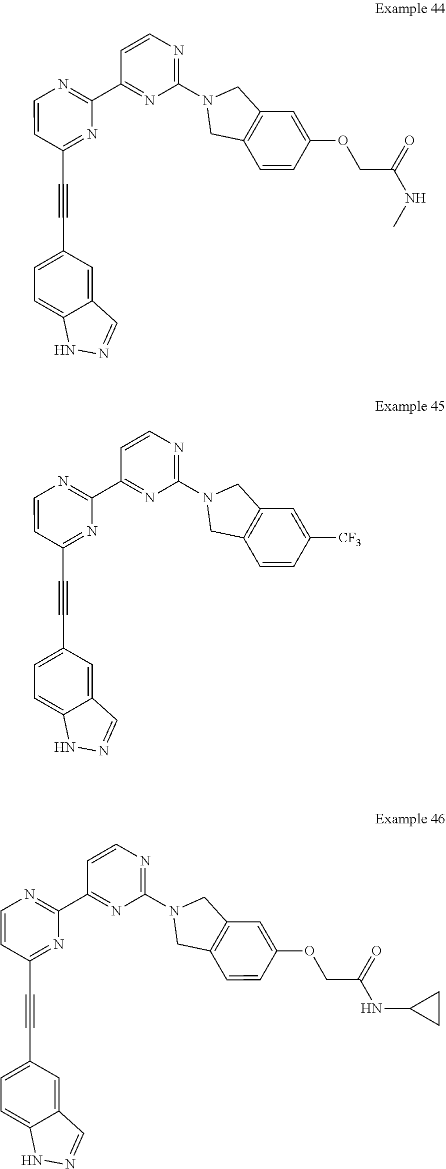 [SMILES: CNC(=O)COc1ccc2c(c1)CN(c1nccc(-c3nccc(C#Cc4ccc5[nH]ncc5c4)n3)n1)C2 ; FC(F)(F)c1ccc2c(c1)CN(c1nccc(-c3nccc(C#Cc4ccc5[nH]ncc5c4)n3)n1)C2 ; O=C(COc1ccc2c(c1)CN(c1nccc(-c3nccc(C#Cc4ccc5[nH]ncc5c4)n3)n1)C2)NC1CC1] 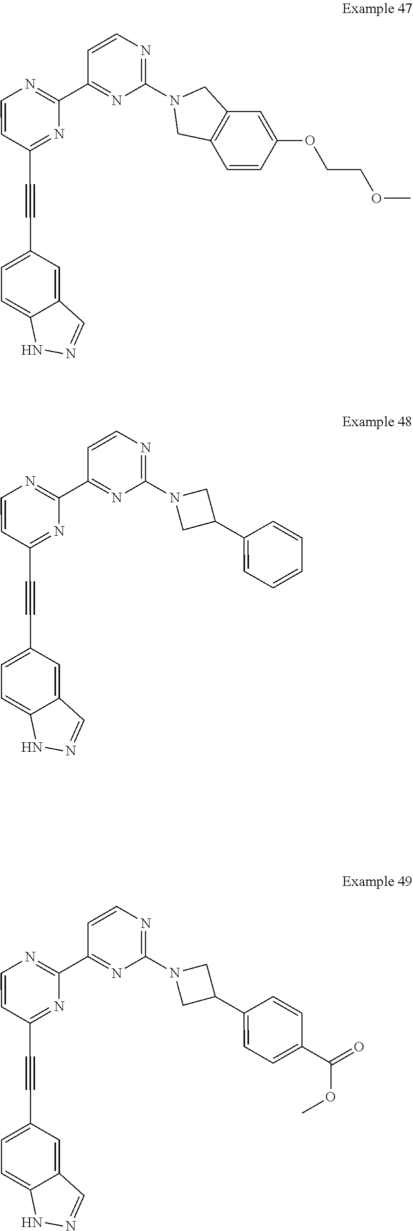 [SMILES: COCCOc1ccc2c(c1)CN(c1nccc(-c3nccc(C#Cc4ccc5[nH]ncc5c4)n3)n1)C2 ; C(#Cc1ccnc(-c2ccnc(N3CC(c4ccccc4)C3)n2)n1)c1ccc2[nH]ncc2c1 ; COC(=O)c1ccc(C2CN(c3nccc(-c4nccc(C#Cc5ccc6[nH]ncc6c5)n4)n3)C2)cc1] 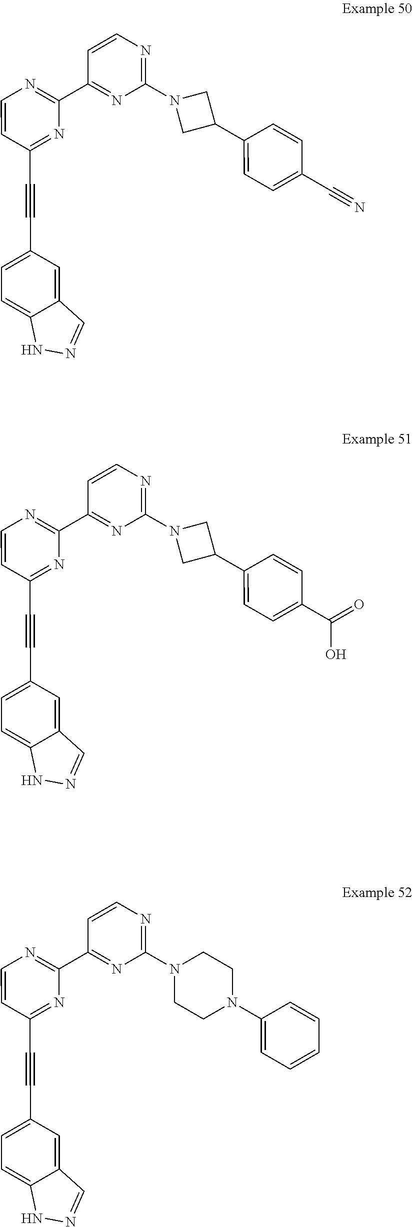 [SMILES: N#Cc1ccc(C2CN(c3nccc(-c4nccc(C#Cc5ccc6[nH]ncc6c5)n4)n3)C2)cc1 ; O=C(O)c1ccc(C2CN(c3nccc(-c4nccc(C#Cc5ccc6[nH]ncc6c5)n4)n3)C2)cc1 ; C(#Cc1ccnc(-c2ccnc(N3CCN(c4ccccc4)CC3)n2)n1)c1ccc2[nH]ncc2c1] 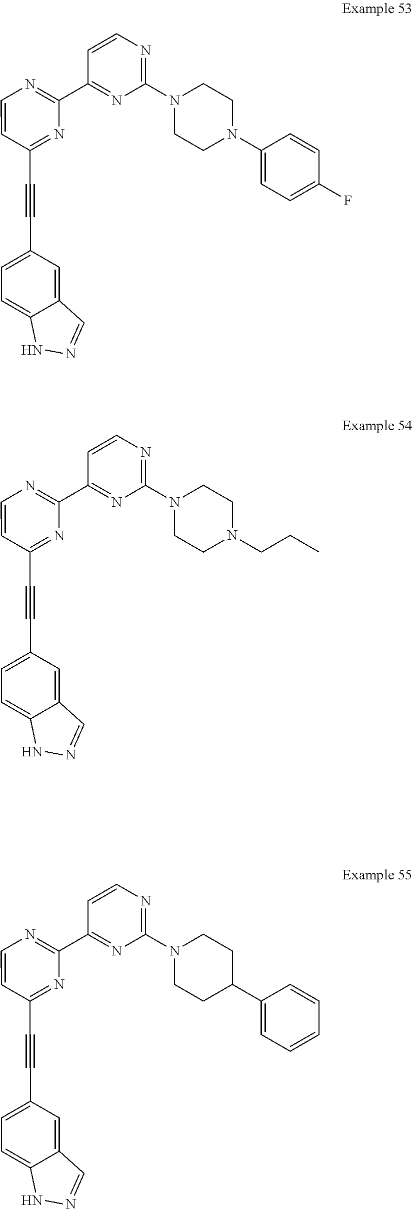 [SMILES: Fc1ccc(N2CCN(c3nccc(-c4nccc(C#Cc5ccc6[nH]ncc6c5)n4)n3)CC2)cc1 ; CCCN1CCN(c2nccc(-c3nccc(C#Cc4ccc5[nH]ncc5c4)n3)n2)CC1 ; C(#Cc1ccnc(-c2ccnc(N3CCC(c4ccccc4)CC3)n2)n1)c1ccc2[nH]ncc2c1] 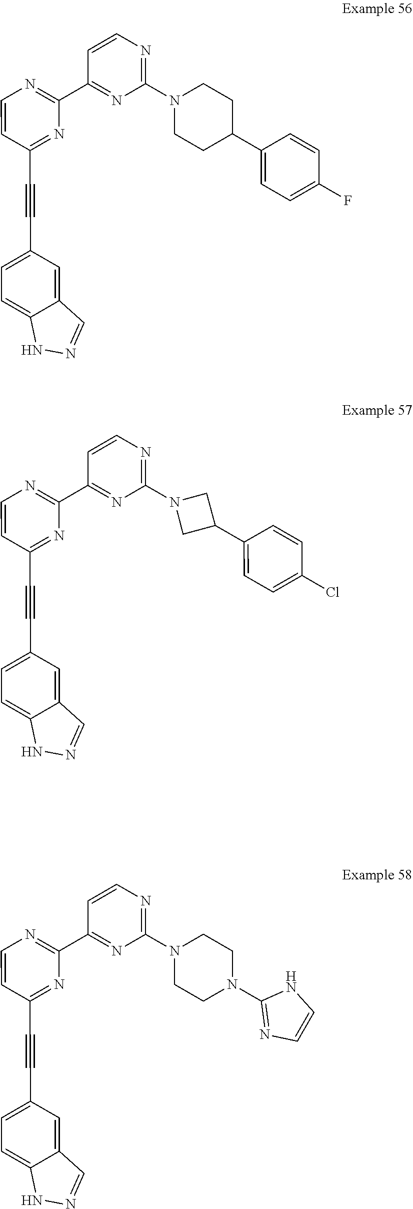 [SMILES: Fc1ccc(C2CCN(c3nccc(-c4nccc(C#Cc5ccc6[nH]ncc6c5)n4)n3)CC2)cc1 ; Clc1ccc(C2CN(c3nccc(-c4nccc(C#Cc5ccc6[nH]ncc6c5)n4)n3)C2)cc1 ; C(#Cc1ccnc(-c2ccnc(N3CCN(c4ncc[nH]4)CC3)n2)n1)c1ccc2[nH]ncc2c1] 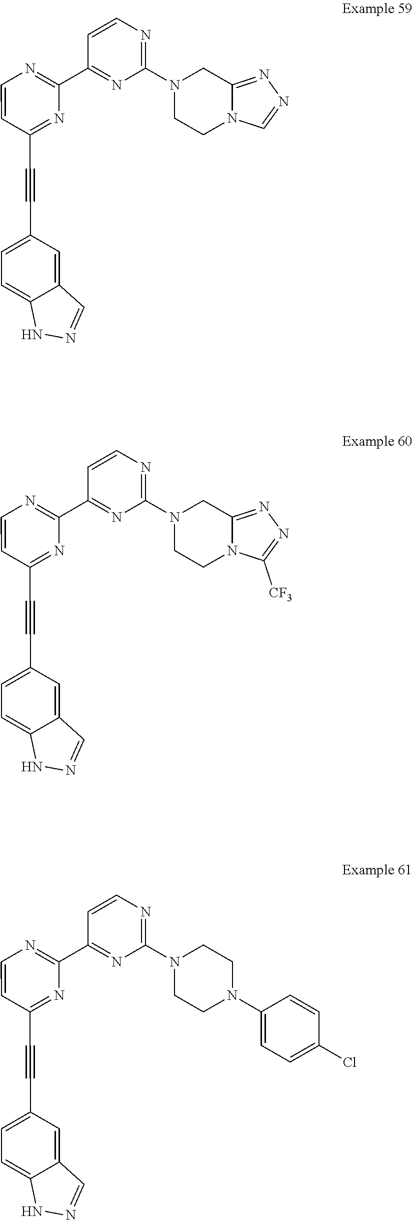 [SMILES: C(#Cc1ccnc(-c2ccnc(N3CCn4cnnc4C3)n2)n1)c1ccc2[nH]ncc2c1 ; FC(F)(F)c1nnc2n1CCN(c1nccc(-c3nccc(C#Cc4ccc5[nH]ncc5c4)n3)n1)C2 ; Clc1ccc(N2CCN(c3nccc(-c4nccc(C#Cc5ccc6[nH]ncc6c5)n4)n3)CC2)cc1] 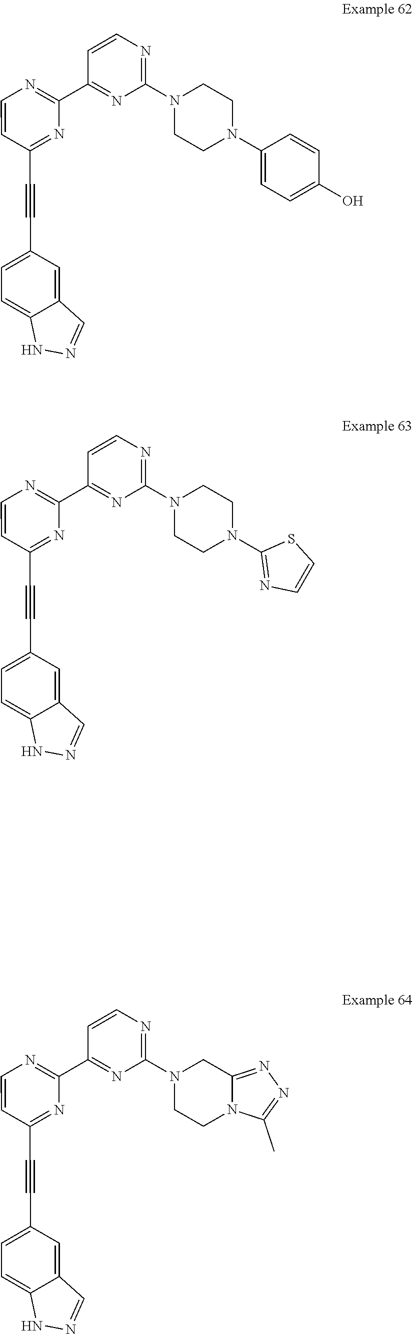 [SMILES: Oc1ccc(N2CCN(c3nccc(-c4nccc(C#Cc5ccc6[nH]ncc6c5)n4)n3)CC2)cc1 ; C(#Cc1ccnc(-c2ccnc(N3CCN(c4nccs4)CC3)n2)n1)c1ccc2[nH]ncc2c1 ; Cc1nnc2n1CCN(c1nccc(-c3nccc(C#Cc4ccc5[nH]ncc5c4)n3)n1)C2] 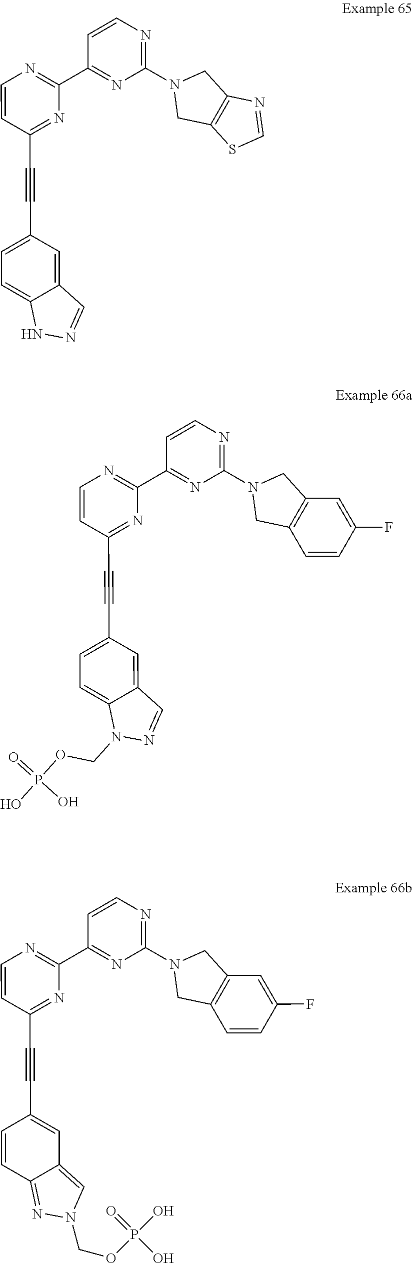 [SMILES: C(#Cc1ccnc(-c2ccnc(N3Cc4ncsc4C3)n2)n1)c1ccc2[nH]ncc2c1 ; O=P(O)(O)OCn1ncc2cc(C#Cc3ccnc(-c4ccnc(N5Cc6ccc(F)cc6C5)n4)n3)ccc21 ; O=P(O)(O)OCn1cc2cc(C#Cc3ccnc(-c4ccnc(N5Cc6ccc(F)cc6C5)n4)n3)ccc2n1] 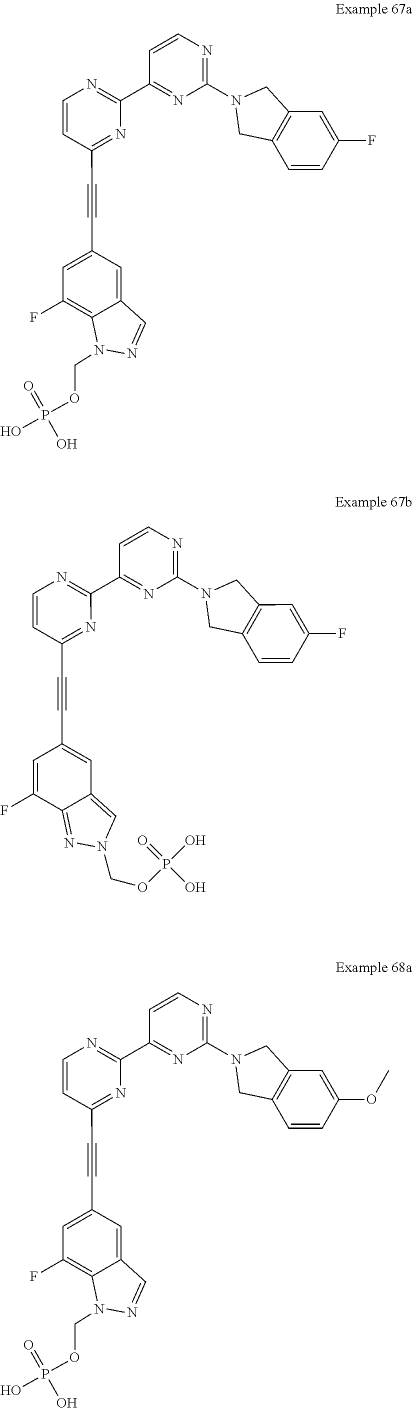 [SMILES: O=P(O)(O)OCn1ncc2cc(C#Cc3ccnc(-c4ccnc(N5Cc6ccc(F)cc6C5)n4)n3)cc(F)c21 ; O=P(O)(O)OCn1cc2cc(C#Cc3ccnc(-c4ccnc(N5Cc6ccc(F)cc6C5)n4)n3)cc(F)c2n1 ; COc1ccc2c(c1)CN(c1nccc(-c3nccc(C#Cc4cc(F)c5c(cnn5COP(=O)(O)O)c4)n3)n1)C2] 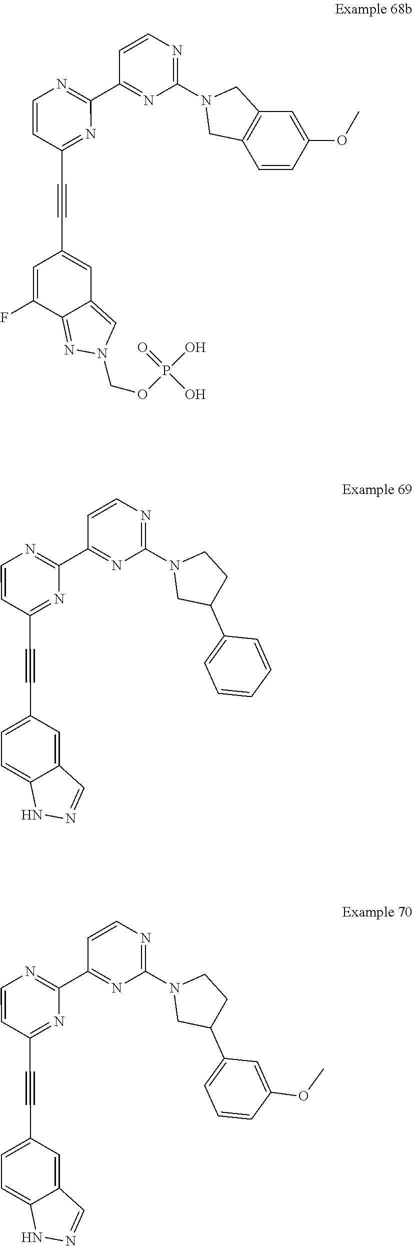 [SMILES: COc1ccc2c(c1)CN(c1nccc(-c3nccc(C#Cc4cc(F)c5nn(COP(=O)(O)O)cc5c4)n3)n1)C2 ; C(#Cc1ccnc(-c2ccnc(N3CCC(c4ccccc4)C3)n2)n1)c1ccc2[nH]ncc2c1 ; COc1cccc(C2CCN(c3nccc(-c4nccc(C#Cc5ccc6[nH]ncc6c5)n4)n3)C2)c1] 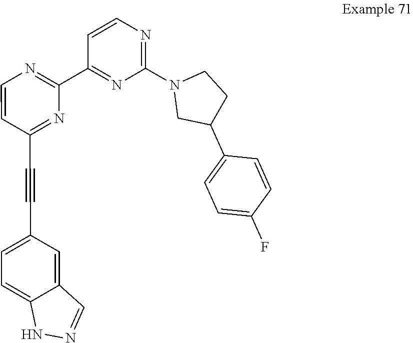 [SMILES: Fc1ccc(C2CCN(c3nccc(-c4nccc(C#Cc5ccc6[nH]ncc6c5)n4)n3)C2)cc1]
or a pharmaceutically acceptable salt thereof.

42. A pharmaceutical composition comprising a compound according to any one of claims 25-41, or a pharmaceutically acceptable salt thereof, and a pharmaceutically acceptable carrier.

43. A method of inhibiting ROCK1 and/or ROCK2, the method comprising contacting a biological sample with a compound according to any one of claims 25-42, or a pharmaceutically acceptable salt thereof.

44. The method according to claim 43, wherein the compound is selective for ROCK2.

45. A method of treating or lessening the severity of a disease or disorder associated with or mediated by Rho-associated coiled-coil kinase (ROCK), the method comprising administering to a patient in need thereof a compound according to any one of claims 25-42, or a pharmaceutically acceptable salt thereof.

46. The method according to claim 45, wherein the compound is selective for ROCK2.

47. The method according to claim 45 or claim 46, wherein the disease or disorder is selected from a hepatic disease, renal disease, stroke, myocardial infarction, an ischemic disease, or a fibrotic disease.

48. The method according to claim 47, wherein the fibrotic disease is liver fibrosis.

49. The method according to claim 47, wherein the fibrotic disease is pulmonary fibrosis.

50. The method according to claim 47, wherein the hepatic disease is hepatic ischemia-reperfusion injury.

51. The method according to claim 47, wherein the disease or disorder is stroke (e.g., cerebral infarction).

52. The method according to claim 47, wherein the ischemic disease is ischemic heart disease.

53. The method according to claim 47, wherein the disease or disorder is renal disease.
